(19) United States
(12) Patent Application Publication
(10) Pub. No.: US 2023/0114434 A1
(43) Pub. Date: Apr. 13, 2023

(54) EXTRACELLULAR VESICLES FOR TREATING NEUROLOGICAL DISORDERS

(71) Applicant: Codiak BioSciences, Inc., Cambridge, MA (US)

(72) Inventors: Ajay VERMA, Wayland, MA (US); Tim SOOS, Boston, MA (US); Nikki ROSS, Philadelphia, PA (US); Christine MCCOY, Cambridge, MA (US); Jonathan FINN, Melrose, MA (US); Sriram SATHYANARAYANAN, Lexington, MA (US); Shailendra PATEL, Boston, MA (US); Ke XU, Belmont, MA (US); Russell E. MCCONNELL, Cambridge, MA (US); Kevin P. DOOLEY, Boston, MA (US)

(73) Assignee: Codiak BioSciences, Inc., Cambridge, MA (US)

(21) Appl. No.: 17/906,177

(22) PCT Filed: Mar. 15, 2021
(86) PCT No.: PCT/US2021/022420

§ 371 Date: Sep. 12, 2022

Related U.S. Application Data
(60) Provisional application No. 63/059,103, filed on Jul. 30, 2020.
(60) Provisional application No. 63/055,657, filed on Jul. 23, 2020.
(60) Provisional application No. 63/050,651, filed on Jul. 10, 2020.
(60) Provisional application No. 63/035,367, filed on Jun. 5, 2020.
(60) Provisional application No. 63/016,213, filed on Apr. 27, 2020.
(60) Provisional application No. 63/010,603, filed on Apr. 15, 2020.
(60) Provisional application No. 62/989,491, filed on Mar. 13, 2020.

(51) Int. Cl. A61K 39/00; A61P 25/28; A61K 39/39; A61P 35/00; A61K 31/436; A61K 31/593; A61K 31/203; A61K 31/573; A61K 31/7088; A61K 47/69; A61K 47/64; A61H 23/00; A61H 23/02

(52) U.S. Cl. CPC A61K 39/0007; A61P 25/28; A61K 39/39; A61P 35/00; A61K 31/436; A61K 31/593; A61K 31/203; A61K 31/573; A61K 31/7088; A61K 47/6911; A61K 47/646; A61H 23/006; A61H 23/02; A61K 2039/575; A61K 2039/55555; A61K 2039/577; A61K 2039/54; A61K 2039/545; A61K 2039/55516; A61H 2201/165; A61H 2201/1623; A61H 2201/1619; A61H 2205/084; A61H 2201/105

(57) ABSTRACT

The present disclosure relates to extracellular vesicles (EVs) that are capable of targeting a cell in the CNS of a subject. Also provided herein are methods for producing the EVs and methods for using the EVs to treat and/or prevent diseases or disorders of the CNS (e.g., neurological disorders).

### CROSS-REFERENCE TO RELATED APPLICATIONS

This PCT application claims the priority benefit of U.S. Provisional Application Nos. 62/989,491, filed on Mar. 13, 2020; 63/010,603, filed on Apr. 15, 2020; 63/016,213, filed on Apr. 27, 2020; 63/035,367, filed on Jun. 5, 2020; 63/050,651, filed on Jul. 10, 2020; 63/055,657, filed on Jul. 23, 2020; and 63/059,103, filed on Jul. 30, 2020, each of which is herein incorporated by reference in its entirety.

### REFERENCE TO SEQUENCE LISTING SUBMITTED ELECTRONICALLY VIA EFS-WEB

The content of the electronically submitted sequence listing (Name: 4000_091PC07_Seqlisting_ST25.txt, Size: 302,012 bytes; and Date of Creation: Mar. 15, 2021) submitted in this application is incorporated herein by reference in its entirety.

### FIELD OF DISCLOSURE

The present disclosure relates to engineered extracellular vesicles (EVs) (e.g., exosomes) that can be used to treat and/or prevent a neurological disorder. The present disclosure also relates to methods of producing such EVs and uses thereof.

### BACKGROUND OF DISCLOSURE

Neuroimmunological disorders are some of the most devastating and difficult to treat. Examples of such diseases include gliomas, peripheral tumors that have metastasized to the brain or meninges (neoplastic meningitis), and chronic infectious meningitis. Gliomas are the most common type of tumors to affect the central nervous system. Ostrom, Q. T., et al., Neuro Oncol 16(7): 896-913 (2014). Gliomas comprise about 30 percent of all brain tumors and central nervous system tumors, and 80 percent of all malignant brain tumors. Gliomas typically begin in the glial cells that surround and support neurons in the brain, including astrocytes, oligodendrocytes, and ependymal cells. Hanif, F., et al., Asian Pac J Cancer Prev 18(1): 3-9 (2017). Of the gliomas, glioblastoma (also known as glioblastoma multiforme (GBM)) is the most common and the most aggressive.

Despite the aggressive standard of care currently used (e.g., surgery, radiation therapy, chemotherapy, and electric field therapy), there remains a need for more effective and comprehensive treatment options for neuroimmunological disorders, e.g., gliomas, e.g., glioblastoma multiforme (GBM). GBM is rarely curable. For instance, the current survival rate for GBM is 14-15 months after diagnosis with less than 3-5% of people surviving longer than five years. Without treatment, most patients succumb to the disease within just a few months. Omuro, A., et al., JAMA 310:1842-1850 (2013). Prognosis generally worsens with age.

EVs are important mediators of intercellular communication. They are also important biomarkers in the diagnosis and prognosis of many diseases, such as cancer. As drug delivery vehicles, EVs offer many advantages over traditional drug delivery methods (e.g., peptide immunization, DNA vaccines) as a new treatment modality in many therapeutic areas. However, despite its advantages, many EVs have had limited clinical efficacy. For example, dendritic-cell derived exosomes (DEX) were investigated in a Phase II clinical trial as maintenance immunotherapy after first line chemotherapy in patients with inoperable non-small cell lung cancer (NSCLC). However, the trial was terminated because the primary endpoint (at least 50% of patients with progression-free survival (PFS) at 4 months after chemotherapy cessation) was not reached. Besse, B., et al., Oncoimmunology 5(4):e1071008 (2015).

Accordingly, new and more effective engineered-EVs are necessary, particularly those that are capable of treating neurological disorders, such as those disclosed herein (e.g., gliomas or leptomeningeal cancer disease (LMD)).

### SUMMARY OF DISCLOSURE

Provided herein is a method of treating a neurological disorder in a subject in need thereof, comprising administering to the subject an extracellular vesicle (EV), which comprises an antigen and wherein the EV is capable of targeting a cell within the central nervous system (CNS) of the subject.

In some aspects, administering the EV to the subject results in the induction of a humoral immune response, comprising antibodies directed against the antigen. In certain aspects, the induction of the humoral immune response improves one or more symptoms associated with the neurological disorder. In some aspects, the antibodies are capable of specifically binding to a neuronal protein that has misfolded (“misfolded neuronal protein”). In some aspects, the binding of the antibodies to the misfolded neuronal protein facilitates the removal of the misfolded neuronal protein from the subject. In certain aspects, administering the EV to the subject results in a decrease in the amount of misfolded neuronal protein present within the CNS of the subject.

Also provided herein is a method for modulating a germinal center response to an antigen in a subject in need thereof, comprising administering to the subject an extracellular vesicle (EV), which comprises an antigen, and wherein the EV is capable of targeting a cell within the central nervous system (CNS) of the subject.

In some aspects, administering the EV to the subject increases the germinal center response in the subject. In some aspects, the increase in the germinal center response results in greater production of antibodies against the antigen. In certain aspects, administering the EV to the subject decreases the germinal center response in the subject. In some aspects, the decrease in the germinal center response results in lower production of antibodies against the antigen.

In some aspects, an EV that can be used with the above methods (i.e., method of treating a neurological disorder or method of modulating a germinal center response to an antigen) further comprises one or more additional payloads. In certain aspects, the additional payload is an adjuvant. In some aspects, the additional payload is an immune modulator.

In some aspects, the antigen expressed in an EV that can be used in the above methods (i.e., method of treating a neurological disorder or method of modulating a germinal center response to an antigen) comprises a neuronal protein that when misfolded can cause a neurological disorder. In certain aspects, the neuronal protein comprises amyloid beta (Aβ), tau, alpha-synuclein, poly-GA, or combinations thereof.

In some aspects, the subject suffers from a neurological disorder. In certain aspects, the neurological disorder comprises a brain tumor, neoplastic meningitis, leptomeningeal cancer disease (LMD), amyotrophic lateral sclerosis (ALS), Parkinson's disease (PD), Huntington's disease (HD), Alzheimer's disease (AD), or combinations thereof. In some aspects, the neurological disorder is leptomeningeal cancer disease (LMD). In certain aspects, the neurological disorder is a brain tumor. In some aspects, the brain tumor is a glioma. In certain aspects, the glioma is a low grade glioma or a high grade glioma. In some aspects, the glioma is oligodendroglioma, anaplastic astrocytomas, glioblastoma multiforme, diffuse intrinsic pontine glioma, IDH1 and IDH2-mutated glioma, or combinations thereof. In certain aspects, the glioma is glioblastoma multiforme.

In some aspects, an adjuvant that can be expressed in an EV used in the above methods comprises a stimulator of interferon genes protein (STING) agonist, toll-like receptor (TLR) agonist, inflammatory mediator, or combinations thereof. In certain aspects, the adjuvant is a STING agonist. In some aspects, the STING agonist comprises a cyclic dinucleotide STING agonist or a non-cyclic dinucleotide STING agonist. In certain aspects, the adjuvant is a TLR agonist. In some aspects, the TLR agonist comprises a TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), or combinations thereof.

In some aspects, a cell within the CNS that the EVs of the above methods can target comprises an immune cell. In certain aspects, the immune cell comprises a dendritic cell, macrophage, T cells, B cells, or combinations thereof. In some aspects, the immune cell is a dendritic cell. In some aspects, the immune cell is a macrophage.

Present disclosure further provides a method for treating an autoimmune disorder in a subject in need thereof, comprising administering to the subject an extracellular vesicle (EV), which comprises an antigen, and wherein the EV is capable of targeting a cell within the central nervous system (CNS) of the subject.

Also provided herein is a method for inducing an immune tolerance in a subject in need thereof, comprising administering to the subject an extracellular vesicle (EV), which comprises an antigen, and wherein the EV is capable of targeting a cell within the central nervous system (CNS) of the subject. In some aspects, the subject suffers from an autoimmune disorder.

In some aspects, administering an EV to the subject in a method described above (i.e., treating an autoimmune disorder or inducing an immune tolerance) results in the induction of tolerogenic cells. In certain aspects, the induction of the tolerogenic cells improves one or more symptoms associated with the autoimmune disorder. In some aspects, the tolerogenic cells comprise regulatory T cells (Tregs), liver sinusoidal endothelial cells (LSECs), Kupffer cells, or combinations thereof. In certain aspects, the tolerogenic cells are Tregs that are specific to the antigen.

In some aspects, the antigen expressed in an EV that can be used in the above methods (i.e., treating an autoimmune disorder or inducing an immune tolerance) comprises a self-antigen that is associated with an autoimmune disorder. In certain aspects, the autoimmune disorder comprises a multiple sclerosis (MS), peripheral neuritis, Sjogren's syndrome, rheumatoid arthritis, alopecia, autoimmune pancreatitis, Behcet's disease, Bullous pemphigoid, Celiac disease, Devic's disease (neuromyelitis optica), Glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, Graves' disease, myasthenia gravis (MG), pemphigus vulgaris, anti-glomerular basement membrane disease (Goodpasture syndrome), Hashimoto's thyroiditis, autoimmune hepatitis, or combinations thereof. In some aspects, the self-antigen comprises beta-cell proteins, insulin, islet antigen 2 (IA-2), glutamic acid decarboxylase (GAD65), zinc transporter 8 (ZNT8), myelin oligodendrocyte glycoprotein (MOG), myelin basic protein (MBP), proteolipid protein (PLP), myelin-associated glycoprotein (MAG), citrullinated antigens, synovial proteins, aquaporin-4 (AQP4), nicotinic acetylcholine receptor (nAChR), desmoglein-1 (DSG1), desoglein-2 (DSG2), thyrotropin receptor, type IV collagen, thyroglobulin, thyroid peroxidase, thyroid-stimulating hormone receptor (TSHR), or combinations thereof.

In some aspects, the self-antigen is AQP4 and the autoimmune disorder is neuromyelitis optica (NMO). In some aspects, the self-antigen is MOG and the autoimmune disorder is multiple sclerosis (MS). In some aspects, the self-antigen is nAChR and the autoimmune disorder is myasthenia gravis (MG).

In some aspects, an EV that can be used in the above methods (i.e., treating an autoimmune disorder or inducing an immune tolerance) further comprises one or more additional payloads.

In some aspects, the additional payload is an immune modulator. In certain aspects, the immune modulator comprises a tolerance inducing agent (“tolerogen”). In certain aspects, the tolerogen comprises a NF-κB inhibitor, COX-2 inhibitor, mTOR inhibitor (e.g., rapamycin and derivatives), prostaglandins, nonsteroidal anti-inflammatory agents (NSAIDS), antileukotriene, aryl hydrocarbon receptor (AhR) ligand, vitamin D3, retinoic acid, steroids, Fas receptor/ligand, CD22 ligand, IL-10, IL-35, IL-27, metabolic regulator (e.g., glutamate), glycans (e.g., ES62, LewisX, LNFPIII), peroxisome proliferator-activated receptor (PPAR) agonists, immunoglobulin-like transcript (ILT) family of receptors (e.g., ILT3, ILT4, HLA-G, ILT-2), dexamethasone, or combinations thereof.

In some aspects, the tolerogen is rapamycin. In some aspects, the tolerogen is vitamin D3. In some aspects, the tolerogen is retinoic acid. In some aspects, the tolerogen is dexamethasone.

In some aspects, the immune modulator comprises a polynucleotide selected from a mRNA, miRNA, siRNA, antisense oligonucleotide (ASO), phosphorodiamidate morpholino oligomer (PMO), peptide-conjugated phosphorodiamidate morpholino oligomer (PPMO), shRNA, lncRNA, dsDNA, or combinations thereof. In certain aspects, the immune modulator is an ASO. In some aspects, the ASO is capable of inhibiting NF-κB, CD40, mTOR, or combinations thereof.

In some aspects, an EV that can be used in any of the methods disclosed herein further comprises a targeting moiety. In certain aspects, the targeting moiety is capable of specifically binding to a marker expressed on the cell within the CNS of the subject. In some aspects, the marker is expressed only on dendritic cells. In some aspects, the marker comprises a C-type lectin domain family 9 member A (Clec9a) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), or combinations thereof. In some aspects, the marker is expressed only on macrophages. In some aspects, the marker comprises CD14, CD16, CD64, CD68, CD71, CCR5, or combinations thereof.

In some aspects, an EV that can be used in any of the methods disclosed herein further comprises a first scaffold moiety. In certain aspects, the antigen, additional payload, and/or targeting moiety is linked to the first scaffold moiety.

In some aspects, an EV that can be used with a method disclosed herein further comprises a second scaffold moiety. In certain aspects, wherein the antigen, additional payload, and/or targeting moiety is linked to the second scaffold moiety.

In some aspects, the first scaffold moiety and the second scaffold moiety are the same. In some aspects, the first scaffold moiety and the second scaffold moiety are different. In some aspects, the first scaffold moiety is Scaffold X. In some aspects, the first scaffold moiety is Scaffold Y. In some aspects, the second scaffold moiety is Scaffold Y. In some aspects, the second scaffold moiety is Scaffold X.

In some aspects, Scaffold X is selected from prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), or combinations thereof. In some aspects, Scaffold Y is selected from myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), or combinations thereof.

In some aspects, the antigen, additional payload, and/or targeting moiety is linked to the first scaffold moiety and/or to the second scaffold moiety by a linker. In certain aspects, the linker is a polypeptide. In some aspects, the linker is a non-polypeptide moiety.

In some aspects, the first scaffold moiety or the second scaffold moiety is PTGFRN protein. In some aspects, the first scaffold moiety or the second scaffold moiety comprises an amino acid sequence as set forth in SEQ ID NO: 33. In some aspects, the first scaffold moiety or the second scaffold moiety comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 1.

In some aspects, the first scaffold moiety or the second scaffold moiety is BASP1 protein. In some aspects, the first scaffold moiety or the second scaffold moiety comprises a peptide of (M)(G)(π)(X)(Φ/π)(π)(+)(+) or (G)(π)(X)(Φ/π)(π)(+)(+), wherein each parenthetical position represents an amino acid, and wherein π is any amino acid selected from the group consisting of Pro, Gly, Ala, and Ser, X is any amino acid, 0 is any amino acid selected from the group consisting of Val, Ile, Leu, Phe, Trp, Tyr, and Met, and (+) is any amino acid selected from the group consisting of Lys, Arg, and His; and wherein position five is not (+) and position six is neither (+) nor (Asp or Glu). In some aspects, first scaffold moiety or the second scaffold moiety comprises an amino acid sequence set forth in any one of SEQ ID NOs: 50-155. In some aspects, the first scaffold moiety or the second scaffold moiety comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 3.

In some aspects, an EV that can be used in a method disclosed herein is not derived from a naturally-existing antigen-presenting cell (APC). In certain aspects, the EV is an exosome.

In some aspects, an EV is administered via intrathecal, intraocular, intracranial, intranasal, perineural, or combinations thereof. In certain aspects, the intrathecal administration is in the spinal canal and/or the subarachnoid space. In some aspects, the intraocular administration is selected from intravitreal, intracameral, subconjunctival, subretinal, subscleral, intrachoroidal, or combinations thereof. In some aspects, the intracranial administration is selected from intracisternal, subarachnoidal, intrahippocampal, intracerebroventricular, intraparenchymal, or combinations thereof. In some aspects, the intranasal administration is by instillation or injection. In some aspects, the perineural administration is by facial intradermal injection.

Also provided herein is a method of administering an EV to a subject in need thereof comprising intrathecally administering an EV described herein to the subject, wherein a mechanical convective force is applied to the torso of the subject. In certain aspects, the mechanical convective force is achieved using a high frequency chest wall or lumbothoracic oscillating respiratory clearance device. In some aspects, the mechanical convective force improves the intrathecal administration. In some aspects, the mechanical convective force results in a less dosing amount of the EVs. In some aspects, the mechanical convective force results in an efficient dosing of the EVs.

In some aspects, an EV useful for any of the methods described above further comprises a targeting moiety that targets a Schwann cell. In certain aspects, the targeting moiety specifically interacts with a transferrin receptor (TfR), apolipoprotein D (ApoD), Galectin 1 (LGALS1), Myelin proteolipid protein (PLP), Glypican 1, Syndecan 3, or any combination thereof. In certain aspects, the targeting moiety comprises a transferrin-receptor-targeting moiety.

In some aspects, an EV that can be used with any of the methods described herein further comprises a targeting moiety that targets a sensory neuron. In certain aspects, the targeting moiety specifically interacts with a Trk receptor. In some aspects, the TRK receptor is selected from TrkA, TrkB, TrkC, and any combination thereof.

In some aspects, an EV that can be used with any of the methods described herein further comprises a targeting moiety that targets a motor neuron. In certain aspects, the targeting moiety comprises a Rabies Virus Glycoprotein (RVG) peptide, a Targeted Axonal Import (TAxI) peptide, a P75R peptide, a Tet-C peptide, or any combination thereof.

In some aspects, an EV described herein comprises an immune modulator, wherein the immune modulator comprises a CD4+ T helper peptide, an inhibitor for a negative checkpoint regulator or an inhibitor for a binding partner of a negative checkpoint regulator, an activator for a positive co-stimulatory molecule or an activator for a binding partner of a positive co-stimulatory molecule, a cytokine or a binding partner of a cytokine, a chemokine, an inhibitor of lysophosphatidic acid (LPA), a protein that supports intracellular interactions required for germinal center responses, a T-cell receptor (TCR) or a derivative thereof, a chimeric antigen receptor (CAR) or a derivative thereof, an activator of a T-cell receptor or co-receptor, a tolerance inducing agent, an agonist, an antagonist, an antibody or an antigen-binding fragment thereof, a polynucleotide, or combinations thereof.

In certain aspects, the immune modulator is a CD4+ T helper peptide. In some aspects, the activator for a positive co-stimulatory molecule comprises CD40L, TNFα, TNF-C, OX40L, FasL, LIGHT, TL1A, CD27L, Siva, CD153, 4-1BB ligand, TRAIL, RANKL, TWEAK, APRIL, BAFF, CAMLG, NGF, BDNF, NT-3, NT-4, GITR ligand, EDA-2, or combinations thereof. In some aspects, the cytokine comprises IL-21, IL-2, IL-4, IL-7, IL-10, IL-12, IL-15, IFN-γ, IL-1α, IL-1β, IL-1ra, IL-18, IL-33, IL-36α, IL-36β, IL-36γ, IL-36ra, IL-37, IL-38, IL-3, IL-5, IL-6, IL-11, IL-13, IL-23, granulocyte-macrophage colony stimulating factor (GM-CSF), granulocyte-colony stimulating factor (G-CSF), leukemia inhibitory factor (LIF), stem cell factor (SCF), thrombopoietin (TPO), macrophage-colony stimulating factor (M-CSF), erythropoieticn (EPO), Flt-3, IFN-α, IFN-β, IFN-γ, IL-19, IL-20, IL-22, IL-24, TNF-α, TNF-β, BAFF, APRIL, lymphotoxin beta (TNF-γ), IL-17A, IL-17B, IL-17C, IL-17D, IL-17E, IL-17F, IL-25, TSLP, IL-35, IL-27, TGF-β, or combinations thereof. In some aspects, the immune modulator is capable of enhancing an antibody immune response induced by the EV.

Provided herein is a method of treating and/or preventing a disease or disorder in a subject in need thereof, comprising administering to the subject (i) a priming dose, which comprises a first EV, and (ii) a boosting dose, which comprises a second EV, wherein the first EV and the second EV are not the same. In some aspects, the first EV comprises an antigen and an adjuvant. In certain aspects, the second EV comprises an antigen but not an adjuvant. In some aspects, the antigen of the first EV and the antigen of the second EV are the same.

In some aspects, the disease or disorder comprises a neurological disorder. In certain aspects, the disease or disorder comprises an autoimmune disorder. In some aspects, the priming dose and the boosting dose are administered via different routes.

In some aspects, an EV useful for the methods described herein comprises an antigen, wherein the antigen is linked to the exterior surface and/or the luminal surface of the EV by an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof.

### BRIEF DESCRIPTION OF FIGURES

FIG. 1A shows an exemplary EV comprising one or more antigens (e.g., associated with a neurological disorder disclosed herein) and one or more additional payloads disclosed herein (e.g., one or more adjuvants), one or more molecules for targeting moiety, or any combination thereof).

FIG. 1B shows an exemplary EV comprising B cell vaccine. B cell vaccines that can be added on the EVs include antibodies directed against mis-folded self proteins, e.g., poly-GA, amyloid β, tau, α-synuclein, or combinations thereof. Such EVs can also be useful in the “tunable” modulation of germinal center responses.

FIG. 1C shows an exemplary EV comprising tolerogenic vaccines. Tolerogenic vaccines include auto-reactive antigens that are related to more than 20 autoimmune diseases. Such auto-reactive antigens include aquaporin 4 (AQP4) NMO; Myelin (MOG) MS; and/or nicotinic acetylcholine receptor (nAchR) MG. Well characterized tolerogens include Rapamycin (Rapa), Vitamin D3 (VitD3), Retinoic Acid (RA), and/or Dexamethasone (Dex). Target APCs & tolerogenic cells include Tregs, LSECs & Kupffer cells.

FIG. 2 provides an illustration of different ways in which an antigen (e.g., neuronal protein disclosed herein) can be present on the exterior surface of an EV. As shown, in some aspects, an antigen can be loaded onto the exterior surface of the EV using a scaffold moiety (e.g., Scaffold X, e.g., PTGFRN). In some aspects, an antigen can be attached directly to the surface of the EV using maleimide chemistry. As also shown, in some aspects, an adjuvant (e.g., STING agonist) can also be loaded into the lumen of the EV.

FIGS. 3A-3D show exoRVG uptake in neuro2A cells. The constructs tested were: RVG-PrX-mCherry-FLAG-HiBiT (construct 2021; FIG. 3A), linker-PrX-mCherry-FLAG-HiBiT (construct 2022; FIG. 3B), RVG-LAMP2B-mCherry-FLAG-HiBiT (construct 2023; FIG. 3C), and linker-LAMP2B-mCherry-FLAG-HiBiT (construct 2024; FIG. 3D). Only the constructs comprising RVG showed uptake by the neuro2A cells. 10⁵ EV particles per cell were used. EV uptake was observed at 5 hours. “RVG” is a tropism moiety of sequence YTIWMPENPRPGTPCDIFTNSRGKRASNG (SEQ ID NO: 389). “Linker” is a linker of sequence GGSSGSGSGSGGGGSGGGGTGTSSSGTGT (SEQ ID NO: 416). “FLAG” is a FLAG® epitope tag. “HiBiT” is the nano luciferase peptide described above. “mCherry” is a red fluorescent protein. “LAMP2B” and “PrX” are protein scaffolds. “ExoRVG” EV are exosomes comprising an RVG tropism moiety.

FIGS. 4A-4B show exoRVG uptake in neuro2A cells 18 hours after the cells were incubated with 5×10⁴ EV particles per neuro2A cell. Measurements were taken 18 hours after uptake. The constructs tested were exoRVG (construct 2021, see FIG. 9) (FIG. 4A) and exoLinker (construct 2020, see FIG. 3) (FIG. 4B).

FIGS. 5A-5X show exoRVG uptake in neuro2A cells 24 hours after incubation with EV comprising one of the four constructs described in FIG. 3. Samples used were negative control (no EV particles;

FIGS. 5A-5D), E5 (10⁵ particles/cell; FIGS. 5E-5H), 5E4 (5×10⁴ particles/cell; FIGS. 5I-5L), E4 (10⁴ particles/cell; FIGS. 5M-5P), 5E3 (5×10³ particles/cell; FIGS. 50-5T), and E3 (10³ particles/cell; FIGS. 5U-5X). The boxed data sets corresponds to the samples used in FIGS. 4A-4B measured at 24 hours after uptake.

FIG. 6 compares EV uptake in neuro2A cells corresponding to negative control (leftmost curve), exoLinker (construct 2020) (center curve), and exoRVG (construct 2021) (rightmost curve), measured 24 hours after the cells were incubated with 5×10⁴ EV particles per neuro2A cell.

FIGS. 7A-7C show exoTransferrin uptake in HeLa cells. Three constructs were tested: Transferrin-PrX-mCherry-FLAG (human transferrin; construct 1597; FIG. 7A), mTransferrin-PrX-mCherry-FLAG (mouse transferrin; construct 1598; FIG. 7B); and linker-PrX-mCherry-FLAG-HiBiT (construct 2022; FIG. 7C). 5×10⁵ EV particles per HeLa cell were used. “ExoTransferrin” EV are exosomes comprising a transferrin tropism moiety. Uptake was measured 3 hours after EV particle incubation started. EV uptake was observed for both human and mouse transferrin-containing EVs.

FIGS. 8A-8C show exoTransferrin uptake in Hep3B cells. Three constructs were tested: Transferrin-PrX-mCherry-FLAG (human transferrin; construct 1597; FIG. 8A), mTransferrin-PrX-mCherry-FLAG (mouse transferrin; construct 1598; FIG. 8B); and linker-PrX-mCherry-FLAG-HiBiT (construct 2022; FIG. 8C). 5×10⁵ EV particles per Hep3B cell were used. Uptake was measured 3 hours after EV particle incubation started. EV uptake was observed for both human and mouse transferring-containing EVs.

FIGS. 9A-9C show exoTransferrin uptake in Hep3G2 cells. Three constructs were tested: Transferrin-PrX-mCherry-FLAG (human transferrin; construct 1597; FIG. 9A), mTransferrin-PrX-mCherry-FLAG (mouse transferrin; construct 1598; FIG. 9B); and linker-PrX-mCherry-FLAG-HiBiT (construct 2022; FIG. 9C). 5×10⁵ EV particles per HepG2 cell were used. Uptake was measured 3 hours after EV particle incubation started. EV uptake was observed for both human and mouse transferrin-containing EVs.

FIG. 10A shows a schematic diagram of exemplary extracellular vesicle targeting Trks using neurotrophin-Scaffold X fusion construct. Neurotrophins bind to Trk receptors as a homo dimer and allow the EV to target a sensory neuron.

FIG. 10B shows a schematic diagram of exemplary extracellular vesicle having (i) neuro-tropism on the exterior surface of the EV disclosed herein.

FIGS. 11A and 11B provide schematic diagrams of exemplary EVs that are useful in treating a neurological disorder such as C9FTD/ALS. In FIG. 11A, the EV is capable of targeting antigen-presenting cells (APCs) and thereby, induce T cell dependent antibody responses. As further described in Example 7, the EV comprises the following components: (1) polyGA B cell antigen (“GA”) linked to a Scaffold X (e.g., PTGFRN) on the exterior surface of the EV; (2) universal CD4 T helper peptide (e.g., tetanus toxin (TT)) linked to a Scaffold Y (e.g., BASP-1) on the luminal surface of the EV; and (3) an adjuvant loaded in the lumen of the EV. In FIG. 11B, the EV is engineered to exhibit enhanced ability to target B cells and/or to induce B cell activation. As further described in Example 8, the EV comprises the following components: (1) polyGA B cell antigen (GA) linked to a Scaffold X (e.g., PTGFRN) on the exterior surface of the EV; (2) CD40L (e.g., as a B cell targeting moiety) linked to a Scaffold X (e.g., PTGFRN); (3) universal CD4 T helper peptide (“TT”) linked to a Scaffold Y (e.g., BASP-1) on the luminal surface of the EV; and (4) an adjuvant loaded in the lumen of the EV.

FIG. 12 provides magnetic resonance imaging (MRI) showing the expression of engineered-EVs after intratumor administration.

FIGS. 13A-13E provide representative immunofluorescence images showing the expression of the EVs with different cell types in the brain of GBM animals at 2 hours post EV administration. In each of the figures shown, the top row shows the expression of the different cell types alone. The bottom row shows the expression of the different cell types in combination with the EVs. The expression of the EVs was determined based on 1G11 expression. The different cell types shown include: tumor cells (FIG. 13A), macrophages (FIG. 13B), microglia (FIG. 13C), M2 macrophages (FIG. 13D), and CSF1R+ myeloid cells (FIG. 13E).

FIGS. 14A-14E provide representative immunofluorescence images showing the interaction of the EVs with various cell types within the brain surface leptomeningeal island of tumor cells in GBM animals. FIG. 14A shows an overlay of the EVs with all the different cell types tested (i.e., M2 macrophages, macrophages and microglia, and astrocytes). FIG. 14B shows an overlay of the EVs with macrophages alone. FIG. 14C shows an overlay of the EVs with microglia alone. FIG. 14D shows an overlay of the EVs with astrocytes alone. FIG. 14E shows an overlay of the EVs with both astrocytes and microglia.

FIG. 15 shows the adsorption of alum by EVs described herein.

FIGS. 16A-16D provide comparison of the ability of exoPolyGA-lumen and exoPolyGA-surface to induce antigen-specific immune response in vivo. FIG. 16A provides a schematic of the exoPolyGA-lumen and exoPolyGA-surface constructs. FIG. 16B provides the administration schedule and experimental design. FIG. 16C shows the anti-PolyGA antibody level in the sera of animals from the different treatment groups. FIG. 16D shows the amount of IFN-γ+ T cells in the spleen of animals from the different treatment groups. In FIGS. 16C and 16D, the animals were treated with one of the following: (1) exoPolyGA-lumen; (2) exoPolyGA-surface; (3) exoPolyGA-surface co-loaded with alum and CpG adjuvants; and (4) soluble PolyGA peptide+soluble alum and CpG adjuvants.

FIGS. 17A-17D provide comparison of the ability of exoOVA-lumen and exoOVA-surface to induce anti-OVA antibodies in vivo. FIG. 17A provides a schematic of the exoOVA-lumen and exoOVA-surface constructs. FIG. 17B provides the administration schedule and experimental design. FIG. 17C shows the anti-OVA IgG antibody level in the sera of animals after a single immunization (i.e., day 14). FIG. 17D shows the anti-OVA IgG antibody level in the sera of animals after the boost (i.e., day 28). In FIGS. 17C and 17D, the animals were immunized with one of the following: (1) soluble OVA+soluble alum; (2) exoOVA-surface; and (3) exoOVA-lumen.

FIG. 18A-18C show the ability of exoOVA-lumen co-loaded with alum and CpG adjuvants to induce anti-OVA IgG antibodies in vivo. FIG. 18A provides a schematic of the exoOVA-lumen construct co-loaded with alum and CpG adjuvants. FIG. 18B provides the administration schedule and experimental design. FIG. 18C provide anti-OVA IgG level in animals from the different treatment groups after a single immunization (i.e., day 14; white bars) or after receiving the boost (i.e., day 28; gray bars). The animals received one of the following: (1) exoOVA-lumen; (2) soluble OVA+soluble alum+soluble CpG; and (3) exoOVA-lumen co-loaded with alum and CpG adjuvants.

FIGS. 19A and 19B provides a schematic of an exemplary “exoRVG” described herein—e.g., EV comprising a PTGFRN on the exterior surface of the EV (represented by the curved lines that protrude out from the exterior surface), wherein the PTGFRN is conjugated to a RVG tropism moiety (represented by triangles) (FIG. 19A). FIG. 19B provides an enhanced view of the PTGFRN conjugated to a RVG tropism moiety and associated with the exterior surface of the EV.

FIG. 20 provides an illustration of how the EVs of the present disclosure (e.g., comprising a RVG tropism moiety) can be used to target the motor neurons.

FIGS. 21A and 21B provide schematic of EVs comprising the ALFA plug and play system described herein. As shown in FIG. 21A, ALFA nanobody (NbALFA) is fused to Scaffold X (e.g., PTGFRN) on the exterior surface of the EV (“NbALFA EV”). The crystal structure shows the stable, non-covalent interaction of the NbALFA with the ALFAtag. FIG. 21B shows the loading of moieties of interest (MOI) onto the exterior surface of the NbALFA EVs. As further described herein (see, e.g., Example 16), the moieties of interest are fused to an ALFAtag and then, mixed with the NbALFA EVs, resulting in the stable association of the ALFAtagged moieties of interest to the exterior surface of the NbALFA EVs. The EVs can be loaded with the same moiety of interest (see top drawing) or a mixture of different moiety of interest (see bottom drawing).

FIGS. 22A-22G show the effect of alum and CpG adjuvants on the ability of EVs described herein to induce antigen-specific antibodies in vivo. FIG. 22A provides a table showing the administration schedule and experimental design. FIGS. 22B, 22C, and 22D provide comparison of anti-OVA IgG levels in the sera of animals from the different treatment groups at days 14, 28, and 42 post initial treatment, respectively. FIGS. 22E, 22F, and 22G provide comparison of anti-OVA IgM levels in the sera of animals from the different treatment groups at days 14, 28, and 42 post initial treatment, respectively.

FIG. 23 provides SDS-PAGE (top) and Western blot (bottom) results demonstrating that NbALFA EVs can be simultaneously loaded with multiple moieties of interest. Wild-type EVs or NbALFA EVs were mixed with 10 μg of NLuc-ALFAtag or molar equivalent of mouse IL-12 fused to ALFAtag (“mIL12-ALFAtag”).

### DETAILED DESCRIPTION OF DISCLOSURE

The present disclosure is directed to an engineered EV, comprising one or more payloads, wherein the one or more payloads can improve at least one property (e.g., such as those disclosed herein) of the EV, and uses thereof. For example, in some aspects, the EVs disclosed herein are capable of targeting an immune cell (e.g., macrophage or dendritic cell) within the central nervous system of a subject. In some aspects, the one or more payloads that can be expressed in an EV disclosed herein comprise an antigen (e.g., associated with a neurological disorder disclosed herein), an adjuvant, an immune modulator, or combinations thereof. In some aspects, the one or more payloads can be attached (or linked) to one or more scaffold moieties on the surface of EVs or on the luminal surface of EVs. In some aspects, the EVs can further comprise a targeting moiety, which can also be attached (or linked) to one or more of the scaffold moieties disclosed herein. Non-limiting examples of the various aspects are shown in the present disclosure.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In certain aspects, an extracellular vehicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome or multi-vesicular body with the plasma membrane. In certain aspects, an exosome comprises a scaffold moiety. As described infra, exosome can be derived from a producer cell, and isolated from the producer cell based on its size, density, biochemical parameters, or a combination thereof. In some aspects, the EVs of the present disclosure are produced by cells that express one or more transgene products. Unless indicated otherwise, the terms “extracellular vesicle” and “exosomes” can be used interchangeably.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In certain aspects, a nanovesicle comprises a scaffold moiety. Nanovesicles, once derived from a producer cell, can be isolated from the producer cell based on its size, density, biochemical parameters, or a combination thereof.

As used herein the term “surface-engineered exosome” (e.g., Scaffold X-engineered exosome) refers to an EV with the membrane or the surface of the EV modified in its composition so that the surface of the engineered EV, is different from that of the EV, prior to the modification or of the naturally occurring EV. The engineering can be on the surface of the EV, or in the membrane of the EV, so that the surface of the EV, is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV, comprises an exogenous protein (i.e., a protein that the EV, does not naturally express) or a fragment or variant thereof that can be exposed to the surface of the EV, or can be an anchoring point (attachment) for a moiety exposed on the surface of the EV. In other aspects, a surface-engineered EV, comprises a higher expression (e.g., higher number) of a natural exosome protein (e.g., Scaffold X) or a fragment or variant thereof that can be exposed to the surface of the EV, or can be an anchoring point (attachment) for a moiety exposed on the surface of the EV.

As used herein the term “lumen-engineered exosome” (e.g., Scaffold Y-engineered exosome) refers to an EV, with the membrane or the lumen of the EV, modified in its composition so that the lumen of the engineered EV, is different from that of the EV, prior to the modification or of the naturally occurring EV. The engineering can be directly in the lumen or in the membrane of the EV so that the lumen of the EV is changed. For example, the membrane is modified in its composition of a protein, a lipid, a small molecule, a carbohydrate, etc. so that the lumen of the EV is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises an exogenous protein (i.e., a protein that the EV does not naturally express) or a fragment or variant thereof that can be exposed in the lumen of the EV or can be an anchoring point (attachment) for a moiety exposed on the inner layer of the EV. In other aspects, a lumen-engineered EV, comprises a higher expression of a natural exosome protein (e.g., Scaffold X or Scaffold Y) or a fragment or variant thereof that can be exposed to the lumen of the exosome or can be an anchoring point (attachment) for a moiety exposed in the lumen of the exosome.

The term “modified,” when used in the context of EVs described herein, refers to an alteration or engineering of an EV and/or its producer cell, such that the modified EV is different from a naturally-occurring EV. In some aspects, a modified EV described herein comprises a membrane that differs in composition of a protein, a lipid, a small molecular, a carbohydrate, etc. compared to the membrane of a naturally-occurring EV (e.g., membrane comprises higher density or number of natural exosome proteins and/or membrane comprises proteins that are not naturally found in exosomes (e.g., antigen, adjuvant, and/or immune modulator). In certain aspects, such modifications to the membrane changes the exterior surface of the EV (e.g., surface-engineered EVs described herein). In certain aspects, such modifications to the membrane changes the lumen of the EV (e.g., lumen-engineered EVs described herein).

As used herein, the term “scaffold moiety” refers to a molecule that can be used to anchor a payload or any other compound of interest (e.g., antigen, adjuvant, and/or immune modulator) to the EV either on the luminal surface or on the exterior surface of the EV. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV. In some aspects, a scaffold moiety comprises a lipid, carbohydrate, or protein that does not naturally exist in the EV. In certain aspects, a scaffold moiety is Scaffold X. In some aspects, a scaffold moiety is Scaffold Y. In further aspects, a scaffold moiety comprises both Scaffold X and Scaffold Y. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin, LAMP2, and LAMP2B.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV). In some aspects, a Scaffold X can anchor a moiety (e.g., antigen, adjuvant, and/or immune modulator) to the external surface or the luminal surface of the exosome.

As used herein, the term “Scaffold Y” refers to exosome proteins that were newly identified within the lumen of exosomes. See, e.g., International Appl. No. PCT/US2018/061679, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety to the luminal surface of the exosome). In some aspects, a Scaffold Y can anchor a moiety (e.g., antigen, adjuvant, and/or immune modulator) to the luminal surface of the EV.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV. Similarly, in certain aspects, a functional fragment of a Scaffold Y protein retains the ability to anchor a moiety on the luminal surface of the EV. Whether a fragment is a functional fragment can be assessed by any art known methods to determine the protein content of EVs including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, antigen, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof. In some aspects, the variant or variant of a fragment of Scaffold X protein disclosed herein retains the ability to be specifically targeted to EVs. In some aspects, the Scaffold X includes one or more mutations, for example, conservative amino acid substitutions.

In some aspects, a variant of a Scaffold Y comprises a variant having at least about 70% identity to MARCKS, MARCKSL1, BASP1 or a fragment of MARCKS, MARCKSL1, or BASP1. In some aspects, variants or variants of fragments of BASP1 share at least about 70%, at least about 80%, at least about 85%, at least about 90%, at least about 95%, at least about 96%, at least about 97%, at least about 98%, or at least about 99% sequence identity with BASP1 according to SEQ ID NO: 49 or with a functional fragment thereof. In some aspects, the variant or variant of a fragment of Scaffold Y protein retains the ability to be specifically targeted to the luminal surface of EVs. In some aspects, the Scaffold Y includes one or more mutations, e.g., conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at worldwideweb.tcoffee.org, and alternatively available, e.g., from the EBI. It will also be appreciated that the final alignment used to calculate percent sequence identity can be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein the term “linked to” or “conjugated to” are used interchangeably and refer to a covalent or non-covalent bond formed between a first moiety and a second moiety, e.g., Scaffold X and an antigen (or adjuvant or immune modulator), respectively, e.g., a scaffold moiety expressed in or on the extracellular vesicle and an antigen, e.g., Scaffold X (e.g., a PTGFRN protein), respectively, in the luminal surface of or on the external surface of the extracellular vesicle.

The term “encapsulated”, or grammatically different forms of the term (e.g., encapsulation, or encapsulating) refers to a status or process of having a first moiety (e.g., antigen, adjuvant, or immune modulator) inside a second moiety (e.g., an EV) without chemically or physically linking the two moieties. In some aspects, the term “encapsulated” can be used interchangeably with “in the lumen of”. Non-limiting examples of encapsulating a first moiety (e.g., antigen, adjuvant, or immune modulator) into a second moiety (e.g., EVs) are disclosed elsewhere herein.

As used herein, the term “producer cell” refers to a cell used for generating an EV. A producer cell can be a cell cultured in vitro, or a cell in vivo. A producer cell includes, but are not limited to, a cell known to be effective in generating EVs, e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In certain aspects, a producer cell is not a naturally-existing antigen-presenting cell (i.e., has been modified). In some aspects, a producer cell is not a naturally-existing dendritic cell, a naturally-existing B cell, a naturally-existing mast cell, a naturally-existing macrophage, a naturally-existing neutrophil, naturally-existing Kupffer-Browicz cell, cell derived from any of these cells, or any combination thereof. Additional disclosures relating to such producer cells are provided elsewhere in the present disclosure. In some aspects, the EVs useful in the present disclosure do not carry an antigen on MHC class I or class II molecule (i.e., antigen is not presented on MHC class I or class II molecule) exposed on the surface of the EV, but instead can carry an antigen in the lumen of the EV, or on the surface of the EV, by attachment to Scaffold X and/or Scaffold Y.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “immune modulator” refers to an agent (i.e., payload) that acts on a target (e.g., a target cell) that is contacted with the extracellular vesicle, and regulates the immune system. Non-limiting examples of immune modulator that can be introduced into an EV and/or a producer cell include agents such as, modulators of checkpoint inhibitors, ligands of checkpoint inhibitors, cytokines, derivatives thereof, or any combination thereof. The immune modulator can also include an agonist, an antagonist, an antibody, an antigen-binding fragment, a polynucleotide, such as siRNA, antisense oligonucleotide, a phosphorodiamidate morpholino oligomer (PMO), a peptide-conjugated phosphorodiamidate morpholino oligomer (PPMO), miRNA, lncRNA, mRNA DNA, or a small molecule.

As used herein, the term a “bio-distribution modifying agent,” which refers to an agent that can modify the distribution of extracellular vesicles in vivo or in vitro (e.g., in a mixed culture of cells of different varieties). In some aspects, the term “targeting moiety” can be used interchangeably with the term bio-distribution modifying agent. In some aspects, the targeting moiety alters the tropism of the EV (“tropism moiety”). As used herein, the term “tropism moiety” refers to a targeting moiety that when expressed on an EV alters and/or enhances the natural movement of the EV. For example, in some aspects, a tropism moiety can promote the EV to move towards a particular cell, tissues, or a stimuli. In certain aspects, a tropism moiety that can be used with the EVs of the present disclosure can include apoE, which bind to endothelial LDLR and promote the transcytosis of the EV across the blood-brain barrier. Non-limiting examples of other tropism moieties that can be used with the present disclosure includes those that can bind to DEC-205 or liver sinusoidal endothelial cells (LSEC). Unless indicated otherwise, the term “targeting moiety,” as used herein, encompasses tropism moieties and therefore, can be used interchangeably. The bio-distribution agent can be a biological molecule, such as a protein, a peptide, a lipid, or a carbohydrate, or a synthetic molecule. For example, the bio-distribution modifying agent can be an antibody, a synthetic polymer (e.g., PEG), a natural ligand (e.g., CD40L, albumin), a recombinant protein (e.g., XTEN), but not limited thereto.

In certain aspects, the bio-distribution modifying agent is displayed on the surface of EVs. The bio-distribution modifying agent can be displayed on the EV surface by being fused to a scaffold protein (e.g., Scaffold X) (e.g., as a genetically encoded fusion molecule). In some aspects, the bio-distribution modifying agent can be displayed on the EV surface by chemical reaction attaching the bio-distribution modifying agent to an EV surface molecule. A non-limiting example is PEGylation. In some aspects, EVs disclosed herein can further comprise a bio-distribution modifying agent (in addition to an antigen, adjuvant, or immune modulator).

Non-limiting examples of a tropism or targeting moiety that can be used with the present disclosure include a C-type lectin domain family 9 member A (Clec9a) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), CD3, CD14, CD16, CD64, CD68, CD71, CCR5, or any combination thereof. In certain aspects, the targeting moiety is Clec9a protein. In some aspects, the targeting moiety is CD14, CD16, CD64, CD68, CD71, CCR5, or any combination thereof. Additional examples of targeting moieties that can be used with the present disclosure are provided elsewhere in the present disclosure.

As used herein, the term “C-type lectin domain family 9 member A” (Clec9a) protein refers to a group V C-type lectin-like receptor (CTLR) that functions as an activation receptor and is expressed on myeloid lineage cells (e.g., DCs). Huysamen et al., J Biol Chem 283(24):16693-701 (2008); U.S. Pat. No. 9,988,431 B2, each of which is herein incorporated by reference in its entirety. Synonyms of Clec9a are known and include CD370, DNGR-1, 5B5, HEEE9341, and C-type lectin domain containing 9A. In some aspects, Clec9a protein is expressed on human cDC1 cells. In some aspects, Clec9a protein is expressed on mouse cDC1 and pDC cells. Unless indicated otherwise, Clec9a, as used herein, can refer to Clec9a from one or more species (e.g., humans, non-human primates, dogs, cats, guinea pigs, rabbits, rats, mice, horses, cattle, and bears).

As used herein, the term “payload” refers to an agent that acts on a target (e.g., a target cell) that is contacted with the EV. Contacting can occur in vitro or in a subject. In some aspects, unless indicated otherwise, the term payload can be used interchangeably with the terms “moiety,” “agents,” and “biologically active molecules.” Non-limiting examples of payload that can be included on the EV, are an antigen, an adjuvant, and/or an immune modulator. Payloads that can be introduced into an EV, and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, siRNA, antisense oligonucleotide, a phosphorodiamidate morpholino oligomer (PMO), a peptide-conjugated phosphorodiamidate morpholino oligomer (PPMO), or combinations thereof), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an antigen. As used herein, the term “antigen” refers to any agent that when introduced into a subject elicits an immune response (cellular or humoral) to itself. As will be apparent from the present disclosure, in some aspects, an antigen is associated with a neurological disorder. Additional disclosure relating to such antigens are provided elsewhere in the present disclosure.

As used herein, the term “central nervous system” (CNS) refers to the part of the nervous system comprising the bran and the spinal cord.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab)₂, F(ab1)₂, Fv, dAb, and Fd fragments, diabodies, and antibody-related polypeptides. Antibody includes bispecific antibodies and multispecific antibodies so long as they exhibit the desired biological activity or function.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects, the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs, comprise less than about 10% of macromolecules by mass/volume (m/v) percentage concentration. Some fractions can contain less than about 0.001%, less than about 0.01%, less than about 0.05%, less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, or less than about 10% (m/v) of macromolecules.

As used herein, the term “macromolecule” means nucleic acids, contaminant proteins, lipids, carbohydrates, metabolites, or a combination thereof.

As used herein, the term “conventional exosome protein” means a protein previously known to be enriched in exosomes, including but is not limited to CD9, CD63, CD81, PDGFR, GPI anchor proteins, lactadherin LAMP2, and LAMP2B, a fragment thereof, or a peptide that binds thereto.

“Administering,” as used herein, means to give a composition comprising an EV disclosed herein to a subject via a pharmaceutically acceptable route. Routes of administration can be intravenous, e.g., intravenous injection and intravenous infusion. Additional routes of administration include, e.g., subcutaneous, intramuscular, intrathecal, intravitreal, intracranial, oral, nasal, and pulmonary administration. Exosomes can also be directly administered to the target tissue, EVs can be administered as part of a pharmaceutical composition comprising at least one excipient.

An “immune response,” as used herein, refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses. In some aspects, an immune response is an “inhibitory” immune response. An “inhibitory” immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A “stimulatory” immune response is an immune response that results in the generation of effectors cells (e.g., cytotoxic T lymphocytes) that can destroy and clear a target antigen (e.g., tumor antigen or viruses).

As used herein, the term “cellular immune response” can be used interchangeably with the term “cell-mediated immune response” and refers to an immune response that does not involve antibodies. Instead, a cellular immune response involves the activation of different immune cells (e.g., phagocytes and antigen-specific cytotoxic T-lymphocytes) that produce various effector molecules (e.g., cytokines, perforin, granzymes) upon activation (e.g., via antigen stimulation). As used herein, the term “humoral immune response” refers to an immune response mediated by macromolecules found in extracellular fluids, such as secreted antibodies, complement proteins, and certain antimicrobial peptides. The term “antibody-mediated immune response” refers to an aspect of a humoral immune response that is mediated by antibodies.

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4+ T cells or CD8+ T cells). In some aspects, an immune cell that can be targeted with a targeting moiety disclosed herein (e.g., anti-CD3) comprises a naïve CD4+ T cell. In some aspects, an immune cell comprises a memory CD4+ T cell. In some aspects, an immune cell comprises an effector CD4+ T cell. In some aspects, an immune cell comprises a naïve CD8+ T cell. In some aspects, an immune cell comprises a memory CD8+ T cell. In some aspects, an immune cell comprises an effector CD8+ T cell. In some aspects, an immune cell is a dendritic cell. In certain aspects, a dendritic cell comprises a plasmacytoid dendritic cell (pDC), a conventional dendritic cell 1 (cDC1), a conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof. Accordingly, in certain aspects, an immune cell that an EV disclosed herein can specifically target includes a conventional dendritic cell 1 (cDC1) and/or plasmacytoid dendritic cells (pDC). In some aspects, an immune cell is a macrophage. In some aspects, the macrophage comprises M1 macrophages, M2 macrophages, or both. In certain aspects, the macrophage is a microglia, meningeal macrophage, perivascular macrophage, choroid plexus macrophage, or combinations thereof.

As used herein, the term “T cell” or “T-cell” refers to a type of lymphocyte that matures in the thymus. T cells play an important role in cell-mediated immunity and are distinguished from other lymphocytes, such as B cells, by the presence of a T-cell receptor on the cell surface. T-cells include all types of immune cells expressing CD3, including T-helper cells (CD4+ cells), cytotoxic T-cells (CD8+ cells), natural killer T-cells, T-regulatory cells (Treg), and gamma-delta T cells.

A “naïve” T cell refers to a mature T cell that remains immunologically undifferentiated (i.e., not activated). Following positive and negative selection in the thymus, T cells emerge as either CD4+ or CD8+naïve T cells. In their naïve state, T cells express L-selectin (CD62L+), IL-7 receptor-α (IL-7R-α), and CD132, but they do not express CD25, CD44, CD69, or CD45RO. As used herein, “immature” can also refers to a T cell which exhibits a phenotype characteristic of either a naïve T cell or an immature T cell, such as a TSCM cell or a TCM cell. For example, an immature T cell can express one or more of L-selectin (CD62L+), IL-7Ra, CD132, CCR7, CD45RA, CD45RO, CD27, CD28, CD95, CXCR3, and LFA-1. Naïve or immature T cells can be contrasted with terminal differentiated effector T cells, such as TEM cells and TEFF cells.

As used herein, the term “effector” T cells or “TEFF” cells refers to a T cell that can mediate the removal of a pathogen or cell without requiring further differentiation. Thus, effector T cells are distinguished from naive T cells and memory T cells, and these cells often have to differentiate and proliferate before becoming effector cells.

As used herein, the term “memory” T cells refer to a subset of T cells that have previously encountered and responded to their cognate antigen. In some aspects, the term is synonymous with “antigen-experienced” T cells. In some aspects, memory T cells can be effector memory T cells or central memory T cells. In some aspects, the memory T cells are tissue-resident memory T cells. As used herein, the term “tissue-resident memory T cells” or “TRM cells” refers to a lineage of T cells that occupies tissues (e.g., skin, lung, gastrointestinal tract) without recirculating. TRM cells are transcriptionally, phenotypically and functionally distinct from central memory and effector memory T cells which recirculate between blood, the T cell zones of secondary lymphoid organs, lymph and nonlymphoid tissues. One of the roles of TRM cells is to provide immune protection against infection in extra-lymphoid tissues.

As used herein, the term “dendritic cells” or “DCs” refers to a class of bone-marrow-derived immune cells that are capable of processing extracellular and intracellular proteins and to present antigens in the context of MHC molecules to prime naïve T cells. In some aspects, dendritic cells can be divided into further subtypes, such as conventional dendritic cell 1 (cDC1), conventional dendritic cell 2 (cDC2), plasmacytoid dendritic cell (pDC), inflammatory monocyte derived dendritic cells, Langerhans cells, dermal dendritic cells, lysozyme-expressing dendritic cells (LysoDCs), Kupffer cells, and combinations thereof. In certain aspects, the different DC subsets can be distinguished based on their phenotypic expression. For example, in some aspects, human cDC1 cells are CD1c⁻ and CD141⁺. In some aspects, human cDC2 cells are CD1c⁺ and CD141⁻. In some aspects, human pDC cells are CD123⁺. In some aspects, mouse cDC1 cells are XCR1⁺, Clec9a⁺, and Sirpa⁻. In some aspects, mouse cDC2 cells are CD8⁺, CD1 Sirpa⁺, XCR1⁻, and CD1c,b⁺. In some aspects, mouse pDC cells are CD137⁺, XCR1⁻, and Sirpa⁻. Other phenotypic markers for distinguishing the different DC subsets are known in the art. See, e.g., Collin et al., Immunology 154(1): 3-20 (2018). In some aspects, the different DC subsets can be distinguished based on their functional properties. For example, in certain aspects, pDCs produce large amounts of IFN-α, while cDC1s and cDC2s produce inflammatory cytokines, such as IL-12, IL-6, and TNF-α. Other methods of distinguishing the different DC subsets are known in the art. See, e.g., U.S. Pat. Nos. 8,426,565 B2 and 9,988,431, each of which is herein incorporated by reference in its entirety.

As used herein, the term “macrophage” refers to a mononuclear phagocyte characterized by the expression of at least CD14 and lack of expression of dendritic cell markers. Macrophages can be typically divided into (i) classically-activated macrophages (“M1 macrophages”) and (ii) alternatively-activated macrophages (“M2 macrophages”). Martinez et al., Annu. Rev. Immunol. 27:451-483 (2009). Generally, M1 macrophages exhibit potent anti-microbial properties, reminiscent of type 1 T-helper lymphocyte (Th1) responses. In contrast, M2 macrophages promote type 2 T-helper lymphocyte (Th2)-like responses, secrete less pro-inflammatory cytokines, and assist resolution of inflammation by trophic factor synthesis and phagocytosis. Mosser et al., Nature Rev. 8:958-969 (2008). M2 macrophages can be further divided into three distinct subclasses, i.e., M2a, M2b, and M2c, defined by specific cytokine profiles. Mantovani et al., Trends Immunol. 25:677-686 (2004). While M2 macrophages are generally characterized by low production of pro-inflammatory cytokines, such as IL-12, and high production of anti-inflammatory cytokines such as IL-10, M2b macrophages retain high levels of inflammatory cytokine production, such as TNF-α and IL-6. Mosser, J. Leukocyte Biol. 73:209-212 (2003).

Macrophages can be polarized by their microenvironment to assume different phenotypes associated with different stages of inflammation and healing. Stout et al., J. Immunol. 175:342-349 (2005). Certain macrophages are indispensable for wound healing. They participate in the early stages of cell recruitment and of tissue defense, as well as the later stages of tissue homeostasis and repair. Pollard, Nature Rev. 9:259-270 (2009). Macrophages derived from peripheral blood monocytes have been used to treat refractory ulcers. Danon et al., Exp. Gerontol. 32:633-641 (1997); Zuloff-Shani et al., Transfus. Apher. Sci. 30:163-167 (2004), each of which is incorporated herein by reference as if set forth in its entirety.

The term “immunoconjugate,” as used herein, refers to a compound comprising a binding molecule (e.g., an antibody) and one or more moieties, e.g., therapeutic or diagnostic moieties, chemically conjugated to the binding molecule. In general, an immunoconjugate is defined by a generic formula: A-(L-M)n, wherein A is a binding molecule (e.g., an antibody), L is an optional linker, and M is a heterologous moiety which can be for example a therapeutic agent, a detectable label, etc., and n is an integer. In some aspects, multiple heterologous moieties can be chemically conjugated to the different attachment points in the same binding molecule (e.g., an antibody). In other aspects, multiple heterologous moieties can be concatenated and attached to an attachment point in the binding molecule (e.g., an antibody). In some aspects, multiple heterologous moieties (being the same or different) can be conjugated to the binding molecule (e.g., an antibody).

Immunoconjugates can also be defined by the generic formula in reverse order. In some aspects, the immunoconjugate is an “antibody-Drug Conjugate” (“ADC”). In the context of the present disclosure, the term “immunoconjugate” is not limited to chemically or enzymatically conjugates molecules. The term “immunoconjugate” as used in the present disclosure also includes genetic fusions. In some aspects of the present disclosure, the biologically active molecule is an immunoconjugate. The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule (i.e., a payload) is an antibody-drug conjugate.

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

“Prevent” or “preventing,” as used herein, refers to decreasing or reducing the occurrence or severity of a particular outcome. In some aspects, preventing an outcome is achieved through prophylactic treatment.

### II. Methods of the Present Disclosure

### Methods of Treating a Neurological Disorder

Disclosed herein are engineered EVs that can be used to treat a neurological disorder in a subject in need thereof. In some aspects, a method of treating a neurological disorder provided herein comprises administering to the subject an EV, which comprises an antigen (e.g., associated with a neurological disorder). In certain aspects, the EV is capable of targeting a cell within the central nervous system (CNS) of the subject.

As described herein, global aging is creating a tsunami of neurodegeneration. As people live longer, the incidence of neurodegenerative disorders is expected to increase dramatically. Accordingly, there is an urgent need for cost effective solutions (both preventive and therapeutic) for large populations. As will be apparent from the present disclosure, the EVs described herein are capable of meeting such needs, e.g., (i) provides cost-effective prophylactic and therapeutic solution, and/or (ii) satisfies the need for epitope specific, safe, and effective active humoral responses.

Previous attempts at treating neurological disorders centered on the administration of antibodies that can specifically bind to different proteins associated with a neurological disorder (e.g., amyloid-β). Examples of such antibodies include aducanumab (AN1792), gantenerumab, LY3002813 (N3pG), and BAN2401. While such antibodies had some early promising results (e.g., decreased amyloid and tau pathology, decreased amount of amyloid-β plaques, and modest disease progression), they failed to meet the necessary endpoints in subsequent clinical trials. See, e.g., worldwideweb.clinicaltrialsarena.com/news/roche-gantenerumab-phaseii-iii-data. Moreover, some of the antibodies also had undesirable side-effects (e.g., T-cell mediated meningoencephalitis). See Nicoll et al., Brain 142(7): 2113-2126 (July 2019). In some aspects, EVs of the present disclosure are both efficacious (e.g., reduces the amount of misfolded neuronal proteins) and have manageable safety profile (e.g., no significant adverse effect).

As used herein, the terms “neurological disorder” and “neuroimmunological disorder” can be used interchangeable and refer to diseases and disorders of either the central or peripheral nervous system. The nervous system represents a privileged immune environment that generally dampens inflammatory responses in the brain spinal cord and nerves. This relative low immunoresponsiveness (anergy) is not only a function of the blood-brain barrier (i.e., to a highly selective semipermeable border of endothelial cells that prevents solutes in the circulating blood from non-selectively crossing into the extracellular fluid of the central nervous system) but also a feature of the resident myeloid cells of the nervous system (e.g., microglia, meningeal macrophages, perivascular macrophages, and choroid plexus macrophages). These cells generally display immunosuppressive phenotypes and are known to become further “immunosilenced” or anergic in the setting of certain pathologies such as cancer or chronic infections. Such features often make it difficult to target a drug or vaccines to the CNS. As described herein, the EVs of the present disclosure can be engineered to cross the blood-brain barrier into the CNS and thereby, modulate the immune response within the CNS. Accordingly, in some aspects, administering an EV disclosed herein can increase an immune response, where the neuroimmunological disorder is due to an inability of a subject's immune system to mount an effective immune response against the disorder. In some aspects, administering an EV disclosed herein can reduce an immune response, where neuroimmunological disorder is due to an aberrant or excessive immune response within the nervous system. Unless specified otherwise, the term “neurological disorders” and “neuroimmunological disorders” comprises all diseases or disorders of the nervous system, including autoimmune disorders.

Not to be bound by any one theory, in some aspects, administering the EV to the subject results in the induction of a humoral immune response, in which the antibodies that are produced can specifically bind to the antigen. Accordingly, in some aspects, administering the EV to the subject can increase the amount of antigen-specific antibodies in the subject. In certain aspects, the amount of antigen-specific antibodies in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold or more, compared to a reference (e.g., amount of antigen-specific antibodies in that subject prior to the EV administration or in a corresponding subject that received the antigen in a non-EV delivery vehicle). As described herein, in some aspects, the humoral immune response is induced in the subject without activating harmful/deleterious T cells.

In some aspects, binding of the antibodies to the antigen can facilitate the reduction and/or elimination of the antigen from the subject. In certain aspects, the amount of the antigen in the subject (e.g., within the CNS) is reduced and/or eliminated by at least about 5%, at least about 10%, at least about 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% after the administration of the EV.

In some aspects, a neurological disorder that can be treated with the present disclosure is associated with a neuronal protein that has misfolded. In certain aspects the neuronal protein is associated with microsatellite repeat expansions or short sequences of genetic code that are repeated too many times (referred to herein as “RAN” proteins). Non-limiting examples of such neuronal proteins include amyloid beta (Aβ), tau, alpha-synuclein, poly-GA, or combinations thereof. Non-limiting examples of neurological disorders that are associated with such misfolded neuronal proteins can include a brain tumor, neoplastic meningitis, leptomeningeal cancer disease (LMD), amyotrophic lateral sclerosis (ALS), frontotemporal dementia (FTD), Parkinson's disease (PD), Huntington's disease (HD), Alzheimer's disease (AD), or combinations thereof. Accordingly, in some aspects, an EV disclosed herein can be used to treat any neurological disorder, including those associated with a misfolded neuronal protein described herein.

Not to be bound by any one theory, in some aspects, an EV disclosed herein treats a neurological disorder by reducing the amount of misfolded neuronal proteins in a subject. In some aspects, an EV disclosed herein is capable of decreasing the amount of Aβ plaques when administered to a subject in need thereof. In certain aspects, the amount of Aβ plaques in the subject is decreased by at least about 5%, at least about 10%, at least about 20%, at least about 30%, at least about 40%, at least about 50%, at least about 60%, at least about 70%, at least about 80%, or at least about 90% or more compared to a reference (e.g., amount of Aβ plaques in the subject prior to administering the EV or amount of Aβ plaques in a corresponding subject not treated with the EV disclosed herein).

In some aspects, an EV of the present disclosure is capable of decreasing the amount of tau aggregates when administered to a subject in need thereof. In certain aspects, the amount of tau aggregates in the subject is decreased by at least about 5%, at least about 10%, at least about 20%, at least about 30%, at least about 40%, at least about 50%, at least about 60%, at least about 70%, at least about 80%, or at least about 90% or more compared to a reference (e.g., amount of tau aggregates in the subject prior to administering the EV or amount of tau aggregates in a corresponding subject not treated with the EV disclosed herein).

In some aspects, an EV described herein is capable of decreasing a neurofilament level when administered to a subject in need thereof. In certain aspects, the amount of neurofilament level in the subject is decreased by at least about 5%, at least about 10%, at least about 20%, at least about 30%, at least about 40%, at least about 50%, at least about 60%, at least about 70%, at least about 80%, or at least about 90% or more compared to a reference (e.g., amount of neurofilament level in the subject prior to administering the EV or amount of neurofilament level in a corresponding subject not treated with the EV disclosed herein).

In some aspects, an EV described herein is capable of slowing the progression of a disease or disorder (e.g., neurological disorder) when administered to a subject in need thereof. In certain aspects, the progression of the disease or disorder in the subject is decreased by at least about 5%, at least about 10%, at least about 20%, at least about 30%, at least about 40%, at least about 50%, at least about 60%, at least about 70%, at least about 80%, or at least about 90% or more compared to a reference (e.g., progression of the disease or disorder in the subject prior to administering the EV or progression of the disease or disorder in a corresponding subject not treated with the EV disclosed herein).

In some aspects, an EV described herein is capable of improving one or more symptoms (e.g., behavioral function) associated with a neurological disorder when administered to a subject in need thereof. Non-limiting examples of such symptoms are provided elsewhere in the present disclosure.

In some aspects, EVs described herein have a manageable safety profile when administered to a subject in need thereof. In certain aspects, manageable safety profile comprises no induction of harmful/deleterious T cells (e.g., such as those that can induce T-cell mediated meningoencephalitis) when administered to a subject.

Accordingly, in some aspects, EVs of the present disclosure are capable of inducing robust B cell responses without deleterious T cell responses. In some aspects, such EVs can have one or more of the following features: (i) comprise B cell activating adjuvants (e.g., CpG-B and monophosphoryl lipid A (MPLA)) (for inducing innate response); (ii) comprise a tolerogenic adjuvant (e.g., rapamycin) (for inducing innate response); (iii) comprise foreign CD4+ T helper cell antigen (e.g., pan HLA-DR, or tetanus or diphtheria toxin peptide) (for inducing cellular response); (iv) express B cell antigens on the surface at a high density, e.g., to enhance B cell receptor crosslinking (for inducing humoral response); (v) comprise co-stimulators and/or cytokines (e.g., CD40-L, ICOS, and IL-21), e.g., to enhance B cell activation (for inducing humoral response); (vi) comprising a tropism/targeting moiety (e.g., anti-CD40, MHCII, and CD180 antibodies), e.g., to enhance B cell targeting (for inducing humoral response).

As described herein, in some aspects, a neurological disorder that can be treated with the present disclosure is associated with repeat associated non-AUG proteins (i.e., RAN proteins). In some aspects, the neurological disorder that can be treated with the disclosures provided herein is ALS, FTD, or both. In certain aspects, the ALS and/or FTD is associated with a protein comprising a hexanucleotide GGGGCC repeat expansion (i.e., mutation) in the C9orf72 gene (i.e., RAN protein). See, e.g., Balendra et al., Nat Rev Neurol 14(9): 544-558 (September 2018). As used herein, the term “C9FTD/ALS” refers to C9orf72-associated diseases with clinical features of FTD, ALS, or both. Some patients with the C9orf72 mutation develop ALS, dementia, or both. In some aspects, the EVs disclosed herein can be used to treat any disorders associated with a RAN protein (e.g., comprising C9orf72 repeat expansion). Non-limiting examples of such disorders include Parkinson's disease, progressive supranuclear palsy, ataxia, corticobasal syndrome, Huntington disease-like syndrome, Creutzfeldt-Jakob disease, and Alzheimer's disease. Additional examples are provided elsewhere in the present disclosure.

Not to be bound by any one theory, in some aspects, the engineered-EVs of the present disclosure can treat a neurological disorder, such as C9FTD/ALS, by inducing the production of antibodies specific for a protein associated with the disorder. In the case of C9FTD/ALS, administering an EV disclosed herein can increase the amount of anti-polyGA antibodies in the subject. In some aspects, the amount of anti-polyGA antibodies in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold or more, compared to a reference (e.g., amount of anti-polyGA antibodies in the subject prior to the administration or the amount of anti-polyGA antibodies in a corresponding subject that did receive the engineered-EV). As described herein, in some aspects, such antibodies are produced in the absence of activation of harmful/deleterious T cells.

The importance of such antibodies has been described previously. See, e.g., Nguyen et al., Neuron 105(4):645-662 (February 2020), which is incorporated herein by reference in its entirety. However, with passive immunization (i.e., antibody therapy), there are significant limitations (e.g., high antibody production costs and frequent administration requirements) that prevent the use of passive immunization to treat diseases, such as C9FTD/ALS, which are lifelong diseases. As described herein, the engineered-EVs of the present disclosure do not share such limitations. Because EVs are naturally produced in nearly all eukaryotes, the engineered-EVs of the present disclosure are non-immunogenic and can stimulate a subject's own immune system to produce the antibodies. Moreover, the engineered-EVs disclosed herein are less costly to manufacture and can be quickly modified to treat various diseases (e.g., modular or “plug and play” EVs described herein). Accordingly, at least for such reasons, the EVs of the present disclosure offer a much superior treatment alternative to what is available in the art.

In some aspects, a neurological disorder that can be treated with the present disclosure is leptomeningeal cancer disease (LMD).

In some aspects, a neurological disorder that can be treated is a brain tumor. In some aspects, the brain tumor is a glioma. In certain aspects, the glioma is a low grade glioma or a high grade glioma. In some aspects, the glioma is oligodendroglioma, anaplastic astrocytomas, glioblastoma multiforme, diffuse intrinsic pontine glioma, IDH1 and IDH2-mutated glioma, or combinations thereof. In some aspects, the glioma is glioblastoma multiforme.

In some aspects, administering an EV disclosed herein can inhibit and/or reduce the growth of a brain tumor in the subject. In some aspects, the growth of a brain tumor (e.g., tumor volume or weight) is reduced by at least about 5%, at least about 10%, at least about 20%, at least about 30%, at least about 40%, at least about 50%, at least about 60%, at least about 70%, at least about 80%, at least about 90%, or about 100% compared to a reference (e.g., tumor volume in the subject prior to the EV administration or in a corresponding subject that received the antigen in a non-EV delivery vehicle).

As used herein, the term “brain tumor” refers to an abnormal growth of cells within the brain (e.g., within the meninges). Brain tumors can be categorized as primary or secondary brain tumor. “Primary brain tumor” refers to brain tumors that originate within the brain. “Secondary brain tumor” refers to brain tumors that are the result of cancer cells originating at primary sites outside the brain that have metastasized (i.e., spread) to the brain. Unless specified otherwise, the term brain tumor can refer to both primary and secondary brain tumors.

In some aspects, a brain tumor that can be treated with the present disclosure comprises an acoustic neuroma, choroid plexus carcinoma, craniopharyngioma, embryonal tumor, glioma, medulloblastoma, meningioma, pediatric brain tumor, pineoblastoma, pituitary tumor, or combinations thereof.

In some aspects, a brain tumor that can be treated with the present disclosure comprises a glioma. As used herein, the term “glioma” refers to a type of tumor that starts in the glial cells of the brain or the spine. In some aspects, a glioma can be classified by specific type of cells with which they share histological features. Accordingly, a glioma that can be treated with EVs disclosed herein can be classified as an ependymoma (ependymal cells), astrocytoma (astrocytes), oligodendroglioma (oligodendrocytes), brainstem glioma (e.g., diffuse intrinsic pontine glioma), optic nerve glioma, mixed glioma, oligoastrocytoma, or any combination thereof. In certain aspects, an astrocytoma comprises glioblastoma multiforme (GBM).

Gliomas disclosed herein can be further categorized according to their grade, which is determined by pathologic evaluation of the tumor. In some aspects, the neuropathological evaluation and diagnostics of brain tumor specimens is performed according to WHO Classification of Tumours of the Central Nervous System. In some aspects, a glioma that can be treated with the present disclosure comprises a low-grade glioma. A “low-grade glioma” [WHO grade II] are well-differentiated (not anaplastic) and tend to exhibit benign tendencies and portend a better prognosis for the patient. However, in some aspects, low-grade gliomas can have a uniform rate of recurrence and increase in grade over time, so should be classified as malignant. In some aspects, a glioma that can be treated comprises a high grade glioma. A “high-grade glioma” [WHO grades III-IV] gliomas are undifferentiated or anaplastic and are malignant and carry a worse prognosis. Of numerous grading systems in use, the most common is the World Health Organization (WHO) grading system for astrocytoma, under which tumors are graded from I (least advanced disease—best prognosis) to IV (most advanced disease—worst prognosis). Non-limiting examples of high-grade gliomas include anaplastic astrocytomas and glioblastoma multiforme.

In some aspects, an EV disclosed herein can be used to treat a glioma grade I, grade II, grade III, grade IV, or combinations thereof, as determined under the WHO grading system. In certain aspects, an EV disclosed herein can be used to treat any type of gliomas.

In some aspects, the glioma treatable by the present methods is a diffuse intrinsic pontine glioma (DIPG), a type of brainstem glioma. Diffuse intrinsic pontine glioma primarily affects children, usually between the ages of 5 and 7. The median survival time with DIPG is under 12 months. Surgery to attempt tumor removal is usually not possible or advisable for DIPG. By their very nature, these tumors invade diffusely throughout the brain stem, growing between normal nerve cells.

In some aspects, the glioma treatable by the present methods is an IDH1 and IDH2-mutated glioma. Patients with glioma carrying mutations in either IDH1 or IDH2 have a relatively favorable survival, compared with patients with glioma with wild-type IDH1/2 genes. In WHO grade III glioma, IDH1/2-mutated glioma have a median prognosis of ˜3.5 years, whereas IDH1/2 wild-type glioma perform poor with a median overall survival of 1.5 years. In glioblastoma, the difference is larger.

In some aspects, an EV that can be used to treat a neurological disorder comprises an antigen (e.g., neuronal protein capable of misfolding) and one or more additional payloads. Non-limiting examples of payloads that are useful for the present disclosure are provided elsewhere in the present disclosure. In certain aspects, the additional payload comprises an adjuvant. In some aspects, the adjuvant comprises a stimulator of interferon genes protein (STING) agonist, toll-like receptor (TLR) agonist, inflammatory mediator, or combinations thereof.

In some aspects, the adjuvant is a STING agonist. In certain aspects, the STING agonist comprises a cyclic dinucleotide STING agonist or a non-cyclic dinucleotide STING agonist.

In some aspects, the adjuvant is a TLR agonist. In certain aspects, the TLR agonist comprises a TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA), or combinations thereof

### Methods of Treating an Autoimmune Disorder

As described herein, many neurological disorders can result when a subject's immune system is unable to mount a sufficient response to the antigen causing the neurological disorder. However, there are also neurological disorders that are caused by an aberrant or excessive immune response within the nervous system (e.g., multiple sclerosis). In some aspects, an EV disclosed herein can be used to treat such autoimmune disorders. As used herein, the term “autoimmune disorders” refers to any of a group of diseases characterized by abnormal functioning of the immune system causing one's immune system to produce antibodies against its own tissue(s). Non-limiting examples of autoimmune disorders include multiple sclerosis (MS), peripheral neuritis, Sjogren's syndrome, rheumatoid arthritis, alopecia, autoimmune pancreatitis, Behcet's disease, Bullous pemphigoid, Celiac disease, Devic's disease (neuromyelitis optica), Glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, Graves' disease, myasthenia gravis (MG), pemphigus vulgaris, anti-glomerular basement membrane disease (Goodpasture syndrome), Hashimoto's thyroiditis, autoimmune hepatitis, or combinations thereof. While the present disclosure is largely directed to neurological disorders, it will be apparent to a skilled artisan that the EVs disclosed herein can be used to treat any autoimmune disorders.

In some aspects, a method of treating an autoimmune disorder in a subject in need thereof, comprises administering to the subject an EV, which comprises an antigen, and wherein the EV is capable of targeting a cell within the CNS of the subject.

In some aspects, administering the EV to the subject can result in the induction of tolerogenic cells. In certain aspects, the induction of tolerogenic cells can improve one or more symptoms associated with the autoimmune disorder. As used herein, the term “tolerogenic cells” refers to cells with immunosuppressive properties (i.e., can reduce and/or inhibit an immune response). In some aspects, the tolerogenic cells can suppress an immune response by various methods, including, but not limited to, (i) production of anti-inflammatory cytokines (e.g., IL-10 or TGF-β), (ii) expression of inhibitory molecules (e.g., PD-L1 or PD-L2), (iii) production of immunosuppressive enzymes (e.g., indoleamine 2,3-dioxygenase (IDO) or heme oxygenase-1 (HO-1)), (iv) production of other immunosuppressive mediators (e.g., retinoic acid (RA), vitamin D3), or (v) combinations thereof. Non-limiting examples of tolerogenic cells that are useful for the present disclosure include tolerogenic dendritic cells, regulatory T cells (Tregs), liver sinusoidal endothelial cells (LSECs), Kupffer cells, or combinations thereof.

As described herein, an EV that can be used to treat an autoimmune disorder comprises an antigen. In some aspects, the antigen comprises a self-antigen, wherein the host's immune response against the self-antigen causes the autoimmune disorder. As used herein, the term “self-antigen” refers to an antigen that is expressed by a host cell or tissue. Under normal healthy state, such antigens are recognized by the body as self and do not elicit an immune response. However, under certain diseased conditions (e.g., autoimmune disorders disclosed herein), a body's own immune system can recognize self-antigens as foreign and mount an immune response against them, resulting in autoimmunity. Non-limiting examples of self-antigens (including the associated disease or disorder that can be treated with the present disclosure) include: (i) beta-cell proteins, insulin, islet antigen 2 (IA-2), glutamic acid decarboxylase (GAD65), and zinc transporter 8 (ZNT8) (type I diabetes), (ii) myelin oligodendrocyte glycoprotein (MOG), myelin basic protein (MBP), proteolipid protein (PLP), and myelin-associated glycoprotein (MAG) (multiple sclerosis), (iii) citrullinated antigens and synovial proteins (rheumatoid arthritis), (iv) aquaporin-4 (AQP4) (neuromyelitis optica), (v) nicotinic acetylcholine receptors (nAChRs) (myasthenia gravis), (vi) desmoglein-1 (DSG1) and desoglein-2 (DSG2) (pemphigus vulgaris), (v) thyrotropin receptor (Graves' disease), (vi) type IV collagen (Goodpasture syndrome), (vii) thyroglobulin, thyroid peroxidase, and thyroid-stimulating hormone receptor (TSHR) (Hashimoto's thyroiditis), or (viii) combinations thereof. In some aspects, the self-antigen is AQP4 and the autoimmune disorder is neuromyelitis optica (NMO). In some aspects, the self-antigen is MOG and the autoimmune disorder is multiple sclerosis (MS). In some aspects, the self-antigen is nAChR and the autoimmune disorder is myasthenia gravis (MG).

Not to be bound by any one theory, in some aspects, EVs disclosed herein (e.g., comprising a self-antigen) can treat an autoimmune disorder by inducing immune tolerance (e.g., against the autoreactive T cells and thereby, suppressing their activity). Accordingly, in some aspects, the present disclosure provides a method of inducing an immune tolerance in a subject in need thereof, comprising administering to the subject an EV, which comprises a payload, and wherein the EV is capable of targeting a cell within the CNS of the subject. In some aspects, the payload comprise an antigen. In certain aspects, the antigen comprises a self-antigen, such as those disclosed herein (e.g., associated with an autoimmune disorder).

As described herein, in some aspects, administering an EV of the present disclosure induces immune tolerance by increasing the amount of tolerogenic cells present in the subject (e.g., within the CNS). In some aspects, the amount of tolerogenic cells present in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold or more, compared to a reference (e.g., amount of tolerogenic cells present in the subject prior to the EV administration or in a corresponding subject that received the payload in a non-EV delivery vehicle). Non-limiting examples of tolerogenic cells that are useful for the present disclosure are provided elsewhere in the present disclosure.

In some aspects, an EV that can be used to treat an autoimmune disorder can comprise an antigen (e.g., self-antigen) and one or more additional payloads. Non-limiting examples of payloads that are useful for the present disclosure are provided elsewhere in the present disclosure. In certain aspects, the additional payload comprises an immune modulator. In some aspects, the immune modulator comprises a tolerance inducing agent (“tolerogen”). Non-limiting examples of tolerogens that can be used with the present disclosure include a NF-κB inhibitor, COX-2 inhibitor, mTOR inhibitor (e.g., rapamycin and derivatives), prostaglandins, nonsteroidal anti-inflammatory agents (NSAIDS), antileukotriene, aryl hydrocarbon receptor (AhR) ligand, vitamin D3, retinoic acid, steroids, Fas receptor/ligand, CD22 ligand, IL-10, IL-35, IL-27, metabolic regulator (e.g., glutamate), glycans (e.g., ES62, LewisX, LNFPIII), peroxisome proliferator-activated receptor (PPAR) agonists, immunoglobulin-like transcript (ILT) family of receptors (e.g., ILT3, ILT4, HLA-G, ILT-2), dexamethasone, or combinations thereof.

In some aspects, the tolerogen is rapamycin. In some aspects, the tolerogen is vitamin D3. In some aspects, the tolerogen is retinoic acid. In some aspects, the tolerogen is dexamethasone.

In some aspects, an immune modulator comprises a polynucleotide selected from a mRNA, miRNA, siRNA, antisense oligonucleotide (ASO), phosphorodiamidate morpholino oligomer (PMO), peptide-conjugated phosphorodiamidate morpholino oligomer (PPMO), shRNA, lncRNA, dsDNA, or combinations thereof. In certain aspects, the immune modulator is an ASO. In some aspects, the ASO is capable of inhibiting NF-κB, CD40, mTOR, or combinations thereof.

### Methods of Delivering a Payload to the CNS

As described herein, in contrast to antibodies, many small molecules, and other vaccines available in the art, EVs disclosed herein are capable of crossing the blood-brain barrier. Accordingly, in some aspects, the present disclosure relates to a method of delivering a payload (e.g., antigen) to the CNS of a subject, In some aspects, such a method comprises administering to the subject an EV, wherein the EV comprises the payload, and wherein the EV is capable of targeting a cell within the CNS of the subject.

In some aspects, administering the EV disclosed herein increases the amount of payloads (e.g., antigen) that are delivered to the CNS (e.g., to a cell within the CNS). In certain aspects, the amount of payloads delivered to the CNS is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold or more, compared to a reference (e.g., amount of payload delivered to the CNS using a non-EV delivery vehicle). In some aspects, increasing the amount of payloads (e.g., antigen) that are delivered to the CNS can treat a neurological disorder and/or reduce/alleviate one or more symptoms of the neurological disorder.

In some aspects, the payload comprises an antigen. In certain aspects, the antigen comprises a neuronal protein that is capable of misfolding (e.g., such as those associated with a neurological disorder described herein). In some aspects, the antigen comprises a self-antigen. In some aspects, the antigen comprises both a neuronal protein and a self-antigen.

In some aspects, an EV that can be used to deliver a payload to the CNS of a subject comprises at least 2, 3, 4, 5, 6, 7, 8, 9, 10 or more payloads. Non-limiting examples of payloads that are useful for the present disclosure are provided elsewhere in the present disclosure.

### Methods of Modulating a Germinal Center Response

In some aspects, EVs of the present disclosure can treat and/or improve one or more symptoms of a neurological disorder, by promoting the induction of antibodies that are capable of specifically binding to an antigen associated with the neurological disorder. Germinal centers play an important role in the production of such antibodies. See Stebegg et al., Front Immunol 9:2469 (October 2018). As used herein, the term “germinal centers” refers to sites within secondary lymphoid organs (e.g., lymph nodes and the spleen) where mature B cells proliferate, differentiate, and mutate their antibody genes through somatic hypermutation, resulting in the production of antibodies with great binding affinity. In some aspects, the present disclosure is related to a method of modulating a germinal center response to an antigen in a subject in need thereof, comprising administering to the subject an EV, which comprise the antigen, and wherein the EV is capable of targeting a cell within the CNS of the subject.

In some aspects, administering the EV to the subject increases the germinal center response in the subject. In some aspects, the germinal center response is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold or more, compared to a reference (e.g., germinal center response in subject prior to the EV administration or in a corresponding subject that received the antigen in a non-EV delivery vehicle). In certain aspects, the increase in the germinal center response results in increased production of antigen-specific antibodies in the subject. In some aspects, the increase in the germinal center response increases the binding affinity of the antigen-specific antibodies produced.

In some aspects, administering the EV disclosed herein decreases the germinal center response in the subject. In some aspects, the germinal center response is decreased by at least about 5%, at least about 10%, at least about 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% after the administration of the EV. In certain aspects, the decrease in the germinal center response results in decreased production of antigen-specific antibodies in the subject. In some aspects, the decrease in the germinal center response decreases the binding affinity of the antigen-specific antibodies produced.

In some aspects, an EV that can be used to modulate a germinal center response comprises an antigen and one or more additional payloads. Non-limiting examples of payloads that are useful for the present disclosure are provided elsewhere in the present disclosure.

As described herein, an EV useful for the present disclosure (e.g., to treat a neurological disorder, to deliver a payload to the CNS, to treat autoimmune disorder, to induce immune tolerance, and/or to modulate a germinal center response) are capable of crossing the blood-brain barrier and targeting a cell within the CNS of a subject. In some aspects, a cell that can be targeted with EVs disclosed herein is an immune cell. In certain aspects, the immune cell comprises a dendritic cell, macrophage, T cells, B cells, or combinations thereof. In certain aspects, the immune cell is a dendritic cell. In some aspects, the immune cell is a macrophage.

In some aspects, an EV that can be used with the methods disclosed herein comprises one or more additional payloads. In certain aspects, the additional payload comprises an antigen, adjuvant, immune modulator, or combinations thereof.

In some aspects, the EV further comprises a targeting moiety. In certain aspects, the targeting moiety is capable of specifically binding to a marker expressed on a cell within the CNS of the subject. In some aspects, the marker is expressed only on dendritic cells. In certain aspects, the marker comprises a C-type lectin domain family 9 member A (Clec9a) protein, a dendritic cell-specific intercellular adhesion molecule grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), BDCA-2, BDCA-3, Dectin-2, Bst-2 (CD317), CD11c, XCR1, or combinations thereof. In some aspects, the marker is expressed only on macrophages. In certain aspects, the marker comprises CD14, CD16, CD64, CD68, CD71, CCR5, or combinations thereof. Additional disclosure regarding payloads that can be used with the present disclosure are provided elsewhere herein.

In the above methods disclosed herein, the EV can be administered to the subject by any relevant method known in the art. Non-limiting examples of different routes of administration that can be used include: intravenous, intraperitoneal, intramuscular, subcutaneous, spinal or other parenteral routes of administration, for example by injection or infusion. The phrase “parenteral administration,” as used herein, refers to modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, an EV described herein can be administered via a non-parenteral route, such as a topical, epidermal or mucosal route of administration, for example, intranasally, orally, vaginally, rectally, sublingually or topically.

In some aspects, the EV is administered via intrathecal, intraocular, intracranial, intranasal, perineural, or combinations thereof. In some aspects, the EVs are administered via an injection into the spinal canal, or into the subarachnoid space so that it reaches the cerebrospinal fluid (CSF). In some aspects, the EVs are administered intratumorally into one or more tumors (e.g., brain tumor) of the subject. In some aspects, the EV is administered intraocularly, wherein the intraocular administration is selected from intravitreal, intracameral, subconjunctival, subretinal, subscleral, intrachoroidal, or combinations thereof. In some aspects, the EV is administered intracranially, wherein the intracranial administration is selected from intracisternal, subarachnoidal, intrahippocampal, intracerebroventricular, intraparenchymal, or combinations thereof. In some aspects, the EV is administered intranasally, wherein the intranasal administration is by instillation or injection. In some aspects, the EV is administered perinerually, wherein in the perineural administration is by facial intradermal injection.

In any of the administering methods described herein, in some aspects, an EV can be administered to a subject using a “prime-pull” dosing regimen. As used herein, the term “prime-pull” dosing regimen refers to an administration schedule in which a subject is first immunized with a first dosing regimen (also referred to herein as the “priming dose”) and then subsequently receives a second dosing regimen (also referred to herein as the “boosting dose”). In certain aspects, the first dosing regimen comprises a first EV and the second dosing regimen comprises a second EV, wherein the first and second EVs differ in their composition. For instance, in certain aspects, the first EV comprises an antigen and one or more of the other moieties described herein (e.g., adjuvant, immunomodulatory, and/or targeting moiety), and the second EV comprises the antigen but not the one or more of the other moieties present in the first EV. In some aspects, the first dosing regimen and the second dosing regimen are administered to the subject by different routes of administration (e.g., any combination of routes of administration that are known in the art and/or disclosed herein).

Accordingly, in some aspects, an administration (or vaccinating) method described herein comprises (i) administering a priming dose to a subject, wherein the priming dose comprises a first EV, which comprises an antigen and an adjuvant, and (ii) administering a boosting dose to the subject, wherein the boosting dose comprises a second EV, which comprises the antigen but not the adjuvant present in the first EV. In some aspects, the second EV does not comprise any adjuvant. Not to be bound by any one theory, in some aspects, the prime-pull dosing regimens can further improve the safety of the EV-based vaccines described herein, e.g., by not requiring the use of adjuvants when administering the boosting dose to the subject and thereby, avoid the risk of non-specific inflammation that can occur with certain adjuvants.

Moreover, in some aspects, the use of prime-pull dosing regimen can enhance the migration of immune cells to the CNS of a subject. For instance, in certain aspects, a subject receives a first dosing regimen to prime or activate one or more immune cells (e.g., B cells), and then subsequently receives a second dosing regimen, wherein the second dosing regimen is capable of promoting the migration of the primed immune cells to the CNS. In some aspects, this can be achieved by (i) administering the second dosing regimen using a CNS-specific route of administration, (ii) modifying the EVs of the second dosing regimen to comprise one or more CNS-specific targeting moieties, or (iii) both (i) and (ii). Non-limiting examples of such routes of administration and targeting moieties are provided throughout the present disclosure.

In some aspects, EVs disclosed herein are compartmentally administered to a subject. As used herein, the “compartmental” administration refers to the localized delivery of an EV to a subject. For example, in some aspects, the compartmental administration comprises administering the EVs directly to the brain, e.g., by intracranial administration. In some aspects, the compartmental administration comprises administering the EVs directly to the spinal cord, e.g., by intrathecal administration.

In some aspects, the EVs are administered by intrathecal administration, followed by application of a mechanical convective force to the torso. See, e.g., Verma et al., Alzheimer's Dement. 12:e12030 (2020); which is incorporated by reference herein in its entirety). As such, certain aspects of the present disclosure are directed to methods of administering an EV to a subject in need thereof, comprising administering the EV to the subject by intrathecal injection, followed by applying a mechanical convective force to the torso of the subject. In some aspects, the mechanical convective force is achieved using a high frequency chest wall or lumbothoracic oscillating respiratory clearance device (e.g., a Smart Vest or Smart Wrap, ELECTROMED INC, New Prague, Minn., USA). In some aspects, the mechanical convective force, e.g., the oscillating vest, facilitates spread of the intrathecally dosed EVs, further down the nerve thus allowing for better EV delivery to nerves.

In some aspects, the intra- and trans-compartmental biodistribution of exosomes can be manipulated by exogenous extracorporeal forces acting upon a subject after compartmental delivery of exosomes. This includes the application of mechanical convection, for example by way of applying percussion, vibration, shaking, or massaging of a body compartment or the entire body. Following intrathecal dosing for example, the application of chest wall vibrations by several means including an oscillating mechanical jacket can spread the biodistribution of the EVs along the neuraxis or along cranial and spinal nerves, which can be helpful in the treatment of nerve disorders by drug carrying exosomes.

In some aspects, the application of external mechanical convective forces via an oscillating jacket or other similar means can be used to remove EVs and other material from the cerebrospinal fluid of the intrathecal space and out to the peripheral circulation. This aspect can help remove endogenous toxic exosomes and other deleterious macromolecules such as beta-amyloid, tau, alpha-synuclein, TDP43, neurofilament and excessive cerebrospinal fluid from the intrathecal space to the periphery for elimination.

In some aspects, exosomes delivered via the intracerebroventricular route can be made to translocate throughout the neuraxis by simultaneously incorporating a lumbar puncture and allowing for ventriculo-lumbar perfusion wherein additional fluid is infused into the ventricles after exosome dosing, while allowing the existing neuraxial column of CSF to exit is the lumbar puncture. Ventriculo-lumbar perfusion can allow ICV dosed EVs to spread along the entire neuraxis and completely cover the subarachnoid space in order to treat leptomeningeal cancer and other diseases.

In some aspects, the application of external extracorporeal focused ultrasound, thermal energy (heat) or cold may be used to manipulate the compartmental pharmacokinetics and drug release properties of exosomes engineered to be sensitive to these phenomena.

In some aspects, the intracompartmental behavior and biodistribution of exosomes engineered to contain paramagnetic material can be manipulated by the external application of magnets or a magnetic field.

In some aspects, a subject that can be treated with the present disclosure is a human. In some aspects, a subject is a non-human mammal (e.g., non-human primates, dogs, cats, guinea pigs, rabbits, rats, mice, horses, cattle, chickens, birds, and bears). Accordingly, in some aspects, the EVs disclosed herein can be used to improve the health of an animal (i.e., non-human mammal).

### Methods of Producing EVs

In some aspects, the present disclosure is also directed to methods of producing EVs described herein (e.g., modular or “plug and play” EVs). In some aspects, the method comprises: obtaining the EV from a producer cell, wherein the producer cell contains two or more components of the EV (e.g., (i) antigen and adjuvant, (ii) antigen and immune modulator, or (iii) antigen, adjuvant, and immune modulator); and optionally isolating the obtained EV. In some aspects, the method comprises: modifying a producer cell by introducing two or more components of an exosome disclosed herein (e.g., (i) antigen and adjuvant, (ii) antigen and immune modulator, or (iii) antigen, adjuvant, and immune modulator); obtaining the EV from the modified producer cell; and optionally isolating the obtained EV. In further aspects, the method comprises: obtaining an exosome from a producer cell; isolating the obtained exosome; and modifying the isolated exosome (e.g., by inserting an exogenous antigen, adjuvant, and/or immune modulator). In certain aspects, the method further comprises formulating the isolated exosome into a pharmaceutical composition.

As will be apparent from the present disclosure, one of the features of the EV-based vaccine platform disclosed herein (e.g., modular or “plug and play” EVs) is that the base EVs can be produced and stored indefinitely until they are to be used with the methods disclosed herein. For instance, as described herein, in producing the base EVs, they can be initially produced to comprise one or more moieties of interest, such as those that could be beneficial in a wide range of diseases or disorders (e.g., adjuvant and/or targeting moiety). Then, when desired, the base EVs can be rapidly modified by simply plugging or clipping on a specific antigen of interest, such as those useful to treat a neurological disorder, and thereby, produce or manufacture a vaccine that can be used to treat a disease or disorder described herein (e.g., neurological disorder). Such antigens can be added to the base EVs at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 2 years, at least about 3 years, at least about 4 years, or at least about 5 year or more after isolating the base EV from the producer cell.

Accordingly, in some aspects, the present disclosure is also directed to methods of producing the base EVs described herein. In some aspects, the method comprises: obtaining the base EV from a producer cell, wherein the producer cell contains one or more molecules of interest described herein (e.g., adjuvant, targeting moiety and/or scaffold moiety); and optionally isolating the obtained EV. In certain aspects, the producer cell contains an adjuvant. In some aspects, the producer cell contains a targeting moiety (e.g., tropism moieties targeting motor neurons described herein). In certain aspects, the producer contains any combination of an adjuvant and/or targeting moiety. In some aspects, the method comprises: modifying a producer cell by introducing one or more molecules of interest (e.g., adjuvant, targeting moiety, and/or scaffold moiety); obtaining the EV from the modified producer cell; and optionally isolating the obtained EV. Any of the producer cells described herein can be used with the methods of producing base EVs described herein.

As will be apparent to those skilled in the arts, the ability to simply “plug” or “clip on” an antigen of interest to a base EV (i.e., EVs that have been isolated from a producer cell) can be greatly advantageous when seeking to treat a disease or disorder that is more regional and/or more prevalent in certain individual or subsets of individuals (e.g., age group). As described herein, a base EV can differ from a naturally existing EV. For example, the base EVs can be genetically modified (e.g., by introducing a moiety of interest into the producer cells during production) or they can be modified after the EVs are produced and isolated from the producer cells.

In some aspects, EV-based vaccines that can be produced or manufactured using the methods described herein are individualized vaccines. As used herein, the term “individualized vaccines” and “personalized vaccines” can be used interchangeably and refer to vaccines that are tailored to a specific individual or subsets of individuals. Such a personalized vaccine could be of particular interest for diseases and disorders that are more prevalent in certain individuals or subsets of individuals (e.g., those who share certain genetic background). In some aspects, the methods disclosed herein can be used to produce or manufacture such individualized or personalized vaccines by adding an antigen to an EV that has been isolated from a producer cell, wherein the antigen has been determined to have (or likely to have) a therapeutic effect (e.g., induces an immune response) in the particular individual or subset of individuals.

As will be apparent to those skilled in the arts, methods of identifying individual-specific antigens are known in the art. See, e.g., US 2009/0169576; US 2014/0178438; Sakkhachornphop, S., et al., J Virol Methods 217: 70-8 (June 2015); and Xu, K., et al., Sci Rep 8(1): 1067 (January 2018), each of which is incorporated herein by reference in its entirety.

In some aspects, an EV-based vaccine that can be prepared or manufactured using the methods described herein can comprise one or more additional moieties, such as those that are capable of enhancing the therapeutic efficacy of the vaccine.

In some aspects, the additional moiety comprises an adjuvant. Accordingly, in some aspects, a base EV that can be used with the methods disclosed herein comprises an adjuvant, such that the adjuvant is present in the EV prior to the addition of the antigen. In certain aspects, the method of preparing or manufacturing an EV-based vaccine provided herein further comprises adding an adjuvant to an EV that has been isolated from a producer cell (i.e., base EV). In some aspects, the adjuvant is added to the EV before adding the antigen. In some aspects, the adjuvant is added to the EV after adding the antigen. In some aspects, the adjuvant is added to the EV together with the antigen. Non-limiting examples of adjuvants that can be used with the present methods are provided elsewhere in the present disclosure.

In some aspects, the additional moiety comprises a targeting moiety. Accordingly, in some aspects, a base EV that can be used with the methods disclosed herein further comprises a targeting moiety, such that one or more of the additional moieties are present in the EV prior to the addition of the antigen. In certain aspects, the method of preparing or manufacturing an EV-based vaccine provided herein further comprises adding a targeting moiety to an EV that has been isolated from a producer cell (i.e., base EV). In some aspects, the targeting moiety is added to the EV before adding the antigen. In some aspects, the targeting moiety is added to the EV after adding the antigen. In some aspects, the targeting moiety is added to the EV together with the antigen. Non-limiting examples of targeting moiety that can be used with the present methods are provided elsewhere in the present disclosure.

As described herein, in producing or manufacturing an EV-based vaccine with the methods disclosed herein, an antigen or any other molecules of interest (e.g., adjuvant and/or targeting moiety) can be added to the base EV, such that the antigen (or any other molecule of interest) is associated with a surface of the EV or in the lumen of the EV. For instance, in some aspects, an antigen is: (i) linked directly to the exterior surface of the EV, (ii) linked directly to the luminal surface of the EV, (iii) in the lumen of the EV, or (iv) any combination thereof. In some aspects, an adjuvant is: (i) linked directly to the exterior surface of the EV, (ii) linked directly to the luminal surface of the EV, (iii) in the lumen of the EV, or (iv) any combination thereof. In some aspects, a targeting moiety is: (i) linked directly to the exterior surface of the EV, (ii) linked directly to the luminal surface of the EV, (iii) in the lumen of the EV, or (iv) any combination thereof.

In some aspects, any suitable method can be used to link an antigen or any other molecules of interest (e.g., adjuvant and/or targeting moiety) to an exterior surface and/or luminal surface of the EV. In certain aspects, the antigen or any other molecules of interest (e.g., adjuvant and/or targeting moiety) is linked to the exterior surface and/or the luminal surface of the EV by any suitable coupling strategies known in the art. In some aspects, the coupling strategy comprises: an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof. In some aspects, the anchoring moiety comprises a cholesterol, fatty acid (e.g., palmitate), tocopherol (e.g., vitamin E), alkyl chain, aromatic ring, or any combination thereof. In some aspects, the chemical conjugation comprises a maleimide moiety, copper-free, biorthogonal click chemistry (e.g., azide/strained alkyne (DIFO)), metal-catalyzed click chemistry (e.g., CUAAC, RUAAC), or any combination thereof. Additional description relating to the different approaches of linking an antigen or any other molecules of interest (e.g., adjuvant and/or targeting moiety) are provided elsewhere in the present disclosure.

In some aspects, an EV that can be prepared or manufactured using the methods described herein can further comprise a scaffold moiety. In certain aspects, the EV comprises the scaffold moiety prior to the addition of the antigen to the EV. In some aspects, the methods of preparing or manufacturing EV-based vaccines described herein further comprise adding a scaffold moiety to an EV that has been isolated from a producer cell (i.e., base EV). In some aspects, the scaffold moiety is added to the EV before adding the antigen. In some aspects, the scaffold moiety is added to the EV after adding the antigen. In some aspects, the scaffold moiety is added to the EV together with the antigen.

Accordingly, in some aspects, an antigen is linked to a scaffold moiety on the exterior surface and/or luminal surface of the EV. In some aspects, an adjuvant is linked to a scaffold moiety on the exterior surface and/or luminal surface of the EV. In some aspects, a targeting moiety is linked to a scaffold moiety on the exterior surface and/or luminal surface of the EV. In some aspects, any combination of an antigen, adjuvant, and/or targeting moiety are linked to a scaffold moiety on the exterior surface and/or luminal surface of the EV.

In some aspects, the scaffold moiety comprises Scaffold X (e.g., PTGFRN or a fragment thereof). In some aspects, the scaffold moiety comprises Scaffold Y (e.g., BASP-1 or a fragment thereof). In some aspects, the scaffold moiety comprises both Scaffold X (e.g., PTGFRN or a fragment thereof) and Scaffold Y (e.g., BASP-1 or a fragment thereof).

Non-limiting examples of scaffold moieties that can be used with the present methods are provided elsewhere in the present disclosure.

### Methods of Modifying a Producer Cell

As described supra, in some aspects, a method of producing an exosome comprises modifying a producer cell with one or more moieties (e.g., antigen, adjuvant, and/or immune modulator). In certain aspects, the one or more moieties comprise an antigen, adjuvant, or immune modulator. In some aspects, the one or more moieties further comprise a scaffold moiety disclosed herein (e.g., Scaffold X or Scaffold Y).

In some aspects, the producer cell can be a mammalian cell line, a plant cell line, an insect cell line, a fungi cell line, or a prokaryotic cell line. In certain aspects, the producer cell is a mammalian cell line. Non-limiting examples of mammalian cell lines include: a human embryonic kidney (HEK) cell line, a Chinese hamster ovary (CHO) cell line, an HT-1080 cell line, a HeLa cell line, a PERC-6 cell line, a CEVEC cell line, a fibroblast cell line, an amniocyte cell line, an epithelial cell line, a mesenchymal stem cell (MSC) cell line, and combinations thereof. In certain aspects, the mammalian cell line comprises HEK-293 cells, BJ human foreskin fibroblast cells, fHDF fibroblast cells, AGE.HN® neuronal precursor cells, CAP® amniocyte cells, adipose mesenchymal stem cells, RPTEC/TERT1 cells, or combinations thereof. In some aspects, the producer cell is a primary cell. In certain aspects, the primary cell can be a primary mammalian cell, a primary plant cell, a primary insect cell, a primary fungi cell, or a primary prokaryotic cell.

In some aspects, the one or more moieties can be a transgene or mRNA, and introduced into the producer cell by transfection, viral transduction, electroporation, extrusion, sonication, cell fusion, or other methods that are known to the skilled in the art.

In some aspects, the one or more moieties is introduced to the producer cell by transfection. In some aspects, the one or more moieties can be introduced into suitable producer cells using synthetic macromolecules, such as cationic lipids and polymers (Papapetrou et al., Gene Therapy 12: S118-S130 (2005)). In some aspects, the cationic lipids form complexes with the one or more moieties through charge interactions. In some of these aspects, the positively charged complexes bind to the negatively charged cell surface and are taken up by the cell by endocytosis. In some other aspects, a cationic polymer can be used to transfect producer cells. In some of these aspects, the cationic polymer is polyethylenimine (PEI). In certain aspects, chemicals such as calcium phosphate, cyclodextrin, or polybrene, can be used to introduce the one or more moieties to the producer cells. The one or more moieties can also be introduced into a producer cell using a physical method such as particle-mediated transfection, “gene gun”, biolistics, or particle bombardment technology (Papapetrou et al., Gene Therapy 12: S118-S130 (2005)). A reporter gene such as, for example, beta-galactosidase, chloramphenicol acetyltransferase, luciferase, or green fluorescent protein can be used to assess the transfection efficiency of the producer cell.

In certain aspects, the one or more moieties are introduced to the producer cell by viral transduction. A number of viruses can be used as gene transfer vehicles, including moloney murine leukemia virus (MMLV), adenovirus, adeno-associated virus (AAV), herpes simplex virus (HSV), lentiviruses, and spumaviruses. The viral mediated gene transfer vehicles comprise vectors based on DNA viruses, such as adenovirus, adeno-associated virus and herpes virus, as well as retroviral based vectors.

In certain aspects, the one or more moieties are introduced to the producer cell by electroporation. Electroporation creates transient pores in the cell membrane, allowing for the introduction of various molecules into the cell. In some aspects, DNA and RNA as well as polypeptides and non-polypeptide therapeutic agents can be introduced into the producer cell by electroporation.

In certain aspects, the one or more moieties introduced to the producer cell by microinjection. In some aspects, a glass micropipette can be used to inject the one or more moieties into the producer cell at the microscopic level.

In certain aspects, the one or more moieties are introduced to the producer cell by extrusion.

In certain aspects, the one or more moieties are introduced to the producer cell by sonication. In some aspects, the producer cell is exposed to high intensity sound waves, causing transient disruption of the cell membrane allowing loading of the one or more moieties.

In certain aspects, the one or more moieties are introduced to the producer cell by cell fusion. In some aspects, the one or more moieties are introduced by electrical cell fusion. In other aspects, polyethylene glycol (PEG) is used to fuse the producer cells. In further aspects, sendai virus is used to fuse the producer cells.

In some aspects, the one or more moieties are introduced to the producer cell by hypotonic lysis. In such aspects, the producer cell can be exposed to low ionic strength buffer causing them to burst allowing loading of the one or more moieties. In other aspects, controlled dialysis against a hypotonic solution can be used to swell the producer cell and to create pores in the producer cell membrane. The producer cell is subsequently exposed to conditions that allow resealing of the membrane.

In some aspects, the one or more moieties are introduced to the producer cell by detergent treatment. In certain aspects, producer cell is treated with a mild detergent which transiently compromises the producer cell membrane by creating pores allowing loading of the one or more moieties. After producer cells are loaded, the detergent is washed away thereby resealing the membrane.

In some aspects, the one or more moieties introduced to the producer cell by receptor mediated endocytosis. In certain aspects, producer cells have a surface receptor which upon binding of the one or more moieties induces internalization of the receptor and the associated moieties.

In some aspects, the one or more moieties are introduced to the producer cell by filtration. In certain aspects, the producer cells and the one or more moieties can be forced through a filter of pore size smaller than the producer cell causing transient disruption of the producer cell membrane and allowing the one or more moieties to enter the producer cell.

In some aspects, the producer cell is subjected to several freeze thaw cycles, resulting in cell membrane disruption allowing loading of the one or more moieties.

### Methods of Modifying an EV

In some aspects, a method of producing an exosome comprises modifying the isolated exosome by directly introducing one or more moieties into the EVs. In certain aspects, the one or more moieties comprise an antigen, adjuvant, or immune modulator. In some aspects, the one or more moieties comprise a scaffold moiety disclosed herein (e.g., Scaffold X or Scaffold Y).

In certain aspects, the one or more moieties are introduced to the exosome by transfection. In some aspects, the one or more moieties can be introduced into the EV using synthetic macromolecules such as cationic lipids and polymers (Papapetrou et al., Gene Therapy 12: S118-S130 (2005)). In certain aspects, chemicals such as calcium phosphate, cyclodextrin, or polybrene, can be used to introduce the one or more moieties to the EV.

In certain aspects, the one or more moieties are introduced to the EV by electroporation. In some aspects, exosomes are exposed to an electrical field which causes transient holes in the EV membrane, allowing loading of the one or more moieties.

In certain aspects, the one or more moieties are introduced to the EV by microinjection. In some aspects, a glass micropipette can be used to inject the one or more moieties directly into the EV at the microscopic level.

In certain aspects, the one or more moieties are introduced to the EV by extrusion.

In certain aspects, the one or more moieties are introduced to the EV by sonication. In some aspects, EVs are exposed to high intensity sound waves, causing transient disruption of the EV membrane allowing loading of the one or more moieties.

In some aspects, one or more moieties can be conjugated to the surface of the EV. Conjugation can be achieved chemically or enzymatically, by methods known in the art.

In some aspects, the EV comprises one or more moieties that are chemically conjugated. Chemical conjugation can be accomplished by covalent bonding of the one or more moieties to another molecule, with or without use of a linker. The formation of such conjugates is within the skill of artisans and various techniques are known for accomplishing the conjugation, with the choice of the particular technique being guided by the materials to be conjugated. In certain aspects, polypeptides are conjugated to the EV. In some aspects, non-polypeptides, such as lipids, carbohydrates, nucleic acids, and small molecules, are conjugated to the EV.

In some aspects, the one or more moieties are introduced to the EV by hypotonic lysis. In such aspects, the EVs can be exposed to low ionic strength buffer causing them to burst allowing loading of the one or more moieties. In other aspects, controlled dialysis against a hypotonic solution can be used to swell the EV and to create pores in the EV membrane. The EV is subsequently exposed to conditions that allow resealing of the membrane.

In some aspects, the one or more moieties are introduced to the EV by detergent treatment. In certain aspects, extracellular vesicles are treated with a mild detergent which transiently compromises the EV membrane by creating pores allowing loading of the one or more moieties. After EVs are loaded, the detergent is washed away thereby resealing the membrane.

In some aspects, the one or more moieties are introduced to the EV by receptor mediated endocytosis. In certain aspects, EVs have a surface receptor which upon binding of the one or more moieties induces internalization of the receptor and the associated moieties.

In some aspects, the one or more moieties are introduced to the EV by mechanical firing. In certain aspects, extracellular vesicles can be bombarded with one or more moieties attached to a heavy or charged particle such as gold microcarriers. In some of these aspects, the particle can be mechanically or electrically accelerated such that it traverses the EV membrane.

In some aspects, extracellular vesicles are subjected to several freeze thaw cycles, resulting in EV membrane disruption allowing loading of the one or more moieties.

### Methods of Isolating an EV

In some aspects, methods of producing EVs disclosed herein comprises isolating the EV from the producer cells. In certain aspects, the EVs released by the producer cell into the cell culture medium. It is contemplated that all known manners of isolation of EVs are deemed suitable for use herein. For example, physical properties of EVs can be employed to separate them from a medium or other source material, including separation on the basis of electrical charge (e.g., electrophoretic separation), size (e.g., filtration, molecular sieving, etc.), density (e.g., regular or gradient centrifugation), Svedberg constant (e.g., sedimentation with or without external force, etc.). Alternatively, or additionally, isolation can be based on one or more biological properties, and include methods that can employ surface markers (e.g., for precipitation, reversible binding to solid phase, FACS separation, specific ligand binding, non-specific ligand binding, affinity purification etc.).

Isolation and enrichment can be done in a general and non-selective manner, typically including serial centrifugation. Alternatively, isolation and enrichment can be done in a more specific and selective manner, such as using EV or producer cell-specific surface markers. For example, specific surface markers can be used in immunoprecipitation, FACS sorting, affinity purification, and magnetic separation with bead-bound ligands.

In some aspects, size exclusion chromatography can be utilized to isolate the EVs. Size exclusion chromatography techniques are known in the art. Exemplary, non-limiting techniques are provided herein. In some aspects, a void volume fraction is isolated and comprises the EVs of interest. Further, in some aspects, the EVs can be further isolated after chromatographic separation by centrifugation techniques (of one or more chromatography fractions), as is generally known in the art. In some aspects, for example, density gradient centrifugation can be utilized to further isolate the extracellular vesicles. In certain aspects, it can be desirable to further separate the producer cell-derived EVs from EVs of other origin. For example, the producer cell-derived EVs can be separated from non-producer cell-derived EVs by immunosorbent capture using an antigen antibody specific for the producer cell.

In some aspects, the isolation of EVs can involve combinations of methods that include, but are not limited to, differential centrifugation, size-based membrane filtration, immunoprecipitation, FACS sorting, and magnetic separation.

### II. Extracellular Vesicles

Disclosed herein are EVs, that can be used with the methods disclosed herein. As described herein, EVs described herein differ from other vaccine platforms for treating diseases and disorders (e.g., neurological disorders described herein) in that the EVs comprise one or more of the following properties: (i) flexibility of moiety (e.g., antigen) display, (ii) diverse adjuvant and immunomodulatory combinations, (iii) enhanced cell-specific tropism, (iv) selectively promoting T-cell, B-cell, or Treg/tolerogenic immune responses, or (v) any combination thereof.

In some aspects, EVs of the present disclosure provide flexibility of moiety display. For instance, the moieties of interest (e.g., antigen) (i) can be directly linked to a surface of the EV (e.g., exterior surface and/or luminal surface), (ii) can be linked to a scaffold moiety (e.g., Scaffold X and/or Scaffold Y) and then expressed on a surface of the EV (e.g., exterior surface and/or luminal surface), (iii) can be expressed in the lumen of the EV, or (iv) combinations thereof. Such ability to rapidly engineer EVs is particularly useful in developing EV-based vaccines for treating the diseases and disorders described herein. For instance, a single EV engineered to express certain payloads and/or targeting moieties can be used in treating a wide range of diseases or disorders by simply “plugging” a moiety (e.g., antigen of interest) into the EVs (or rapidly attaching a moiety (e.g., antigen of interest) as a “clip-on” attachment to the EVs). Methods of producing such modular or “plug and play” EVs are provided elsewhere in the present disclosure.

In some aspects, EVs of the present disclosure allow for the diverse combinations of different moieties of interest (e.g., antigens, adjuvants, immunomodulators, and/or targeting moieties). In certain aspects, the EVs allow for the combination of a wide range of adjuvants and immunomodulators. Non-limiting examples of adjuvants and immunomodulators that can be combined in a single EV include small molecule agonists (e.g., STING), small molecule antagonists, co-stimulatory proteins, anti-sense and bacterial adjuvant oligonucleotides. Additional disclosure relating to the different moieties that can be combined together are provided elsewhere in the present disclosure.

In some aspects, EVs described herein can be engineered to exhibit enhanced cell-specific tropism. For instance, the EVs can be engineered to express on their exterior surface a targeting moiety (e.g., antibodies and/or proteins) that can specifically bind to a marker on a specific cell. In some aspects, EVs described herein can be engineered to induce certain types of immune responses (e.g., T cell, B cell, and/or Treg/tolerogenic immune responses). Additional disclosure relating to such properties are provided elsewhere in the present disclosure.

As described herein, in some aspects, EVs described herein are capable of inducing a humoral immune response when administered to a subject (e.g., suffering from a neurological disorder). In some aspects, the EVs are capable of inducing B cell-mediated immunity (e.g., comprising robust antibody responses that can be prophylactic and/or therapeutic). In some aspects, the EVs disclosed herein can specifically target proteinopathy antigens (e.g., B cells antigens, such as Aβ, tau, and alpha-synuclein). In some aspects, the EVs disclosed herein do not express T cell epitopes. In some aspects, the EVs comprise TLR4 or TLR9 agonists (i.e., adjuvants) and thereby include Th1 antibody responses.

In some aspects, the EVs useful in the present disclosure have been engineered to produce multiple agents (i.e., payloads) together (e.g., an antigen and an adjuvant in a single EV; an antigen and an immune modulator in a single EV; and an antigen, an adjuvant, and an immune modulator in a single EV; instead of a single agent, e.g., an antigen alone, an adjuvant alone, or an immune modulator alone). As described herein, in some aspects, the EVs are capable of targeting a cell (e.g., immune cell) within the CNS of a subject.

In some aspects, an EV comprises (i) an antigen and (ii) an adjuvant. In other aspects, an EV comprises (i) an antigen and (ii) an immune modulator. In some aspects, an EV comprises (i) an antigen, (ii) an adjuvant, and (iii) an immune modulator. In certain aspects, an EV disclosed herein can also comprise a targeting moiety.

As will be apparent to those skilled in the art, EVs disclosed herein do not need to comprise an antigen and can instead comprise multiple other payloads disclosed herein. For example, in some aspects, an EV can comprise multiple different adjuvants. In some aspects, an EV can comprise multiple different immune modulators. In some aspects, an EV can comprise one or more adjuvants in combination with one or more immune modulators. Such antigen-less EVs can be useful in inducing and/or increasing an innate immune response. Non-limiting examples of therapeutic settings where such antigen-less EVs could be useful include: to treat bacterial and/or viral infections, such as Pseudomonas aeruginosa for ventilator-associated pneumonia, influenza and RSV, and staph aureus for surgical site infection. Unless indicated otherwise, the relevant disclosures provided herein are equally applicable regardless of whether an EV comprises an antigen or not.

As described supra, EVs described herein are extracellular vesicles with a diameter between about 20-300 nm. In certain aspects, an EV of the present disclosure has a diameter between about 20-290 nm, between about 20-280 nm, between about 20-270 nm, between about 20-260 nm, between about 20-250 nm, between about 20-240 nm, between about 20-230 nm, between about 20-220 nm, between about 20-210 nm, between about 20-200 nm, between about 20-190 nm, between about 20-180 nm, between about 20-170 nm, between about 20-160 nm, between about 20-150 nm, between about 20-140 nm, between about 20-130 nm, between about 20-120 nm, between about 20-110 nm, between about 20-100 nm, between about 20-90 nm, between about 20-80 nm, between about 20-70 nm, between about 20-60 nm, between about 20-50 nm, between about 20-40 nm, between about 20-30 nm. The size of the EV described herein can be measured according to methods described, infra.

In some aspects, an EV of the present disclosure comprises a bi-lipid membrane (“EV membrane”), comprising an interior surface and an exterior surface. In certain aspects, the interior surface faces the inner core (i.e., lumen) of the EV. In certain aspects, the exterior surface can be in contact with the endosome, the multivesicular bodies, or the membrane/cytoplasm of a producer cell or a target cell

In some aspects, the EV membrane further comprises one or more scaffold moieties, which are capable of anchoring, e.g., an antigen and/or an adjuvant and/or an immune modulator, to the EV (e.g., either on the luminal surface or on the exterior surface). In certain aspects, scaffold moieties are polypeptides (“exosome proteins”). In other aspects, scaffold moieties are non-polypeptide moieties. In some aspects, exosome proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety (e.g., exosome protein) comprises Scaffold X. In other aspects, a scaffold moiety (e.g., exosome protein) comprises Scaffold Y. In further aspects, a scaffold moiety (e.g., exosome protein) comprises both a Scaffold X and a Scaffold Y.

As demonstrated herein, in some aspects, EVs of the present disclosure are capable of inducing effector and memory T cells. In certain aspects, the memory T cells are tissue-resident memory T cells (e.g., within the CNS). In some aspects, EVs disclosed herein are capable of inducing a broad immunity (e.g., induce immune response to multiple epitopes on an antigen, or produce different antibody isotypes) against particular pathogens. Accordingly, in some aspects, the EVs are capable of being used as a “universal” vaccine.

In some aspects, EVs disclosed herein are inherently capable of inducing the activation of a signaling pathway involved in an immune response. In certain aspects, the signaling pathway involved in an immune response comprises toll-like receptors (TLRs), retinoid acid-inducible gene I (RIG-I)-like receptors (RLRs), stimulator of interferon genes (STING) pathway, or combinations thereof. In some aspects, the activation of such signaling pathway can result in the production of a type I interferon. For example, in certain aspects, the bi-lipid membrane of an EV disclosed herein comprises one or more lipids that share one of the following features: (i) unsaturated lipid tail, (ii) dihydroimidazole linker, (iii) cyclic amine head groups, and (iv) combinations thereof. Lipids with such features have been shown to activate the TLR/RLR-independent STING pathway. See Miao et al., Nature Biotechnology 37:1174-1185 (October 2019).

### Antigen

In some aspects, the payload is an antigen, which is capable of inducing an immune response in a subject. In some aspects, the payload is an antigen, which is capable of suppressing an immune response in a subject. In some aspects, an EV disclosed herein comprises a single antigen. In some aspects, an EV disclosed herein comprises multiple antigens. In certain aspects, each of the multiple antigens is different. In some aspects, an EV disclosed herein comprises at least 2, 3, 4, 5, 6, 7, 8, 9, 10 or more different antigens. In certain aspects, an EV comprises the one or more antigens in combination with one or more additional payloads (e.g., adjuvant and/or immune modulator) and/or one or more additional targeting moieties described herein. In certain aspects, an EV comprises an antigen but not an adjuvant. In some aspects, an EV comprises only the antigen. As demonstrated herein (see, e.g., Examples 13-15), by modifying the particular location at which an antigen is associated with the EV (e.g., on the exterior surface, on the luminal surface, and/or in the lumen), an EV-based vaccine of the present disclosure is capable of eliciting a strong immune response without the need for additional moieties described herein (e.g., adjuvant, immune modulator, and/or targeting moieties). Accordingly, in some aspects, an EV described herein comprises an antigen, wherein the antigen is associated with the exterior surface of the EV. As further described elsewhere in the present disclosure, in some aspects, the antigen is fused to the N-terminus of a Scaffold X moiety (e.g., PTGFRN) expressed on the exterior surface of the EV. In some aspects, the antigen is associated with the exterior surface of the EV using any of the coupling strategies described herein (e.g., ALFA-tag). In certain aspects, the EV comprising the antigen does not comprise an adjuvant, immune modulator, and/or targeting moiety.

In some aspects, the antigen comprises a neuronal protein. In certain aspects, the neuronal protein can be misfolded, wherein the misfolded neuronal protein can result in a neurological disorder. In some aspects, the neuronal protein comprises amyloid beta (Aβ), tau, alpha-synuclein (αSyn), dipeptide repeat (DPR) proteins (e.g., poly-Gly-Ala (poly-GA)), mutant Huntingtin (HTT) protein, TDP-43, or combinations thereof. Non-limiting examples of neurological disorders that are associated with such misfolded neuronal proteins can include a brain tumor, neoplastic meningitis, leptomeningeal cancer disease (LMD), amyotrophic lateral sclerosis (ALS), frontotemporal dementia (FTD), Parkinson's disease (PD), Huntington's disease (HD), Alzheimer's disease (AD), Lewy body dementia (LTD), spinocerebellar ataxia (e.g., type 8 (SCAB), SCA10, SCA12, SCA31, SCA36), Huntington's disease like-2, ataxia (e.g., Friedreich ataxia), muscular dystrophy (e.g., oculopharyngeal muscular dystrophy), or combinations thereof.

Accordingly, in some aspects, an EV disclosed herein comprises a neuronal protein as an antigen, wherein the antigen comprises dipeptide repeat (DPR) proteins. In certain aspects, the dipeptide DPR proteins are derived from repeat associated non-ATG (RAN) translation (referred to herein as RAN protein) (e.g., C9 RAN proteins). In some aspects, the dipeptide DPR proteins comprise poly-GA (i.e., repeats of glycine-alanine residues). In some aspects, the poly-GA comprises at least about 2, at least about 3, at least about 4, at least about 5, at least about 6, at least about 7, at least about 8, at least about 9, at least about 10, at least about 15, at least about 20, at least about 25, at least about 30 or more repeats of glycine-alanine residues). In certain aspects, the poly-GA comprises 10 repeats of glycine-alanine residues. In certain aspects, the poly-GA are specific for B cells (referred to herein as “poly-GA B cell antigen”). In certain aspects, the dipeptide DPR protein comprises poly-GR (i.e., repeats of glycine-arginine residues). In some aspects, the dipeptide DPR protein comprises poly-GP (i.e., repeats of glycine-proline residues). In some aspects, the dipeptide DPR protein comprises poly-PA (i.e., repeats of proline-alanine residues). In some aspects, the dipeptide DPR protein comprises poly-PR (i.e., repeats of proline-arginine residues). In some aspects, the dipeptide DPR protein comprises poly-G (i.e., repeats of glutamine residues). It will be apparent to those skilled in the art that, unless indicated otherwise, disclosures provided herein relating to poly-GA can equally apply to the other dipeptide DPR proteins provided herein. As described herein (see, e.g., section titled “Methods of Treating a Neurological Disorder”), such EVs can be used to treat neurological disorders associated with a hexanucleotide GGGGCC repeat expansion in the C9orf72 gene (e.g., C9FTD/ALS). A pathological hallmark that can be observed in C9orf72 repeat expansion carriers include formation of RNA foci and deposition of dipeptide repeat (DPR) proteins derived from repeat associated non-ATG (RAN) translation. See Nguyen et al., Annu Rev Neurosci 42:227-247 (July 2019), which is incorporated herein by reference in its entirety.

In some aspects, an EV comprising a neuronal protein described above (e.g., C9 RAN proteins, e.g., poly GA B cell antigen) are capable of inducing a B cell-specific immune response (e.g., does not induce a T cell immune response that is harmful to the subject that receives the EV). Accordingly, in some aspects, an EV disclosed herein (e.g., comprising a C9 RAN protein, e.g., poly GA B cell antigen) is capable of stimulating antibody producing B cells without activating harmful T cells. In some aspects, harmful T cells comprise T cells that exhibit excessive cytotoxicity activity (e.g., produces excessive inflammatory mediators resulting in damage to healthy cells/tissues), that induces an autoimmune response, that are not involved in treatment of the disease or disorder (e.g., does not play a role in the activation of the antibody producing B cells), or combinations thereof. In certain aspects, an EV comprising a neuronal protein described herein (e.g., C9 RAN proteins, e.g., poly GA B cell antigen) can exhibit one or more of the following when administered to a subject: (i) decrease GA protein aggregate; (ii) improve proteasome function, (iii) increase autophagy, (iv) decrease neuroinflammation, (v) decrease motor neuron loss, (vi) increase anti-polyGA antibody production, (vii) improve behavioral function, and (viii) combinations thereof.

In some aspects, the antigen comprises a fragment of the amyloid-β protein. In certain aspects, the fragment of the amyloid-β protein comprises the amino acid sequence DAEFRHDSGYEVHHQKLVFFAEDVGSNKGAIIGLMVGGVVIA (SEQ ID NO: 379) (i.e., amino acid residues 1-42 of amyloid-β protein). Accordingly, in some aspects, an EV disclosed herein comprises an antigen, wherein the antigen comprises one or more epitopes of an amyloid-β protein fragment set forth in SEQ ID NO: 379. In some aspects, the one or more epitopes comprise amino acid residues 1-42 of SEQ ID NO: 379. In some aspects, the one or more epitopes comprise amino acid residues 1-6 of SEQ ID NO: 379. In some aspects, the one or more epitopes comprise amino acid residues 1-7 of SEQ ID NO: 379. In some aspects, the one or more epitopes comprise amino acid residues 1-12 of SEQ ID NO: 379. In some aspects, the one or more epitopes comprise amino acid residues 1-14 of SEQ ID NO: 379. In certain aspects, the one or more epitopes comprise amino acid residues 1-15 of SEQ ID NO: 379. In some aspects, the one or more epitopes comprise amino acid residues 1-11 and 18-27 of SEQ ID NO: 379. In some aspects, the one or more epitopes comprise amino acid residues 13-28 of SEQ ID NO: 379. In some aspects, the one or more epitopes comprise amino acid residues 12-23 of SEQ ID NO: 379. In some aspects, the one or more epitopes comprise amino acid residues 3-6 of SEQ ID NO: 379. In some aspects, the one or more epitopes comprise amino acid residues 1-5 of SEQ ID NO: 379. In some aspects, the one or more epitopes comprise amino acid residues 35-40 of SEQ ID NO: 379. In some aspects, an EV of the present disclosure comprises an antigen, wherein the antigen comprises a combination (e.g., 2, 3, 4, 5, 6, 7, 8, 9, 10, 11, 12, or 13) of the epitopes described above.

As described herein, in some aspects, an EV that can be used in treating a neurological disorder comprises an antigen (e.g., expressed on the outer surface of the EV), wherein the antigen comprises a B cell epitope, CD4+ T cell epitope, or both. In certain aspects, the B cell epitope comprises the amino acid sequence AEFRHD (SEQ ID NO: 380) (i.e., amino acid residues 2-7 of amyloid-β protein fragment set forth in SEQ ID NO: 379). In some aspects, the B cell epitope comprises the amino acid sequence (GA)₁₀₋₂₀ (polyGA) (SEQ ID NO: 381). In some aspects, the CD4+ T cell epitope comprises the amino acid sequence AKFVAAWTLKAAA (SEQ ID NO: 382) (PADRE). In some aspects, the CD4+ T cell epitope comprises the amino acid sequence QYIKANSKFIGITE (SEQ ID NO: 383) (amino acid residues 830-843 of tetanus). In some aspects, the CD4+ T cell epitope comprises the amino acid sequence QSIALSSLMVAQAIP (SEQ ID NO: 384) (amino acid residues 356-370 of diphtheria toxin).

In some aspects, an antigen comprises a self-antigen. Non-limiting examples of self-antigens are provided elsewhere in the present disclosure (see, e.g., section titled “Methods of Treating an Autoimmune Disorder”).

As described herein, in some aspects, an EV described herein can comprise an antagonist that specifically targets any of the antigens described above. For example, in certain aspects, the antagonist can target any of the neuronal proteins described herein (e.g., C9 RAN proteins, e.g., poly GA B cell antigen). Non-limiting examples of antagonists include antibodies, mRNA, miRNA, siRNA, antisense oligonucleotide (ASO), phosphorodiamidate morpholino oligomer (PMO), peptide-conjugated phosphorodiamidate morpholino oligomer (PPMO), shRNA, lncRNA, dsDNA, or combinations thereof.

### Adjuvants

As described supra, EVs of the present disclosure can comprise an adjuvant (e.g., in combination with an antigen and/or other payloads disclosed herein). In some aspects, an EV disclosed herein comprises multiple adjuvants. In certain aspects, each of the multiple adjuvants is different. In some aspects, an EV disclosed herein comprises at least 2, 3, 4, 5, 6, 7, 8, 9, 10 or more different adjuvants. In certain aspects, an EV comprises the one or more adjuvants in combination with one or more additional payloads (e.g., antigen and/or immune modulator) and/or one or more targeting moieties described herein.

As used herein, the term “adjuvant” refers to any substance that enhances the therapeutic effect of the payload (e.g., increasing an immune response to the antigen). Accordingly, EVs described herein comprising an adjuvant are capable of increasing an immune response, e.g., to an antigen, by at least about 5%, at least about 10%, at least about 20%, at least about 30%, at least about 40%, at least about 50%, at least about 60%, at least about 70%, at least about 80%, at least about 90%, at least about 100%, at least about 250%, at least about 500%, at least about 750%, at least about 1,000% or more or more, compared to a reference (e.g., corresponding EV without the adjuvant or a non-EV delivery vehicle comprising an antigen alone or in combination with the adjuvant). In some aspects, incorporating an adjuvant disclosed herein to an EV can increase an immune response, e.g., to an antigen, by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to a reference (e.g., corresponding EV comprising the antigen alone or a non-EV delivery vehicle comprising an antigen alone or in combination with the adjuvant).

Non-limiting examples of adjuvants that can be used with the present disclosure include: Stimulator of Interferon Genes (STING) agonist, a toll-like receptor (TLR) agonist, an inflammatory mediator, RIG-I agonists, alpha-gal-cer (NKT agonist), heat shock proteins (e.g., HSP65 and HSP70), C-type lectin agonists (e.g., beta glucan (Dectin 1), chitin, and curdlan), and combinations thereof. Additional examples of adjuvants that can be used with the EVs described herein are provided throughout the present disclosure.

In some aspects, the adjuvant is a TLR9 agonist. In some aspects, the TLR9 agonist comprises a CpG oligonucleotide. As used herein, the term “CpG oligonucleotide” (CpG ODN) refers to a short synthetic single-stranded nucleic acid molecules that contain unmethylated CpG dinucleotides in particular sequence contexts (CpG motifs). There are three major classes of CpG ODNs: Class A (Type D), Class B (Type K), and Class C. In some aspects, the adjuvant is a CpG-A ODN. “CpG-A” ODNs are characterized by a phosphodiester (PO) central CpG-containing palindromic motif and a phosphorothioated (PS)-modified 3′ poly-G string. They induce high IFN-α production from pDCs but are weak stimulators of TLR9-dependent NF-κB signaling and pro-inflammatory cytokine (e.g. IL-6) production. In some aspects, the adjuvant is a CpG-B ODN. “CpG-B” ODNs contain a full PS backbone with one or more CpG dinucleotides. They strongly activate B cells and TLR9-dependent NF-κB signaling but weakly stimulate IFN-α secretion. In some aspects, the adjuvant is a CpG-C ODN. “CpG-C” ODNs combine features of both classes A and B. They contain a complete PS backbone and a CpG-containing palindromic motif. C-Class CpG ODNs induce strong IFN-α production from pDC as well as B cell stimulation.

In some aspects, the adjuvant is a TLR4 agonist. In certain aspects, the TLR4 agonist comprises monophosphoryl lipid A (MPLA), e.g., a derivative of lipid A from Salmonella minnesota R595 lipopolysaccharide (LPS or endotoxin).

In some aspects, incorporating an adjuvant (e.g., such as those disclosed herein) to an EV can broaden an immune response induced by the EV. As used herein, to “broaden an immune response” refers to enhancing the diversity of an immune response. In some aspects, the diversity of an immune response can be enhanced through epitope spreading (i.e., inducing and/or increasing an immune response (cellular and/or humoral immune response) against a greater number/variety of epitopes on an antigen). In some aspects, the diversity of an immune response can be enhanced through the production of different and/or multiple antibody isotypes (e.g., IgG, IgA, IgD, IgM, and/or IgE).

In some aspects, an adjuvant (e.g., such as those disclosed herein) can also help regulate the type of immune response induced by the EV. For example, in some aspects, incorporating an adjuvant to an EV can help drive an immune response towards a more Th1 phenotype. As used herein, a “Th1” immune response is generally characterized by the production of IFN-γ, which can activate the bactericidal activities of innate cells (e.g., macrophages), help induce B cells to make opsonizing (marking for phagocytosis) and complement-fixing antibodies, and/or lead to cell-mediated immunity (i.e., not mediated by antibodies). In general, Th1 responses are more effective against intracellular pathogens (viruses and bacteria that are inside host cells) and/or cancers.

In some aspects, incorporating an adjuvant to an EV can help drive an immune response towards a more Th2 phenotype. As used herein, a “Th2” immune response can be characterized by the release of certain cytokines, such as IL-5 (induces eosinophils in the clearance of parasites) and IL-4 (facilitates B cell isotype switching). In general, Th2 responses are more effective against extracellular bacteria, parasites including helminths and toxins.

In some aspects, incorporating an adjuvant to an EV can help drive an immune response towards a more Th17 phenotype. As used herein, a “Th17” immune response is mediated by Th17 cells. As used herein, “Th17 cells” refer to a subset of CD4+ T cells characterized by the production of pro-inflammatory cytokines, such as IL-17A, IL-17F, IL-21, IL-22, and granulocyte-macrophage colony-stimulating factor (GM-CSF). Th17 cells are generally thought to play an important role in host defense against infection, by recruiting neutrophils and macrophages to infected tissues.

In some aspects, incorporating an adjuvant to an EV can help drive an immune response towards a more cellular immune response (e.g., T-cell mediated). In some aspects, incorporating an adjuvant to an EV can help drive an immune response towards a more humoral immune response (e.g., antibody-mediated). For instance, in certain aspects, an EV described herein comprises a neuronal peptide as an antigen (e.g., a poly-GA B cell antigen) and an adjuvant (e.g., CpG-B), wherein the adjuvant allows the EV to induce a strong B cell immune response without activating a T cell immune response that can be harmful to a subject (see, e.g., Examples 12 and 13).

In some aspects, an adjuvant induces the activation of a cytosolic pattern recognition receptor. In some aspects, such adjuvants are viral nucleic acid mimetics. Not to be bound by any one theory, EVs comprising such adjuvants are capable of preferentially inducing Th1 (e.g., IFN) and/or antibody-mediated immune responses. Non-limiting examples of cytosolic pattern recognition receptor includes: stimulator of interferon genes (STING), retinoic acid-inducible gene I (RIG-1), Melanoma Differentiation-Associated protein 5 (MDA5), Nucleotide-binding oligomerization domain, Leucine rich Repeat and Pyrin domain containing (NLRP), inflammasomes, or combinations thereof. In certain aspects, an adjuvant is a STING agonist. Stimulator of Interferon Genes (STING) is a cytosolic sensor of cyclic dinucleotides that is typically produced by bacteria. Upon activation, it leads to the production of type I interferons (e.g., IFN-α (alpha), IFN-β (beta), IFN-κ (kappa), IFN-δ (delta), IFN-ε (epsilon), IFN-τ (tau), IFN-ω (omega), and IFN- (zeta, also known as limitin)) and initiates an immune response. In certain aspects, the STING agonist comprises a cyclic dinucleotide STING agonist or a non-cyclic dinucleotide STING agonist.

Cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. The CDNs can have 2′2′, 2′3′, 2′5′, 3′3′, or 3'S′ bonds linking the cyclic dinucleotides, or any combination thereof.

Cyclic purine dinucleotides can be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides can be modified analogues. Any suitable modification known in the art can be used, including, but not limited to, phosphorothioate, biphosphorothioate, fluorinate, and difluorinate modifications.

Non cyclic dinucleotide agonists can also be used, such as 5,6-Dimethylxanthenone-4-acetic acid (DMXAA), or any other non-cyclic dinucleotide agonist known in the art.

Non-limiting examples of STING agonists that can be used with the present disclosure include: DMXAA, STING agonist-1, ML RR-S2 CDA, ML RR-52c-di-GMP, ML-RR-52 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(PS)2, 3′3′-cGAMP, and combinations thereof. Non-limiting examples of the STING agonists can be found at U.S. Pat. No. 9,695,212, WO 2014/189805 A1, WO 2014/179335 A1, WO 2018/100558 A1, U.S. Pat. No. 10,011,630 B2, WO 2017/027646 A1, WO 2017/161349 A1, WO 2015/077354, and WO 2016/096174 A1, each of which is incorporated by reference in its entirety. In some aspects, non-limiting examples of cyclic nucleotides STING agonist include any CDN disclosed in WO 2016/096174A1, which is incorporated by reference in its entirety.

In some aspects, the STING agonist useful for the present disclosure comprises c-di-AMP, c-di-GMP, c-di-IMP, c-AMP-GMP, c-AMP-IMP, and c-GMP-IMP, described in WO 2013/185052 and Sci. Transl. Med. 283,283ra52 (2015), which are incorporated herein by reference in their entireties.

In some aspects, the STING agonist useful for the present disclosure comprises a compound or a pharmaceutically acceptable salt thereof disclosed in WO 2014/189806, US 2018/186828, US 2014/329889, US 2018/118777, US 2016/210400, US 2017/340658, US 2018/002369, US 2018/064745, US 2018/230178, US 2019/062365, US 2018/230178, WO 2018/100558, US 2018/105514, or WO 2017/175156, each of which is incorporated herein by reference in its entirety.

In some aspects, an adjuvant is a TLR agonist. Non-limiting examples of TLR agonists include: TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C, MPLA), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA, CpG-B), and combinations thereof. Non-limiting examples of TLR agonists can be found at WO2008115319A2, US20130202707A1, US20120219615A1, US20100029585A1, WO2009030996A1, WO2009088401A2, and WO2011044246A1, each of which are incorporated by reference in its entirety. Not to be bound by any one theory, in some aspects, EVs comprising a TLR agonist as an adjuvant is capable of preferentially inducing a Th1 and/or antibody-mediated immune response.

In some aspects, an adjuvant is an inflammatory mediator.

In some aspects, an adjuvant comprises an aluminum-containing adjuvant (also referred to herein as “alum”). In some aspects, an adjuvant comprises an aluminum salt. In certain aspects, the aluminum salt is aluminum hydroxide. Not to be bound by any one theory, in some aspects, an EV comprising an aluminum salt as an adjuvant is capable of mediating damage-associated molecular pattern (DAMP) (e.g., NLRP3) activation of antigen-presenting cells (APCs). In certain aspects, such EVs are capable of preferentially inducing a Th2-cell and/or antibody-mediated immune response. In some aspects, the aluminum-containing adjuvant can be used in combination with one or more adjuvants, such as CpG.

In some aspects, an adjuvant that can be used with the EVs of the present disclosure comprises emulsions (water-in-oil). In certain aspects, the emulsions include MF59 and AS03. Not to be bound by any one theory, in some aspects, an EV comprising an emulsion as an adjuvant is capable of enhancing APC antigen uptake. In certain aspects, such EVs are capable of inducing robust neutralizing antibodies. In some aspects, such EVs are useful for inducing both Th1 and Th2-mediated immune responses.

In some aspects, an antigen is expressed on the exterior surface or in the lumen (e.g., on the luminal surface) of the EV. In some aspects, an adjuvant is expressed on the exterior surface or in the luminal surface of the EVs, directly connected to the lipid bilayer. In such aspects, the antigen and/or the adjuvant can be linked to a scaffold moiety (e.g., Scaffold X and/or Scaffold Y).

In some aspects, an EVs, described herein comprises a first scaffold moiety. In certain aspects, the antigen is linked to the first scaffold moiety. In other aspects, the adjuvant is linked to the first scaffold moiety. In further aspects, both the antigen and the adjuvant are linked to the first scaffold moiety. In some aspects, an EVs, further comprises a second scaffold moiety. In certain aspects, the antigen is linked to the first scaffold moiety, and the adjuvant is linked to the second scaffold moiety. In some aspects, the first scaffold moiety and the second scaffold moiety are the same (e.g., both Scaffold X or both Scaffold Y). In other aspects, the first scaffold moiety and the second scaffold moiety are different (e.g., first scaffold moiety is Scaffold X and the second scaffold moiety is Scaffold Y; or first scaffold moiety is Scaffold Y and the second scaffold moiety is Scaffold X).

Non-limiting examples of Scaffold X include: prostaglandin F2 receptor negative regulator (PTGFRN); basigin (BSG); immunoglobulin superfamily member 2 (IGSF2); immunoglobulin superfamily member 3 (IGSF3); immunoglobulin superfamily member 8 (IGSF8); integrin beta-1 (ITGB1); integrin alpha-4 (ITGA4); 4F2 cell-surface antigen heavy chain (SLC3A2); and a class of ATP transporter proteins (ATP1A1, ATP1A2, ATP1A3, ATP1A4, ATP1B3, ATP2B1, ATP2B2, ATP2B3, ATP2B). In certain aspects, Scaffold X is a whole protein. In other aspects, Scaffold X is a protein fragment (e.g., functional fragment).

In other aspects, the scaffold moiety useful for the present disclose, a first scaffold moiety, a second scaffold moiety, and/or a third scaffold moiety, includes a conventional exosome protein, including, but not limiting, tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin and fragments thereof, peptides that have affinity to any of these proteins or fragments thereof, or any combination thereof.

Non-limiting examples of Scaffold Y include: the myristoylated alanine rich Protein Kinase C substrate (MARCKS) protein; myristoylated alanine rich Protein Kinase C substrate like 1 (MARCKSL1) protein; and brain acid soluble protein 1 (BASP1) protein. In some aspects, Scaffold Y is a whole protein. In certain aspects, Scaffold Y is a protein fragment (e.g., functional fragment).

In some aspects, the antigen is linked to a first scaffold moiety on the luminal surface of the EVs, and the adjuvant is in the lumen of the EV. As used herein, when a molecule (e.g., antigen or adjuvant) is described as “in the lumen” of the e.g. EV, it means that the molecule is not linked to a scaffold moiety described herein. In some aspects, the antigen is in the lumen of the EV, and the adjuvant is linked to a first scaffold moiety on the luminal surface of the EV. In such aspects, the first scaffold moiety can be Scaffold X or Scaffold Y.

In some aspects, an adjuvant and/or antigen can be modified to increase encapsulation in an EV. This modification can include the addition of a lipid binding tag by treating the agonist with a chemical or enzyme, or by physically or chemically altering the polarity or charge of the adjuvant and/or antigen. The adjuvant and/or antigen can be modified by a single treatment, or by a combination of treatments, e.g., adding a lipid binding tag only, or adding a lipid binding tag and altering the polarity. The previous example is meant to be a non-limiting illustrative instance. It is contemplated that any combination of modifications can be practiced. The modification can increase encapsulation of the adjuvant and/or antigen in the EV by between about 2-fold and about 10,000-fold, between about 10-fold and 1,000-fold, or between about 100-fold and about 500-fold compared to encapsulation of an unmodified agonist. The modification can increase encapsulation of the adjuvant and/or antigen in the EV by at least about 2-fold, about 5-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold, about 700-fold, about 800-fold, about 900-fold, about 1,000-fold, about 2,000-fold, about 3,000-fold, about 4,000-fold, about 5,000-fold, about 6,000-fold, about 7,000-fold, about 8,000-fold, about 9,000-fold, or about 10,000-fold compared to encapsulation of an unmodified adjuvant and/or antigen.

In some aspects, the EV is further modified to display an additional protein (or fragment thereof) that can help direct EV uptake (e.g., targeting moiety), activate, or block cellular pathways to enhance the combinatorial effects associated with the EV (e.g., effect of a payload loaded into an exosome, e.g., STING agonist). In certain aspects, the EV disclosed herein further comprises a targeting moiety that can modify the distribution of the EVs in vivo or in vitro. In some aspects, the targeting moiety can be a biological molecule, such as a protein, a peptide, a lipid, or a synthetic molecule.

In some aspects, a targeting moiety of the present disclosure specifically binds to a marker for a dendritic cell. In certain aspects, the marker is expressed only on dendritic cells. In some aspects, dendritic cells comprise a plasmacytoid dendritic cell (pDC), a myeloid/conventional dendritic cell 1 (cDC1), a myeloid/conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof. In some aspects, the targeting moiety is a protein, wherein the protein is an antibody or a fragment thereof that can specifically bind to a marker selected from DEC205, CLEC9A, CLEC6, DCIR, DC-SIGN, LOX-1, MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), BDCA-2, BDCA-3, Dectin-2, Bst-2 (CD317), CD11c, XCR1, Langerin, or any combination thereof. In some aspects, a marker useful for the present disclosure comprises a C-type lectin like domain. In certain aspects, a marker is Clec9a and the dendritic cell is cDC1.

In some aspects, a targeting moiety disclosed herein can bind to both human and mouse Clec9a, including any variants thereof. In some aspects, a targeting moiety of the present disclosure can bind to Clec9a from other species, including but not limited to chimpanzee, rhesus monkey, dog, cow, horse, or rat. Sequences for such Clec9a protein are known in the art. See, e.g., U.S. Pat. No. 8,426,565 B2, which is herein incorporated by reference in its entirety.

In some aspects, a targeting moiety of the present disclosure specifically binds to a marker for a T cell. In certain aspects, the T cell is a CD4+ T cell. In some aspects, the T cell is a CD8+ T cell.

In some aspects, a targeting moiety disclosed herein binds to human CD3 protein or a fragment thereof. Sequences for human CD3 protein are known in the art.

In some aspects, a targeting moiety disclosed herein can bind to both human and mouse CD3, including any variants thereof. In some aspects, a targeting moiety of the present disclosure can bind to CD3 from other species, including but not limited to chimpanzee, rhesus monkey, dog, cow, horse, or rat. Sequences for such CD3 protein are also known in the art.

In some aspects, a targeting moiety that can be used with the EVs of the present disclosure can specifically bind to a marker for a B cell. Non-limiting examples of such targeting moieties include CD40L molecule, ICOS, or binding agents (e.g., antibodies) against CD40, CD21, CD19, CD20, ICOSL, or MHCII.

In some aspects, a targeting moiety disclosed herein can allow for greater uptake of an EV by a cell expressing a marker specific for the targeting moiety (e.g., CD3: CD4+ T cell and/or CD8+ T cell; Clec9a: dendritic cells; CD40, MHCII: B cells). In some aspects, the uptake of an EV is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to a reference (e.g., corresponding EV without the targeting moiety or a non-EV delivery vehicle). In some aspects, a reference comprises an EV that does not express a targeting moiety disclosed herein.

In some aspects, the increased uptake of an EV disclosed herein can allow for greater immune response. Accordingly, in certain aspects, an EV expressing a targeting moiety disclosed herein can increase an immune response (e.g., against a tumor antigen loaded onto the exosome) by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to a reference (e.g., corresponding EV without the targeting moiety or a non-EV delivery vehicle). In some aspects, a reference comprises an EV that does not express a targeting moiety disclosed herein. In certain aspects, an immune response is mediated by T cells (e.g., CD8+ T cells or CD4+ T cells) and/or B cells.

As described supra, a targeting moiety disclosed herein can comprise a peptide, an antibody or an antigen binding fragment thereof, a chemical compound, or any combination thereof.

In some aspects, the targeting moiety is a peptide that can specifically bind to Clec9a. See, e.g., Yan et al., Oncotarget 7(26): 40437-40450 (2016). For example, in certain aspects, the peptide comprises a soluble fragment of Clec9a. A non-limiting example of such a peptide is described in U.S. Pat. No. 9,988,431 B2, which is herein incorporated by reference in its entirety. In certain aspects, the peptide comprises a ligand (natural or synthetic) of Clec9a, such as those described in Ahrens et al., Immunity 36(4): 635-45 (2012); and Zhang et al., Immunity 36(4): 646-57 (2012). A non-limiting example of a peptide comprising a Clec9a ligand is described in International Publ. No. WO 2013/053008 A2, which is herein incorporated by reference in its entirety.

In some aspects, the targeting moiety is a peptide that can specifically bind to CD3. For example, in certain aspects, the peptide comprises a soluble fragment of CD3. In certain aspects, the peptide comprises a ligand (natural or synthetic) of CD3.

In some aspects, the targeting moiety is a peptide that can specifically bind to a marker on B cells. In certain aspects, the targeting moiety is a peptide that can specifically bind to CD40 or MHCII expressed on B cells.

In some aspects, the targeting moiety is an antibody or an antigen binding fragment thereof. In certain aspects, a targeting moiety is a single-chain Fv antibody fragment. In certain aspects, a targeting moiety is a single-chain F(ab) antibody fragment. In certain aspects, a targeting moiety is a nanobody. In certain aspects, a targeting moiety is a monobody.

In some aspects, an EV disclosed herein comprises one or more (e.g., 2, 3, 4, 5, or more) targeting moieties. In certain aspects, the one or more targeting moieties are expressed in combination with other exogenous biologically active molecules disclosed herein (e.g., therapeutic molecule, adjuvant, or immune modulator). In some aspects, the one or more targeting moieties can be expressed on the exterior surface of the EV. Accordingly, in certain aspects, the one or more targeting moieties are linked to a scaffold moiety (e.g., Scaffold X) on the exterior surface of the EV. When the one or more targeting moieties are expressed in combination with other exogenous biologically active molecules (e.g., therapeutic molecule, adjuvant, or immune modulator), the other exogenous biologically active molecules can be expressed on the surface (e.g., exterior surface or luminal surface) or in the lumen of the EV.

The producer cell can be modified to comprise an additional exogenous sequence encoding for the additional protein or fragment thereof. Alternatively, the additional protein or fragment thereof can be covalently linked or conjugated to the EV via any appropriate linking chemistry known in the art. Non-limiting examples of appropriate linking chemistry include amine-reactive groups, carboxyl-reactive groups, sulfhydryl-reactive groups, aldehyde-reactive groups, photoreactive groups, ClickIT chemistry, biotin-streptavidin or other avidin conjugation, or any combination thereof.

### Immune Modulator

In some aspects, an EV of the present disclosure can comprise an immune modulator (e.g., along with an antigen and/or other payloads disclosed herein). In some aspects, an EV disclosed herein comprises multiple immune modulators. In certain aspects, each of the multiple immune modulators is different. In some aspects, an EV disclosed herein comprises at least 2, 3, 4, 5, 6, 7, 8, 9, 10 or more different immune modulators. In certain aspects, an EV comprises the one or more immune modulators in combination with one or more additional payloads described herein (e.g., antigen and/or adjuvants. In some aspects, an EV disclosed herein can further comprise a targeting moiety. For example, in certain aspects, an EV comprises (i) one or more immune modulators, (ii) one or more additional payloads (e.g., antigen and/or adjuvants), and (iii) one or more additional targeting moieties.

In some aspects, an immune modulator can be expressed on the surface (e.g., exterior surface or luminal surface) or in the lumen of the EV. Accordingly, in certain aspects, the immune modulator is linked to a scaffold moiety (e.g., Scaffold X) on the exterior surface of the EV or on the luminal surface of the EV. In other aspects, the immune modulator is linked to a scaffold moiety (e.g., Scaffold Y) on the luminal surface of the EV. In further aspects, the immune modulator is in the lumen of the exosome (i.e., not linked to either Scaffold X or Scaffold Y).

In some aspects, an immune modulator that can be used with the EVs described herein has anti-tumor activity. In other aspects, an immune modulator useful for the present disclosure has tolerogenic activity. In some aspects, an immune modulator can regulate innate immune response. In certain aspects, an immune modulator regulates innate immune response by targeting natural killer cells. In some aspects, an immune modulator can regulate adaptive immune response. In some aspects, the immune modulator regulates adaptive immune response by targeting cytotoxic T cells. In further aspects, the immune modulator regulates adaptive immune response by targeting B cells. In certain aspects, an immune modulator disclosed herein can modulate the distribution of an exosome to a cytotoxic T cell or a B cell (i.e., bio-distribution modifying agent).

In some aspects, an immune modulator useful for the present disclosure can specifically induce the activation of certain lymphocyte subsets. For instance, in some aspects, an immune modulator can specifically induce the activation of CD4+ T helper cells. CD4+ T helper cells are arguably the most important cells in adaptive immunity, as they are required for almost all adaptive immune responses. They not only help activate B cells to secrete antibodies and macrophages to destroy ingested microbes, but they also help activate cytotoxic T cells to kill infected target cells. Crott S., Nat Rev Immunol 15(3): 185-189 (March 2015). In certain aspects, the immune modulator is a peptide that can specifically induce the activation of CD4+ helper T cells. In some aspects, such peptides are referred to herein as “CD4+ T helper peptide”. In some aspects, the CD4+T helper peptides are derived from tetanus, measles, diphtheria toxins, or combinations thereof. The CD4+T help peptides that are useful for the present disclosure can also comprise the PADRE peptide. In certain aspects, such peptides are referred to herein as “universal CD4+ T helper peptide,” as they are capable of inducing the activation of CD4+ helper T cells in an antigen-independent manner (i.e., non-specific activation).

In some aspects, an immune modulator comprises an inhibitor for a negative checkpoint regulator or an inhibitor for a binding partner of a negative checkpoint regulator. In certain aspects, the negative checkpoint regulator comprises cytotoxic T-lymphocyte-associated protein 4 (CTLA-4), programmed cell death protein 1 (PD-1), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, or any combination thereof.

In some aspects, the immune modulator is an inhibitor of cytotoxic T-lymphocyte-associate protein 4 (CTLA-4). In certain aspects, the CTLA-4 inhibitor is a monoclonal antibody of CTLA-4 (“anti-CTLA-4 antibody”). In certain aspects, the inhibitor is a fragment of a monoclonal antibody of CTLA-4. In certain aspects, the antibody fragment is a scFv, (scFv)₂, Fab, Fab′, and F(ab′)₂, F(ab1)₂, Fv, dAb, or Fd of a monoclonal antibody of CTLA-4. In certain aspects, the inhibitor is a nanobody, a bispecific antibody, or a multispecific antibody against CTLA-4. In some aspects, the anti-CTLA-4 antibody is ipilimumab. In other aspects, the anti-CTLA-4 antibody is tremelimumab.

In some aspects, the immune modulator is an inhibitor of programmed cell death protein 1 (PD-1). In some aspects, the immune modulator is an inhibitor of programmed death-ligand 1 (PD-L1). In some aspects, the immune modulator is an inhibitor of programmed death-ligand 2 (PD-L2). In certain aspects, the inhibitor of PD-1, PD-L1, or PD-L2 is a monoclonal antibody of PD-1 (“anti-PD-1 antibody”), PD-L1 (“anti-PD-L1 antibody”), or PD-L2 (“anti-PD-L2 antibody”). In some aspects, the inhibitor is a fragment of an anti-PD-1 antibody, anti-PD-L1 antibody, or anti-PD-L2 antibody. In certain aspects, the antibody fragment is a scFv, (scFv)₂, Fab, Fab′, and F(ab′)₂, F(ab1)₂, Fv, dAb, or Fd of a monoclonal antibody of PD-1, PD-L1, or PD-L2. In certain aspects, the inhibitor is a nanobody, a bispecific antibody, or a multispecific antibody against PD-1, PD-L1, or PD-L2. In some aspects, the anti-PD-1 antibody is nivolumab. In some aspects, the anti-PD-1 antibody is pembrolizumab. In some aspects, the anti-PD-1 antibody is pidilizumab. In some aspects, the anti-PD-L1 antibody is atezolizumab. In other aspects, the anti-PD-L1 antibody is avelumab.

In some aspects, the immune modulator is an inhibitor of lymphocyte-activated gene 3 (LAG3). In certain aspects, the inhibitor of LAG3 is a monoclonal antibody of LAG3 (“anti-LAG3 antibody”). In some aspects, the inhibitor is a fragment of an anti-LAG3 antibody, e.g., scFv, (scFv)₂, Fab, Fab′, and F(ab′)₂, F(ab1)₂, Fv, dAb, or Fd. In certain aspects, the inhibitor is a nanobody, a bispecific antibody, or a multispecific antibody against LAG3.

In some aspects, the immune modulator is an inhibitor of T-cell immunoglobulin mucin-containing protein 3 (TIM-3). In some aspects, the immune modulator is an inhibitor of B and T lymphocyte attenuator (BTLA). In some aspects, the immune modulator is an inhibitor of T cell immunoreceptor with Ig and ITIM domains (TIGIT). In some aspects, the immune modulator is an inhibitor of V-domain Ig suppressor of T cell activation (VISTA). In some aspects, the immune modulator is an inhibitor of adenosine A2a receptor (A2aR). In some aspects, the immune modulator is an inhibitor of killer cell immunoglobulin like receptor (KIR). In some aspects, the immune modulator is an inhibitor of indoleamine 2,3-dioxygenase (IDO). In some aspects, the immune modulator is an inhibitor of CD20, CD39, or CD73.

In some aspects, the immune modulator comprises an activator for a positive co-stimulatory molecule or an activator for a binding partner of a positive co-stimulatory molecule. In certain aspects, the positive co-stimulatory molecule comprises a TNF receptor superfamily member (e.g., CD120a, CD120b, CD18, OX40, CD40, Fas receptor, M68, CD27, CD30, 4-1BB, TRAILR1, TRAILR2, TRAILR3, TRAILR4, RANK, OCIF, TWEAK receptor, TACI, BAFF receptor, ATAR, CD271, CD269, AITR, TROY, CD358, TRAMP, and XEDAR). In some aspects, the activator for a positive co-stimulatory molecule is a TNF superfamily member (e.g., TNFα, TNF-C, OX40L, CD40L, FasL, LIGHT, TL1A, CD27L, Siva, CD153, 4-1BB ligand, TRAIL, RANKL, TWEAK, APRIL, BAFF, CAMLG, NGF, BDNF, NT-3, NT-4, GITR ligand, and EDA-2).

In some aspects, the immune modulator is an activator of TNF Receptor Superfamily Member 4 (OX40). In certain aspects, the activator of OX40 is an agonistic anti-OX40 antibody. In further aspects, the activator of OX40 is a OX40 ligand (OX40L).

In some aspects, the immune modulator is an activator of CD27. In certain aspects, the activator of CD27 is an agonistic anti-CD27 antibody. In other aspects, the activator of CD27 is a CD27 ligand (CD27L).

In some aspects, the immune modulator is an activator of CD40. In certain aspects, the activator of CD40 is an agonistic anti-CD40 antibody. In some aspects, the activator of CD40 is a CD40 ligand (CD40L). In certain aspects, the CD40L is a monomeric CD40L. In other aspects, the CD40L is a trimeric CD40L.

In some aspects, the immune modulator is an activator of glucocorticoid-induced TNFR-related protein (GITR). In certain aspects, the activator of GITR is an agonistic anti-GITR antibody. In other aspects, the activator of GITR is a natural ligand of GITR.

In some aspects, the immune modulator is an activator of 4-1BB. In specific aspects, the activator of 4-1BB is an agonistic anti-4-1BB antibody. In certain aspects, the activator of 4-1BB is a natural ligand of 4-1BB.

In some aspects, the immune modulator is a Fas receptor (Fas). In such aspects, the Fas receptor is displayed on the surface of the EV. In some aspects, the immune modulator is Fas ligand (FasL). In certain aspects, the Fas ligand is displayed on the surface of the EV. In some aspects, the immune modulator is an anti-Fas antibody or an anti-FasL antibody.

In some aspects, the immune modulator is an activator of a CD28-superfamily co-stimulatory molecule. In certain aspects, the CD28-superfamily co-stimulatory molecule is ICOS or CD28. In certain aspects, the immunomodulating component is ICOSL, CD80, or CD86.

In some aspects, the immune modulator is an activator of inducible T cell co-stimulator (ICOS). In certain aspects, the activator of ICOS is an agonistic anti-ICOS antibody. In other aspects, the activator of ICOS is a ICOS ligand (ICOSL).

In some aspects, the immune modulator is an activator of CD28. In some aspects, the activator of CD28 is an agonistic anti-CD28 antibody. In other aspects, the activator of CD28 is a natural ligand of CD28. In certain aspects, the ligand of CD28 is CD80.

In some aspects, the immune modulator comprises a cytokine or a binding partner of a cytokine. In some aspects, the cytokine is selected from (i) common gamma chain family of cytokines; (ii) IL-1 family of cytokines; (iii) hematopoietic cytokines; (iv) interferons (e.g., type I, type II, or type III); (v) TNF family of cytokines; (vi) IL-17 family of cytokines; (vii) damage-associated molecular patterns (DAMPs); (viii) tolerogenic cytokines; or (ix) combinations thereof. In certain aspects, the cytokine comprises IL-2, IL-4, IL-7, IL-10, IL-12, IL-15, IL-21, IFN-γ, IL-1α, IL-1β, IL-1ra, IL-18, IL-33, IL-36α, IL-36β, IL-36γ, IL-36ra, IL-37, IL-38, IL-3, IL-5, IL-6, IL-11, IL-13, IL-23, granulocyte-macrophage colony stimulating factor (GM-CSF), granulocyte-colony stimulating factor (G-CSF), leukemia inhibitory factor (LIF), stem cell factor (SCF), thrombopoietin (TPO), macrophage-colony stimulating factor (M-CSF), erythropoieticn (EPO), Flt-3, IFN-α, IFN-β, IFN-γ, IL-19, IL-20, IL-22, IL-24, TNF-α, TNF-β, BAFF, APRIL, lymphotoxin beta (TNF-γ), IL-17A, IL-17B, IL-17C, IL-17D, IL-17E, IL-17F, IL-25, TSLP, IL-35, IL-27, TGF-β, or combinations thereof.

In some aspects, the immune modulator comprises a chemokine. In certain aspects, chemokine comprises a (i) CC chemokine (e.g., CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28); (ii) CXC chemokine (e.g., CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17); (iii) C chemokine (e.g., XCL1, XCL2); (iv) CX3C chemokine (e.g., CX3CL1); (v) or combinations thereof.

In some aspects, the immune modulator comprises an inhibitor of lysophosphatidic acid (LPA). LPA is a highly potent endogenous lipid mediator that protects and rescues cells from programmed cell death. LPA, through its high affinity LPA-1 receptor, is an important mediator of fibrogenesis.

In some aspects, the LPA-1 inhibitor comprises AM095, which is a potent and orally bioavailable antagonist of LPA-1 with IC₅₀ values of 0.73 and 0.98 μM for mouse or recombinant human LPA-1, respectively. In vitro, AM095 has been shown to inhibit LPA-1-induced chemotaxis of both mouse LPA-1/CHO cells and human A2058 melanoma cells with IC₅₀ values of 0.78 μM and 0.23 μM. In vivo, AM095 can dose-dependently block LPA-induced histamine release with an ED₅₀ value of 8.3 mg/kg in mice. Additionally, AM095 has been revealed to remarkably reduce the BALF collagen and protein with an ED₅₀ value of 10 mg/kg in lungs. AM095 has also been shown to decrease both macrophage and lymphocyte infiltration induced by bleomycin in mice. See Swaney et al. (2018) Mol. Can. Res. 16:1601-1613, which is herein incorporated by reference in its entirety.

In some aspects, the LPA-1 inhibitor comprises AM152 (also known as BMS-986020). AM152 is a high-affinity LPA-1 antagonist which inhibits bile acid and phospholipid transporters with IC₅₀s of 4.8 μM, 6.2 μM, and 7.5 μM for BSEP, MRP4, and MDR3, respectively. AM152 can be used for the treatment of idiopathic pulmonary fibrosis (IPF). See Kihara et al. (2015) Exp. Cell Res. 333:171-7; Rosen et al. (2017) European Respiratory Journal 50: PA1038; and, Palmer et al. (2018) Chest 154:1061-1069, which are herein incorporated by reference in their entireties. The Phase 2 study of AM152 (described in Palmer 2018) was terminated early due to gall bladder toxicity and early signs of liver toxicity liver transporter (2 specific transporters).

In some aspects, the immune modulator that can be used with the present disclosure comprises a protein that supports intracellular interactions required for germinal center responses. In certain aspects, such a protein comprises a signaling lymphocyte activation molecule (SLAM) family member or a SLAM-associated protein (SAP). In some aspects, a SLAM family members comprises SLAM, CD48, CD229 (Ly9), Ly108, 2B4, CD84, NTB-A, CRACC, BLAME, CD2F-10, or combinations thereof. Non-limiting examples of other immune modulators that can play a role in germinal center response includes: ICOS-ICOSL, CD40-40L, CD28/B7, PD-1/L1, IL-4/IL4R, IL21/IL21R, TLR4, TLR7, TLR8, TLR9, CD180, CD22, and combinations thereof.

In some aspects, the immune modulator comprises a T-cell receptor (TCR) or a derivative thereof. In certain aspects, the immune modulator is a TCR α-chain or a derivative thereof. In other aspects, the immune modulator is a TCR β-chain or a derivative thereof. In further aspects, the immune modulator is a co-receptor of the T-cell or a derivative thereof.

In some aspects, the immune modulator comprises a chimeric antigen receptor (CAR) or a derivative thereof. In certain aspects, the CAR binds to one or more of the antigens disclosed herein (e.g., tumor antigen, e.g., alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen, MART-1 gp100, and TNF-related apoptosis-inducing ligand).

In some aspects, the immune modulator comprises an activator of a T-cell receptor or co-receptor. In certain aspects, the immunomodulating component is an activator of CD3. In certain aspects, the activator is a fragment of a monoclonal antibody of CD3. In certain aspects, the antibody fragment is a scFv, (scFv)₂, Fab, Fab′, and F(ab′)₂, F(ab1)₂, Fv, dAb, or Fd of a monoclonal antibody against CD3. In certain aspects, the activator is a nanobody, a bispecific antibody, or a multispecific antibody against CD3. In certain aspects, the immunomodulating component is an activator of CD28. In certain aspects, the activator is a fragment of a monoclonal antibody of CD28. In certain aspects, the antibody fragment is a scFv, (scFv)₂, Fab, Fab′, and F(ab′)₂, F(ab1)₂, Fv, dAb, or Fd of a monoclonal antibody of CD28. In certain aspects, the activator is a nanobody, a bispecific antibody, or a multispecific antibody against CD28.

In some aspects, the immune modulator comprises a tolerance inducing agent. In certain aspects, the tolerance inducing agent comprises a NF-κB inhibitor. Non-limiting examples of NF-κB inhibitors that can be used with the present disclosure includes: IKK complex inhibitors (e.g., TPCA-1, NF-κB Activation Inhibitor VI (BOT-64), BMS 345541, Amlexanox, SC-514 (GK 01140), IMD 0354, IKK-16), IκB degradation inhibitor (e.g., BAY 11-7082, MG-115, MG-132, Lactacystin, Epoxomicin, Parthenolide, Carfilzomib, MLN-4924 (Pevonedistat)), NF-κB nuclear translocation inhibitor (e.g., JSH-23, Rolipram), p65 acetylation inhibitor (e.g., Gallic acid, Anacardic acid), NF-κB-DNA binding inhibitor (e.g., GYY 4137, p-XSC, CV 3988, Prostaglandin E2 (PGE2)), NF-κB transactivation inhibitor (e.g., LY 294002, Wortmannin, Mesalamine), or combinations thereof. See also Gupta, S.C., et al., Biochim Biophys Acta 1799:775-787 (2010), which is herein incorporated by reference in its entirety. In some aspects, an immune modulator that can inhibit NF-κB activity and be used with the EVs disclosed herein comprises an antisense-oligonucleotide that specifically targets NF-κB. In further aspects, an immune modulator capable of inducing tolerance comprises a COX-2 inhibitor, mTOR inhibitor (e.g., rapamycin and derivatives), prostaglandins, nonsteroidal anti-inflammatory agents (NSAIDS), antileukotriene, aryl hydrocarbon receptor (AhR) ligand, vitamin D, retinoic acid, steroids, Fas receptor/ligand, CD22 ligand, IL-10, TGF-β, IL-2, GM-CSF, IL-35, IL-27, metabolic regulator (e.g., glutamate), glycans (e.g., ES62, LewisX, LNFPIII), peroxisome proliferator-activated receptor (PPAR) agonists, immunoglobulin-like transcript (ILT) family of receptors (e.g., ILT3, ILT4, HLA-G, ILT-2), or combinations thereof.

In some aspects, the immune modulator is an agonist. In certain aspects, the agonist is an endogenous agonist, such as a hormone, or a neurotransmitter. In other aspects, the agonist is an exogenous agonist, such as a drug. In some aspects, the agonist is a physical agonist, which can create an agonist response without binding to the receptor. In some aspects, the agonist is a superagonist, which can produce a greater maximal response than the endogenous agonist. In certain aspects, the agonist is a full agonist with full efficacy at the receptor. In other aspects, the agonist is a partial agonist having only partial efficacy at the receptor relative to a full agonist. In some aspects, the agonist is an inverse agonist that can inhibit the constitutive activity of the receptor. In some aspects, the agonist is a co-agonist that works with other co-agonists to produce an effect on the receptor. In certain aspects, the agonist is an irreversible agonist that binds permanently to a receptor through formation of covalent bond. In certain aspects, the agonist is selective agonist for a specific type of receptor

In some aspects, the immune modulator is an antagonist. In specific aspects, the antagonist is a competitive antagonist, which reversibly binds to the receptor at the same binding site as the endogenous ligand or agonist without activating the receptor. Competitive antagonist can affect the amount of agonist necessary to achieve a maximal response. In other aspects, the antagonist is a non-competitive antagonist, which binds to an active site of the receptor or an allosteric site of the receptor. Non-competitive antagonist can reduce the magnitude of the maximum response that can be attained by any amount of agonist. In further aspects, the antagonist is an uncompetitive antagonist, which requires receptor activation by an agonist before its binding to a separate allosteric binding site.

In some aspects, the immune modulator comprises an antibody or an antigen-binding fragment. The immunomodulating component can be a full length protein or a fragment thereof. The antibody or antigen-binding fragment can be derived from natural sources, or partly or wholly synthetically produced. In some aspects, the antibody is a monoclonal antibody. In some of these aspects, the monoclonal antibody is an IgG antibody. In certain aspects, the monoclonal antibody is an IgG1, IgG2, IgG3, or IgG4. In some other aspects, the antibody is a polyclonal antibody. In certain aspects, the antigen-binding fragment is selected from Fab, Fab′, and F(ab′)₂, F(ab1)₂, Fv, dAb, and Fd fragments. In certain aspects, the antigen-binding fragment is an scFv or (scFv)₂ fragment. In certain other aspects, the antibody or antigen-binding fragment is a NANOBODY® (single-domain antibody). In some aspects, the antibody or antigen-binding fragment is a bispecific or multispecific antibody.

In various aspects, the antibody or antigen-binding fragment is fully human. In some aspects, the antibody or antigen-binding fragment is humanized. In some aspects, the antibody or antigen-binding fragment is chimeric. In some of these aspects, the chimeric antibody has non-human V region domains and human C region domains. In some aspects, the antibody or antigen-binding fragment is non-human, such as murine or veterinary.

In certain aspects, the immunomodulating component is a polynucleotide. In some of these aspects, the polynucleotide includes, but is not limited to, an mRNA, a miRNA, an siRNA, an antisense oligonucleotide (e.g., antisense RNA or antisense DNA), a phosphorodiamidate morpholino oligomer (PMO), a peptide-conjugated phosphorodiamidate morpholino oligomer (PPMO), an shRNA, a lncRNA, a dsDNA, and combinations thereof. In some aspects, the polynucleotide is an RNA (e.g., an mRNA, a miRNA, an siRNA, an antisense oligonucleotide (e.g., antisense RNA), an shRNA, or an lncRNA). In some of these aspects, when the polynucleotide is an mRNA, it can be translated into a desired polypeptide. In some aspects, the polynucleotide is a microRNA (miRNA) or pre-miRNA molecule. In some of these aspects, the miRNA is delivered to the cytoplasm of the target cell, such that the miRNA molecule can silence a native mRNA in the target cell. In some aspects, the polynucleotide is a small interfering RNA (siRNA) or a short hairpin RNA (shRNA) capable of interfering with the expression of an oncogene or other dysregulating polypeptides. In some of these aspects, the siRNA is delivered to the cytoplasm of the target cell, such that the siRNA molecule can silence a native mRNA in the target cell. In some aspects, the polynucleotide is an antisense oligonucleotide (e.g., antisense RNA) that is complementary to an mRNA. In some aspects, the polynucleotide is a long non-coding RNA (lncRNA) capable of regulating gene expression and modulating diseases. In some aspects, the polynucleotide is a DNA that can be transcribed into an RNA. In some of these aspects, the transcribed RNA can be translated into a desired polypeptide.

In some aspects, the immunomodulating component is a protein, a peptide, a glycolipid, or a glycoprotein.

In various aspects, the EV composition comprises two or more above mentioned immunomodulating components, including mixtures, fusions, combinations and conjugates, of atoms, molecules, etc. In some aspects, the composition comprises one, two, three, four, five, six, seven, eight, nine, ten, eleven, or twelve different immunomodulating components associated with the membrane or enclosed within the enclosed volume of the extracellular vesicle. In certain aspects, the composition comprises a nucleic acid combined with a polypeptide. In certain aspects, the composition comprises two or more polypeptides conjugated to each other. In certain aspects, the composition comprises a protein conjugated to a biologically active molecule. In some of these aspects, the biologically active molecule is a prodrug.

In some aspects, any suitable method can be used to link an antigen or any other molecules of interest (e.g., adjuvant, immune modulator, and/or targeting moiety described herein) to an exterior surface and/or luminal surface of the EV. In certain aspects, the antigen or any other molecules of interest is linked to the exterior surface and/or the luminal surface of the EV by any suitable coupling strategies known in the art. In some aspects, the coupling strategy comprises: an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof. In some aspects, the anchoring moiety comprises a cholesterol, fatty acid (e.g., palmitate), tocopherol (e.g., vitamin E), alkyl chain, aromatic ring, or any combination thereof. In some aspects, the chemical conjugation comprises a maleimide moiety, copper-free, biorthogonal click chemistry (e.g., azide/strained alkyne (DIFO)), metal-catalyzed click chemistry (e.g., CUAAC, RUAAC), or any combination thereof. Additional description relating to the different approaches of linking an antigen or any other molecules of interest are provided elsewhere in the present disclosure. For instance, in some aspects, any of the coupling strategies described above can be used in combination with a scaffold moiety described herein (e.g., Scaffold X, e.g., PTGFRN).

### Scaffold X-Engineered EVs

In some aspects, EVs of the present disclosure comprise a membrane modified in its composition. For example, their membrane compositions can be modified by changing the protein, lipid, or glycan content of the membrane.

In some aspects, the surface-engineered EVs are generated by chemical and/or physical methods, such as PEG-induced fusion and/or ultrasonic fusion. In other aspects, the surface-engineered EVs are generated by genetic engineering. EVs produced from a genetically-modified producer cell or a progeny of the genetically-modified cell can contain modified membrane compositions. In some aspects, surface-engineered EVs have scaffold moiety at a higher or lower density (e.g., higher number) or include a variant or a fragment of the scaffold moiety.

For example, surface (e.g., Scaffold X)-engineered EVs, can be produced from a cell (e.g., HEK293 cells) transformed with an exogenous sequence encoding a scaffold moiety or a variant or a fragment thereof. EVs including scaffold moiety expressed from the exogenous sequence can include modified membrane compositions.

Various modifications or fragments of the scaffold moiety can be used for the aspects, of the present invention. For example, scaffold moiety modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used.

Scaffold moieties can be engineered to be expressed as a fusion molecule, e.g., fusion molecule of Scaffold X to an antigen, an adjuvant, and/or an immune modulator. For example, the fusion molecule can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to an antigen, an adjuvant, and/or an immune modulator. In case of the fusion molecule, the antigen, adjuvant, and/or immune modulator can be a natural peptide, a recombinant peptide, a synthetic peptide, or any combination thereof.

In some aspects, the surface (e.g., Scaffold X)-engineered EVs described herein demonstrate superior characteristics compared to EVs known in the art. For example, surface (e.g., Scaffold X)-engineered contain modified proteins more highly enriched on their surface than naturally occurring EVs or the EVs produced using conventional exosome proteins. Moreover, the surface (e.g., Scaffold X)-engineered EVs of the present invention can have greater, more specific, or more controlled biological activity compared to naturally occurring EVs or the EVs produced using conventional exosome proteins.

In some aspects, the Scaffold X comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at TABLE 7 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the fragments of PTGFRN polypeptide lack one or more functional or structural domains, such as IgV.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 33. In other aspects, the Scaffold X comprises the amino acid sequence of SEQ ID NO: 33, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 33 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 33.

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2, 3, 4, 5, 6, or 7. In other aspects, the Scaffold X comprises the amino acid sequence of SEQ ID NO: 2, 3, 4, 5, 6, or 7, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 2, 3, 4, 5, 6, or 7 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 2, 3, 4, 5, 6, or 7.

TABLE 1
Exemplary Scaffold X Protein Sequences

| Protein | Sequence |
|---|---|
| The PTGFRN | MGRLASRPLLLALLSLALCRGRVVRVPTATLVRVVGTELVIPCNVSDYDGPSEQNFDWSF |
| Protein | SSLGSSFVELASTWEVGFPAQLYQERLQRGEILLRRTANDAVELHIKNVQPSDQGHYKCS |
| (SEQ ID NO: | TPSTDATVQGNYEDTVQVKVLADSLHVGPSARPPPSLSLREGEPFELRCTAASASPLHTH |
| 1) | LALLWEVHRGPARRSVLALTHEGRFHPGLGYEQRYHSGDVRLDTVGSDAYRLSVSRALSA |
| | DQGSYRCIVSEWIAEQGNWQEIQEKAVEVATVVIQPSVLRAAVPKNVSVAEGKELDLTCN |
| | ITTDRADDVRPEVTWSFSRMPDSTLPGSRVLARLDRDSLVHSSPHVALSHVDARSYHLLV |
| | RDVSKENSGYYYCHVSLWAPGHNRSWHKVAEAVSSPAGVGVTWLEPDYQVYLNASKVPGF |
| | ADDPTELACRVVDTKSGEANVRFTVSWYYRMNRRSDNVVTSELLAVMDGDWTLKYGERSK |
| | QRAQDGDFIFSKEHTDTFNFRIQRTTEEDRGNYYCVVSAWTKQRNNSWVKSKDVFSKPVN |
| | IFWALEDSVLVVKARQPKPFFAAGNTFEMTCKVSSKNIKSPRYSVLIMAEKPVGDLSSPN |
| | ETKYIISLDQDSVVKLENWTDASRVDGVVLEKVQEDEFRYRMYQTQVSDAGLYRCMVTAW |
| | SPVRGSLWREAATSLSNPIEIDFQTSGPIFNASVHSDTPSVIRGDLIKLFCIITVEGAAL |
| | DPDDMAFDVSWFAVHSFGLDKAPVLLSSLDRKGIVTTSRRDWKSDLSLERVSVLEFLLQV |
| | HGSEDQDFGNYYCSVTPWVKSPTGSWQKEAEIHSKPVFITVKMDVLNAFKYPLLIGVGLS |
| | TVIGLLSCLIGYCSSHWCCKKEVQETRRERRRLMSMEMD |
| The PTGFRN | GPIFNASVHSDTPSVIRGDLIKLFCIITVEGAALDPDDMAFDVSWFAVHSFGLDKAPVLL |
| protein | SSLDRKGIVTTSRRDWKSDLSLERVSVLEFLLQVHGSEDQDFGNYYCSVTPWVKSPTGSW |
| Fragment | QKEAEIHSKPVFITVKMDVLNAFKYPLLIGVGLSTVIGLLSCLIGYCSSHWCCKKEVQET |
| (SEQ ID NO: | RRERRRLMSMEM |
| 33) | 687-878 of SEQ ID NO: 1 |

Non-limiting examples of other Scaffold X proteins can be found at U.S. patent Ser. No. 10/195,290B1, issued Feb. 5, 2019, which is incorporated by reference in its entireties.

In some aspects, the sequence encodes a fragment of the scaffold moiety lacking at least 5, 10, 50, 100, 200, 300, 400, 500, 600, 700, or 800 amino acids from the N-terminus of the native protein. In some aspects, the sequence encodes a fragment of the scaffold moiety lacking at least 5, 10, 50, 100, 200, 300, 400, 500, 600, 700, or 800 amino acids from the C-terminus of the native protein. In some aspects, the sequence encodes a fragment of the scaffold moiety lacking at least 5, 10, 50, 100, 200, 300, 400, 500, 600, 700, or 800 amino acids from both the N-terminus and C-terminus of the native protein. In some aspects, the sequence encodes a fragment of the scaffold moiety lacking one or more functional or structural domains of the native protein.

In some aspects, the scaffold moieties, e.g., Scaffold X, e.g., a PTGFRN protein, are linked to one or more heterologous proteins. The one or more heterologous proteins can be linked to the N-terminus of the scaffold moieties. The one or more heterologous proteins can be linked to the C-terminus of the scaffold moieties. In some aspects, the one or more heterologous proteins are linked to both the N-terminus and the C-terminus of the scaffold moieties. In some aspects, the heterologous protein is a mammalian protein. In some aspects, the heterologous protein is a human protein.

In some aspects, Scaffold X can be used to link any moiety to the luminal surface and on the exterior surface of the EV at the same time. For example, the PTGFRN polypeptide can be used to link one or more payloads disclosed herein (e.g., an antigen, an adjuvant, and/or an immune modulator) inside the lumen (e.g., on the luminal surface) in addition to the exterior surface of the EV. Therefore, in certain aspects, Scaffold X can be used for dual purposes, e.g., an antigen on the luminal surface and an adjuvant or immune modulator on the exterior surface of the EV, an antigen on the exterior surface of the EV, and the adjuvant or immune modulator on the luminal surface, an adjuvant on the luminal surface and an immune modulator on the exterior surface of the EV, or an immune modulator on the luminal surface and an adjuvant on the exterior surface of the EV.

### Scaffold Y-Engineered EVs

In some aspects, EVs of the present disclosure comprise an internal space (i.e., lumen) that is different from that of the naturally occurring EVs. For example, the EV can be changed such that the composition in the luminal surface of the EV has the protein, lipid, or glycan content different from that of the naturally-occurring exosomes.

In some aspects, engineered EVs can be produced from a cell transformed with an exogenous sequence encoding a scaffold moiety (e.g., exosome proteins, e.g., Scaffold Y) or a modification or a fragment of the scaffold moiety that changes the composition or content of the luminal surface of the EV. Various modifications or fragments of the exosome protein that can be expressed on the luminal surface of the EV can be used for the aspects of the present disclosure.

In some aspects, the exosome proteins that can change the luminal surface of the EVs, include, but are not limited to, the myristoylated alanine rich Protein Kinase C substrate (MARCKS) protein, the myristoylated alanine rich Protein Kinase C substrate like 1 (MARCKSL1) protein, the brain acid soluble protein 1 (BASP1) protein, or any combination thereof.

TABLE 2
Exemplary Scaffold Y Protein Sequences

| Protein | Sequence |
|---|---|
| The BASP1 | MGGKLSKKKK GYNVNDEKAK EKDKKAEGAA TEEEGTPKES EPQAAAEPAE |
| protein | AKEGKEKPDQ DAEGKAEEKE GEKDAAAAKE EAPKAEPEKT EGAAEAKAEP |
| (SEQ ID NO: | PKAPEQEQAA PGPAAGGEAP KAAEAAAAPA ESAAPAAGEE PSKEEGEPKK |
| 49) | TEAPAAPAAQ ETKSDGAPAS DSKPGSSEAA PSSKETPAAT EAPSSTPKAQ |
| | GPAASAEEPK PVEAPAANSD QTVTVKE |

The mature BASP1 protein sequence is missing the first Met from SEQ ID NO: 49 and thus contains amino acids 2 to 227 of SEQ ID NO: 49. Similarly, the mature MARCKS and MARCKSL1 proteins also lack the first Met from SEQ ID NOs: 47 and 48, respectively. Accordingly, the mature MARCKS protein contains amino acids 2 to 332 of SEQ ID NO: 47. The mature MARCKSL1 protein contains amino acids 2 to 227 of SEQ ID NO: 48.

In other aspects, Scaffold Y useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 2 to 227 of SEQ ID NO: 49. In other aspects, the Scaffold Y comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of SEQ ID NOs: 50-155. In other aspects, a Scaffold Y useful for the present disclosure comprises the amino acid sequence of SEQ ID NO: 49, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, a Scaffold Y useful for the present disclosure comprises the amino acid sequence of any one of SEQ ID NOs: 50-155 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NOs: 50-155.

In some aspects, the protein sequence of any of SEQ ID NOs: 47-155 is sufficient to be a Scaffold Y for the present disclosure (e.g., scaffold moiety linked to an antigen and/or an adjuvant and/or an immune modulator).

Non-limiting examples of scaffold proteins can be found at WO/2019/099942, published May 23, 2019 and WO/2020/101740, published May 22, 2020, which are incorporated by reference in their entireties.

In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached post-translationally to internal serine/threonine or tyrosine residues via a hydroxyester linkage. Membrane anchors known in the art are presented in the following table:

| Modification | Modifying Group |
|---|---|
| S-Palmitoylation | 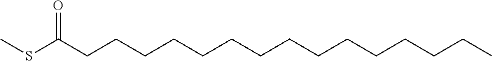 [SMILES: CCCCCCCCCCCCCCCC(=O)SC] |
| N-Palmitoylation | 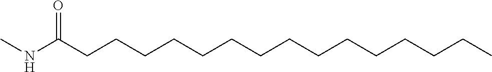 [SMILES: CCCCCCCCCCCCCCCC(=O)NC] |
| N-Myristoylation | 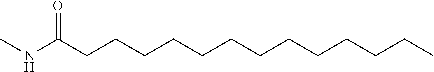 [SMILES: CCCCCCCCCCCCCC(=O)NC] |
| O-Acylation | 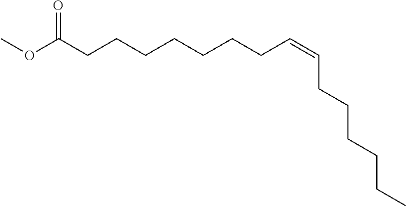 [SMILES: CCCCCC/C=C\CCCCCCCC(=O)OC] |
| Farnesylation | 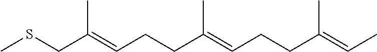 [SMILES: C/C=C(\C)CC/C=C(\C)CC/C=C(\C)CSC] |
| Geranylgeranylation | 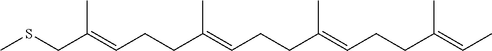 [SMILES: C/C=C(\C)CC/C=C(\C)CC/C=C(\C)CC/C=C(\C)CSC] |
| Cholesterol | 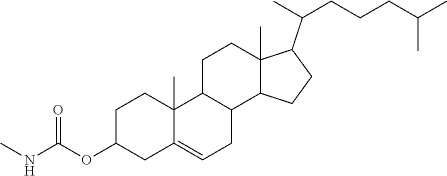 [SMILES: CNC(=O)OC1CCC2(C)C(=CCC3C2CCC2(C)C(C(C)CCCC(C)C)CCC32)C1] |

### Linkers

As described supra, extracellular vesicles (EVs) of the present disclosure can comprises one or more linkers that link one or more exogenous biologically active molecules disclosed herein (e.g., targeting moiety, therapeutic molecule (e.g., antigen), adjuvant, or immune modulator) to the EVs (e.g., to the exterior surface or on the luminal surface). In some aspects, the one or more exogenous biologically active molecules (e.g., targeting moiety, therapeutic molecule, adjuvant, or immune modulator) are linked to the EVs directly or via one or more scaffold moieties (e.g., Scaffold X or Scaffold Y). For example, in certain aspects, one or more exogenous biologically active molecules are linked to the exterior surface of an exosome via Scaffold X. In further aspects, one or more exogenous biologically active molecules are linked to the luminal surface of an exosome via Scaffold X or Scaffold Y. The linker can be any chemical moiety known in the art.

As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

Linkers can be susceptible to cleavage (“cleavable linker”) thereby facilitating release of the exogenous biologically active molecule (e.g., targeting moiety, therapeutic molecule, adjuvant, or immune modulator).

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the linker comprises a non-cleavable linker.

### Tropism

In some aspects, an EV disclosed herein can be engineered to adjust its properties, e.g., biodistribution, e.g., via incorporation of immuno-affinity ligands or cognate receptor ligands. For example, EVs disclosed herein can be engineered to direct them to a specific cellular type, e.g., Schwann cells, sensory neurons, motor neurons, or meningeal macrophages, or can surface engineered to enhance their migration to a specific compartment, e.g., to the CNS in order to improve intrathecal compartment retention.

In some aspects, an EV for delivery to the CNS disclosed herein comprises a bio-distribution modifying agent or targeting moiety. As used here, the terms “bio-distribution modifying agent” and “targeting moiety” are used interchangeably and refer to an agent that can modify the distribution of extracellular vesicles (e.g., exosomes, nanovesicles) in vivo or in vitro (e.g., in a mixed culture of cells of different varieties). In some aspects, the targeting moiety alters the tropism of the EV, i.e., the target moiety is a “tropism moiety”. As used herein, the term “tropism moiety” refers to a targeting moiety that when expressed on an EV alters and/or enhances the natural movement of the EV. For example, in some aspects, a tropism moiety can promote the EV to be taken up by a particular cell, tissue, or organ.

EVs exhibit preferential uptake in discrete cell types and tissues, and their tropism can be directed by adding proteins to their surface that interact with receptors on the surface of target cells. The tropism moiety can comprise a biological molecule, such as a protein, a peptide, a lipid, or a carbohydrate, or a synthetic molecule. For example, in some aspects the tropism moiety can comprise an affinity ligand, e.g., an antibody (such as an anti-CD19 nanobody, an anti-CD22 nanobody, an anti-CLEC9A nanobody, or an anti-CD3 nanobody), a VHH domain, a phage display peptide, a fibronectin domain, a camelid nanobody, and/or a vNAR. In some aspects, the tropism moiety can comprise, e.g., a synthetic polymer (e.g., PEG), a natural ligand/molecule (e.g., CD40L, albumin, CD24, CD55, CD59), and/or a recombinant protein (e.g., XTEN).

In some aspects, a tropism moiety can increase uptake of the EV by a cell. In some aspects, the tropism moiety that can increase uptake of the EV by a cell comprises a lymphocyte antigen 75 (also known as DEC205 or CD205), C-type lectin domain family 9 member A (CLEC9A), C-type lectin domain family 6 (CLEC6), C-type lectin domain family 4 member A (also known as DCIR or CLEC4A), Dendritic Cell-Specific Intercellular adhesion molecule-3-Grabbing Non-integrin (also known as DC-SIGN or CD209), lectin-type oxidized LDL receptor 1(LOX-1), macrophage receptor with collagenous structure (MARCO), C-type lectin domain family 12 member A (CLEC12A), C-type lectin domain family 10 member A (CLEC10A), DC-asialoglycoprotein receptor (DC-ASGPR), DC immunoreceptor 2 (DCIR2), Dectin-1, macrophage mannose receptor (MMR), BDCA-2 (CD303, CLEC4C), BDCA-1, BDCA-2, Dectin-2, BST-2 (CD317), Langerin, CD206, CD11b, CD11c, CD123, CD304, XCR1, AXL, SIGLEC 6, CD209, SIRPA, CX3CR1, GPR182, CD14, CD16, CD32, CD34, CD38, CD10, anti-CD3 antibody, or any combination thereof.

In some aspects, a tropism moiety useful for the present disclosure can increase the uptake of the EV by B cells. In certain aspects, a tropism moiety that can increase the uptake of the EV by B cells comprises an antibody or an antigen-binding fragment thereof. In some aspects, the antibody or antigen-binding fragment thereof specifically targets a marker expressed on a B cell. Non-limiting examples of such a marker include CD21, CD19, CD20, MHCII, CD40, ICOSL, or combinations thereof. In certain aspects, a tropism moiety that can increase the uptake of the EV by cells comprise CD40L, ICOS, or both. In some aspects, the addition of such a tropism moiety can increase the uptake of the EV by B cells

As described herein, a tropism moiety can increase the uptake of an EV by dendritic cells (DCs). In some aspects, a tropism moiety that can increase the uptake of the EV by DCs comprises an antibody or an antigen-binding fragment thereof. In certain aspects, the antibody or antigen-binding fragment thereof specifically targets a marker expressed on a DC. Non-limiting examples of such markers include DEC205, CD11c, Clec9a, XCR1, DCIR2, BDCA1, BDCA2, BDCA3, or combinations thereof. Additional examples are provided elsewhere in the present disclosure (see, e.g., Section titled “Methods of Modulating a Germinal Center Response”).

In some aspects, a tropism moiety can increase the uptake of an EV by follicular DCs. As used herein, “follicular” DCs (FDCs) are non-migratory population of immune cells found in primary and secondary follicles of the B cell areas of lymphoid tissues (e.g., lymph nodes, spleen, and mucosa-associated lymphoid tissue (MALT)). FDCs differ from DCs in that they are not derived from bone-marrow hematopoietic stem cells but are of mesenchymal origin. FDCs present antigen to B cells within the germinal center and regulate B cell antibody affinity maturation and B cell memory responses. Non-limiting examples of such tropism moieties include IgG, IgG-antigen complex, IgG-Fc, S aureus D domain dimer, anti-CR1 antibody, anti-CR2 antibody, or combinations thereof.

In some aspects, when tropism to the central nervous system is desired, an EV of the present disclosure can comprise a tissue or cell-specific target ligand, which increases EV tropism to a specific central nervous system tissue or cell. In some aspects, the cell is a glial cell. In some aspects, the glial cell is an oligodendrocyte, an astrocyte, an ependymal cell, a microglia cell, a Schwann cell, a satellite glial cell, an olfactory ensheathing cell, or a combination thereof. In some aspects, the cell is a neural stem cell. In some aspects, the cell-specific target ligand, which increases EV tropism to a Schwann cells binds to a Schwann cell surface marker such as Myelin Basic Protein (MBP), Myelin Protein Zero (P0), P75NTR, NCAM, PMP22, or any combination thereof. In some aspects, the cell-specific tropism moiety comprises an antibody or an antigen-binding portion thereof, an aptamer, or an agonist or antagonist of a receptor expressed on the surface of the Schwann cell.

In principle, the EVs of the present disclosure comprising at least one tropism moiety that can direct the EV to a specific target cell or tissue (e.g., a cell in the CNS or a Schwann cell in peripheral nerves) can be administered using any suitable administration method known in the art (e.g., intravenous injection or infusion) since the presence of the tropism moiety (alone or in combination with the use of a specific administration route) will induce a tropism of the EVs towards the desired target cell or tissue. In some aspects, the specific target cell comprises an antigen presenting cell (APC), B cell, or both. In certain aspects, an EV of the present disclosure with the ability to target an antigen presenting cell comprises (i) a cognate B cell antigen (e.g., polyGA), (ii) a universal CD4 T helper peptide (e.g., tetanus and diphtheria toxins (TT/DT)), and (iii) an adjuvant. In certain aspects, an EV of the present disclosure with the ability to target B cells comprises (i) a cognate B cell antigen (e.g., polyGA), (ii) CD40L (e.g., as a B cell targeting moiety), and (iii) an adjuvant. As disclosed herein, in some aspects, such an EV with the ability to target B cells can further comprise a co-stimulator to help enhance B cell activation (e.g., IL-21). Non-limiting examples of additional combinations of antigen, adjuvant, immunomodulatory, and targeting moieties that can be used to modify the EVs of the present disclosure are provided in Table 3 (below).

TABLE 3
Exemplary Combinations for EV-Based Vaccine Engineering

| Antigen | Adjuvant | Immunomodulator | Targeting Moiety | Vaccine Examples |
|---|---|---|---|---|
| Cognate B cell | | | B cells: | Pan Alzheimer's |
| peptide antigens | | | Antibodies | Ab and Tan |
| involved in | | | against: | peptide |
| Alzheimer’s disease, | | | CD21, | antigens |
| Parkinson’s disease, | | | CD19, | engineered on |
| Lewy body dementia: | | | CD20, | exosome |
| Amyloid-beta | | | MHCII, | surface or |
| (Aβ) | | | CD40, | attached (e.g. |
| Alpha | | | ICOSL | ALFA-tag) |
| synuclein | | | Recombinant | Pan Alzheimer's, |
| (αSyn) | | | CD40L or | Parkinson's, & |
| Tau | | | ICOS | LBD |
| | | | | Ab, aSyn, Tau |
| | | | | peptide |
| | | | | antigens |
| | | | | engineered on |
| | | | | exosome |
| | | | | surface or |
| | | | | attached |
| Dipeptide repeat | CpG (and | B cell & DC | Dendritic cells | PAN DPR for |
| (DPR) antigens | other | activator: | (DC): | ALS/FTD: |
| involved in ALS/FTD | TLR9 | CD40-L or anti- | Antibodies | Poly DPR |
| and other RAN | agonists) | CD40 agonist | or ligands | antigens |
| (repeat associated | | antibody, | against: | engineered on |
| non-AUG) involved in | | ICOS or anti- | DEC205, | exosome |
| repeat expansion | | ICOSL agonist | CD11c, | surface or |
| disorders: | | antibody | Clec9A, | attached (e.g. |
| PolyGA (10, | | | XCR1, | ALFA-tag) |
| 15, 20, 25 | | | DCIR2, | Pan peptide repeats |
| amino acid | | | BDCA1, | for repeat |
| repeats of GA) | | | BDCA2, | expansion |
| Other | | | BDCA3 | disorders (e.g., |
| dipeptides, | | | | ALS/FTD, HD): |
| such as | | | | Poly DPR and |
| PolyGR, | | | | polyglutamine |
| PolyGP, | | | | and other |
| PolyPA, | | | | RAN (repeat |
| PolyPR | | | | associated |
| PolyGlutamine | | | | non-AUG) |
| | | | | antigens |
| | | | | engineered on |
| | | | | exosome |
| | | | | surface or |
| | | | | attac |
| Huntington’s Disease | R848 | Cytokine | Follicular DC: | Pan Huntington's |
| (HD) antigens: | (and other | promoting | IgG, IgG- | Mutant HTT |
| Mutant HTT | TLR 7/8 | antibody | antigen | and |
| protein | agonists) | responses: | complex, | polyglutamine |
| PolyGlutamine | | IL-21, IL-4, IL-10, | IgG Fc, S | antigens |
| | | Tolerogenic B cell | aureus D | engineered on |
| | | responses: | domain | exosome |
| | | IL-10, TGF-b, IL-2, | dimer, anti- | surfaceor |
| | | GM-CSF | CR1 & CR2 | attached (e.g. |
| | | | (complement | ALFA-tag) |
| | | | receptor) | |
| | | | antibodies | |
| Familial and sporadic | MPLA | | | Pan ALS: |
| ALS: | (and other | | | DPR antigens |
| TDP-43 | TLR4 | | | plus TDP-43 |
| antigen | agonists) | | | antigens |
| | | | | engineered on |
| | | | | exosome |
| | | | | surface or |
| | | | | attached |

In certain aspects, the tropism moiety is linked, e.g., chemically linked via a maleimide moiety, to a scaffold moiety, e.g., a Scaffold X protein or a fragment thereof, on the exterior surface of the EV. Tropism can be further improved by the attachment of a half-life extension moiety (e.g., albumin or PEG) to the external surface of an EV of the present disclosure. In certain aspects, the half-life extension moiety (e.g., albumin or PEG) is linked, e.g., chemically linked via a maleimide moiety, to a scaffold moiety, e.g., a Scaffold X protein or a fragment thereof, on the exterior surface of the EV.

Pharmacokinetics, biodistribution, and in particular tropism and retention in the desired tissue or anatomical location can also be accomplished by selecting the appropriate administration route (e.g., intrathecal administration or intraocular administration to improve tropism to the central nervous system).

In some aspects, the EV comprises at least two different tropism moieties. In some aspects, the EV comprises three different tropism moieties. In some aspects, the EV comprises four different tropism moieties. In some aspects, the EV comprises five or more different tropism moieties. In some aspects, one or more of the tropism moieties increases uptake of the EV by a cell. In some aspects, each tropism moiety is attached to a scaffold moiety, e.g., a Scaffold X protein or a fragment thereof. In some aspects, multiple tropism moieties can be attached to the same scaffold moiety, e.g., a Scaffold X protein or a fragment thereof. In some aspects, several tropism moieties can be attached in tandem to a scaffold moiety, e.g., a Scaffold X protein or a fragment thereof. In some aspects, a tropism moiety disclosed herein or a combination thereof is attached to a scaffold moiety, e.g., a Scaffold X protein or a fragment thereof, via a linker or spacer. In some aspects, a linker or spacer or a combination thereof is interposed between two tropism moieties disclosed herein.

Non-limiting examples of tropism moieties capable of directing EVs of the present disclosure to different nervous system cell types are disclosed below.

Tropism moieties targeting Schwann cells: In some aspects, a tropism moiety can target a Schwann cell. In some aspects, the tropism moiety that directs an EV disclosed herein to a Schwann cell targets, e.g., a transferrin receptor (TfR), apolipoprotein D (ApoD), Galectin 1 (LGALS1), Myelin proteolipid protein (PLP), Glypican 1, or Syndecan 3. In some aspects, the tropism moiety directing an EV of the present disclosure to a Schwann cell is a transferrin, or a fragment, variant or derivative thereof.

In some aspects, a tropism moiety of the present disclosure targets a transferrin receptor (TfR). Transferrin receptors, e.g., TfR1 or TfR2, are carrier proteins for transferrin. Transferrin receptors import iron by internalizing the transferrin-ion complex through receptor-mediated endocytosis.

TfR1 (see, e.g., UniProt P02786 TFR1 Human) or transferrin receptor 1 (also known as cluster of differentiation 71 or CD71) is expressed on the endothelial cells of the blood-brain barrier (BBB). TfR1 is known to be expressed in a variety of cells such as red blood cells, monocytes, hepatocytes, intestinal cells, and erythroid cells, and is upregulated in rapidly dividing cells such as tumor cells (non small cell lung cancer, colon cancer, and leukemia) as well as in tissue affected by disorders such as acute respiratory distress syndrome (ARDS). TfR2 is primarily expressed in liver and erythroid cells, is found to a lesser extent in lung, spleen and muscle, and has a 45% identity and 66% similarity with TfR1. TfR1 is a transmembrane receptor that forms a homodimer of 760 residues with disulfide bonds and a molecular weight of 90 kDa. Affinity for transferrin varies between the two receptor types, with the affinity for TfR1 being at least 25-30 fold higher than that of TfR2.

Binding to TfR1 allows the transit of large molecules, e.g., antibodies, into the brain. Some TfR1-targeting antibodies have been shown to cross the blood-brain barrier, without interfering with the uptake of iron. Amongst those are the mouse anti rat-TfR antibody OX26 and the rat anti mouse-TfR antibody 8D3. The affinity of the antibody-TfR interaction is important to determine the success of transcytotic transport over endothelial cells of the BBB. Monovalent TfR interaction favors BBB transport due to altered intracellular sorting pathways. Avidity effects of bivalent interactions redirecting transport to the lysosome. Also, reducing TfR binding affinity directly promote dissociation from the TfR which increase brain parenchymal exposure of the TfR binding antibody. See, e.g., U.S. Pat. No. 8,821,943, which is herein incorporated by reference in its entirety. Accordingly, in some aspects, a tropism moiety of the present disclosure can comprise a ligand that can target TfR, e.g., target TfR1, such as transferrin, or an antibody or other binding molecule capable of specifically binding to TfR. In some aspects, the antibody targeting a transferrin receptor is a low affinity anti-transferring receptor antibody (see, e.g., US20190202936A1, which is herein incorporated by reference in its entirety).

In some aspects, the tropism moiety comprises all or a portion (e.g., a binding portion) of a ligand for a transferrin receptor, for example a human transferrin available in GenBank as Accession numbers NM001063, XM002793, XM039847, NM002343 or NM013900, among others, or a variant, fragment, or derivative thereof.

In some aspects, the tropism moiety comprises a transferrin-receptor-targeting moiety, i.e., a targeting moiety directed to a transferrin receptor. Suitable transferrin-receptor-targeting moieties include a transferrin or transferrin variant, such as, but not limited to, a serum transferrin, lacto transferrin (lactoferrin) ovotransferrin, or melanotransferrin. Transferrins are a family of nonheme iron-binding proteins found in vertebrates, including serum transferrins, lacto transferrins (lactoferrins), ovotransferrins, and melanotransferrins. Serum transferrin is a glycoprotein with a molecular weight of about 80 kDa, comprising a single polypeptide chain with two N-linked polysaccharide chains that are branched and terminate in multiple antennae, each with terminal sialic acid residues. There are two main domains, the N domain of about 330 amino acids, and the C domain of about 340 amino acids, each of which is divided into two subdomains, N1 and N2, and C1 and C2. Receptor binding of transferrin occurs through the C domain, regardless of glycosylation.

In some aspects, the tropism moiety is a serum transferrin or transferrin variant such as, but not limited to a hexasialo transferrin, a pentasialo transferrin, a tetrasialo transferrin, a trisialo transferrin, a disialo transferrin, a monosialo transferrin, or an asialo transferrin, or a carbohydrate-deficient transferrin (CDT) such as an asialo, monosialo or disialo transferrin, or a carbohydrate-free transferrin (CFT) such as an asialo transferrin. In some aspects, the tropism moiety is a transferrin variant having the N-terminal domain of transferrin, the C-terminal domain of transferrin, the glycosylation of native transferrin, reduced glycosylation as compared to native (wild-type) transferrin, no glycosylation, at least two N terminal lobes of transferrin, at least two C terminal lobes of transferrin, at least one mutation in the N domain, at least one mutation in the C domain, a mutation wherein the mutant has a weaker binding avidity for transferrin receptor than native transferrin, and/or a mutation wherein the mutant has a stronger binding avidity for transferrin receptor than native transferrin, or any combination of the foregoing.

In some aspects, the tropism moiety targeting a transferrin receptor comprises an anti-trasferrin receptor variable new antigen receptor (vNAR), e.g., a binding domain with a general motif structure (FW1-CDR1-FW2-3-CDR3-FW4). See, e.g., U.S. 2017-0348416, which is herein incorporated by reference in its entirety. vNARs are a key component of the adaptive immune system of sharks. At only 11 kDa, these single-domain structures are the smallest IgG-like proteins in the animal kingdom and provide an excellent platform for molecular engineering and biologics drug discovery. vNAR attributes include high affinity for target, ease of expression, stability, solubility, multi-specificity, and increased potential for solid tissue penetration. See Ubah et al. Biochem. Soc. Trans. (2018) 46(6):1559-1565.

In some aspects, the tropism moiety comprises a vNAR domain capable of specifically binding to TfR1, wherein the vNAR domain comprises or consists essentially of a vNAR scaffold with any one CDR1 peptide in Table 1 of U.S. 2017-0348416 in combination with any one CDR3 peptide in Table 1 of U.S. 2017-0348416.

In some aspects, a tropism moiety of the present disclosure targets ApoD. Unlike other lipoproteins, which are mainly produced in the liver, apolipoprotein D is mainly produced in the brain, cerebellum, and peripheral nerves. ApoD is 169 amino acids long, including a secretion peptide signal of 20 amino acids. It contains two glycosylation sites (aspargines 45 and 78) and the molecular weight of the mature protein varies from 20 to 32 kDa. ApoD binds steroid hormones such as progesterone and pregnenolone with a relatively strong affinity, and to estrogen with a weaker affinity. Arachidonic acid (AA) is an ApoD ligand with a much better affinity than that of progesterone or pregnenolone. Other ApoD ligands include E-3-methyl-2-hexenoic acid, retinoic acid, sphingomyelin and sphingolipids. Accordingly, in some aspects, a tropism moiety of the present disclosure comprises a ligand that can target ApoD, e.g., an antibody or other binding molecule capable of specifically binding to ApoD.

In some aspects, a tropism moiety of the present disclosure targets Galectin 1. The galectin-1 protein is 135 amino acids in length. Accordingly, in some aspects, a tropism moiety of the present disclosure comprises a ligand that can target Galectin 1, e.g., an antibody or other binding molecule capable of specifically binding to Galectin 1.

In some aspects, a tropism moiety of the present disclosure targets PLP. PLP is the major myelin protein from the CNS. It plays an important role in the formation or maintenance of the multilamellar structure of myelin. The myelin sheath is a multi-layered membrane, unique to the nervous system that functions as an insulator to greatly increase the efficiency of axonal impulse conduction. PLP is a highly conserved hydrophobic protein of 276 to 280 amino acids which contains four transmembrane segments, two disulfide bonds and which covalently binds lipids (at least six palmitate groups in mammals). Accordingly, in some aspects, a tropism moiety of the present disclosure comprises a ligand that can target PLP, e.g., an antibody or other binding molecule capable of specifically binding to PLP.

In some aspects, a tropism moiety of the present disclosure targets Glypican 1. Accordingly, in some aspects, a tropism moiety of the present disclosure comprises a ligand that can target Glypican 1, e.g., an antibody or other binding molecule capable of specifically binding to Glypican 1. In some aspects, a tropism moiety of the present disclosure targets Syndecan 3. Accordingly, in some aspects, a tropism moiety of the present disclosure comprises a ligand that can target Syndecan 3, e.g., an antibody or other binding molecule capable of specifically binding to Syndecan 3.

Tropism moieties targeting sensory neurons: In some aspects, a tropism moiety disclosed herein can direct an EV disclosed herein to a sensory neuron. In some aspects, the tropism moiety that directs an EV disclosed herein to a sensory neuron targets a Trk receptor, e.g., TrkA, TrkB, TrkC, or a combination thereof.

Trk (tropomyosin receptor kinase) receptors are a family of tyrosine kinases that regulates synaptic strength and plasticity in the mammalian nervous system. The common ligands of Trk receptors are neurotrophins, a family of growth factors critical to the functioning of the nervous system. The binding of these molecules is highly specific. Each type of neurotrophin has different binding affinity toward its corresponding Trk receptor. Accordingly, in some aspects, the tropism moiety directing an EV disclosed herein to a sensory neuron, comprises a neurotrophin.

Neurotrophins bind to Trk receptors as homodimers. Accordingly, in some aspects, the tropism moiety comprises at least two neurotrophins disclosed herein, e.g., in tandem. In some aspects, the tropism moiety comprises at least two neurotrophins disclosed herein, e.g., in tandem, that are attached to a scaffold protein, for example, Protein X, via a linker. In some aspects, the linker connecting the scaffold protein, e.g., Protein X, to the neurotrophin (e.g., a neurotrophin homodimer) has a length of at least 10 amino acids. In some aspects, the linker connecting the scaffold protein, e.g., Protein X, to the neurotrophin (e.g., a neurotrophin homodimer) has a length of at least about 25 amino acids, about 30 amino acids, about 35 amino acids, about 40 amino acids, about 45 amino acids, or about 50 amino acids.

In some aspects, the neurotrophin is a neurotrophin precursor, i.e., a proneurotrophin, which is later cleaved to produce a mature protein.

Nerve growth factor (NGF) is the first identified and probably the best characterized member of the neurotrophin family. It has prominent effects on developing sensory and sympathetic neurons of the peripheral nervous system. Brain-derived neurotrophic factor (BDNF) has neurotrophic activities similar to NGF, and is expressed mainly in the CNS and has been detected in the heart, lung, skeletal muscle and sciatic nerve in the periphery (Leibrock, J. et al., Nature, 341:149-152 (1989)). Neurotrophin-3 (NT-3) is the third member of the NGF family and is expressed predominantly in a subset of pyramidal and granular neurons of the hippocampus, and has been detected in the cerebellum, cerebral cortex and peripheral tissues such as liver and skeletal muscles (Ernfors, P. et al., Neuron 1: 983-996 (1990)). Neurotrophin-4 (also called NT-415) is the most variable member of the neurotrophin family. Neurotrophin-6 (NT-5) was found in teleost fish and binds to p75 receptor.

In some aspects, the neurotrophin targeting TrkB comprises, e.g., NT-4 or BDNF, or a fragment, variant, or derivative thereof. In some aspects, the neurotrophin targeting TrkA comprises, e.g., NGF or a fragment, variant, or derivative thereof. In some aspects, the neurotrophin targeting TrkC comprises, e.g., NT-3 or a fragment, variant, or derivative thereof.

In some aspects, the tropism moiety comprises brain derived neurotrophic factor (BDNF). In some aspects, the BDNF is a variant of native BDNF, such as a two amino acid carboxyl-truncated variant. In some aspects, the tropism moiety comprises the full-length 119 amino acid sequence of BDNF (HSDPARRGELSVCDSISEWVTAADKKTAVDMSGGTVTVLEKVPVSKGQLKQYFYETKCNPMGYTK EGCRGIDKRHWNSQCRTTQSYVRALTMDSKKRIGWRFIRIDTSCVCTLTIKRGR; SEQ ID NO: 385). In some aspects, a one amino-acid carboxy-truncated variant of BDNF is utilized (amino acids 1-118 of SEQ ID NO: 385).

In some aspects, the tropism moiety comprises a carboxy-truncated variant of the native BDNF, e.g., a variant in which 1, 2, 3, 4, 5, 6, 7, 8, 9, 10, or more than 10 amino acids are absent from the carboxy-terminus of the BDNF. BDNF variants include the complete 119 amino acid BDNF, the 117 or 118 amino acid variant with a truncated carboxyl terminus, variants with a truncated amino terminus, or variants with up to about 20%, about 30, or about 40% change in amino acid composition, as long as the protein variant still binds to the TrkB receptor with high affinity.

In some aspects, the tropism moiety comprises a two amino-acid carboxy-truncated variant of BDNF (amino acids 1-117 of SEQ ID NO: 385). In some aspects, the tropism moiety comprises a three amino-acid carboxy-truncated variant of BDNF (amino acids 1-116 of SEQ ID NO: 385). In some aspects, the tropism moiety comprises a four amino-acid carboxy-truncated variant of BDNF (amino acids 1-115 of SEQ ID NO: 385). In some aspects, the tropism moiety comprises a five amino-acid carboxy-truncated variant of BDNF (amino acids 1-114 of SEQ ID NO: 385). In some aspects, the tropism moiety comprises a BDNF that is at least about 60%, at least about 65%, at least about 70%, at least about 75%, at least about 80%, at least about 85%, at least about 90%, at least about 95%, at least about 99%, or about 100% identical with the sequence of SEQ ID NO: 385, or a truncated version thereof, e.g., the 117 or 118 amino acid variant with a one- or two-amino acid truncated carboxyl terminus, or variants with a truncated amino terminus. See, e.g., U.S. Pat. No. 8,053,569B2, which is herein incorporated by reference in its entirety.

In some aspects, the tropism moiety comprises nerve growth factor (NGF). In some aspects, the NGF is a variant of native NGF, such as a truncated variant. In some aspects, the tropism moiety comprises the 26-kDa beta subunit of protein, the only component of the 7S NGF complex that is biologically active. In some aspects, the tropism moiety comprises the full-length 120 amino acid sequence of beta NGF (SSSHPIFHRGEFSVCDSVSVWVGDKTTATDIKGKEVMVLGEVNINNSVFKQYFFETKCRDPNPVDSG CRGIDSKHWNSYCTTTHTFVKALTMDGKQAAWRFIRIDTACVCVLSRKAVRRA; SEQ ID NO: 386). In some aspects, the tropism moiety comprises a carboxy-truncated variant of the native NGF, e.g., a variant in which 1, 2, 3, 4, 5, 6, 7, 8, 9, 10, or more than 10 amino acids are absent from the carboxy-terminus of NGF. NGF variants include the complete 120 amino acid NGF, the shorter amino acid variants with a truncated carboxyl terminus, variants with a truncated amino terminus, or variants with up to about 20%, about 30%, or about 40% change in amino acid composition, as long as the tropism moiety still binds to the TrkB receptor with high affinity. In some aspects, the tropism moiety comprises an NGF that is at least about 60%, at least about 65%, at least about 70%, at least about 75%, at least about 80%, at least about 85%, at least about 90%, at least about 95%, at least about 99%, or about 100% identical with the sequence of SEQ ID NO: 386, or a truncated version thereof.

In some aspects, the tropism moiety comprises neurotrophin-3 (NT-3). In some aspects, the NT-3 is a variant of native NT-3, such as a truncated variant. In some aspects, the tropism moiety comprises the full-length 119 amino acid sequence of NT-3 (YAEHKSHRGEYSVCDSESLWVTDKSSAIDIRGHQVTVLGEIKTGNSPVKQYFYETRCKEARPVKNG CRGIDDKHWNSQCKTSQTYVRALTSENNKLVGWRWIRIDTSCVCALSRKIGRT; SEQ ID NO: 387). In some aspects, the tropism moiety comprises a carboxy-truncated variant of the native NT-3, e.g., a variant in which 1, 2, 3, 4, 5, 6, 7, 8, 9, 10, or more than 10 amino acids are absent from the carboxy-terminus of NT-3. NT-3 variants include the complete 119 amino acid NT-3, the shorter amino acid variants with a truncated carboxyl terminus, variants with a truncated amino terminus, or variants with up to about 20%, about 30%, or about 40% change in amino acid composition, as long as the tropism moiety still binds to the TrkC receptor with high affinity. In some aspects, the tropism moiety comprises an NT-3 that is at least about 60%, at least about 65%, at least about 70%, at least about 75%, at least about 80%, at least about 85%, at least about 90%, at least about 95%, at least about 99%, or about 100% identical with the sequence of SEQ ID NO: 387, or a truncated version thereof.

In some aspects, the tropism moiety comprises neurotrophin-4 (NT-4). In some aspects, the NT-4 is a variant of native NT-4, such as a truncated variant. In some aspects, the tropism moiety comprises the full-length 130 amino acid sequence of NT-4 (GVSETAPASRRGELAVCDAVSGWVTDRRTAVDLRGREVEVLGEVPAAGGSPLRQYFFETRCKADN AEEGGPGAGGGGCRGVDRRHWVSECKAKQSYVRALTADAQGRVGWRWIRIDTACVCTLLSRTGR A; SEQ ID NO: 388). In some aspects, the tropism moiety comprises a carboxy-truncated variant of the native NT-4, e.g., a variant in which 1, 2, 3, 4, 5, 6, 7, 8, 9, 10, or more than 10 amino acids are absent from the carboxy-terminus of NT-4. NT-4 variants include the complete 130 amino acid NT-4, the shorter amino acid variants with a truncated carboxyl terminus, variants with a truncated amino terminus, or variants with up to about 20%, about 30%, or about 40% change in amino acid composition, as long as the tropism moiety still binds to the TrkB receptor with high affinity. In some aspects, the tropism moiety comprises an NT-4 that is at least about 60%, at least about 65%, at least about 70%, at least about 75%, at least about 80%, at least about 85%, at least about 90%, at least about 95%, at least about 99%, or about 100% identical with the sequence of SEQ ID NO: 388, or a truncated version thereof.

Structure/function relationship studies of NGF and NGF-related recombinant molecules demonstrated that mutations in NGF region 25-36, along with other β-hairpin loop and non-loop regions, significantly influenced NGF/NGF-receptor interactions (Ibanez et al., EMBO J., 10, 2105-2110, (1991)). Small peptides derived from this region have been demonstrated to mimic NGF in binding to Mock receptor and affecting biological responses (LeSauteur et al. J. Biol. Chem. 270, 6564-6569, 1995). Dimers of cyclized peptides corresponding to β-loop regions of NGF were found to act as partial NGF agonists in that they had both survival-promoting and NGF-inhibiting activity while monomer and linear peptides were inactive (Longo et al., J. Neurosci. Res., 48, 1-17, 1997). Accordingly, in some aspects, a tropism moiety of the present disclosure comprises such peptides.

Cyclic peptides have also been designed and synthesized to mimic the β-loop regions of NGF, BDNF, NT3 and NT-4/5. Certain monomers, dimers or polymers of these cyclic peptides can have a three-dimensional structure, which binds to neurotrophin receptors under physiological conditions. All of these structural analogs of neurotrophins that bind to nerve cell surface receptors and are internalized can serve as the binding agent B of the compound according to the present disclosure to deliver the conjugated therapeutic moiety TM to the nervous system. Accordingly, in some aspects, a tropism moiety of the present disclosure comprises such cyclic peptides or combinations thereof.

In some aspects, antibodies against nerve cell surface receptors that are capable of binding to the receptors and being internalized can also serve as tropism moieties binding to a Trk receptor. For example, monoclonal antibody (MAb) 5C3 is specific for the NGF docking site of the human p140 TrkA receptor, with no cross-reactivity with human TrkB receptor. MAb 5C3 and its Fab mimic the effects of NGF in vitro, and image human Trk-A positive tumors in vivo (Kramer et al., Eur. J. Cancer, 33, 2090-2091, (1997)). Molecular cloning, recombination, mutagenesis and modeling studies of Mab 5C3 variable region indicated that three or less of its complementarity determining regions (CDRs) are relevant for binding to TrkA. Assays with recombinant CDRs and CDR-like synthetic polypeptides demonstrated that they had agonistic bioactivities similar to intact Mab 5C3. Monoclonal antibody MC192 against p75 receptor has also been demonstrated to have neurotrophic effects. Therefore, these antibodies and their functionally equivalent fragments can also serve as tropism moieties of the present disclosure.

In some aspects, peptidomimetics that are synthesized by incorporating unnatural amino acids or other organic molecules can also serve tropism moieties of the present disclosure.

Other neurotrophins are known in the art. Accordingly, in some aspects, the target moiety comprises a neurotrophin selected from the group consisting of fibroblast growth factor (FGF)-2 and other FGFs, erythropoietin (EPO), hepatocyte growth factor (HGF), epidermal growth factor (EGF), transforming growth factor (TGF)-a, TGF-(3, vascular endothelial growth factor (VEGF), interleukin-1 receptor antagonist (IL-1ra), ciliary neurotrophic factor (CNTF), glial-derived neurotrophic factor (GDNF), neurturin, platelet-derived growth factor (PDGF), heregulin, neuregulin, artemin, persephin, interleukins, granulocyte-colony stimulating factor (CSF), granulocyte-macrophage-CSF, netrins, cardiotrophin-1, hedgehogs, leukemia inhibitory factor (LIF), midlcine, pleiotrophin, bone morphogenetic proteins (BMPs), netrins, saposins, semaphorins, and stem cell factor (SCF).

In some aspects, the tropism moiety directing an EV disclosed herein to a sensory neuron, comprises a varicella zoster virus (VZV) peptide.

Tropism moieties targeting motor neurons: In some aspects, a tropism moiety disclosed herein can direct an EV disclosed herein to a motor neuron. In some aspects, the tropism moiety that directs an EV disclosed herein to a motor comprises a Rabies Virus Glycoprotein (RVG) peptide, a Targeted Axonal Import (TAxI) peptide, a P75R peptide, or a Tet-C peptide.

In some aspects, the tropism moiety comprises a Rabies Virus Glycoprotein (RVG) peptide. See, e.g., U.S. Pat. App. Publ. 2014-00294727, which is herein incorporated by reference in its entirety. In some aspects, the RVG peptide comprises amino acid residues 173-202 of the RVG (YTIWMPENPRPGTPCDIFTNSRGKRASNG; SEQ ID NO: 389) or a variant, fragment, or derivative thereof. In some aspects, the tropism moiety is a fragment of SEQ ID NO: 389. Such a fragment of SEQ ID NO: 389 can have, for example, 1, 2, 3, 4, 5, 6, 7, 8, 9, or 10 amino acids deleted from the N-terminal and/or the C-terminal of SEQ ID NO: 389. A functional fragment derived from SEQ ID NO: 389 can be identified by sequentially deleting N- and/or C-terminal amino acids from SEQ ID NO: 389 and assessing the function of the resulting peptide fragment, such as function of the peptide fragment to bind acetylcholine receptor and/or ability to transmit through the blood brain barrier. In some aspects, the tropism moiety comprises a fragment of SEQ ID NO: 389 28, 27, 26, 25, 24, 23, 22, 21, 20, 19, 18, 17, 16 or 15 amino acids in length. In some aspects, the tropism moiety comprises a fragment of SEQ ID NO: 389 less than 15 peptides in length.

A “variant” of a RVG peptide, for example SEQ ID NO: 389, is meant to refer to a molecule substantially similar in structure and function, i.e., where the function is the ability to pass or transit through the BBB, to either the entire molecule, or to a fragment thereof. A variant of an RVG peptide can contain a mutation or modification that differs from a reference amino acid in SEQ ID NO: 389. In some aspects, a variant of SEQ ID NO: 389 is a fragment of SEQ ID NO: 389 as disclosed herein. In some aspects, an RVG variant can be a different isoform of SEQ ID NO: 389 or can comprise different isomer amino acids. Variants can be naturally-occurring, synthetic, recombinant, or chemically modified polynucleotides or polypeptides isolated or generated using methods well known in the art. RVG variants can include conservative or non-conservative amino acid changes. See, e.g., U.S. Pat. No. 9,757,470, which is herein incorporated by reference in its entirety.

In some aspects, the tropism moiety comprises a Targeted Axonal Import (TAxI) peptide. In some aspects, the TAxI peptide is cyclized TAxI peptide of sequence SACQSQSQMRCGGG (SEQ ID NO: 390). See, e.g., Sellers et al. (2016) Proc. Natl. Acad. Sci. USA 113:2514-2519, and U.S. Pat. No. 9,056,892, which are herein incorporated by reference in their entireties. TAxI transport peptides as described herein may be of any length. Typically, the transport peptide will be between 6 and 50 amino acids in length, more typically between 10 and 20 amino acids in length. In some aspects, the TAxI transport peptide comprises the amino acid sequence QSQSQMR (SEQ ID NO: 391), ASGAQAR (SEQ ID NO: 392), PF, or TSTAPHLRLRLTSR (SEQ ID NO: 393). Optionally, the TAxI transport peptide further includes a flanking sequence to facilitate incorporation into a delivery construct or carrier, e.g., a linker. In one aspect, the peptide is flanked with cysteines. In some aspects, the TAxI transport peptide further comprises additional sequence selected to facilitate delivery into nuclei. For example, a peptide that facilitates nuclear delivery is a nuclear localizing signal (NLS). Typically, this signal consists of a few short sequences of positively charged lysines or arginines, such as PPKKRKV (SEQ ID NO: 394). In one aspect, the NLS has the amino acid sequence PKKRKV (SEQ ID NO: 395).

In some aspects, a tropism moiety of the present disclosure comprises a peptide BBB shuttle disclosed in the table below. See, e.g., Oller-Salvia et al. (2016) Chem. Soc. Rev. 45, 4690-4707, and Jafari et al. (2019) Expert Opinion on Drug Delivery 16:583-605 which are herein incorporated by reference in their entireties.

TABLE 4

| SEQ ID NO | Peptide | Sequence |
|---|---|---|
| 396 | Angiopep-2 | TFFYGGSRGKRNNFKTEEY-OH |
| 397 | ApoB (3371-3409) | SSVIDALQYKLEGTTRLTRK-RGLKLATALSLSNKFVEGS |
| 398 | ApoE (159-167)₂ | (LRKLRKRLL)₂ |
| 399 | Peptide-22 | Ac-C(&)MPRLRGC(&)-NH₂ |
| 400 | THR | THRPPMWSPVWP-NH₂ |
| 401 | THR retro-enantio | pwvpswmpprht-NH₂ |
| 402 | CRT | C(&)RTIGPSVC(&) |
| 403 | Leptin30 | YQQILTSMPSRNVIQISND-LENLRDLLHVL |
| 404 | RVG29 | YTIWMPENPRPGTPCDIFT-NSRGKRASNG-OH |
| 405 | ^{D}CDX | GreirtGraerwsekf-OH |
| 406 | Apamin | C(&₁)NC(&₂)KAPETALC(&₁)-AR-RC(&₂)QQH-NH₂ |
| 407 | MiniAp-4 | [Dap](&)KAPETALD(&) |
| 408 | GSH | y-L-glutamyl-CG-OH |
| 409 | G23 | HLNILSTLWKYRC |
| 410 | g7 | GFtGFLS(O-β-Glc)-NH₂ |
| 411 | TGN | TGNYKALHPHNG |
| 412 | TAT (47-57) | YGRKKRRQRRR-NH₂ |
| 413 | SynBI | RGGRLSYSRRRFSTSTGR |
| 414 | Diketopiperazines | &(N-MePhe)-(N-MePhe)Diketo-piperazines |
| 415 | PhPro | (Phenylproline)₄-NH₂ |

Nomenclature for cyclic peptides (&) is adapted to the 3-letter amino acid code from the one described by Spengler et at. Pept. Res., 2005, 65, 550-555
[Dap] stands for diaminopropionic acid.

### III. Pharmaceutical Compositions

Provided herein are pharmaceutical compositions comprising an EV of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an exosome described herein. In certain aspects, the EVs are co-administered with of one or more additional therapeutic agents, in a pharmaceutically acceptable carrier. In some aspects, the pharmaceutical composition comprising the EV is administered prior to administration of the additional therapeutic agents. In other aspects, the pharmaceutical composition comprising the EV is administered after the administration of the additional therapeutic agents. In further aspects, the pharmaceutical composition comprising the EV is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising exosomes is administered intravenously, e.g. by injection. The EVs can optionally be administered in combination with other therapeutic agents that are at least partly effective in treating the disease, disorder or condition for which the EVs are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as manitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs in an effective amount and in an appropriate solvent with one or a combination of ingredients enumerated herein, as desired. Generally, dispersions are prepared by incorporating the EVs into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs can be administered in the form of a depot injection or implant preparation which can be formulated in such a manner to permit a sustained or pulsatile release of the EVs.

Systemic administration of compositions comprising exosomes can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects, the pharmaceutical composition comprising exosomes is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising exosomes is administered as a liquid suspension. In certain aspects, the pharmaceutical composition is administered as a formulation that is capable of forming a depot following administration. In certain preferred aspects, the depot slowly releases the EVs into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs described herein. In some aspects, the dosage form is formulated as a liquid suspension for intravenous injection. In some aspects, the dosage form is formulated as a liquid suspension for intratumoral injection.

In certain aspects, the preparation of exosomes is subjected to radiation, e.g., X rays, gamma rays, beta particles, alpha particles, neutrons, protons, elemental nuclei, UV rays in order to damage residual replication-competent nucleic acids.

In certain aspects, the preparation of exosomes is subjected to gamma irradiation using an irradiation dose of more than 1, 5, 10, 15, 20, 25, 30, 35, 40, 50, 60, 70, 80, 90, 100, or more than 100 kGy.

In certain aspects, the preparation of exosomes is subjected to X-ray irradiation using an irradiation dose of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, 10000, or greater than 10000 mSv.

### IV. Kits

Also provided herein are kits comprising one or more exosomes described herein. In some aspects, provided herein is a pharmaceutical pack or kit comprising one or more containers filled with one or more of the ingredients of the pharmaceutical compositions described herein, such as one or more exosomes provided herein, optional an instruction for use. In some aspects, the kits contain a pharmaceutical composition described herein and any prophylactic or therapeutic agent, such as those described herein.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1: Generation of Engineered-EVs

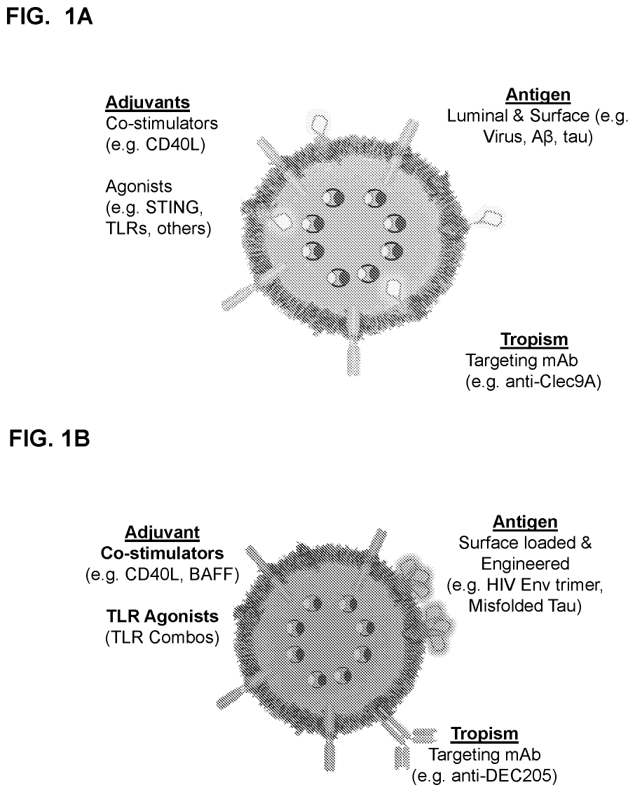

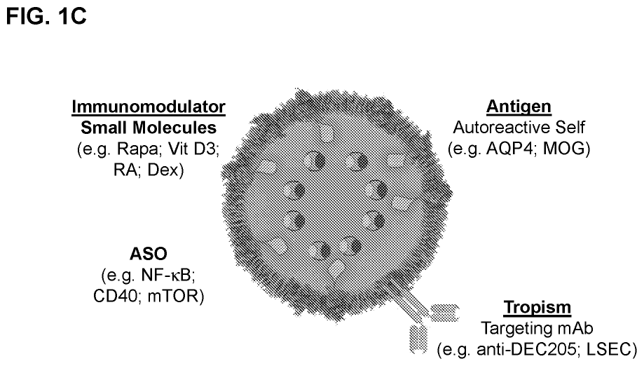

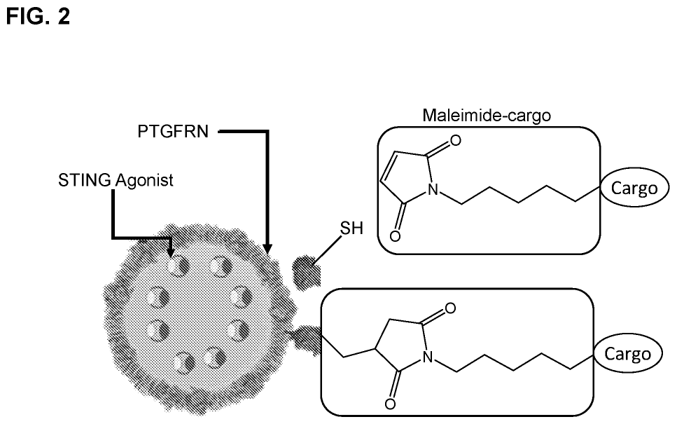

To generate EVs described herein, human embryonic kidney (HEK) cell line (HEK293SF) was used. The cells were then stably transfected with Scaffold X and/or Scaffold Y linked to an agent of interest (e.g., antigen, adjuvant, targeting moiety, and/or immune modulator). See FIGS. 1A, 1B, and 1C. Additionally, as shown in FIG. 2, in some of the EVs, an agent of interest (e.g., STING agonist) was loaded in the lumen of the EV, e.g., using methods described herein.

Upon transfection, HEK293SF cells were grown to high density in chemically defined medium for 7 days. Conditioned cell culture media was collected and centrifuged at 300-800×g for 5 minutes at room temperature to remove cells and large debris. Media supernatant was then supplemented with 1000 U/L BENZONASE® and incubated at 37° C. for 1 hour in a water bath. Supernatant was collected and centrifuged at 16,000×g for 30 minutes at 4° C. to remove residual cell debris and other large contaminants. Supernatant was then ultracentrifuged at 133,900×g for 3 hours at 4° C. to pellet the exosomes. Supernatant was discarded and any residual media was aspirated from the bottom of the tube. The pellet was resuspended in 200-1000 μL PBS (—Ca —Mg).

To further enrich exosome populations, the pellet was processed via density gradient purification (sucrose or OPTIPREP™).

The gradient was spun at between 150,000×g to 200,000×g (e.g., 150,000×g) for 16 hours at 4° C. in a 12 mL Ultra-Clear (344059) tube placed in a SW 41 Ti rotor to separate the exosome fraction.

The exosome layer was gently removed from the top layer and diluted in ˜32.5 mL PBS in a 38.5 mL Ultra-Clear (344058) tube and ultracentrifuged again at 133,900×g for 3 hours at 4° C. to pellet the purified exosomes. The resulting pellet was resuspended in a minimal volume of PBS (˜200 μL) and stored at 4° C.

For OPTIPREP™ gradient, a 3-tier sterile gradient is prepared with equal volumes of 10%, 30%, and 45% OPTIPREP™ in a 12 mL Ultra-Clear (344059) tube for a SW 41 Ti rotor. The pellet was added to the OPTIPREP™ gradient and ultracentrifuged at between 150,000×g to 200,000×g (e.g., 150,000×g) for 16 hours at 4° C. to separate the exosome fraction. The exosome layer was then gently collected from the top ˜3 mL of the tube.

The exosome fraction was diluted in ˜32 mL PBS in a 38.5 mL Ultra-Clear (344058) tube and ultracentrifuged at 133,900×g for 3 hours at 4° C. to pellet the purified exosomes. The pelleted exosomes were then resuspended in a minimal volume of PBS (˜200 μL) and stored at 4° C. until ready to be used.

### Example 2: Efficacy of Engineered-EVs to Induce Antigen-Specific T Cell Responses within the CNS

To assess the ability of the exosomes disclosed herein to induce antigen-specific immune responses within the CNS, an engineered-EV comprising an antigen associated with a neurological disorder will be constructed. As shown in FIGS. 1A, 1B, and 1C, the antigen will be expressed on either the external surface or the luminal surface of the EV. As described herein, the antigens will be linked to a scaffold moiety (e.g., Scaffold X and/or Scaffold Y) or conjugated directly to the EVs. Some of the EVs will comprise additional payloads disclosed herein (e.g., adjuvant, e.g., STING agonist or TLR agonist). These additional payloads will be expressed in the EVs linked to a scaffold moiety (e.g., Scaffold X and/or Scaffold Y) or loaded into the lumen of the EVs.

The above-engineered EVs will be administered (e.g., via intrathecal administration) to an experimental animal model for a neurological disorder. Then, the antigen-specific immune responses will be assessed in the animals (e.g., within the CNS) using assays, such as flow cytometry, ELISA, and/or ELISPOT.

### Example 3: Efficacy of Engineered-EVs to Induce Immune Tolerance

To assess the tolerogenic potential of EVs disclosed herein, engineered-EVs comprising antigens associated with an autoimmune disease (e.g., myelin oligodendrocyte glycoprotein (MOG, multiple sclerosis)) will be constructed. As in Example 2, in some of the EVs the antigen will be linked to a scaffold moiety (e.g., Scaffold X and/or Scaffold Y protein) described herein. In other EVs, the antigen will be conjugated directed to the EVs. Some of the engineered-EVs will further comprise one or more immune modulators that are capable of inducing immune tolerance (e.g., involved in the NFkB inhibition class, such as rapamycin and/or its derivatives, e.g., an ASO that targets NF-κB). These immune modulators will be expressed in the EVs linked to a scaffold moiety (e.g., Scaffold X and/or Scaffold Yprotein) or loaded exogenously into the lumen of the EVs.

The above-engineered EVs will be administered to an experimental animal model for an autoimmune disorder, such as experimental autoimmune encephalomyelitis (EAE). Then, the tolerogenic/regulatory T cell responses to the target antigen will be assessed in the animals using assays, such as flow cytometry, ELISA, and ELISPOT assay.

### Example 4: Construction of Engineered-EVs for the Treatment of Neurological Disorders (e.g., Alzheimer's Disease)

To assess the ability of the EVs disclosed herein to treat a neurological disorder, an engineered-EV comprising both B and T cell epitopes of a neuronal protein will be constructed. As shown in FIG. 2, in some aspects, the B and T cell epitopes will be expressed on the surface of the EVs via a scaffold moiety (e.g., Scaffold X, e.g., PTGFRN). In some aspects, the B and T cell epitopes will be loaded on the EV surface with maleimide chemistry. In some aspects, the EVs will further comprise different adjuvants (e.g., STING, CpG and MPLA), B cell co-stimulators, and/or targeting moieties.

Then, the EVs will be tested to determine their in vitro profiling and immunogenicity in vivo. In some aspects, the immunogenicity of the engineered EVs will be tested in vivo using human PBMC samples. B cell antigens will be validated using patient sera. Once validated, the antigens will be used to confirm the lack of T cell responses to the antigens and the production of robust antibody responses. In some aspects, the immunogenicity will be confirmed using human HLA mice.

Next, the engineered EVs will be administered to a mouse model of neurological disorders described herein (e.g., Alzheimer's disease). In determining the efficacy of the EVs, the localization of the antibodies induced by the EVs to sites of CNS pathology will be assessed. Other clinical factors (e.g., attenuation of disease and lack of T cell responses to antibody targets) will also be assessed.

### Example 5: Construction and Characterization of Exosomes with Tropism Moieties

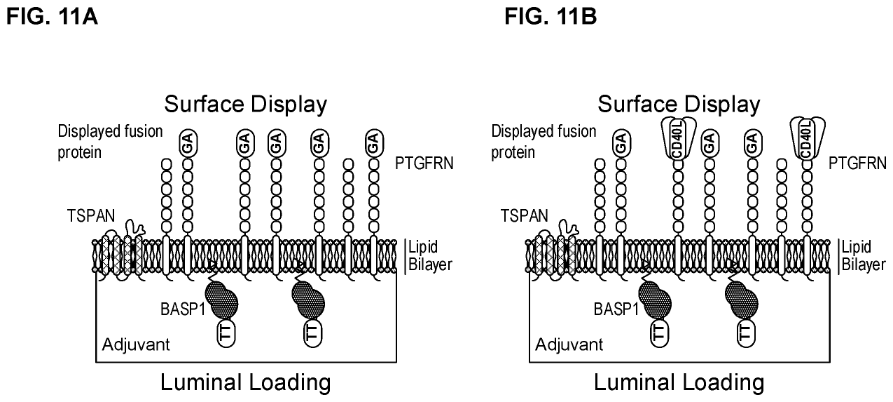

In order to direct EVs to specific cellular types, various constructs were created to express different tropism moieties. For instance, as shown in FIGS. 11A and 11B, some of the EVs were engineered to display a RVG peptide conjugated to PTGFRN on the exterior surface of the EV (referred to herein as “exoRVG”). To determine whether the RVG peptide could be used to direct EVs to neurons, several constructs were tested. The constructs tested were: RVG-PrX-mCherry-FLAG-HiBiT (construct 2021), linker-PrX-mCherry-FLAG-HiBiT (construct 2022), RVG-LAMP2B-mCherry-FLAG-HiBiT (construct 2023), and linker-LAMP2B-mCherry-FLAG-HiBiT (construct 2024).

“RVG” refers to a tropism moiety of having the amino acid sequence YTIWMPENPRPGTPCDIFTNSRGKRASNG (SEQ ID NO: 389). “Linker” refers to a linker having the amino acid sequence GGSSGSGSGSGGGGSGGGGTGTSSSGTGT (SEQ ID NO: 416). “FLAG” refers to a FLAG® epitope tag. “HiBiT” refers to a nano luciferase peptide. “mCherry” is a red fluorescent protein. “LAMP2B” and “PrX” (also referred to herein as “Scaffold X,” e.g., PTGFRN) are protein scaffolds, e.g., as described above. “ExoRVG” EVs are exosomes comprising an RVG tropism moiety.

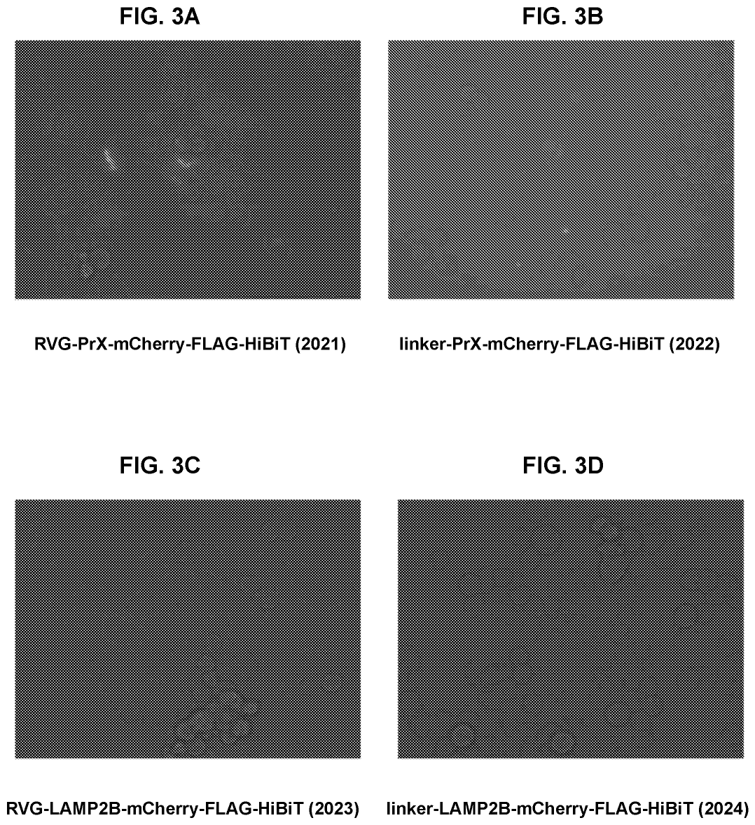

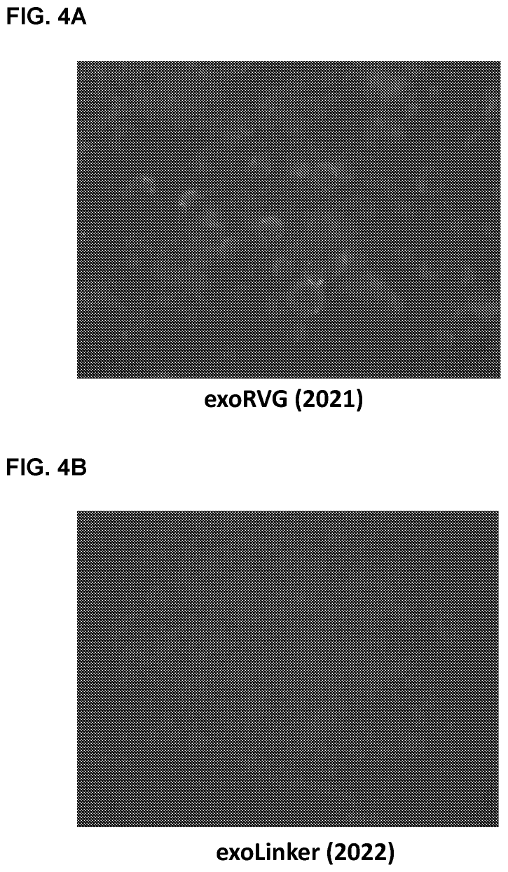

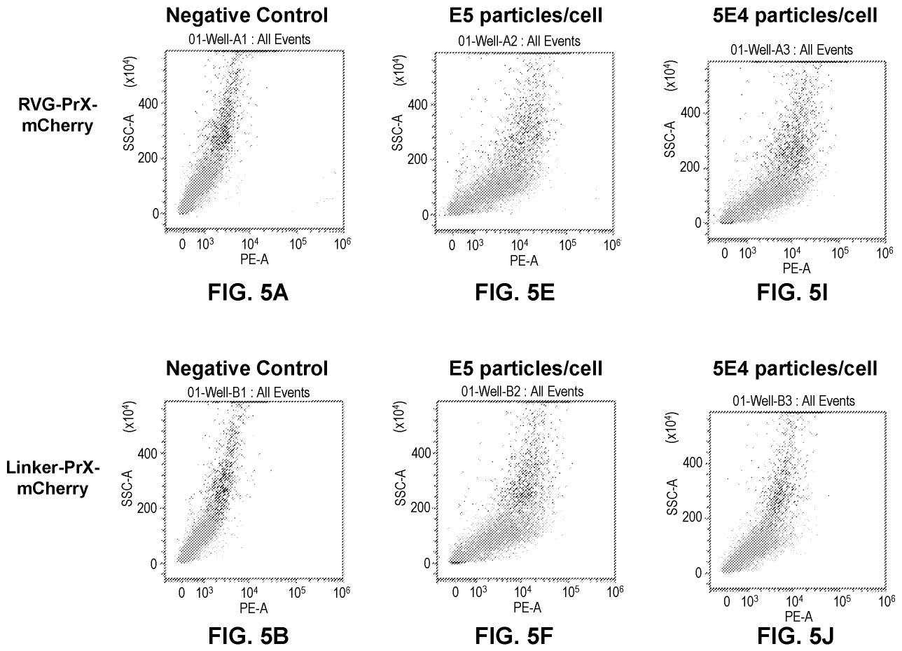

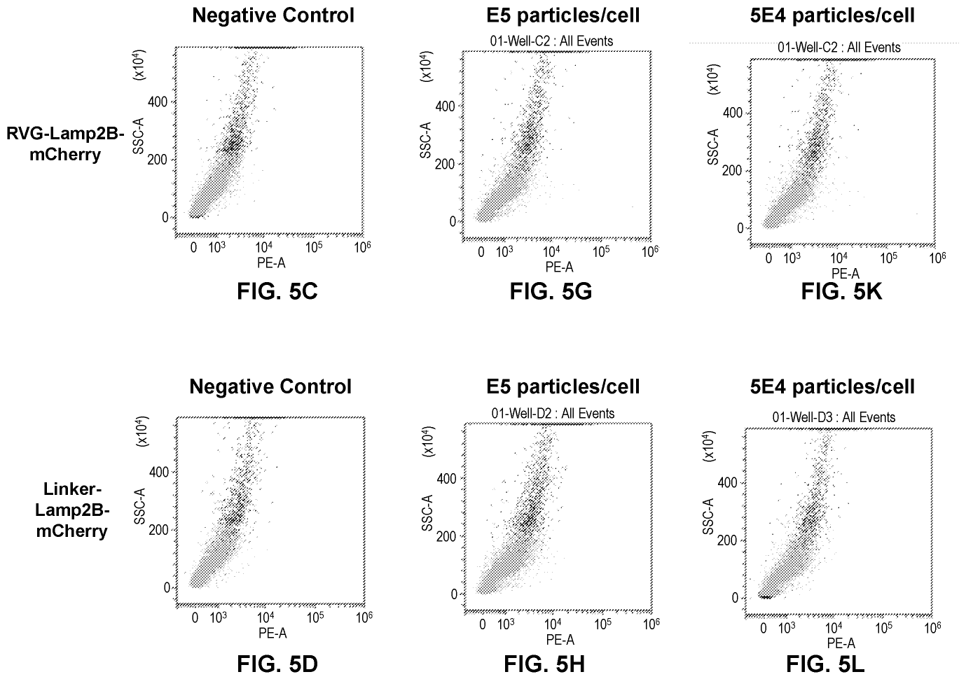

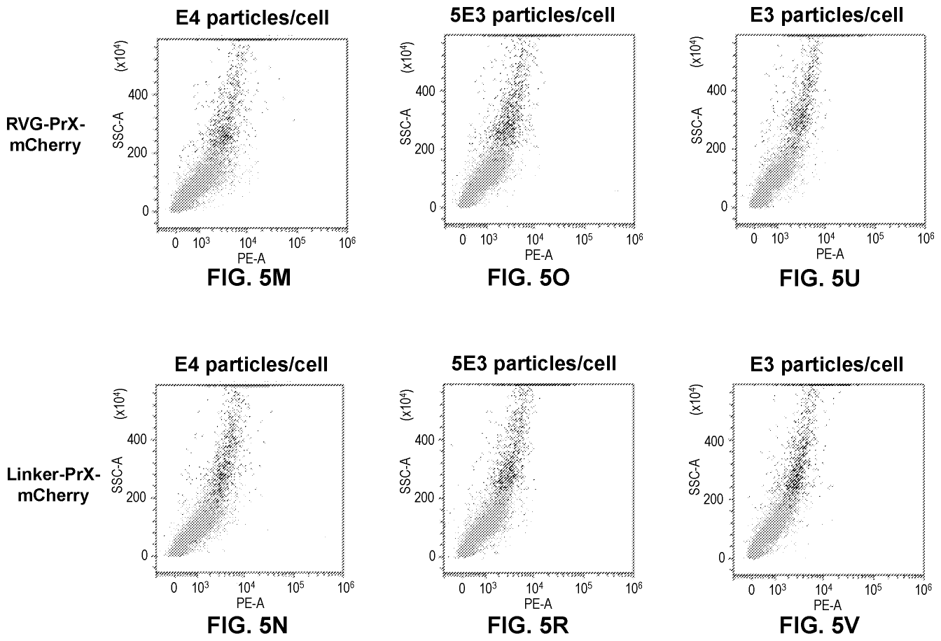

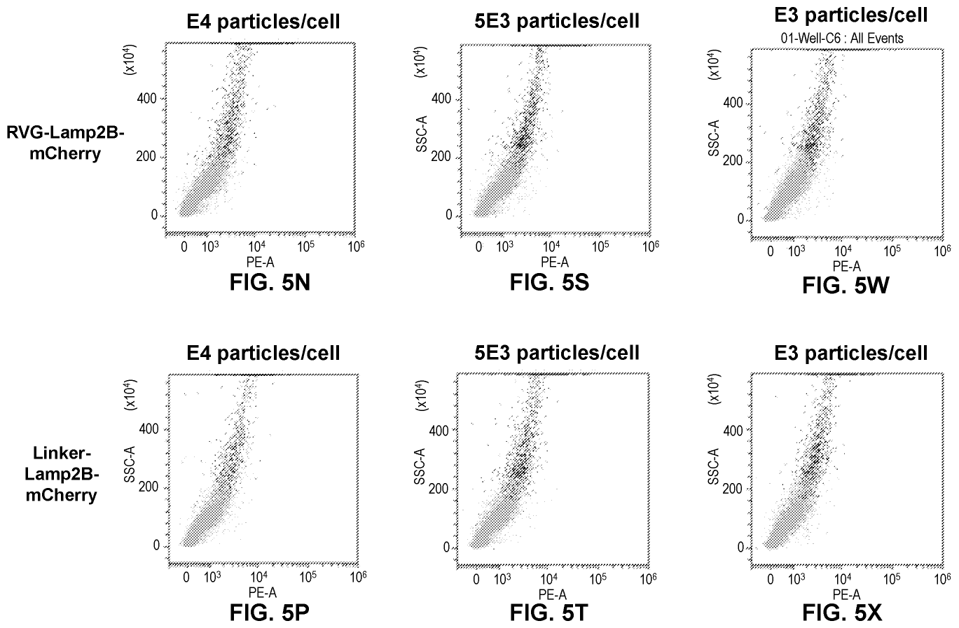

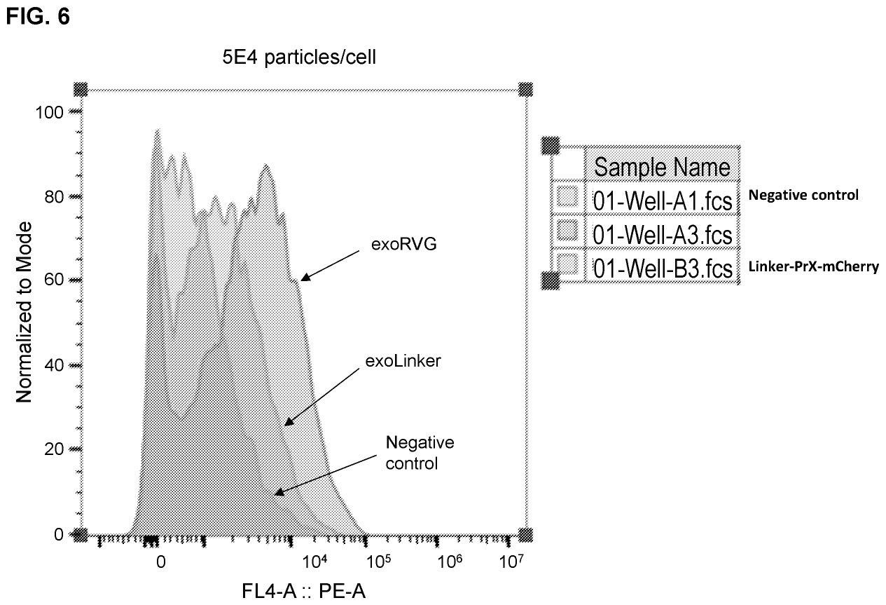

Neuro2A cells were incubated with 10⁵, 5×10⁴, 10⁴, 5×10³, or 10³ EV particles comprising the constructs disclosed above per neuro2A cell, and mCherry fluorescence was measured using microscopy. No obvious signal was observed at 1 hour or 2 hours after adding the EVs. However, EV uptake was observed at 5 hours with 10⁵ EV particles/neuro2A cell (FIGS. 3A-3D). Only the constructs comprising RVG showed uptake by the neuro2A cells. Increased uptake was observed after 18 hours (FIGS. 4A-4B). Flow cytometry showed significant uptake of UVs comprising RVG after 24 hours, both at 10⁵ EV particles/neuro2A cell and at 5×10⁴ EV particles/neuro2A cell (FIGS. 5A-5X and 6). These results indicated that attaching an RVG peptide to the external surface of an EV can target the EVs to neurons. Not to be bound by any one theory, in some aspects, the exoRVG can target the neurons as the RVG peptide can interact with receptors expressed on the neurons (e.g., nicotinic acetylcholine receptor (nAChR)).

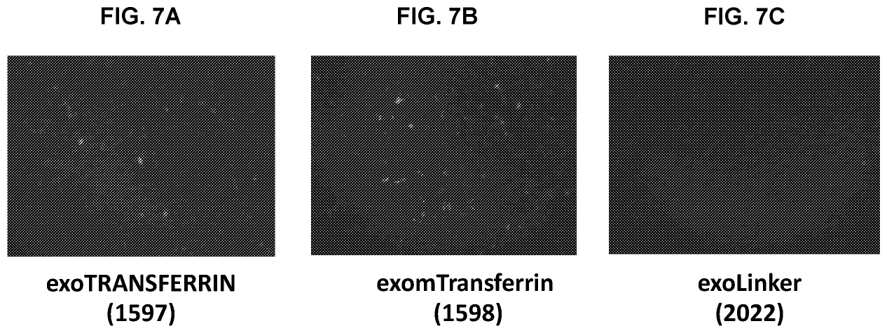

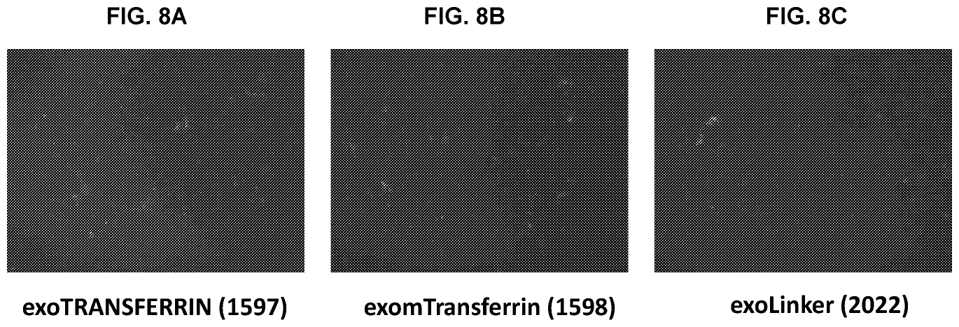

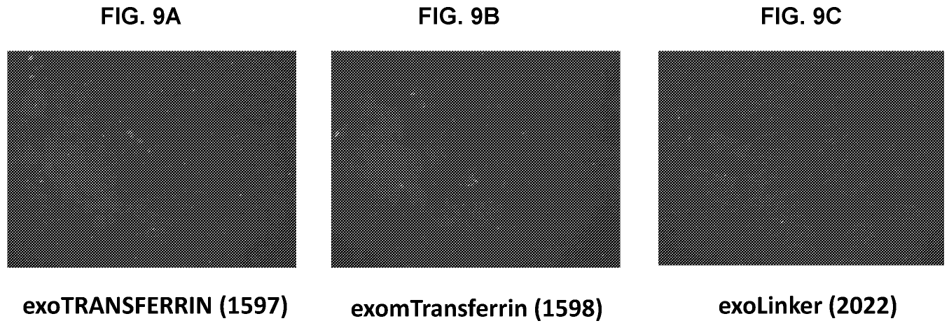

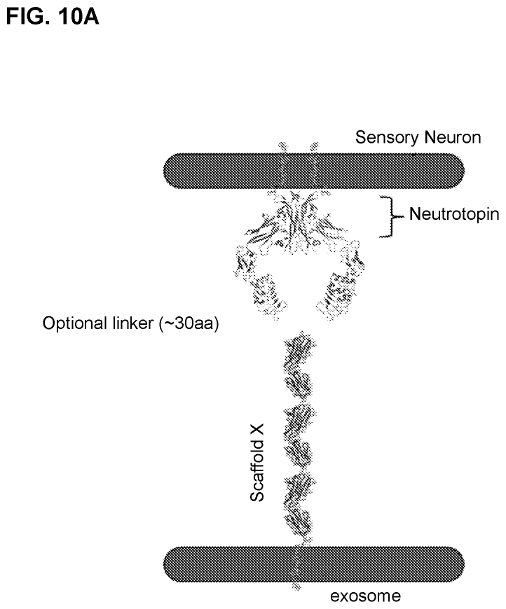

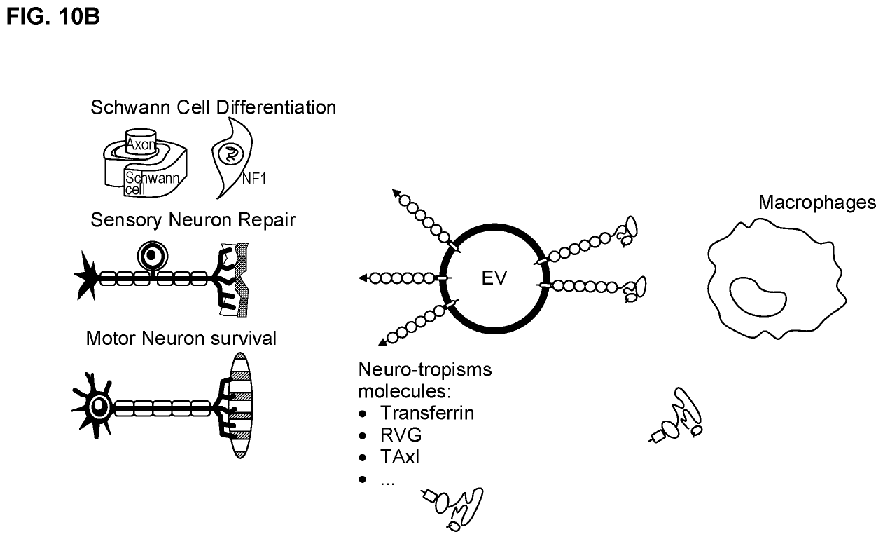

A second tropism moiety, transferrin, was also evaluated. Several constructs were tested: Transferrin-PrX-mCherry-FLAG (comprising human transferrin) (“exoTransferrin”; construct 1597), mTransferrin-PrX-mCherry-FLAG (comprising mouse transferrin) (“exomTransferrin”; construct 1598); and linker-PrX-mCherry-FLAG-HiBiT (“exoLinker”; construct 2022). 5×10⁵ EV particles per cell were used. Uptake was measured 3 hours after EV particle incubation started. Uptake was measured using microscopy. EV uptake by HeLa cells (FIGS. 7A-7C), Hep3B cells (FIG. 8A-8C) and Hep3G2 cells (FIGS. 9A-9C) was observed for both human and mouse transferrin-containing EVs, indicating that transferrin can be used to target EVs to these three cell types.

### Example 6: Construction of Engineered-EVs for the Treatment of C9FTD/ALS

To assess the ability of the EVs disclosed herein to treat C9FTD/ALS, an engineered-EV comprising B and/or T cell epitopes of a neuronal protein will be constructed. In certain aspects, the neuronal protein will be associated with a hexanucleotide GGGGCC repeat expansion in the C9orf72 gene (e.g., C9 RNA protein). As shown in FIG. 2, in some aspects, the B and/or T cell epitopes will be expressed on the surface of the EVs via a scaffold moiety (e.g., Scaffold X, e.g., PTGFRN). In some aspects, the B and/or T cell epitopes will be loaded on the EV surface with maleimide chemistry. In some aspects, the EVs will further comprise different adjuvants (e.g., STING, CpG and MPLA), B cell co-stimulators, and/or targeting moieties. As described herein, the EVs will be engineered to stimulate antibody-producing B cells without activating harmful T cells.

Then, the EVs will be tested to determine their in vitro profiling and immunogenicity in vivo. In some aspects, the immunogenicity of the engineered EVs will be tested in vivo using human PBMC samples. B cell antigens will be validated using patient sera. Once validated, the antigens will be used to confirm the lack of T cell responses to the antigens and the production of robust antibody responses. In some aspects, the immunogenicity will be confirmed using human HLA mice.

Next, the engineered EVs will be administered to a mouse model of neurological disorders described herein (e.g., ALS). In determining the efficacy of the EVs, the localization of the antibodies induced by the EVs to sites of CNS pathology will be assessed. Other clinical factors (e.g., attenuation of disease and lack of T cell responses to antibody targets, e.g., formation of RNA foci and deposition of dipeptide repeat (DPR) proteins derived from repeat associated non-ATG (RAN) translation) will also be assessed.

### Example 7: Use of Antigen Presenting Cell (APC) Targeting Engineered-EVs to Treat C9FTD/ALS

Further to Example 6 provided above, in some aspects, an engineered-EV that can target APCs to induce T cell dependent antibody response will be used to assess the efficacy of the EVs in treating C9FTD/ALS. In some aspects, the EVs will comprise one or more of the following components: (1) cognate (e.g., polyGA) B cell antigen; (2) universal CD4 T helper peptide with epitopes from common vaccine antigens (e.g., tetanus and diphtheria toxins (TT/DT); and (3) an adjuvant. As shown in FIG. 11A, in some aspects, the polyGA B cell antigens will be displayed on the exterior surface of the EV via Scaffold X (e.g., PTGFRN). In certain aspects, the EVs will comprise poly GA B cell antigen of different repeat lengths (e.g., about 10, about 15, or about 20 repeats). In some aspects, the universal CD4 T helper peptide will be displayed on the luminal surface, e.g., linked to Scaffold Y (e.g., BASP-1). In certain aspects, the universal CD4 T helper peptide will consist of amino acids 830-844 of tetanus toxin; and/or amino acids 271-290 and 331-350 of diphtheria toxin protein). In some aspects, an adjuvant will be loaded in the lumen of the EV (i.e., not associated with a scaffold moiety). Non-limiting examples of adjuvants that will be tested include: a TLR9 agonist (e.g., CpG class C), a STING agonist (e.g., CL-656), a TLR4 agonist (e.g., MPLA), and combinations thereof. In some aspects, the EVs will further comprise a FLAG tag to allow for poly GA B cell antigen quantification.

To characterize the above engineered-EVs, various suitable methods known in the art will be used. For instance, in some aspects, to detect and/or quantitate the polyGA B cell antigen expression in the EVs, an ELISA assay will be used to detect the FLAG tag. In some aspects, the expression of the universal CD4 T helper peptide fused to BASP-1 will be assessed using western blot and looking for shift in BASP-1 size. In some aspects, the successful loading of the adjuvant in the lumen of the EVs will be assessed by mass spectrometry.

Once the engineered-EVs have been validated, they will be used to further validate the EVs in vivo by vaccinating (e.g., via subcutaneous administration) mice. Various doses and dosing intervals will be tested. In some aspects, after the initial administration, the animals will be boosted two weeks later with a second administration of the engineered-EVs. Then, approximately two weeks after the boost, sera will be collected from the animals, and the amount of polyGA-specific antibody isotypes will be assessed, e.g., with an ELISA. In some aspects, neutralizing antibody activity will also be assessed using an in vitro assay that can measure antibody-mediated inhibition of polyGA aggregation and/or cell toxicity. In some aspects, both tetanus/diphtheria and polyGA-specific T cells will also be assessed, e.g., using an ELISPOT. In certain aspects, to assess the repeat motif and length specificity of the EV induced anti-polyGA antibodies, cells expressing individual RAN proteins using alternative codon constructs and RAN proteins of different repeat lengths will be used.

In some aspects, engineered-EVs that induce approximately 1 mg/mL of antigen-specific antibodies in the sera (estimated to be the amount needed for CNS penetration and efficacy based on passive immunization studies) will be administered to an animal model for ALS/FTD, e.g., C9-BAC mice, such as that described in Liu et al., Neuron 90: 521-534 (May 2016), which is incorporated herein by reference in its entirety. As described in Example 6, one or more of the following will be assessed to determine the efficacy of the EVs: (i) localization of the antibodies induced by the EVs to sites of CNS pathology and whether the antibodies can recognize RAN aggregates; (ii) attenuation of disease and lack of T cell responses to antibody targets, e.g., formation of RNA foci and deposition of dipeptide repeat (DPR) proteins derived from repeat associated non-ATG (RAN) translation.

### Example 8: Use of Engineered-EVs with Enhanced B Cell Targeting and/or B Cell Activating Capability to Treat C9FTD/ALS

To further assess the ability of the EVs disclosed herein to treat neurodegenerative diseases, such as C9FTD/ALS, in some aspects, engineered-EVs with an enhanced ability to target B cells and/or to induce B cell activation will be used. In some aspects, the EVs will comprise one or more of the following components: (1) polyGA B cell antigen; (2) CD40L (e.g., as a B cell targeting moiety) and/or IL-21 (e.g., co-stimulator to promote enhanced B cell activation); and (3) an adjuvant. In certain aspects, the EVs will additionally comprise a universal CD4 helper peptide (e.g., such as that described in Example 7). In some aspects, the polyGA B cell antigen will be displayed on the exterior surface of the EV in combination with the CD40L and/or IL-21. As shown in FIG. 11B, in some aspects, the polyGA B cell antigen, CD40L, and/or IL-21 will be displayed as a fusion linked to a Scaffold X (e.g., PTGFRN). As in Example 7, in some aspects, the polyGA B cell antigen will be of different repeat lengths. In some aspects, the adjuvant will be loaded in the lumen of the EV (i.e., not associated with a scaffold moiety). Non-limiting examples of adjuvants that will be tested include a TLR9 agonist (e.g., CpG class B), a TLR4 agonist (e.g., MPLA), or both. In some aspects, the EVs will further comprise a FLAG tag to allow for poly GA B cell antigen quantification.

In some aspects, prior to in vivo administration, the above-described engineered-EVs will be characterized using any suitable methods known in the art (e.g., such as those described in Example 7). In some aspects, the CD40L and/or IL-21 expression in the EVs will be assessed using western blot. In certain aspects, whether the CD40L and/or IL-21 displayed on the exterior surface of the EVs is functional, a primary murine B cell activation assay will be used.

Once characterized, the efficacy of the engineered-EVs will be assessed in vivo, e.g., as described in Example 7.

### Example 9: Effect of Engineered-EVs Comprising a Neuronal Protein on Neurological and Behavioral Function

Further to the examples provided above (e.g., Examples 7 and 8), the ability of the EVs disclosed herein to improve neurological and/or behavioral functions will also be assessed, e.g., in the C9-BAC mice. Briefly, C9-BAC mice and non-transgenic (NT) littermates will be treated with either an engineered-EV disclosed herein (e.g., such as that described in Example 7 or Example 8) or a mock injection (e.g., administration of a non-engineered empty EV). The treatments will begin at around eight weeks post-birth or later because that is when the C9-BAC mice show RAN aggregates but no overt phenotypes. The EVs will be administered at various doses, dosing intervals, and routes of administration. In certain aspects, the EVs will be administered to the animals every two weeks for 24 weeks. At various time points post administration, anti-polyGA antibody titers, RAN protein levels, and adverse responses will be monitored in the serum of the animals. In some aspects, at around 32 weeks post administration, behavioral function of the animals will be assessed using different suitable methods known in the art, such as DIGIGAIT™ and openfield analysis. In some aspects, at around 36 weeks post administration, tissues will be harvested from the animals for molecular and histological comparisons.

### Example 10: Expression Analysis of Engineered-EVs in Glioblastoma Multiforme (GBM)

To further assess the ability of the EVs disclosed herein to treat different neurological disorders, the expression pattern of the EVs was assessed in the brain of a GBM animal model. Briefly, mice were inoculated with syngeneic GL261-Luc GBM cancer cells. Upon disease onset, engineered-EVs comprising a STING agonist were administered (via intratumoral injection) to the animals. Then, the distribution of the EVs was assessed.

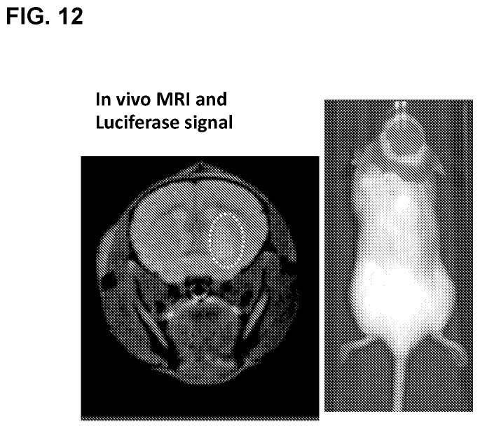

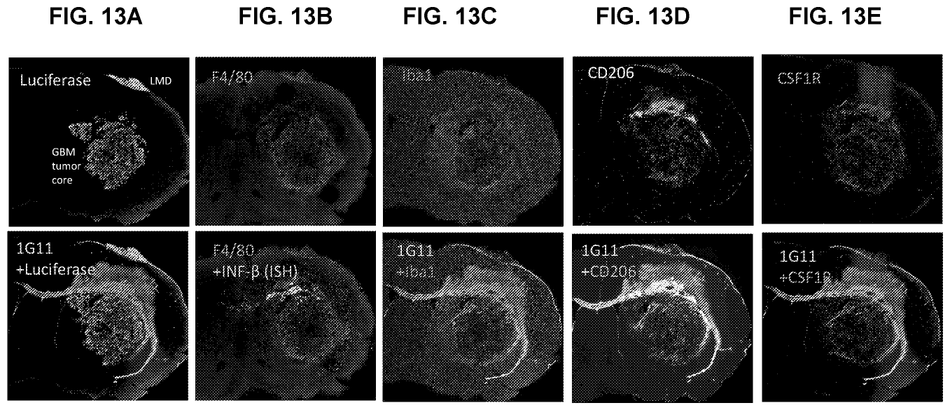

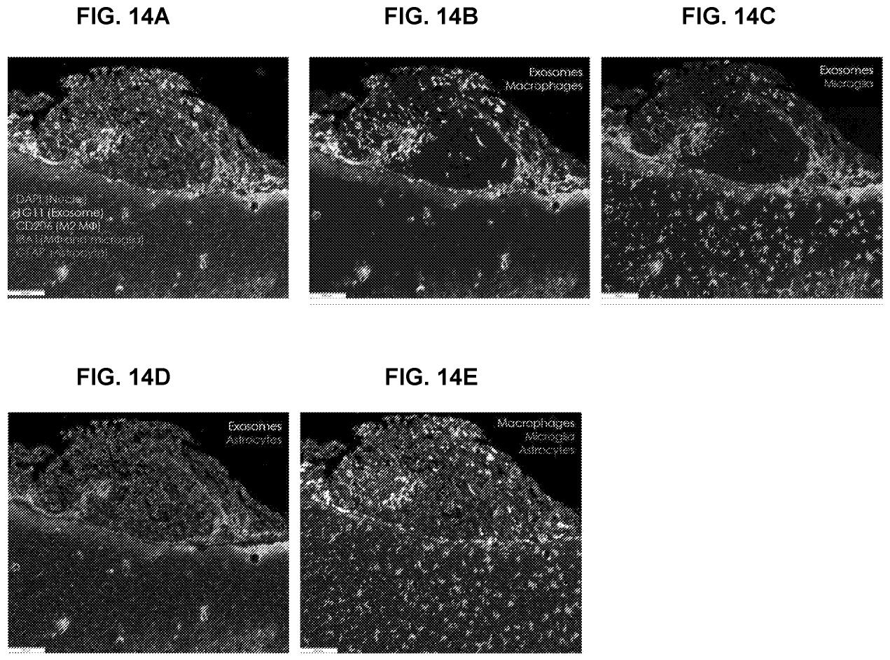

As shown in FIGS. 12 and 13A, intratumoral injection of the EVs resulted in specific expression of the EVs within the brain region of the GBM animals. And, as shown in FIGS. 13B-13E, upon GBM onset, there was a significant influx of macrophages, particularly those with M2 polarization markers and/or producing INF-β. And, as shown in FIGS. 13A-13E (bottom row), intratumoral dosed EVs were expressed both in the brain parenchyma and white matter of the GBM animals. In particular, the EVs had spread through the extra-cellular matrix of the brain and along perivascular spaces (see FIGS. 14A-14E). There was significant overlap in expression of the EVs and the above-described macrophages. There was also overlap in the expression of the EVs with both microglia and astrocytes (see FIG. 14E), suggesting a possible association between the EVs and these cell types.

The above data demonstrate that the EVs described herein can successfully target the CNS and could be useful in treating a wide range of neurological disorders, including GBM and leptomeningeal disease (LMD).

### Example 11: Analysis of Alum Adsorption by EVs

As described herein, EVs of the present disclosure can be rapidly engineered to comprise an antigen and one or more of the following moieties of interest: adjuvant, immunomodulatory, and targeting moiety. To help illustrate, the loading of alum onto the EVs was assessed. Briefly, alum was mixed with different concentrations of EVs (i.e., 1×10¹⁰, 1×10¹¹, and 1×10¹²) at a 1:1 volume ratio. Then, the mixture was vortexed and incubated for 5 minutes, 30 minutes, or 60 minutes. After the incubation, the mixture was spun down at 10,000×g to pellet the alum (including EVs that were loaded with the alum). The amount of free EVs (i.e., did not adsorb alum) in the supernatant was measured using a BCA assay.

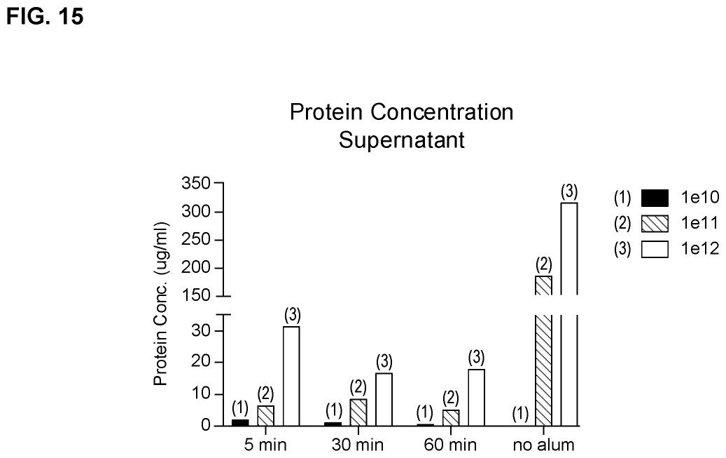

As shown in FIG. 15, alum was loaded onto the EVs just after 5 minutes of incubation. At higher EV concentrations, longer incubation resulted in greater fraction of the EVs adsorbing the alum. Such results demonstrate that the EVs of the present can be modified to comprise one or more moieties of interest (e.g., adjuvant) that can be useful in treating a disease or disorder described herein.

### Example 12: Analysis of Immune Response after Administration of exoPolyGA

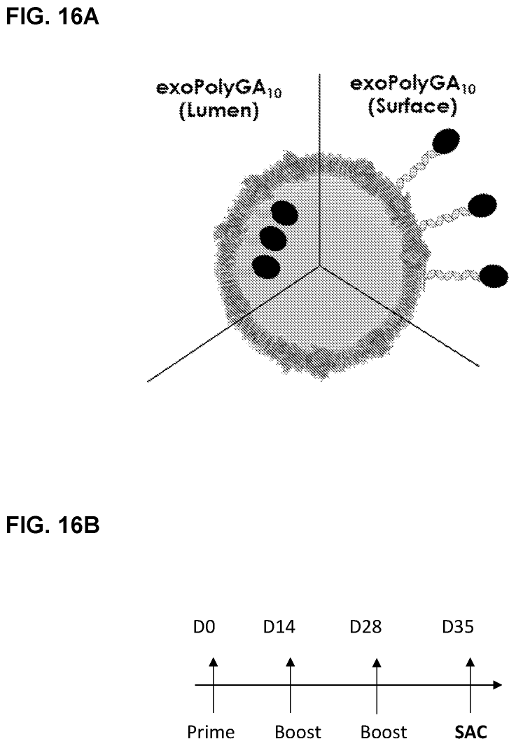

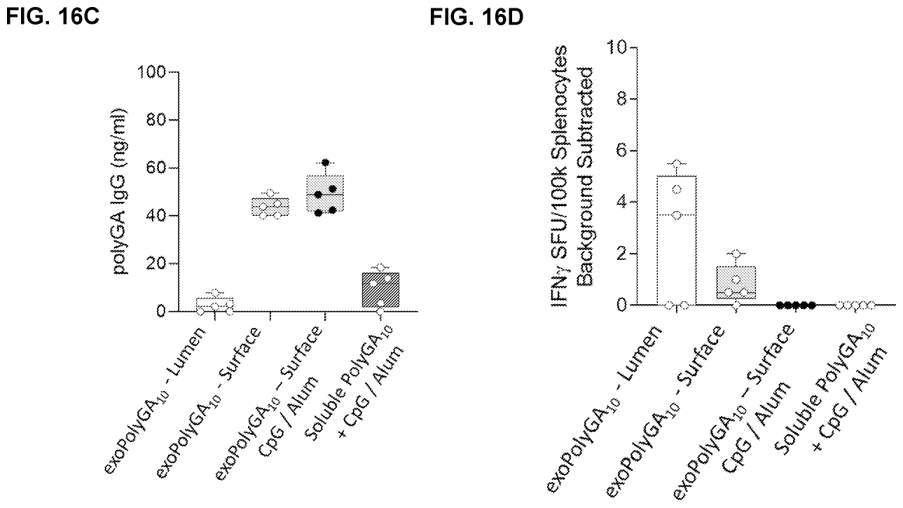

Further to Examples 6 and 7 provided above, the ability of the EVs described herein to induce immune response was assessed in vivo. Briefly, EVs comprising a B cell antigen (i.e., 10 GA amino acid repeats; “PolyGA”) were constructed using methods described herein. As shown in FIG. 16A, in some of the EVs, the polyGA was associated with the luminal surface of the EV fused to the C terminus of PTGFRN (“exoPolyGA-lumen”). In some of the EVs, the polyGA was associated with the exterior surface fused to the N terminus of PTGFRN (“exoPolyGA-surface”). Some of the EVs were further co-loaded with alum and CpG adjuvants on the exterior surface, e.g., anchored using a cholesterol moiety. The different engineered EVs were then used to immunize C57BL/6 mice via intramuscular injection. As shown in FIG. 16B, the mice received total of three immunizations: primed on day 0, and then boosted on days 14 and 28. At day 35 post initial immunization, the animals were sacrificed and polyGA-specific IgG (in sera using ELISA) and IFN-γ+ T cells (in spleen using ELISPOT) were assessed. The different treatment groups were as follows: (1) exoPolyGA-lumen; (2) exoPolyGA-surface; (3) exoPolyGA-surface co-loaded with alum and CpG adjuvants; and (4) soluble PolyGA peptide+soluble alum and CpG adjuvants.

As shown in FIG. 16C, anti-PolyGA antibodies were detected in mice immunized with exoPolyGA-surface, with or without the alum and CpG adjuvants. In contrast, no detectable level of anti-PolyGA was observed in mice that received exoPolyGA-lumen or soluble polyGA+soluble alum and CpG adjuvants. No significant antigen-specific T cell response (i.e., >25 spots/100,000 splenocytes) was observed in any of the treatment groups (see FIG. 16D).

The above results demonstrate that the polyGA on the exterior surface of the EVs (fused to the N terminus of PTGFRN) is capable of inducing a strong antibody-mediated response without the induction of a polyGA-specific T cell response. The further co-loading of the alum and CpG adjuvants was not necessary for the induction of the anti-PolyGA antibody response. Moreover, the results also demonstrate the increased potency of the EV-based vaccines described herein, e.g., compared to traditional (soluble) vaccine formulations.

### Example 13: Analysis of Immune Response after Administration of exoOVA

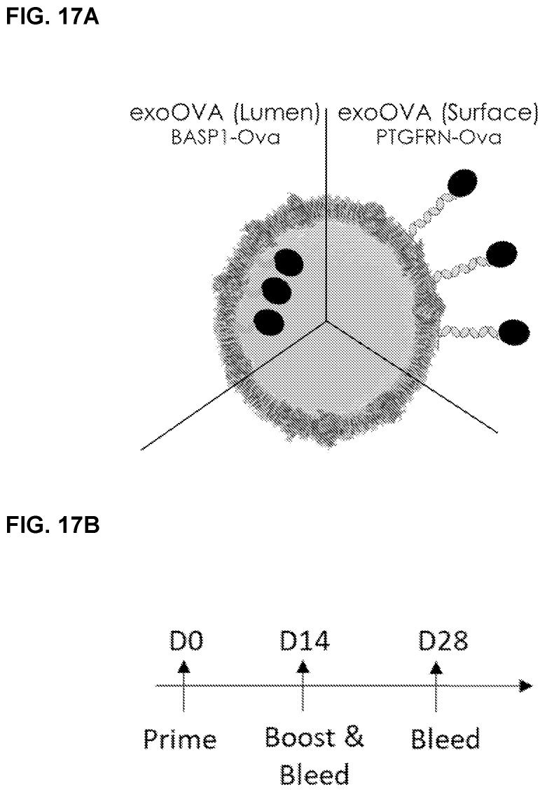

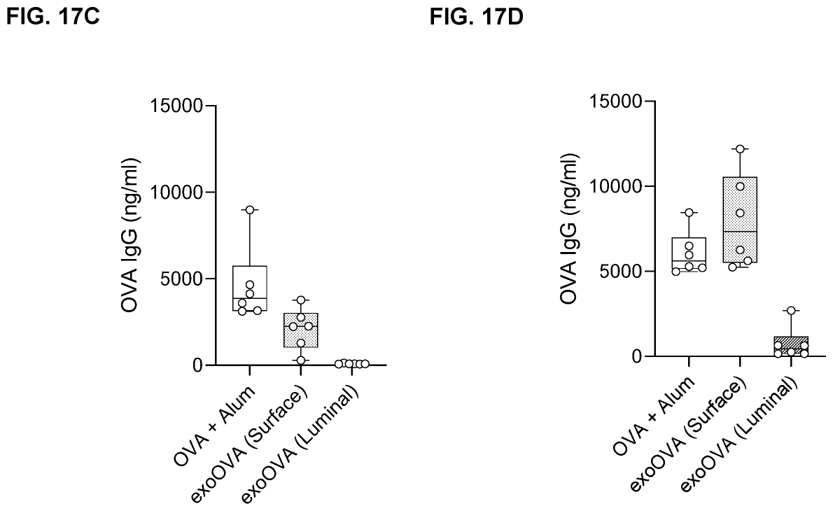

To further demonstrate the ability of the EVs of the present disclosure to induce an immune response in vivo, EVs comprising ovalbumin (OVA) were constructed using methods described herein. As shown in FIG. 17A, the full length OVA protein was fused either to BASP-1 on the luminal surface of the EV (“exoOVA-lumen”) or to PTGFRN and displayed on the exterior surface (“exoOVA-surface”). The EVs were then administered via intramuscular injection to C57BL/6 mice as shown in FIG. 17B. Specifically, the mice received a total of two doses of the EVs: primed on day 0 and boosted on day 14. Control animals received soluble OVA with alum adjuvant. The animals were bled at days 14 and 28 post initial administration, and the level of anti-OVA IgG was assessed in the sera using ELISA.

As shown in FIGS. 17C and 17D, animals treated with the exoOVA-lumen did not have significant amount of anti-OVA IgG both at days 14 and 28 post initial administration. In animals that received the exoOVA-surface, detectable level of anti-OVA IgG was observed after a single administration (i.e., day 14). After the boost (i.e., day 28), the level of anti-OVA IgG in animals treated with the exoOVA-surface was comparable to that observed in animals that were treated with soluble OVA with alum adjuvant.

The above results further confirm the potency of the EV-based vaccines described herein, and show that the exoOVA-surface, even in the absence of adjuvant, can elicit antigen-specific antibody response comparable to that observed with adjuvanted recombinant immunization. The lack of antigen-specific antibodies with the exoOVA-lumen demonstrate that an antigen of interest can be protected from undesired immune responses, e.g., by loading the antigens within the lumen of the EVs, e.g., on the luminal surface.

### Example 14: Comparison of Immune Response after Administration of exoOVA-Surface and exoOVA-Lumen

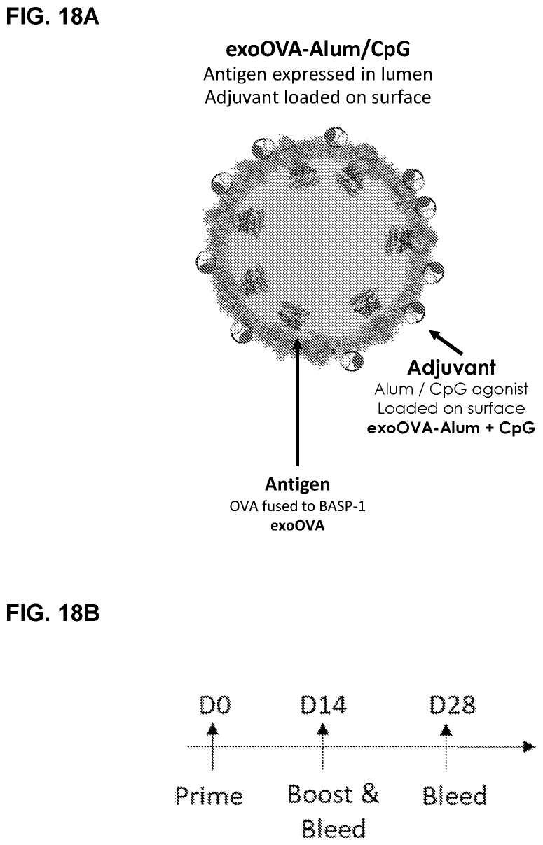

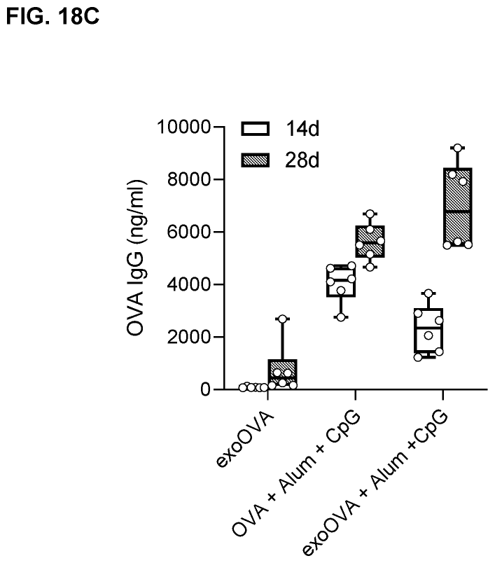

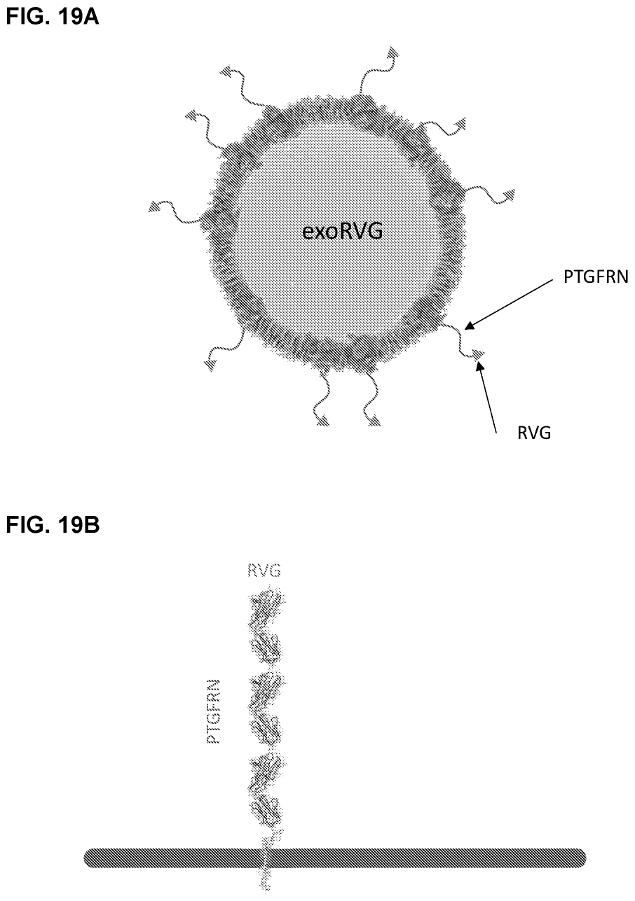

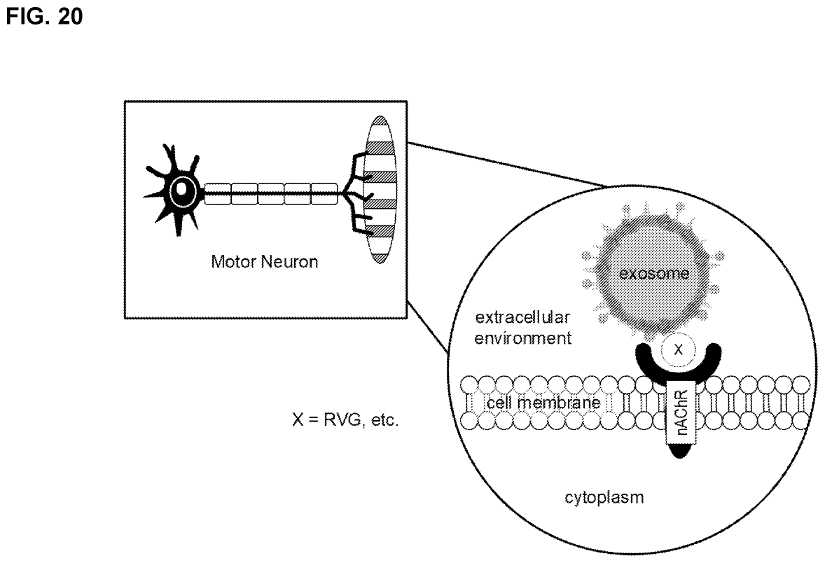

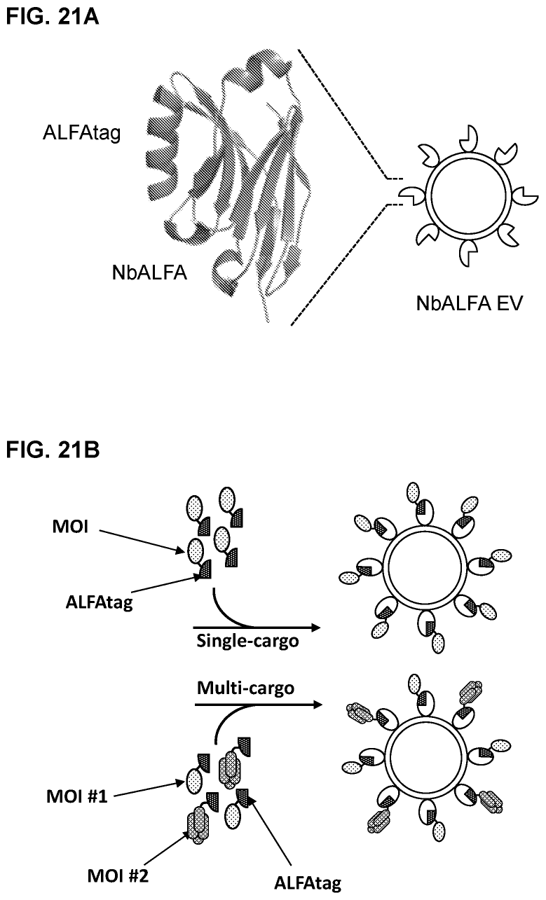

To further assess the differences in antibody response observed with exoOVA-surface and exOVA-lumen (see Example 13), a further EV construct was generated using the methods described herein. Specifically, an exOVA-lumen co-loaded with alum and CpG adjuvants on the exterior surface, e.g., anchored using a cholesterol moiety, was produced (see FIG. 18A). Then, C57BL/6 mice were immunized (via intramuscular injection) using the different EVs. As shown in FIG. 18B, the mice received a total of two doses of the EVs: primed on day 0 and boosted on day 14. Control animals received soluble OVA with alum adjuvant. The animals were bled at days 14 and 28 post initial administration, and the level of anti-OVA IgG was assessed in the sera using ELISA.

In agreement with the earlier data (see Example 12), animals treated with exoOVA-lumen did not have significant level of anti-OVA IgG antibodies both at days 14 and 28 post initial administration (FIG. 18C). However, in animals treated with the exoOVA-lumen co-loaded with alum and CpG adjuvants had significant level of anti-OVA IgG antibodies even after a single administration (i.e., day 14). After the boost (i.e., day 28), the level of anti-OVA IgG antibodies was significantly greater than that observed in animals that received the soluble OVA with alum adjuvant.

The above results confirm that by loading an antigen within the lumen of EVs (e.g., on the luminal surface), the antigens can be protected from undesired immune responses against the antigen. And, by co-loading such EVs with alum and CpG adjuvants (e.g., on the exterior surface), the EVs can be used to elicit strong antibody responses that are much greater in magnitude compared to adjuvanted recombinant immunization.

### Example 15: Analysis of the Effect of Adjuvants on EV-Based Vaccines

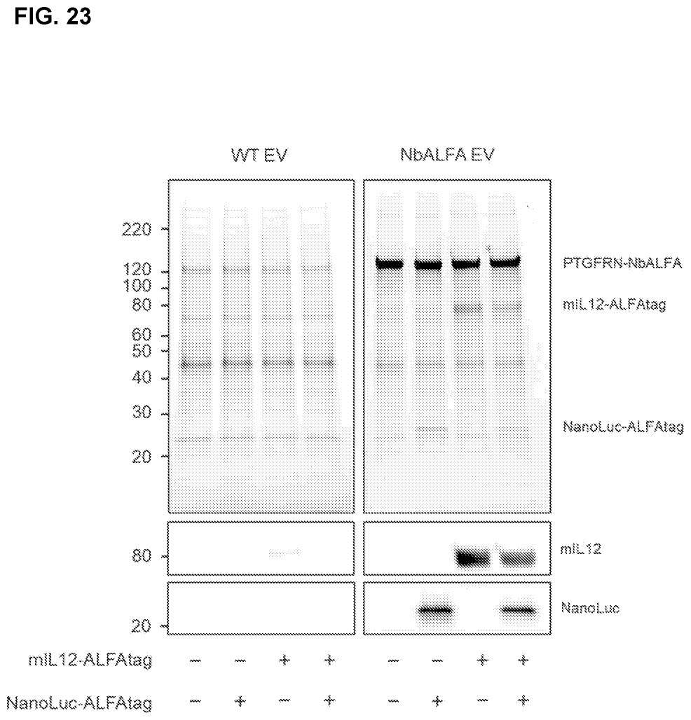

To better understand the role that different adjuvants can have on the EVs described herein, the ability of alum and CpG adjuvants to enhance the therapeutic efficacy of EV-based vaccines was assessed in vivo. Briefly, as shown in FIG. 23A, mice (e.g., FVB mice) were immunized with one of the following: (1) soluble OVA+soluble alum; (2) soluble OVA+soluble alum+soluble CpG; (3) exoOVA-PrX (also referred to herein as “exoOVA-surface”; see Example 13); (4) exoOVA-PrX co-loaded with alum on the exterior surface; (5) exoOVA-PrX co-loaded with alum and CpG on the exterior surface; (6) exoOVA-PrY (also referred to herein as “exoOVA-lumen”; see Example 13) co-loaded with alum on the exterior surface; (7) exoOVA-PrY; and (8) exoOVA-PrY co-loaded with both alum and CpG on the exterior surface. Animals from the different groups received total of three doses of the treatment regimen (at days 0, 14, and 28). At days 14, 28, and 42 post initial treatment administration, sera were collected and the amount of anti-OVA antibodies (both IgG and IgM) was quantified using ELISA.

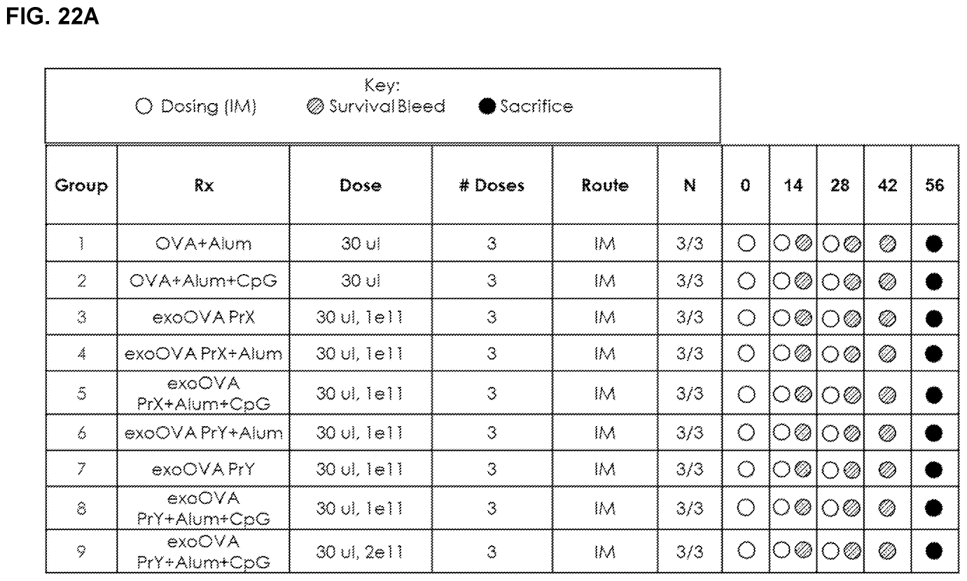

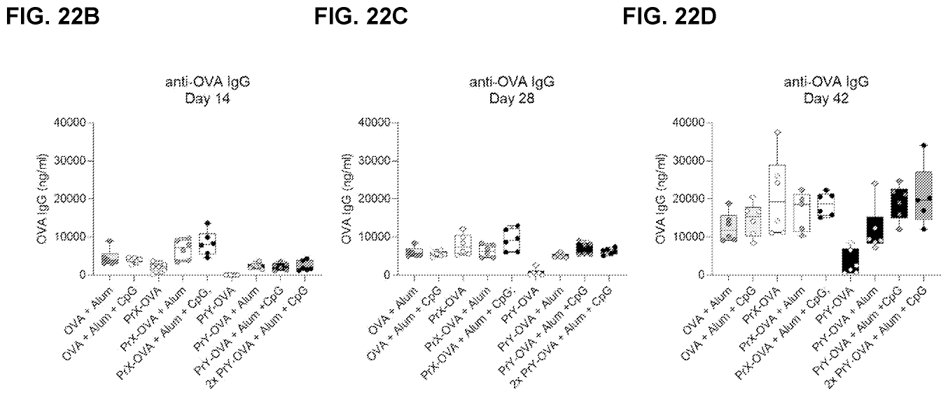

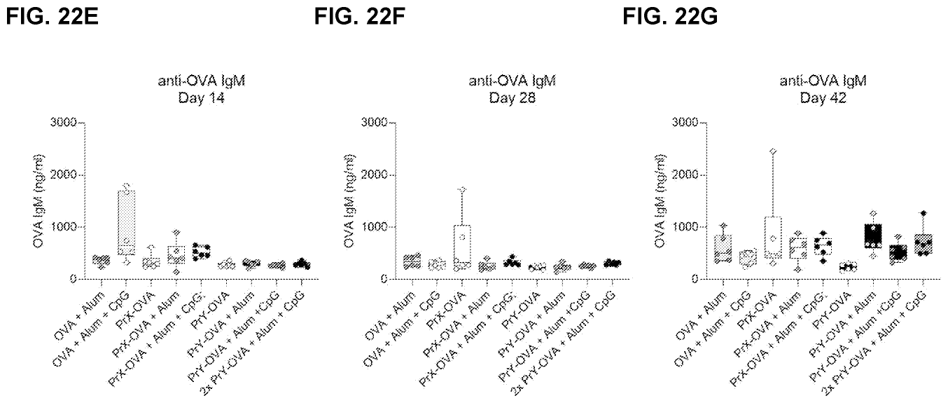

As shown in FIGS. 22E-22G, none of the animals from the various treatment groups exhibited significant levels of anti-OVA IgM antibodies for all time points tested. However, in agreement with the earlier results (see Examples 13 and 14), animals treated with exoOVA-PrX (i.e., OVA on the exterior surface of the EV) had high levels of anti-OVA IgM antibodies even in the absence of adjuvant (FIGS. 22B-22D). With the exoOVA-PrY (i.e., OVA on the luminal surface of the EV), only animals treated with exoOVA-PrY that was further co-loaded with an adjuvant had significant amount of anti-OVA IgM antibodies. At least at days 28 and 42 post initial administration, exoOVA-PrY that was further loaded with alum and CpG adjuvants induced greater anti-OVA IgM antibody response, compared to exoOVA-PrY that was further loaded with alum alone.

The above results further demonstrate the versatility of the EV-based vaccines described herein. At least with certain antigens, by engineering the EVs to comprise the antigen on the exterior surface (e.g., fused to PTGFRN), a potent antigen-specific immune response can be induced without the need for an adjuvant.

### Example 16: Analysis of the Loading of Multiple Payloads in EVs

Next, the ability of the EVs described herein to comprise multiple payloads (e.g., antigen, adjuvant, immunomodulatory, and/or targeting moiety) was assessed. Briefly, NANOLUC™ luciferase (Nluc) fused to the ALFAtag peptide (10 μg) (Nluc-ALFAtag) and molar equivalent of mouse IL-12 fused to ALFAtag peptide (mIL12-ALFAtag) were mixed with the following EVs individually or simultaneously: (1) native EVs; or (2) engineered-EVs overexpressing ALFA-specific nanobody (NbALFA) fused to PTGFRN (NbALFA-EVs). The mixture was incubated for 30 minutes at room temperature. Then, unbound Nluc-ALFAtag and/or mIL12-ALFAtag was removed by ultracentrifugation (20 minutes at 100,000×g). The EV pellets were resuspended in PBS and analyzed by SDS-PAGE and Western blot.

As shown in FIG. 23, in native EVs, no meaningful loading was observed for either Nluc-ALFAtag or mIL12-ALFAtag. However, in the NbALFA-EVs, significant loading of Nluc-ALFAtag and mIL12-ALFAtag was observed, as measured by both Western blot and SDS-PAGE. Similar results were observed whether Nluc-ALFAtag and mIL12-ALFAtag were loaded individually or simultaneously.

The above results further demonstrate that the EVs of the present disclosure can be readily modified to simultaneously comprise multiple payloads. As described herein, such EVs can be useful in treating various diseases and disorders, such as the neurological disorders described herein.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the invention(s). Many variations will become apparent to those skilled in the art upon review of this specification.

What is claimed is:

1. A method of treating a neurological disorder in a subject in need thereof, comprising administering to the subject an extracellular vesicle (EV), which comprises an antigen and wherein the EV is capable of targeting a cell within the central nervous system (CNS) of the subject.

2. The method of claim 1, wherein administering the EV to the subject results in the induction of a humoral immune response, comprising antibodies directed against the antigen.

3. The method of claim 2, wherein the induction of the humoral immune response improves one or more symptoms associated with the neurological disorder.

4. The method of claim 2 or 3, wherein the antibodies are capable of specifically binding to a neuronal protein that has misfolded (“misfolded neuronal protein”).

5. The method of claim 4, wherein the binding of the antibodies to the misfolded neuronal protein facilitates the removal of the misfolded neuronal protein from the subject.

6. The method of claim 5, wherein administering the EV to the subject results in a decrease in the amount of misfolded neuronal protein present within the CNS of the subject.

7. A method for delivering an extracellular vesicle (EV) to a cell within the central nervous system (CNS) of a subject in need thereof, comprising administering to the subject the EV, wherein the EV comprises an antigen and wherein the EV is capable of targeting the cell.

8. A method for modulating a germinal center response to an antigen in a subject in need thereof, comprising administering to the subject an extracellular vesicle (EV), which comprises an antigen, and wherein the EV is capable of targeting a cell within the central nervous system (CNS) of the subject.

9. The method of claim 8, wherein administering the EV to the subject increases the germinal center response in the subject.

10. The method of claim 9, wherein the increase in the germinal center response results in greater production of antibodies against the antigen.

11. The method of claim 8, wherein administering the EV to the subject decreases the germinal center response in the subject.

12. The method of claim 11, wherein the decrease in the germinal center response results in lower production of antibodies against the antigen.

13. The method of any one of claims 1 to 12, wherein the EV further comprises one or more additional payloads.

14. The method of claim 13, wherein the additional payload is an adjuvant.

15. The method of claim 13 or 14, wherein the additional payload is an immune modulator.

16. The method of any one of claims 1 to 15, wherein the antigen comprises a neuronal protein that when misfolded can cause a neurological disorder.

17. The method of claim 16, wherein the neuronal protein comprises amyloid beta (Aβ), tau, alpha-synuclein, poly-GA, or combinations thereof.

18. The method of any one of claims 7 to 17, wherein the subject suffers from a neurological disorder.

19. The method of any one of claims 1 to 6 and 16 to 18, wherein the neurological disorder comprises a brain tumor, neoplastic meningitis, leptomeningeal cancer disease (LMD), amyotrophic lateral sclerosis (ALS), frontotemporal dementia (FTD), Parkinson's disease (PD), Huntington's disease (HD), Alzheimer's disease (AD), or combinations thereof.

20. The method of claim 19, wherein the neurological disorder is leptomeningeal cancer disease (LMD).

21. The method of claim 19, wherein the neurological disorder is a brain tumor.

22. The method of claim 21, wherein the brain tumor is a glioma.

23. The method of claim 22, wherein the glioma is a low grade glioma or a high grade glioma.

24. The method of claim 22 or 23, wherein the glioma is oligodendroglioma, anaplastic astrocytomas, glioblastoma multiforme, diffuse intrinsic pontine glioma, IDH1 and IDH2-mutated glioma, or combinations thereof.

25. The method of claim 24, wherein the glioma is glioblastoma multiforme.

26. The method of claim 19, wherein the ALS and/or FTD is associated with a hexanucleotide GGGGCC repeat expansion in the C9orf72 gene.

27. The method of any one of claims 14 to 25, wherein the adjuvant comprises a stimulator of interferon genes protein (STING) agonist, toll-like receptor (TLR) agonist, inflammatory mediator, or combinations thereof.

28. The method of claim 26, wherein the adjuvant is a STING agonist.

29. The method of claim 27, wherein the STING agonist comprises a cyclic dinucleotide STING agonist or a non-cyclic dinucleotide STING agonist.

30. The method of claim 26, wherein the adjuvant is a TLR agonist.

31. The method of claim 29, wherein the TLR agonist comprises a TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C, MPLA), a TLR5 agonist (e.g., flagellin), a TLR6 agonist, a TLR7/8 agonist (e.g., single-stranded RNA, CpG-A, Poly G10, Poly G3, Resiquimod), a TLR9 agonist (e.g., unmethylated CpG DNA, CpG class C), or combinations thereof.

32. The method of any one of claims 1 to 30, wherein the cell comprises an immune cell.

33. The method of claim 31, wherein the immune cell comprises a dendritic cell, macrophage, T cells, B cells, or combinations thereof.

34. The method of claim 32, wherein the immune cell is a dendritic cell.

35. The method of claim 32, wherein the immune cell is a macrophage.

36. A method for treating an autoimmune disorder in a subject in need thereof, comprising administering to the subject an extracellular vesicle (EV), which comprises an antigen, and wherein the EV is capable of targeting a cell within the central nervous system (CNS) of the subject.

37. A method for inducing an immune tolerance in a subject in need thereof, comprising administering to the subject an extracellular vesicle (EV), which comprises an antigen, and wherein the EV is capable of targeting a cell within the central nervous system (CNS) of the subject.

38. The method of claim 36, wherein the subject suffers from an autoimmune disorder.

39. The method of any one of claims 35 to 37, wherein administering the EV to the subject results in the induction of tolerogenic cells.

40. The method of claim 38, wherein the induction of the tolerogenic cells improves one or more symptoms associated with the autoimmune disorder.

41. The method of claim 38 or 39, wherein the tolerogenic cells comprise regulatory T cells (Tregs), liver sinusoidal endothelial cells (LSECs), Kupffer cells, or combinations thereof.

42. The method of claim 40, wherein the tolerogenic cells are Tregs that are specific to the antigen.

43. The method of any one of claims 35 to 41, wherein the antigen comprises a self-antigen that is associated with an autoimmune disorder.

44. The method of claim 42, wherein the autoimmune disorder comprises a multiple sclerosis (MS), peripheral neuritis, Sjogren's syndrome, rheumatoid arthritis, alopecia, autoimmune pancreatitis, Behcet's disease, Bullous pemphigoid, Celiac disease, Devic's disease (neuromyelitis optica), Glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, Graves' disease, myasthenia gravis (MG), pemphigus vulgaris, anti-glomerular basement membrane disease (Goodpasture syndrome), Hashimoto's thyroiditis, autoimmune hepatitis, or combinations thereof.

45. The method of claim 42 or 43, wherein the self-antigen comprises beta-cell proteins, insulin, islet antigen 2 (IA-2), glutamic acid decarboxylase (GAD65), zinc transporter 8 (ZNT8), myelin oligodendrocyte glycoprotein (MOG), myelin basic protein (MBP), proteolipid protein (PLP), myelin-associated glycoprotein (MAG), citrullinated antigens, synovial proteins, aquaporin-4 (AQP4), nicotinic acetylcholine receptor (nAChR), desmoglein-1 (DSG1), desoglein-2 (DSG2), thyrotropin receptor, type IV collagen, thyroglobulin, thyroid peroxidase, thyroid-stimulating hormone receptor (TSHR), or combinations thereof.

46. The method of claim 42, wherein the self-antigen is AQP4 and the autoimmune disorder is neuromyelitis optica (NMO).

47. The method of claim 42, wherein the self-antigen is MOG and the autoimmune disorder is multiple sclerosis (MS).

48. The method of claim 42, wherein the self-antigen is nAChR and the autoimmune disorder is myasthenia gravis (MG).

49. The method of any one of claims 35 to 47, wherein the EV further comprises one or more additional payloads.

50. The method of claim 48, wherein the additional payload is an immune modulator.

51. The method of claim 49, wherein the immune modulator comprises a tolerance inducing agent (“tolerogen”).

52. The method of claim 50, wherein the tolerogen comprises a NF-κB inhibitor, COX-2 inhibitor, mTOR inhibitor (e.g., rapamycin and derivatives), prostaglandins, nonsteroidal anti-inflammatory agents (NSAIDS), antileukotriene, aryl hydrocarbon receptor (AhR) ligand, vitamin D3, retinoic acid, steroids, Fas receptor/ligand, CD22 ligand, IL-10, IL-35, IL-27, metabolic regulator (e.g., glutamate), glycans (e.g., ES62, LewisX, LNFPIII), peroxisome proliferator-activated receptor (PPAR) agonists, immunoglobulin-like transcript (ILT) family of receptors (e.g., ILT3, ILT4, HLA-G, ILT-2), dexamethasone, or combinations thereof.

53. The method of claim 51, wherein the tolerogen is rapamycin.

54. The method of claim 51, wherein the tolerogen is vitamin D3.

55. The method of claim 51, wherein the tolerogen is retinoic acid.

56. The method of claim 51, wherein the tolerogen is dexamethasone.

57. The method of any one of claims 50 to 55, wherein the immune modulator comprises a polynucleotide selected from a mRNA, miRNA, siRNA, antisense oligonucleotide (ASO), phosphorodiamidate morpholino oligomer (PMO), peptide-conjugated phosphorodiamidate morpholino oligomer (PPMO), shRNA, lncRNA, dsDNA, or combinations thereof.

58. The method of claim 56, wherein the immune modulator is an ASO.

59. The method of claim 57, wherein the ASO is capable of inhibiting NF-κB, CD40, mTOR, or combinations thereof.

60. The method of any one of claims 1 to 58, wherein the EV further comprises a targeting moiety.

61. The method of claim 59, wherein the targeting moiety is capable of specifically binding to a marker expressed on the cell within the CNS of the subject.

62. The method of claim 60, wherein the marker is expressed only on dendritic cells.

63. The method of claim 61, wherein the marker comprises a C-type lectin domain family 9 member A (Clec9a) protein, a dendritic cell-specific intercellular adhesion molecule-3-grabbing non-integrin (DC-SIGN), CD207, CD40, Clec6, dendritic cell immunoreceptor (DCIR), DEC-205, lectin-like oxidized low-density lipoprotein receptor-1 (LOX-1), MARCO, Clec12a, DC-asialoglycoprotein receptor (DC-ASGPR), DC immunoreceptor 2 (DCIR2), Dectin-1, macrophage mannose receptor (MMR), BDCA-1 (CD303, Clec4c), Dectin-2, Bst-2 (CD317), or combinations thereof.

64. The method of claim 60, wherein the marker is expressed only on macrophages.

65. The method of claim 63, wherein the marker comprises CD14, CD16, CD64, CD68, CD71, CCR5, or combinations thereof.

66. The method of any one of claims 1 to 64, wherein the EV further comprises a first scaffold moiety.

67. The method of claim 65, wherein the antigen, additional payload, and/or targeting moiety is linked to the first scaffold moiety.

68. The method of claim 66, wherein the EV further comprises a second scaffold moiety.

69. The method of claim 67, wherein the antigen, additional payload, and/or targeting moiety is linked to the second scaffold moiety.

70. The method of claim 67 or 68, wherein the first scaffold moiety and the second scaffold moiety are the same.

71. The method of claim 67 or 68, wherein the first scaffold moiety and the second scaffold moiety are different.

72. The method of any one of claims 65 to 70, wherein the first scaffold moiety is Scaffold X.

73. The method of any one of claims 65 to 70, wherein the first scaffold moiety is Scaffold Y.

74. The method of any one of claims 67 to 72, wherein the second scaffold moiety is Scaffold Y.

75. The method of any one of claims 67 to 72, wherein the second scaffold moiety is Scaffold X.

76. The method of any one of claims 71 to 74, wherein Scaffold X is selected from prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), or combinations thereof.

77. The method of any one of claims 72 to 75, wherein Scaffold Y is selected from myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), or combinations thereof.

78. The method of any one of claims 65 to 76, wherein the antigen, additional payload, and/or targeting moiety is linked to the first scaffold moiety and/or to the second scaffold moiety by a linker.

79. The method of claim 77, wherein the linker is a polypeptide.

80. The method of claim 77, wherein the linker is a non-polypeptide moiety.

81. The method of any one of claims 65 to 79, wherein the first scaffold moiety or the second scaffold moiety is PTGFRN protein.

82. The method of any one of claims 65 to 80, wherein the first scaffold moiety or the second scaffold moiety comprises an amino acid sequence as set forth in SEQ ID NO: 33.

83. The method of any one of claims 65 to 80, wherein the first scaffold moiety or the second scaffold moiety comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 1.

84. The method of any one of claims 65 to 82, wherein the first scaffold moiety or the second scaffold moiety is BASP1 protein.

85. The method of any one of claims 65 to 83, wherein the first scaffold moiety or the second scaffold moiety comprises a peptide of (M)(G)(π)(X)(Φ/π)(π)(+)(+) or (G)(π)(X)(Φ/π)(π)(+)(+), wherein each parenthetical position represents an amino acid, and wherein π is any amino acid selected from the group consisting of Pro, Gly, Ala, and Ser, X is any amino acid, Φ is any amino acid selected from the group consisting of Val, Ile, Leu, Phe, Trp, Tyr, and Met, and (+) is any amino acid selected from the group consisting of Lys, Arg, and His; and wherein position five is not (+) and position six is neither (+) nor (Asp or Glu).

86. The method of any one of claims 65 to 84, wherein the first scaffold moiety or the second scaffold moiety comprises an amino acid sequence set forth in any one of SEQ ID NOs: 50-155.

87. The method of any one of claims 65 to 84, wherein the first scaffold moiety or the second scaffold moiety comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 3.

88. The method of any one of claims 1 to 86, wherein the EV is not derived from a naturally-existing antigen-presenting cell (APC).

89. The method of any one of claims 1 to 87, wherein the EV is an exosome.

90. The method of any one of claims 1 to 88, wherein the EV is administered via intrathecal, intraocular, intracranial, intranasal, perineural, or combinations thereof.

91. The method of claim 89, wherein the intrathecal administration is in the spinal canal and/or the subarachnoid space.

92. The method of claim 89, wherein the intraocular administration is selected from intravitreal, intracameral, subconjunctival, subretinal, subscleral, intrachoroidal, or combinations thereof.

93. The method of claim 89, wherein the intracranial administration is selected from intracisternal, subarachnoidal, intrahippocampal, intracerebroventricular, intraparenchymal, or combinations thereof.

94. The method of claim 89, wherein the intranasal administration is by instillation or injection.

95. The method of claim 89, wherein the perineural administration is by facial intradermal injection.

96. A method of administering an EV to a subject in need thereof, comprising intrathecally administering the EV to the subject and applying a mechanical convective force to the torso of the subject.

97. A method of administering an EV to a subject in need thereof, comprising intrathecally administering the EV to the subject wherein a mechanical convective force is applied to the torso of the subject.

98. The method of claim 95, wherein the mechanical convective force is achieved using a high frequency chest wall or lumbothoracic oscillating respiratory clearance device.

99. The method of any one of claims 95 to 97, wherein the mechanical convective force improves the intrathecal administration.

100. The method of any one of claims 95 to 98, wherein the mechanical convective force results in a less dosing amount of the EVs.

101. The method of any one of claims 95 to 98, wherein the mechanical convective force results in an efficient dosing of the EVs.

102. The method of any one of claims 1 to 100, wherein the EV further comprises a targeting moiety that targets a Schwann cell.

103. The method of claim 101, wherein the targeting moiety specifically interacts with a transferrin receptor (TfR), apolipoprotein D (ApoD), Galectin 1 (LGALS1), Myelin proteolipid protein (PLP), Glypican 1, Syndecan 3, or any combination thereof.

104. The method of claim 101 or 102, wherein the targeting moiety comprises a transferrin-receptor-targeting moiety.

105. The method of any one of claims 1 to 100, wherein the EV further comprises a targeting moiety that targets a sensory neuron.

106. The method of claim 104, wherein the targeting moiety specifically interacts with a Trk receptor.

107. The method of claim 105, wherein the TRK receptor is selected from TrkA, TrkB, TrkC, and any combination thereof.

108. The method of any one of claims 1 to 100, wherein the EV further comprises a targeting moiety that targets a motor neuron.

109. The method of claim 107, wherein the targeting moiety comprises a Rabies Virus Glycoprotein (RVG) peptide, a Targeted Axonal Import (TAxI) peptide, a P75R peptide, a Tet-C peptide, or any combination thereof.

110. The method of claim 15, wherein the immune modulator comprises a CD4+ T helper peptide, an inhibitor for a negative checkpoint regulator or an inhibitor for a binding partner of a negative checkpoint regulator, an activator for a positive co-stimulatory molecule or an activator for a binding partner of a positive co-stimulatory molecule, a cytokine or a binding partner of a cytokine, a chemokine, an inhibitor of lysophosphatidic acid (LPA), a protein that supports intracellular interactions required for germinal center responses, a T-cell receptor (TCR) or a derivative thereof, a chimeric antigen receptor (CAR) or a derivative thereof, an activator of a T-cell receptor or co-receptor, a tolerance inducing agent, an agonist, an antagonist, an antibody or an antigen-binding fragment thereof, a polynucleotide, or combinations thereof.

111. The method of claim 16, wherein the immune modulator is a CD4+ T helper peptide.

112. The method of claim 16, wherein the activator for a positive co-stimulatory molecule comprises CD40L, TNFα, TNF-C, OX40L, FasL, LIGHT, TL1A, CD27L, Siva, CD153, 4-1BB ligand, TRAIL, RANKL, TWEAK, APRIL, BAFF, CAMLG, NGF, BDNF, NT-3, NT-4, GITR ligand, EDA-2, or combinations thereof.

113. The method of claim 16, wherein the cytokine comprises IL-21, IL-2, IL-4, IL-7, IL-10, IL-12, IL-15, IFN-γ, IL-1α, IL-1β, IL-1ra, IL-18, IL-33, IL-36α, IL-36β, IL-36γ, IL-36ra, IL-37, IL-38, IL-3, IL-5, IL-6, IL-11, IL-13, IL-23, granulocyte-macrophage colony stimulating factor (GM-CSF), granulocyte-colony stimulating factor (G-CSF), leukemia inhibitory factor (LIF), stem cell factor (SCF), thrombopoietin (TPO), macrophage-colony stimulating factor (M-CSF), erythropoieticn (EPO), Flt-3, IFN-α, IFN-β, IFN-γ, IL-19, IL-20, IL-22, IL-24, TNF-α, TNF-β, BAFF, APRIL, lymphotoxin beta (TNF-γ), IL-17A, IL-17B, IL-17C, IL-17D, IL-17E, IL-17F, IL-25, TSLP, IL-35, IL-27, TGF-β, or combinations thereof.

114. The method of any one of claims 15 to 19, wherein the immune modulator is capable of enhancing an antibody immune response induced by the EV.

115. A method of treating and/or preventing a disease or disorder in a subject in need thereof, comprising administering to the subject (i) a priming dose, which comprises a first EV, and (ii) a boosting dose, which comprises a second EV, wherein the first EV and the second EV are not the same.

116. The method of claim 115, wherein the first EV comprises an antigen and an adjuvant.

117. The method of claim 115 or 116, wherein the second EV comprises an antigen but not an adjuvant.

118. The method of claim 117, wherein the antigen of the first EV and the antigen of the second EV are the same.

119. The method of any one of claims 115 to 118, wherein the disease or disorder comprises a neurological disorder.

120. The method of any one of claims 115 to 119, wherein the disease or disorder comprises an autoimmune disorder.

121. The method of any one of claims 115 to 120, wherein the priming dose and the boosting dose are administered via different routes.

122. The method of any one of claims 1 to 104 and 116 to 121, wherein the antigen is linked to the exterior surface and/or the luminal surface of the EV by an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof.


SEQUENCE LISTING (416 sequences)

SEQ ID NO: 1 - protein, 879 aa, Homo sapiens
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 731 aa, Homo sapiens
PSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 3 - protein, 611 aa, Homo sapiens
VATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 4 - protein, 485 aa, homo sapien
SPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 5 - protein, 343 aa, Homo sapiens
KPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 6 - protein, 217 aa, Homo sapiens
VRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 7 - protein, 66 aa, homo sapiens
SKPVFITVKMDVLNAFKYPLLIGVGLSTVIGLLSCLIGYCSSHWCCKKEVQETRRERRRLMSMEMD

SEQ ID NO: 8 - protein, 21 aa, Homo sapiens
MGRLASRPLLLALLSLALCRG

SEQ ID NO: 9 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 10 - protein, 247 aa, Homo sapiens
PGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 11 - protein, 168 aa, Homo sapiens
HGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 12 - protein, 66 aa, homo sapiens
SHLAALWPFLGIVAEVLVLVTIIFIYEKRRKPEDVLDDDDAGSAPLKSSGQHQNDKGKNVRQRNSS

SEQ ID NO: 13 - protein, 18 aa, homo sapiens
MAAALFVLLGFALLGTHG

SEQ ID NO: 14 - protein, 613 aa, homo sapiens
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 15 - protein, 456 aa, homo sapiens
APPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 16 - protein, 320 aa, Homo sapiens
AHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 17 - protein, 179 aa, homo sapiens
REEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 18 - protein, 24 aa, Homo sapiens
VALVTGATVLGTITCCFMKRLRKR

SEQ ID NO: 19 - protein, 27 aa, Homo sapiens
MGALRPTLLPPSLPLLLLLMLGMGCWA

SEQ ID NO: 20 - protein, 1195 aa, homo sapiens
MKCFFPVLSCLAVLGVVSAQRQVTVQEGPLYRTEGSHITIWCNVSGYQGPSEQNFQWSIYLPSSPEREVQIVSTMDSSFPYAIYTQRVRGGKIFIERVQGNSTLLHITDLQARDAGEYECHTPSTDKQYFGSYSAKMNLVVIPDSLQTTAMPQTLHRVEQDPLELTCEVASETIQHSHLSVAWLRQKVGEKPVEVISLSRDFMLHSSSEYAQRQSLGEVRLDKLGRTTFRLTIFHLQPSDQGEFYCEAAEWIQDPDGSWYAMTRKRSEGAVVNVQPTDKEFTVRLETEKRLHTVGEPVEFRCILEAQNVPDRYFAVSWAFNSSLIATMGPNAVPVLNSEFAHREARGQLKVAKESDSVFVLKIYHLRQEDSGKYNCRVTEREKTVTGEFIDKESKRPKNIPIIVLPLKSSISVEVASNASVILEGEDLRFSCSVRTAGRPQGRFSVIWQLVDRQNRRSNIMWLDRDGTVQPGSSYWERSSFGGVQMEQVQPNSFSLGIFNSRKEDEGQYECHVTEWVRAVDGEWQIVGERRASTPISITALEMGFAVTAISRTPGVTYSDSFDLQCIIKPHYPAWVPVSVTWRFQPVGTVEFHDLVTFTRDGGVQWGDRSSSFRTRTAIEKAESSNNVRLSISRASDTEAGKYQCVAELWRKNYNNTWTRLAERTSNLLEIRVLQPVTKLQVSKSKRTLTLVENKPIQLNCSVKSQTSQNSHFAVLWYVHKPSDADGKLILKTTHNSAFEYGTYAEEEGLRARLQFERHVSGGLFSLTVQRAEVSDSGSYYCHVEEWLLSPNYAWYKLAEEVSGRTEVTVKQPDSRLRLSQAQGNLSVLETRQVQLECVVLNRTSITSQLMVEWFVWKPNHPERETVARLSRDATFHYGEQAAKNNLKGRLHLESPSPGVYRLFIQNVAVQDSGTYSCHVEEWLPSPSGMWYKRAEDTAGQTALTVMRPDASLQVDTVVPNATVSEKAAFQLDCSIVSRSSQDSRFAVAWYSLRTKAGGKRSSPGLEEQEEEREEEEEEEEDDDDDDPTERTALLSVGPDAVFGPEGSPWEGRLRFQRLSPVLYRLTVLQASPQDTGNYSCHVEEWLPSPQKEWYRLTEEESAPIGIRVLDTSPTLQSIICSNDALFYFVFFYPFPIFGILIITILLVRFKSRNSSKNSDGKNGVPLLWIKEPHLNYSPTCLEPPVLSIHPGAID

SEQ ID NO: 21 - protein, 798 aa, homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 22 - protein, 1032 aa, homo sapiens [sequence omitted]

SEQ ID NO: 23 - protein, 660 aa, homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTETGFHHVSQADIEFLTSIDPTASASGSAGITGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYA

SEQ ID NO: 24 - protein, 1023 aa, homo sapiens
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 25 - protein, 1020 aa, homo sapiens
MGRGAGREYSPAATTAENGGGKKKQKEKELDELKKEVAMDDHKLSLDELGRKYQVDLSKGLTNQRAQDVLARDGPNALTPPPTTPEWVKFCRQLFGGFSILLWIGAILCFLAYGIQAAMEDEPSNDNLYLGVVLAAVVIVTGCFSYYQEAKSSKIMDSFKNMVPQQALVIREGEKMQINAEEVVVGDLVEVKGGDRVPADLRIISSHGCKVDNSSLTGESEPQTRSPEFTHENPLETRNICFFSTNCVEGTARGIVIATGDRTVMGRIATLASGLEVGRTPIAMEIEHFIQLITGVAVFLGVSFFVLSLILGYSWLEAVIFLIGIIVANVPEGLLATVTVCLTLTAKRMARKNCLVKNLEAVETLGSTSTICSDKTGTLTQNRMTVAHMWFDNQIHEADTTEDQSGATFDKRSPTWTALSRIAGLCNRAVFKAGQENISVSKRDTAGDASESALLKCIELSCGSVRKMRDRNPKVAEIPFNSTNKYQLSIHEREDSPQSHVLVMKGAPERILDRCSTILVQGKEIPLDKEMQDAFQNAYMELGGLGERVLGFCQLNLPSGKFPRGFKFDTDELNFPTEKLCFVGLMSMIDPPRAAVPDAVGKCRSAGIKVIMVTGDHPITAKAIAKGVGIISEGNETVEDIAARLNIPMSQVNPREAKACVVHGSDLKDMTSEQLDEILKNHTEIVFARTSPQQKLIIVEGCQRQGAIVAVTGDGVNDSPALKKADIGIAMGISGSDVSKQAADMILLDDNFASIVTGVEEGRLIFDNLKKSIAYTLTSNIPEITPFLLFIIANIPLPLGTVTILCIDLGTDMVPAISLAYEAAESDIMKRQPRNSQTDKLVNERLISMAYGQIGMIQALGGFFTYFVILAENGFLPSRLLGIRLDWDDRTMNDLEDSYGQEWTYEQRKVVEFTCHTAFFASIVVVQWADLIICKTRRNSVFQQGMKNKILIFGLLEETALAAFLSYCPGMGVALRMYPLKVTWWFCAFPYSLLIFIYDEVRKLILRRYPGGWVEKETYY

SEQ ID NO: 26 - protein, 1026 aa, homo sapiens
MGSGGSDSYRIATSQDKKDDKDSPKKNKGKERRDLDDLKKEVAMTEHKMSVEEVCRKYNTDCVQGLTHSKAQEILARDGPNALTPPPTTPEWVKFCRQLFGGFSILLWIGAILCFLAYGIQAGTEDDPSGDNLYLGIVLAAVVIITGCFSYYQEAKSSKIMESFKNMVPQQALVIREGEKMQVNAEEVVVGDLVEIKGGDRVPADLRIISAHGCKVDNSSLTGESEPQTRSPDCTHDNPLETRNITFFSTNCVEGTARGVVVATGDRTVMGRIATLASGLEVGKTPIAIEIEHFIQLITGVAVFLGVSFFILSLILGYTWLEAVIFLIGIIVANVPEGLLATVTVCLTLTAKRMARKNCLVKNLEAVETLGSTSTICSDKTGTLTQNRMTVAHMWFDNQIHEADTTEDQSGTSFDKSSHTWVALSHIAGLCNRAVFKGGQDNIPVLKRDVAGDASESALLKCIELSSGSVKLMRERNKKVAEIPFNSTNKYQLSIHETEDPNDNRYLLVMKGAPERILDRCSTILLQGKEQPLDEEMKEAFQNAYLELGGLGERVLGFCHYYLPEEQFPKGFAFDCDDVNFTTDNLCFVGLMSMIDPPRAAVPDAVGKCRSAGIKVIMVTGDHPITAKAIAKGVGIISEGNETVEDIAARLNIPVSQVNPRDAKACVIHGTDLKDFTSEQIDEILQNHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLLFIMANIPLPLGTITILCIDLGTDMVPAISLAYEAAESDIMKRQPRNPRTDKLVNERLISMAYGQIGMIQALGGFFSYFVILAENGFLPGNLVGIRLNWDDRTVNDLEDSYGQQWTYEQRKVVEFTCHTAFFVSIVVVQWADLIICKTRRNSVFQQGMKNKILIFGLFEETALAAFLSYCPGMDVALRMYPLKPSWWFCAFPYSFLIFVYDEIRKLILRRNPGGWVEKETYY

SEQ ID NO: 27 - protein, 1029 aa, Homo sapiens
MGLWGKKGTVAPHDQSPRRRPKKGLIKKKMVKREKQKRNMEELKKEVVMDDHKLTLEELSTKYSVDLTKGHSHQRAKEILTRGGPNTVTPPPTTPEWVKFCKQLFGGFSLLLWTGAILCFVAYSIQIYFNEEPTKDNLYLSIVLSVVVIVTGCFSYYQEAKSSKIMESFKNMVPQQALVIRGGEKMQINVQEVVLGDLVEIKGGDRVPADLRLISAQGCKVDNSSLTGESEPQSRSPDFTHENPLETRNICFFSTNCVEGTARGIVIATGDSTVMGRIASLTSGLAVGQTPIAAEIEHFIHLITVVAVFLGVTFFALSLLLGYGWLEAIIFLIGIIVANVPEGLLATVTVCLTLTAKRMARKNCLVKNLEAVETLGSTSTICSDKTGTLTQNRMTVAHMWFDMTVYEADTTEEQTGKTFTKSSDTWFMLARIAGLCNRADFKANQEILPIAKRATTGDASESALLKFIEQSYSSVAEMREKNPKVAEIPFNSTNKYQMSIHLREDSSQTHVLMMKGAPERILEFCSTFLLNGQEYSMNDEMKEAFQNAYLELGGLGERVLGFCFLNLPSSFSKGFPFNTDEINFPMDNLCFVGLISMIDPPRAAVPDAVSKCRSAGIKVIMVTGDHPITAKAIAKGVGIISEGTETAEEVAARLKIPISKVDASAAKAIVVHGAELKDIQSKQLDQILQNHPEIVFARTSPQQKLIIVEGCQRLGAVVAVTGDGVNDSPALKKADIGIAMGISGSDVSKQAADMILLDDNFASIVTGVEEGRLIFDNLKKSIMYTLTSNIPEITPFLMFIILGIPLPLGTITILCIDLGTDMVPAISLAYESAESDIMKRLPRNPKTDNLVNHRLIGMAYGQIGMIQALAGFFTYFVILAENGFRPVDLLGIRLHWEDKYLNDLEDSYGQQWTYEQRKVVEFTCQTAFFVTIVVVQWADLIISKTRRNSLFQQGMRNKVLIFGILEETLLAAFLSYTPGMDVALRMYPLKITWWLCAIPYSILIFVYDEIRKLLIRQHPDGWVERETYY

SEQ ID NO: 28 - protein, 279 aa, homo sapiens
MTKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARA

SEQ ID NO: 29 - protein, 1258 aa, Homo sapiens
MGDMANNSVAYSGVKNSLKEANHDGDFGITLAELRALMELRSTDALRKIQESYGDVYGICTKLKTSPNEGLSGNPADLERREAVFGKNFIPPKKPKTFLQLVWEALQDVTLIILEIAAIVSLGLSFYQPPEGDNALCGEVSVGEEEGEGETGWIEGAAILLSVVCVVLVTAFNDWSKEKQFRGLQSRIEQEQKFTVIRGGQVIQIPVADITVGDIAQVKYGDLLPADGILIQGNDLKIDESSLTGESDHVKKSLDKDPLLLSGTHVMEGSGRMVVTAVGVNSQTGIIFTLLGAGGEEEEKKDEKKKEKKNKKQDGAIENRNKAKAQDGAAMEMQPLKSEEGGDGDEKDKKKANLPKKEKSVLQGKLTKLAVQIGKAGLLMSAITVIILVLYFVIDTFWVQKRPWLAECTPIYIQYFVKFFIIGVTVLVVAVPEGLPLAVTISLAYSVKKMMKDNNLVRHLDACETMGNATAICSDKTGTLTMNRMTVVQAYINEKHYKKVPEPEAIPPNILSYLVTGISVNCAYTSKILPPEKEGGLPRHVGNKTECALLGLLLDLKRDYQDVRNEIPEEALYKVYTFNSVRKSMSTVLKNSDGSYRIFSKGASEIILKKCFKILSANGEAKVFRPRDRDDIVKTVIEPMASEGLRTICLAFRDFPAGEPEPEWDNENDIVTGLTCIAVVGIEDPVRPEVPDAIKKCQRAGITVRMVTGDNINTARAIATKCGILHPGEDFLCLEGKDFNRRIRNEKGEIEQERIDKIWPKLRVLARSSPTDKHTLVKGIIDSTVSDQRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTLASLALATEPPTESLLLRKPYGRNKPLISRTMMKNILGHAFYQLVVVFTLLFAGEKFFDIDSGRNAPLHAPPSEHYTIVFNTFVLMQLFNEINARKIHGERNVFEGIFNNAIFCTIVLGTFVVQIIIVQFGGKPFSCSELSIEQWLWSIFLGMGTLLWGQLISTIPTSRLKFLKEAGHGTQKEEIPEEELAEDVEEIDHAERELRRGQILWFRGLNRIQTQMDVVNAFQSGSSIQGALRRQPSIASQHHDVTNISTPTHIRVVNAFRSSLYEGLEKPESRSSIHNFMTHPEFRIEDSEPHIPLIDDTDAEDDAPTKRNSSPPPSPNKNNNAVDSGIHLTIEMNKSATSSSPGSPLHSLETSL

SEQ ID NO: 30 - protein, 1272 aa, Homo sapiens
MGDMTNSDFYSKNQRNESSHGGEFGCTMEELRSLMELRGTEAVVKIKETYGDTEAICRRLKTSPVEGLPGTAPDLEKRKQIFGQNFIPPKKPKTFLQLVWEALQDVTLIILEIAAIISLGLSFYHPPGEGNEGCATAQGGAEDEGEAEAGWIEGAAILLSVICVVLVTAFNDWSKEKQFRGLQSRIEQEQKFTVVRAGQVVQIPVAEIVVGDIAQVKYGDLLPADGLFIQGNDLKIDESSLTGESDQVRKSVDKDPMLLSGTHVMEGSGRMLVTAVGVNSQTGIIFTLLGAGGEEEEKKDKKGVKKGDGLQLPAADGAAASNAADSANASLVNGKMQDGNVDASQSKAKQQDGAAAMEMQPLKSAEGGDADDRKKASMHKKEKSVLQGKLTKLAVQIGKAGLVMSAITVIILVLYFTVDTFVVNKKPWLPECTPVYVQYFVKFFIIGVTVLVVAVPEGLPLAVTISLAYSVKKMMKDNNLVRHLDACETMGNATAICSDKTGTLTTNRMTVVQAYVGDVHYKEIPDPSSINTKTMELLINAIAINSAYTTKILPPEKEGALPRQVGNKTECGLLGFVLDLKQDYEPVRSQMPEEKLYKVYTFNSVRKSMSTVIKLPDESFRMYSKGASEIVLKKCCKILNGAGEPRVFRPRDRDEMVKKVIEPMACDGLRTICVAYRDFPSSPEPDWDNENDILNELTCICVVGIEDPVRPEVPEAIRKCQRAGITVRMVTGDNINTARAIAIKCGIIHPGEDFLCLEGKEFNRRIRNEKGEIEQERIDKIWPKLRVLARSSPTDKHTLVKGIIDSTHTEQRQVVAVTGDGTNDGPALKKADVGFAMGIAGTDVAKEASDIILTDDNFSSIVKAVMWGRNVYDSISKFLQFQLTVNVVAVIVAFTGACITQDSPLKAVQMLWVNLIMDTFASLALATEPPTETLLLRKPYGRNKPLISRTMMKNILGHAVYQLALIFTLLFVGEKMFQIDSGRNAPLHSPPSEHYTIIFNTFVMMQLFNEINARKIHGERNVFDGIFRNPIFCTIVLGTFAIQIVIVQFGGKPFSCSPLQLDQWMWCIFIGLGELVWGQVIATIPTSRLKFLKEAGRLTQKEEIPEEELNEDVEEIDHAERELRRGQILWFRGLNRIQTQIEVVNTFKSGASFQGALRRQSSVTSQSQDIRVVKAFRSSLYEGLEKPESRTSIHNFMAHPEFRIEDSQPHIPLIDDTDLEEDAALKQNSSPPSSLNKNNSAIDSGINLTTDTSKSATSSSPGSPIHSLETSL

SEQ ID NO: 31 - protein, 1241 aa, Homo sapiens
MTNPSDRVLPANSMAESREGDFGCTVMELRKLMELRSRDALTQINVHYGGVQNLCSRLKTSPVEGLSGNPADLEKRRQVFGHNVIPPKKPKTFLELVWEALQDVTLIILEIAAIISLVLSFYRPAGEENELCGQVATTPEDENEAQAGWIEGAAILFSVIIVVLVTAFNDWSKEKQFRGLQCRIEQEQKFSIIRNGQLIQLPVAEIVVGDIAQVKYGDLLPADGILIQGNDLKIDESSLTGESDHVKKSLDKDPMLLSGTHVMEGSGRMVVTAVGVNSQTGIILTLLGVNEDDEGEKKKKGKKQGVPENRNKAKTQDGVALEIQPLNSQEGIDNEEKDKKAVKVPKKEKSVLQGKLTRLAVQIGKAGLLMSALTVFILILYFVIDNFVINRRPWLPECTPIYIQYFVKFFIIGITVLVVAVPEGLPLAVTISLAYSVKKMMKDNNLVRHLDACETMGNATAICSDKTGTLTMNRMTVVQAYIGGIHYRQIPSPDVFLPKVLDLIVNGISINSAYTSKILPPEKEGGLPRQVGNKTECALLGFVTDLKQDYQAVRNEVPEEKLYKVYTFNSVRKSMSTVIRNPNGGFRMYSKGASEIILRKCNRILDRKGEAVPFKNKDRDDMVRTVIEPMACDGLRTICIAYRDFDDTEPSWDNENEILTELTCIAVVGIEDPVRPEVPDAIAKCKQAGITVRMVTGDNINTARAIATKCGILTPGDDFLCLEGKEFNRLIRNEKGEVEQEKLDKIWPKLRVLARSSPTDKHTLVKGIIDSTVGEHRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTFASLALATEPPTESLLKRRPYGRNKPLISRTMMKNILGHAFYQLIVIFILVFAGEKFFDIDSGRKAPLHSPPSQHYTIVFNTFVLMQLFNEINSRKIHGEKNVFSGIYRNIIFCSVVLGTFICQIFIVEFGGKPFSCTSLSLSQWLWCLFIGIGELLWGQFISAIPTRSLKFLKEAGHGTTKEEITKDAEGLDEIDHAEMELRRGQILWFRGLNRIQTQIDVINTFQTGASFKGVLRRQNMGQHLDVKLVPSSSYIKVVKAFHSSLHESIQKPYNQKSIHSFMTHPEFAIEEELPRTPLLDEEEEENPDKASKFGTRVLLLDGEVTPYANTNNNAVDCNQVQLPQSDSSLQSLETSV

SEQ ID NO: 32 - protein, 1241 aa, homo sapiens; identical to SEQ ID NO: 31

SEQ ID NO: 33 - protein, 193 aa, homo sapiens
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 34 - protein, 1021 aa, homo sapiens
MAGISYVASFFLLLTKLSIGQREVTVQKGPLFRAEGYPVSIGCNVTGHQGPSEQHFQWSVYLPTNPTQEVQIISTKDAAFSYAVYTQRVRSGDVYVERVQGNSVLLHISKLQMKDAGEYECHTPNTDEKYYGSYSAKTNLIVIPDTLSATMSSQTLGKEEGEPLALTCEASKATAQHTHLSVTWYLTQDGGGSQATEIISLSKDFILVPGPLYTERFAASDVQLNKLGPTTFRLSIERLQSSDQGQLFCEATEWIQDPDETWMFITKKQTDQTTLRIQPAVKDFQVNITADSLFAEGKPLELVCLVVSSGRDPQLQGIWFFNGTEIAHIDAGGVLGLKNDYKERASQGELQVSKLGPKAFSLKIFSLGPEDEGAYRCVVAEVMKTRTGSWQVLQRKQSPDSHVHLRKPAARSVVMSTKNKQQVVWEGETLAFLCKAGGAESPLSVSWWHIPRDQTQPEFVAGMGQDGIVQLGASYGVPSYHGNTRLEKMDWATFQLEITFTAITDSGTYECRVSEKSRNQARDLSWTQKISVTVKSLESSLQVSLMSRQPQVMLTNTFDLSCVVRAGYSDLKVPLTVTWQFQPASSHIFHQLIRITHNGTIEWGNFLSRFQKKTKVSQSLFRSQLLVHDATEEETGVYQCEVEVYDRNSLYNNRPPRASAISHPLRIAVTLPESKLKVNSRSQVQELSINSNTDIECSILSRSNGNLQLAIIWYFSPVSTNASWLKILEMDQTNVIKTGDEFHTPQRKQKFHTEKVSQDLFQLHILNVEDSDRGKYHCAVEEWLLSTNGTWHKLGEKKSGLTELKLKPTGSKVRVSKVYWTENVTEHREVAIRCSLESVGSSATLYSVMWYWNRENSGSKLLVHLQHDGLLEYGEEGLRRHLHCYRSSSTDFVLKLHQVEMEDAGMYWCRVAEWQLHGHPSKWINQASDESQRMVLTVLPSEPTLPSRICSSAPLLYFLFICPFVLLLLLLISLLCLYWKARKLSTLRSNTRKEKALWVDLKEAGGVTTNRREDEEEDEGN

SEQ ID NO: 35 - protein, 20 aa, homo sapiens
MAGISYVASFFLLLTKLSIG

SEQ ID NO: 36 - DNA, 20 bp, Artificial Sequence
  Synthetic  Oligonucleotide
cgttggcagtccgccttaac

SEQ ID NO: 37 - DNA, 20 bp, Artificial Sequence
  Synthetic  oligonucleotide
catagtcactgacgttgcag

SEQ ID NO: 38 - DNA, 20 bp, Artificial Sequence
  Synthetic  oligonucleotide
ttgtggagcttgcaagcacc

SEQ ID NO: 39 - DNA, 20 bp, Artificial Sequence
  Synthetic  oligonucleotide
gttctttatgtggagctcca

SEQ ID NO: 40 - DNA, 20 bp, Artificial Sequence
  Synthetic  oligonucleotide
tatcccttgctgatcggcgt

SEQ ID NO: 41 - DNA, 20 bp, Artificial Sequence
  Synthetic  oligonucleotide
gctgcagtacccgatgagac

SEQ ID NO: 42 - protein, 38 aa, Artificial Sequence
  Synthetic Polypeptide
EHSAGGGGSDYKDDDDKGGGGSLSNPIEIDFQTSGPIF

SEQ ID NO: 43 - protein, 34 aa, Artificial Sequence
  Synthetic Polypeptide
EHSAGGGGSDYKDDDDKGGGGSIEIDFQTSGPIF

SEQ ID NO: 44 - protein, 30 aa, Artificial Sequence
  Synthetic Polypeptide
EHSAGGGGSDYKDDDDKGGGGSFQTSGPIF

SEQ ID NO: 45 - protein, 26 aa, Artificial Sequence
  Synthetic Polypeptide
EHSAGGGGSDYKDDDDKGGGGSGPIF

SEQ ID NO: 46 - protein, 42 aa, Artificial Sequence
  Synthetic Polypeptide
FITVKMDTLDPRSFLLRNPNDKYEPFWEDEEKNESGSDKTHT

SEQ ID NO: 47 - protein, 332 aa, Homo sapiens
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 48 - protein, 195 aa, Homo sapiens
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 49 - protein, 227 aa, Homo sapiens
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 50 - protein, 30 aa, Artificial Sequence
  Description of Artificial Sequence: Synthetic peptide
MGGKLSKKKKGYNVNDEKAKEKDKKAEGAA

SEQ ID NO: 51 - protein, 27 aa, Artificial Sequence
  Description of Artificial Sequence: Synthetic peptide
MGGKLSKKKKGYNVNDEKAKEKDKKAE

SEQ ID NO: 52 - protein, 24 aa, Artificial Sequence
  Description of Artificial Sequence: Synthetic peptide
MGGKLSKKKKGYNVNDEKAKEKDK

SEQ ID NO: 53 - protein, 21 aa, Artificial Sequence
  Description of Artificial Sequence: Synthetic peptide
MGGKLSKKKKGYNVNDEKAKE

SEQ ID NO: 54 - protein, 18 aa, Artificial Sequence
  Description of Artificial Sequence: Synthetic peptide
MGGKLSKKKKGYNVNDEK

SEQ ID NO: 55 - protein, 15 aa, Artificial Sequence
  Description of Artificial Sequence: Synthetic peptide
MGGKLSKKKKGYNVN

SEQ ID NO: 56 - protein, 12 aa, Artificial Sequence
  Description of Artificial Sequence: Synthetic peptide
MGGKLSKKKKGY

SEQ ID NO: 57 - protein, 11 aa, Artificial Sequence
  Description of Artificial Sequence: Synthetic peptide
MGGKLSKKKKG

SEQ ID NO: 58 - protein, 10 aa, Artificial Sequence
  Description of Artificial Sequence: Synthetic peptide
MGGKLSKKKK

SEQ ID NO: 59 - protein, 9 aa, Artificial Sequence
  Description of Artificial Sequence: Synthetic peptide
MGGKLSKKK

SEQ ID NO: 60 - protein, 8 aa, Artificial Sequence
  Description of Artificial Sequence: Synthetic peptide
MGGKLSKK

SEQ ID NO: 61 - protein, 7 aa, Artificial Sequence
  Description of Artificial Sequence: Synthetic peptide
MGGKLSK

SEQ ID NO: 62 - protein, 8 aa, Artificial Sequence
  Description of Artificial Sequence: Synthetic peptide
MGGKLAKK

SEQ ID NO: 63 - protein, 8 aa, Artificial Sequence
  Description of Artificial Sequence: Synthetic peptide
MGGKFSKK

SEQ ID NO: 64 - protein, 8 aa, Artificial Sequence
  Description of Artificial Sequence: Synthetic peptide
MGGKFAKK

SEQ ID NO: 65 - protein, 8 aa, Artificial Sequence
  Description of Artificial Sequence: Synthetic peptide
MGGKSSKK

SEQ ID NO: 66 - protein, 8 aa, Artificial Sequence
  Description of Artificial Sequence: Synthetic peptide
MGGKSAKK

SEQ ID NO: 67 - protein, 8 aa, Artificial Sequence
  Description of Artificial Sequence: Synthetic peptide
MGGKQSKK

SEQ ID NO: 68 - protein, 8 aa, Artificial Sequence
  Description of Artificial Sequence: Synthetic peptide
MGGKQAKK

SEQ ID NO: 69 - protein, 8 aa, Artificial Sequence
  Description of Artificial Sequence: Synthetic peptide
MGGQLSKK

SEQ ID NO: 70 - protein, 8 aa, Artificial Sequence
  Description of Artificial Sequence: Synthetic peptide
MGGQLAKK

SEQ ID NO: 71 - protein, 8 aa, Artificial Sequence
  Description of Artificial Sequence: Synthetic peptide
MGGQFSKK

SEQ ID NO: 72 - protein, 8 aa, Artificial Sequence
  Description of Artificial Sequence: Synthetic peptide
MGGQFAKK

SEQ ID NO: 73 - protein, 8 aa, Artificial Sequence
  Description of Artificial Sequence: Synthetic peptide
MGGQSSKK

SEQ ID NO: 74 - protein, 8 aa, Artificial Sequence
  Description of Artificial Sequence: Synthetic peptide
MGGQSAKK

SEQ ID NO: 75 - protein, 8 aa, Artificial Sequence
  Description of Artificial Sequence: Synthetic peptide
MGGQQSKK

SEQ ID NO: 76 - protein, 8 aa, Artificial Sequence
  Description of Artificial Sequence: Synthetic peptide
MGGQQAKK

SEQ ID NO: 77 - protein, 8 aa, Artificial Sequence
  Description of Artificial Sequence: Synthetic peptide
MGAKLSKK

SEQ ID NO: 78 - protein, 8 aa, Artificial Sequence
  Description of Artificial Sequence: Synthetic peptide
MGAKLAKK

SEQ ID NO: 79 - protein, 8 aa, Artificial Sequence
  Description of Artificial Sequence: Synthetic peptide
MGAKFSKK

SEQ ID NO: 80 - protein, 8 aa, Artificial Sequence
  Description of Artificial Sequence: Synthetic peptide
MGAKFAKK

SEQ ID NO: 81 - protein, 8 aa, Artificial Sequence
  Description of Artificial Sequence: Synthetic peptide
MGAKSSKK

SEQ ID NO: 82 - protein, 8 aa, Artificial Sequence
  Description of Artificial Sequence: Synthetic peptide
MGAKSAKK

SEQ ID NO: 83 - protein, 8 aa, Artificial Sequence
  Description of Artificial Sequence: Synthetic peptide
MGAKQSKK

SEQ ID NO: 84 - protein, 8 aa, Artificial Sequence
  Description of Artificial Sequence: Synthetic peptide
MGAKQAKK

SEQ ID NO: 85 - protein, 8 aa, Artificial Sequence
  Description of Artificial Sequence: Synthetic peptide
MGAQLSKK

SEQ ID NO: 86 - protein, 8 aa, Artificial Sequence
  Description of Artificial Sequence: Synthetic peptide
MGAQLAKK

SEQ ID NO: 87 - protein, 8 aa, Artificial Sequence
  Description of Artificial Sequence: Synthetic peptide
MGAQFSKK

SEQ ID NO: 88 - protein, 8 aa, Artificial Sequence
  Description of Artificial Sequence: Synthetic peptide
MGAQFAKK

SEQ ID NO: 89 - protein, 8 aa, Artificial Sequence
  Description of Artificial Sequence: Synthetic peptide
MGAQSSKK

SEQ ID NO: 90 - protein, 8 aa, Artificial Sequence
  Description of Artificial Sequence: Synthetic peptide
MGAQSAKK

SEQ ID NO: 91 - protein, 8 aa, Artificial Sequence
  Description of Artificial Sequence: Synthetic peptide
MGAQQSKK

SEQ ID NO: 92 - protein, 8 aa, Artificial Sequence
  Description of Artificial Sequence: Synthetic peptide
MGAQQAKK

SEQ ID NO: 93 - protein, 8 aa, Artificial Sequence
  Description of Artificial Sequence: Synthetic peptide
MGSKLSKK

SEQ ID NO: 94 - protein, 8 aa, Artificial Sequence
  Description of Artificial Sequence: Synthetic peptide
MGSKLAKK

SEQ ID NO: 95 - protein, 8 aa, Artificial Sequence
  Description of Artificial Sequence: Synthetic peptide
MGSKFSKK

SEQ ID NO: 96 - protein, 8 aa, Artificial Sequence
  Description of Artificial Sequence: Synthetic peptide
MGSKFAKK

SEQ ID NO: 97 - protein, 8 aa, Artificial Sequence
  Description of Artificial Sequence: Synthetic peptide
MGSKSSKK

SEQ ID NO: 98 - protein, 8 aa, Artificial Sequence
  Description of Artificial Sequence: Synthetic peptide
MGSKSAKK

SEQ ID NO: 99 - protein, 8 aa, Artificial Sequence
  Description of Artificial Sequence: Synthetic peptide
MGSKQSKK

SEQ ID NO: 100 - protein, 8 aa, Artificial Sequence
  Description of Artificial Sequence: Synthetic peptide
MGSKQAKK

SEQ ID NO: 101 - protein, 8 aa, Artificial Sequence
  Description of Artificial Sequence: Synthetic peptide
MGSQLSKK

SEQ ID NO: 102 - protein, 8 aa, Artificial Sequence
  Description of Artificial Sequence: Synthetic peptide
MGSQLAKK

SEQ ID NO: 103 - protein, 8 aa, Artificial Sequence
  Description of Artificial Sequence: Synthetic peptide
MGSQFSKK

SEQ ID NO: 104 - protein, 8 aa, Artificial Sequence
  Description of Artificial Sequence: Synthetic peptide
MGSQFAKK

SEQ ID NO: 105 - protein, 8 aa, Artificial Sequence
  Description of Artificial Sequence: Synthetic peptide
MGSQSSKK

SEQ ID NO: 106 - protein, 8 aa, Artificial Sequence
  Description of Artificial Sequence: Synthetic peptide
MGSQSAKK

SEQ ID NO: 107 - protein, 8 aa, Artificial Sequence
  Description of Artificial Sequence: Synthetic peptide
MGSQQSKK

SEQ ID NO: 108 - protein, 8 aa, Artificial Sequence
  Description of Artificial Sequence: Synthetic peptide
MGSQQAKK

SEQ ID NO: 109 - protein, 7 aa, Artificial Sequence
  Description of Artificial Sequence: Synthetic peptide
MGGKLAK

SEQ ID NO: 110 - protein, 7 aa, Artificial Sequence
  Description of Artificial Sequence: Synthetic peptide
MGGKFSK

SEQ ID NO: 111 - protein, 7 aa, Artificial Sequence
  Description of Artificial Sequence: Synthetic peptide
MGGKFAK

SEQ ID NO: 112 - protein, 7 aa, Artificial Sequence
  Description of Artificial Sequence: Synthetic peptide
MGGKSSK

SEQ ID NO: 113 - protein, 7 aa, Artificial Sequence
  Description of Artificial Sequence: Synthetic peptide
MGGKSAK

SEQ ID NO: 114 - protein, 7 aa, Artificial Sequence
  Description of Artificial Sequence: Synthetic peptide
MGGKQSK

SEQ ID NO: 115 - protein, 7 aa, Artificial Sequence
  Description of Artificial Sequence: Synthetic peptide
MGGKQAK

SEQ ID NO: 116 - protein, 7 aa, Artificial Sequence
  Description of Artificial Sequence: Synthetic peptide
MGGQLSK

SEQ ID NO: 117 - protein, 7 aa, Artificial Sequence
  Description of Artificial Sequence: Synthetic peptide
MGGQLAK

SEQ ID NO: 118 - protein, 7 aa, Artificial Sequence
  Description of Artificial Sequence: Synthetic peptide
MGGQFSK

SEQ ID NO: 119 - protein, 7 aa, Artificial Sequence
  Description of Artificial Sequence: Synthetic peptide
MGGQFAK

SEQ ID NO: 120 - protein, 7 aa, Artificial Sequence
  Description of Artificial Sequence: Synthetic peptide
MGGQSSK

SEQ ID NO: 121 - protein, 7 aa, Artificial Sequence
  Description of Artificial Sequence: Synthetic peptide
MGGQSAK

SEQ ID NO: 122 - protein, 7 aa, Artificial Sequence
  Description of Artificial Sequence: Synthetic peptide
MGGQQSK

SEQ ID NO: 123 - protein, 7 aa, Artificial Sequence
  Description of Artificial Sequence: Synthetic peptide
MGGQQAK

SEQ ID NO: 124 - protein, 7 aa, Artificial Sequence
  Description of Artificial Sequence: Synthetic peptide
MGAKLSK

SEQ ID NO: 125 - protein, 7 aa, Artificial Sequence
  Description of Artificial Sequence: Synthetic peptide
MGAKLAK

SEQ ID NO: 126 - protein, 7 aa, Artificial Sequence
  Description of Artificial Sequence: Synthetic peptide
MGAKFSK

SEQ ID NO: 127 - protein, 7 aa, Artificial Sequence
  Description of Artificial Sequence: Synthetic peptide
MGAKFAK

SEQ ID NO: 128 - protein, 7 aa, Artificial Sequence
  Description of Artificial Sequence: Synthetic peptide
MGAKSSK

SEQ ID NO: 129 - protein, 7 aa, Artificial Sequence
  Description of Artificial Sequence: Synthetic peptide
MGAKSAK

SEQ ID NO: 130 - protein, 7 aa, Artificial Sequence
  Description of Artificial Sequence: Synthetic peptide
MGAKQSK

SEQ ID NO: 131 - protein, 7 aa, Artificial Sequence
  Description of Artificial Sequence: Synthetic peptide
MGAKQAK

SEQ ID NO: 132 - protein, 7 aa, Artificial Sequence
  Description of Artificial Sequence: Synthetic peptide
MGAQLSK

SEQ ID NO: 133 - protein, 7 aa, Artificial Sequence
  Description of Artificial Sequence: Synthetic peptide
MGAQLAK

SEQ ID NO: 134 - protein, 7 aa, Artificial Sequence
  Description of Artificial Sequence: Synthetic peptide
MGAQFSK

SEQ ID NO: 135 - protein, 7 aa, Artificial Sequence
  Description of Artificial Sequence: Synthetic peptide
MGAQFAK

SEQ ID NO: 136 - protein, 7 aa, Artificial Sequence
  Description of Artificial Sequence: Synthetic peptide
MGAQSSK

SEQ ID NO: 137 - protein, 7 aa, Artificial Sequence
  Description of Artificial Sequence: Synthetic peptide
MGAQSAK

SEQ ID NO: 138 - protein, 7 aa, Artificial Sequence
  Description of Artificial Sequence: Synthetic peptide
MGAQQSK

SEQ ID NO: 139 - protein, 7 aa, Artificial Sequence
  Description of Artificial Sequence: Synthetic peptide
MGAQQAK

SEQ ID NO: 140 - protein, 7 aa, Artificial Sequence
  Description of Artificial Sequence: Synthetic peptide
MGSKLSK

SEQ ID NO: 141 - protein, 7 aa, Artificial Sequence
  Description of Artificial Sequence: Synthetic peptide
MGSKLAK

SEQ ID NO: 142 - protein, 7 aa, Artificial Sequence
  Description of Artificial Sequence: Synthetic peptide
MGSKFSK

SEQ ID NO: 143 - protein, 7 aa, Artificial Sequence
  Description of Artificial Sequence: Synthetic peptide
MGSKFAK

SEQ ID NO: 144 - protein, 7 aa, Artificial Sequence
  Description of Artificial Sequence: Synthetic peptide
MGSKSSK

SEQ ID NO: 145 - protein, 7 aa, Artificial Sequence
  Description of Artificial Sequence: Synthetic peptide
MGSKSAK

SEQ ID NO: 146 - protein, 7 aa, Artificial Sequence
  Description of Artificial Sequence: Synthetic peptide
MGSKQSK

SEQ ID NO: 147 - protein, 7 aa, Artificial Sequence
  Description of Artificial Sequence: Synthetic peptide
MGSKQAK

SEQ ID NO: 148 - protein, 7 aa, Artificial Sequence
  Description of Artificial Sequence: Synthetic peptide
MGSQLSK

SEQ ID NO: 149 - protein, 7 aa, Artificial Sequence
  Description of Artificial Sequence: Synthetic peptide
MGSQLAK

SEQ ID NO: 150 - protein, 7 aa, Artificial Sequence
  Description of Artificial Sequence: Synthetic peptide
MGSQFSK

SEQ ID NO: 151 - protein, 7 aa, Artificial Sequence
  Description of Artificial Sequence: Synthetic peptide
MGSQFAK

SEQ ID NO: 152 - protein, 7 aa, Artificial Sequence
  Description of Artificial Sequence: Synthetic peptide
MGSQSSK

SEQ ID NO: 153 - protein, 7 aa, Artificial Sequence
  Description of Artificial Sequence: Synthetic peptide
MGSQSAK

SEQ ID NO: 154 - protein, 7 aa, Artificial Sequence
  Description of Artificial Sequence: Synthetic peptide
MGSQQSK

SEQ ID NO: 155 - protein, 7 aa, Artificial Sequence
  Description of Artificial Sequence: Synthetic peptide
MGSQQAK

SEQ ID NO: 156 - protein, 9 aa, Artificial Sequence
  Description of Artificial Sequence: Synthetic peptide
MGAKLSKKK

SEQ ID NO: 157 - protein, 167 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 158 - protein, 167 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 159 - protein, 296 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGGSGGSGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 160 - protein, 296 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIYGSGGSGGSGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 161 - RNA, 680 nt, Artificial Sequence
  Description of Artificial Sequence: Synthetic polynucleotide
augaagcccaccgagaacaacgaagacuucaacaucguggccguggccagcaacuucgcgaccacggaucucgaugcugaccgcgggaaguugcccggcaagaagcugccgcuggaggugcucaaagaguuggaagccaaugcccggaaagcuggcugcaccaggggcugucugaucugccugucccacaucaagugcacgcccaagaugaagaaguucaucccaggacgcugccacaccuacgaaggcgacaaagaguccgcacagggcggcauaggcgaggcgaucgucgacauuccugagauuccuggguucaaggacuuggagcccuuggagcaguucaucgcacaggucgaucuguguguggacugcacaacuggcugccucaaagggcuugccaacgugcaguguucugaccugcucaagaaguggcugccgcaacgcugugcgaccuuugccagcaagauccagggccagguggacaagaucaagggggccgguggugacuaaggauccaucgauaagcuucaucgaaacaugaggaucacccauaucugcagucgacaucgaaacaugaggaucacccaugucugcagucgacaucgaaacaugaggaucacccaugucugcagucgacaucgaaacaugaggaucacccaugucugcagucgacaucgaaa

SEQ ID NO: 162 - protein, 9 aa, Artificial Sequence
  Description of Artificial Sequence: Synthetic peptide
  MOD_RES (3)..(3): Any Naturally Occuring Amino Acid
MGXKLSKKK

SEQ ID NO: 163 - protein, 9 aa, Artificial Sequence
  Description of Artificial Sequence: Synthetic peptide
  MOD_RES (3)..(3): See specification as filed for detailed description of substitutions and preferred embodiments
MGXKLSKKK

SEQ ID NO: 164 - protein, 8 aa, Artificial Sequence
  Description of Artificial Sequence: Synthetic peptide
  MOD_RES (3)..(3): Gly, Ala, or Ser
  MOD_RES (4)..(4): Lys or Gln
  MOD_RES (5)..(5): Leu, Phe, Ser, or Gln
  MOD_RES (6)..(6): Ser or Ala
  MOD_RES (6)..(6): Ser or Ala, See specification as filed for detailed description of substitutions and preferred embodiments
MGXXXXKK

SEQ ID NO: 165 - DNA, 20 bp, Artificial Sequence
  Description of Artificial Sequence: Synthetic primer
tggaggtgctcaaagagttg

SEQ ID NO: 166 - DNA, 17 bp, Artificial Sequence
  Description of Artificial Sequence: Synthetic primer
ttgggcgtgcacttgat

SEQ ID NO: 167 - DNA, 13 bp, Artificial Sequence
  Description of Artificial Sequence: Synthetic probe
gggcattggcttc

SEQ ID NO: 168 - protein, 60 aa, Artificial Sequence
  Description of Artificial Sequence: Synthetic polypeptide
MGGKLSKKKKGYNVNDEKAKEKDKKAEGAASAGGGGSDYKDDDDKGGGGSVSKGEELFTG

SEQ ID NO: 169 - protein, 60 aa, Artificial Sequence
  Description of Artificial Sequence: Synthetic polypeptide
MAGKLSKKKKGYNVNDEKAKEKDKKAEGAASAGGGGSDYKDDDDKGGGGSVSKGEELFTG

SEQ ID NO: 170 - protein, 60 aa, Artificial Sequence
  Description of Artificial Sequence: Synthetic polypeptide
MGAKLSKKKKGYNVNDEKAKEKDKKAEGAASAGGGGSDYKDDDDKGGGGSVSKGEELFTG

SEQ ID NO: 171 - protein, 60 aa, Artificial Sequence
  Description of Artificial Sequence: Synthetic polypeptide
MAAKLSKKKKGYNVNDEKAKEKDKKAEGAASAGGGGSDYKDDDDKGGGGSVSKGEELFTG

SEQ ID NO: 172 - protein, 57 aa, Artificial Sequence
  Description of Artificial Sequence: Synthetic polypeptide
MGGKLSKKKKGYNVNDEKAKEKDKKAESAGGGGSDYKDDDDKGGGGSVSKGEELFTG

SEQ ID NO: 173 - protein, 54 aa, Artificial Sequence
  Description of Artificial Sequence: Synthetic polypeptide
MGGKLSKKKKGYNVNDEKAKEKDKSAGGGGSDYKDDDDKGGGGSVSKGEELFTG

SEQ ID NO: 174 - protein, 51 aa, Artificial Sequence
  Description of Artificial Sequence: Synthetic polypeptide
MGGKLSKKKKGYNVNDEKAKESAGGGGSDYKDDDDKGGGGSVSKGEELFTG

SEQ ID NO: 175 - protein, 48 aa, Artificial Sequence
  Description of Artificial Sequence: Synthetic polypeptide
MGGKLSKKKKGYNVNDEKSAGGGGSDYKDDDDKGGGGSVSKGEELFTG

SEQ ID NO: 176 - protein, 45 aa, Artificial Sequence
  Description of Artificial Sequence: Synthetic polypeptide
MGGKLSKKKKGYNVNSAGGGGSDYKDDDDKGGGGSVSKGEELFTG

SEQ ID NO: 177 - protein, 42 aa, Artificial Sequence
  Description of Artificial Sequence: Synthetic polypeptide
MGGKLSKKKKGYSAGGGGSDYKDDDDKGGGGSVSKGEELFTG

SEQ ID NO: 178 - protein, 39 aa, Artificial Sequence
  Description of Artificial Sequence: Synthetic polypeptide
MGGKLSKKKSAGGGGSDYKDDDDKGGGGSVSKGEELFTG

SEQ ID NO: 179 - protein, 36 aa, Artificial Sequence
  Description of Artificial Sequence: Synthetic polypeptide
MGGKLSSAGGGGSDYKDDDDKGGGGSVSKGEELFTG

SEQ ID NO: 180 - protein, 33 aa, Artificial Sequence
  Description of Artificial Sequence: Synthetic polypeptide
MGGSAGGGGSDYKDDDDKGGGGSVSKGEELFTG

SEQ ID NO: 181 - protein, 54 aa, Artificial Sequence
  Description of Artificial Sequence: Synthetic polypeptide
MGGKLSKKKKGYNVNDEKAKEKDKKAEGAASAGGGGSDYKDDDDKGGGGSVSKG

SEQ ID NO: 182 - protein, 36 aa, Artificial Sequence
  Description of Artificial Sequence: Synthetic polypeptide
MGGKLSKKKKGYSAGGGGSDYKDDDDKGGGGSVSKG

SEQ ID NO: 183 - protein, 35 aa, Artificial Sequence
  Description of Artificial Sequence: Synthetic polypeptide
MGGKLSKKKKGSAGGGGSDYKDDDDKGGGGSVSKG

SEQ ID NO: 184 - protein, 34 aa, Artificial Sequence
  Description of Artificial Sequence: Synthetic polypeptide
MGGKLSKKKKSAGGGGSDYKDDDDKGGGGSVSKG

SEQ ID NO: 185 - protein, 33 aa, Artificial Sequence
  Description of Artificial Sequence: Synthetic polypeptide
MGGKLSKKKSAGGGGSDYKDDDDKGGGGSVSKG

SEQ ID NO: 186 - protein, 32 aa, Artificial Sequence
  Description of Artificial Sequence: Synthetic polypeptide
MGGKLSKKSAGGGGSDYKDDDDKGGGGSVSKG

SEQ ID NO: 187 - protein, 31 aa, Artificial Sequence
  Description of Artificial Sequence: Synthetic polypeptide
MGGKLSKSAGGGGSDYKDDDDKGGGGSVSKG

SEQ ID NO: 188 - protein, 30 aa, Artificial Sequence
  Description of Artificial Sequence: Synthetic polypeptide
MGGKLSSAGGGGSDYKDDDDKGGGGSVSKG

SEQ ID NO: 189 - protein, 30 aa, Artificial Sequence
  Description of Artificial Sequence: Synthetic polypeptide
MGGKLDKKKKGYNVNDEKAKEKDKKAEGAA

SEQ ID NO: 190 - protein, 30 aa, Artificial Sequence
  Description of Artificial Sequence: Synthetic polypeptide
MGGKLAKKKKGYNVNDEKAKEKDKKAEGAA

SEQ ID NO: 191 - protein, 30 aa, Artificial Sequence
  Description of Artificial Sequence: Synthetic polypeptide
MGGKQSKKKKGYNVNDEKAKEKDKKAEGAA

SEQ ID NO: 192 - protein, 30 aa, Artificial Sequence
  Description of Artificial Sequence: Synthetic polypeptide
MGAKKKKKRFSFKKSFKLSGFSFKKNKKEA

SEQ ID NO: 193 - protein, 30 aa, Artificial Sequence
  Description of Artificial Sequence: Synthetic polypeptide
MAAKKKKKRFSFKKSFKLSGFSFKKNKKEA

SEQ ID NO: 194 - protein, 30 aa, Artificial Sequence
  Description of Artificial Sequence: Synthetic polypeptide
MGAKKSKKRFSFKKSFKLSGFSFKKNKKEA

SEQ ID NO: 195 - protein, 30 aa, Artificial Sequence
  Description of Artificial Sequence: Synthetic polypeptide
MGAKKAKKRFSFKKPFKLSGFSFKKNKKEA

SEQ ID NO: 196 - protein, 153 aa, Artificial Sequence
  Description of Artificial Sequence: Synthetic polypeptide
MGGKLSKKKKSAGGSGGSTSGSGDYKDDDDKGSGFEMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 197 - protein, 4 aa, Artificial Sequence
  Peptide Linker
GGGG

SEQ ID NO: 198 - protein, 7 aa, Artificial Sequence
  Peptide Linker
SGGSGGS

SEQ ID NO: 199 - protein, 15 aa, Artificial Sequence
  Peptide Linker
GGSGGSGGSGGSGGG

SEQ ID NO: 200 - protein, 16 aa, Artificial Sequence
  Peptide Linker
GGSGGSGGGGSGGGGS

SEQ ID NO: 201 - protein, 18 aa, Artificial Sequence
  Peptide Linker
GGSGGSGGSGGSGGSGGS

SEQ ID NO: 202 - protein, 15 aa, Artificial Sequence
  Peptide Linker
GGGGSGGGGSGGGGS

SEQ ID NO: 203 - protein, 5 aa, Artificial Sequence
  Peptide Linker
  MISC_FEATURE: X can be an integer from 1-100
  misc_feature (5)..(5): X can be any integer from 1-100
GGGSX

SEQ ID NO: 204 - protein, 10 aa, Artificial Sequence
  Peptide Linker
  MISC_FEATURE (4)..(4): X can be any integer from 1-100
  MISC_FEATURE (10)..(10): X can be any integer from 1-100
GGSXGGGGSX

SEQ ID NO: 205 - protein, 4 aa, Artificial Sequence
  Effector Domain Sequence
KKKK

SEQ ID NO: 206 - protein, 5 aa, Artificial Sequence
  Effector Domain Sequence
KKKKK

SEQ ID NO: 207 - protein, 4 aa, Artificial Sequence
  Effector Domain Sequence
RRRR

SEQ ID NO: 208 - protein, 5 aa, Artificial Sequence
  Effector Domain Sequence
RRRRR

SEQ ID NO: 209 - protein, 4 aa, Artificial Sequence
  Effector Domain Sequence
  misc_feature (1)..(4): Xaa can be Lys or Arg
XXXX

SEQ ID NO: 210 - protein, 5 aa, Artificial Sequence
  Effector Domain Sequence
  misc_feature (1)..(5): Xaa can be Lys or Arg
XXXXX

SEQ ID NO: 211 - protein, 7 aa, Artificial Sequence
  N Terminus Domain Sequence
GGKLSKK

SEQ ID NO: 212 - protein, 7 aa, Artificial Sequence
  N Terminus Domain Sequence
GAKLSKK

SEQ ID NO: 213 - protein, 7 aa, Artificial Sequence
  N Terminus Domain Sequence
GGKQSKK

SEQ ID NO: 214 - protein, 7 aa, Artificial Sequence
  N Terminus Domain Sequence
GGKLAKK

SEQ ID NO: 215 - protein, 6 aa, Artificial Sequence
  N Terminus Domain Sequence
GGKLSK

SEQ ID NO: 216 - protein, 6 aa, Artificial Sequence
  N Terminus Domain Sequence
GAKLSK

SEQ ID NO: 217 - protein, 6 aa, Artificial Sequence
  N Terminus Domain Sequence
GGKQSK

SEQ ID NO: 218 - protein, 6 aa, Artificial Sequence
  N Terminus Domain Sequence
GGKLAK

SEQ ID NO: 219 - protein, 4 aa, Artificial Sequence
  N Terminus Domain Sequence
KKKG

SEQ ID NO: 220 - protein, 5 aa, Artificial Sequence
  N Terminus Domain Sequence
KKKGY

SEQ ID NO: 221 - protein, 6 aa, Artificial Sequence
  N Terminus Domain Sequence
KKKGYN

SEQ ID NO: 222 - protein, 7 aa, Artificial Sequence
  N Terminus Domain Sequence
KKKGYNV

SEQ ID NO: 223 - protein, 8 aa, Artificial Sequence
  N Terminus Domain Sequence
KKKGYNVN

SEQ ID NO: 224 - protein, 6 aa, Artificial Sequence
  N Terminus Domain Sequence
KKKGYS

SEQ ID NO: 225 - protein, 6 aa, Artificial Sequence
  N Terminus Domain Sequence
KKKGYG

SEQ ID NO: 226 - protein, 7 aa, Artificial Sequence
  N Terminus Domain Sequence
KKKGYGG

SEQ ID NO: 227 - protein, 5 aa, Artificial Sequence
  N Terminus Domain Sequence
KKKGS

SEQ ID NO: 228 - protein, 6 aa, Artificial Sequence
  N Terminus Domain Sequence
KKKGSG

SEQ ID NO: 229 - protein, 7 aa, Artificial Sequence
  N Terminus Domain Sequence
KKKGSGS

SEQ ID NO: 230 - protein, 4 aa, Artificial Sequence
  N Terminus Domain Sequence
KKKS

SEQ ID NO: 231 - protein, 5 aa, Artificial Sequence
  N Terminus Domain Sequence
KKKSG

SEQ ID NO: 232 - protein, 6 aa, Artificial Sequence
  N Terminus Domain Sequence
KKKSGG

SEQ ID NO: 233 - protein, 7 aa, Artificial Sequence
  N Terminus Domain Sequence
KKKSGGS

SEQ ID NO: 234 - protein, 8 aa, Artificial Sequence
  N Terminus Domain Sequence
KKKSGGSG

SEQ ID NO: 235 - protein, 8 aa, Artificial Sequence
  N Terminus Domain Sequence
KKSGGSGG

SEQ ID NO: 236 - protein, 10 aa, Artificial Sequence
  N Terminus Domain Sequence
KKKSGGSGGS

SEQ ID NO: 237 - protein, 8 aa, Artificial Sequence
  N Terminus Domain Sequence
KRFSFKKS

SEQ ID NO: 238 - protein, 8 aa, Artificial Sequence
  N Terminus Domain Sequence
GGKLSKKK

SEQ ID NO: 239 - protein, 8 aa, Artificial Sequence
  N Terminus Domain Sequence
GGKLSKKS

SEQ ID NO: 240 - protein, 8 aa, Artificial Sequence
  N Terminus Domain Sequence
GAKLSKKK

SEQ ID NO: 241 - protein, 8 aa, Artificial Sequence
  N Terminus Domain Sequence
GAKLSKKS

SEQ ID NO: 242 - protein, 8 aa, Artificial Sequence
  N Terminus Domain Sequence
GGKQSKKK

SEQ ID NO: 243 - protein, 8 aa, Artificial Sequence
  N Terminus Domain Sequence
GGKQSKKS

SEQ ID NO: 244 - protein, 8 aa, Artificial Sequence
  N Terminus Domain Sequence
GGKLAKKK

SEQ ID NO: 245 - protein, 8 aa, Artificial Sequence
  N Terminus Domain Sequence
GGKLAKKS

SEQ ID NO: 246 - protein, 14 aa, Artificial Sequence
  Scaffold Y Sequence
GGKLSKKKKGYNVN

SEQ ID NO: 247 - protein, 14 aa, Artificial Sequence
  Scaffold Y Sequence
GAKLSKKKKGYNVN

SEQ ID NO: 248 - protein, 14 aa, Artificial Sequence
  Scaffold Y Sequence
GGKQSKKKKGYNVN

SEQ ID NO: 249 - protein, 14 aa, Artificial Sequence
  Scaffold Y Sequence
GGKLAKKKKGYNVN

SEQ ID NO: 250 - protein, 14 aa, Artificial Sequence
  Scaffold Y Sequence
GGKLSKKKKGYSGG

SEQ ID NO: 251 - protein, 14 aa, Artificial Sequence
  Scaffold Y Sequence
GGKLSKKKKGSGGS

SEQ ID NO: 252 - protein, 14 aa, Artificial Sequence
  Scaffold Y Sequence
GGKLSKKKKSGGSG

SEQ ID NO: 253 - protein, 14 aa, Artificial Sequence
  Scaffold Y Sequence
GGKLSKKKSGGSGG

SEQ ID NO: 254 - protein, 14 aa, Artificial Sequence
  Scaffold Y Sequence
GGKLSKKSGGSGGS

SEQ ID NO: 255 - protein, 14 aa, Artificial Sequence
  Scaffold Y Sequence
GGKLSKSGGSGGSV

SEQ ID NO: 256 - protein, 14 aa, Artificial Sequence
  Scaffold Y Sequence
GAKKSKKRFSFKKS

SEQ ID NO: 257 - protein, 29 aa, Artificial Sequence
  Scaffold Y Sequence
GGKLSKKKKGYNVNDEKAKEKDKKAEGAA

SEQ ID NO: 258 - protein, 28 aa, Artificial Sequence
  Scaffold Y Sequence
GGKLSKKKKGYNVNDEKAKEKDKKAEGA

SEQ ID NO: 259 - protein, 27 aa, Artificial Sequence
  Scaffold Y Sequence
GGKLSKKKKGYNVNDEKAKEKDKKAEG

SEQ ID NO: 260 - protein, 26 aa, Artificial Sequence
  Scaffold Y Sequence
GGKLSKKKKGYNVNDEKAKEKDKKAE

SEQ ID NO: 261 - protein, 25 aa, Artificial Sequence
  Scaffold Y Sequence
GGKLSKKKKGYNVNDEKAKEKDKKA

SEQ ID NO: 262 - protein, 24 aa, Artificial Sequence
  Scaffold Y Sequence
GGKLSKKKKGYNVNDEKAKEKDKK

SEQ ID NO: 263 - protein, 23 aa, Artificial Sequence
  Scaffold Y Sequence
GGKLSKKKKGYNVNDEKAKEKDK

SEQ ID NO: 264 - protein, 22 aa, Artificial Sequence
  Scaffold Y Sequence
GGKLSKKKKGYNVNDEKAKEKD

SEQ ID NO: 265 - protein, 21 aa, Artificial Sequence
  Scaffold Y Sequence
GGKLSKKKKGYNVNDEKAKEK

SEQ ID NO: 266 - protein, 20 aa, Artificial Sequence
  Scaffold Y Sequence
GGKLSKKKKGYNVNDEKAKE

SEQ ID NO: 267 - protein, 19 aa, Artificial Sequence
  Scaffold Y Sequence
GGKLSKKKKGYNVNDEKAK

SEQ ID NO: 268 - protein, 18 aa, Artificial Sequence
  Scaffold Y Sequence
GGKLSKKKKGYNVNDEKA

SEQ ID NO: 269 - protein, 17 aa, Artificial Sequence
  Scaffold Y Sequence
GGKLSKKKKGYNVNDEK

SEQ ID NO: 270 - protein, 16 aa, Artificial Sequence
  Scaffold Y Sequence
GGKLSKKKKGYNVNDE

SEQ ID NO: 271 - protein, 15 aa, Artificial Sequence
  Scaffold Y Sequence
GGKLSKKKKGYNVND

SEQ ID NO: 272 - protein, 13 aa, Artificial Sequence
  Scaffold Y Sequence
GGKLSKKKKGYNV

SEQ ID NO: 273 - protein, 12 aa, Artificial Sequence
  Scaffold Y Sequence
GGKLSKKKKGYN

SEQ ID NO: 274 - protein, 11 aa, Artificial Sequence
  Scaffold Y Sequence
GGKLSKKKKGY

SEQ ID NO: 275 - protein, 10 aa, Artificial Sequence
  Scaffold Y Sequence
GGKLSKKKKG

SEQ ID NO: 276 - protein, 9 aa, Artificial Sequence
  Scaffold Y Sequence
GGKLSKKKK

SEQ ID NO: 277 - protein, 28 aa, Artificial Sequence
  Scaffold Y Sequence
GAKKSKKRFSFKKSFKLSGFSFKKNKKE

SEQ ID NO: 278 - protein, 27 aa, Artificial Sequence
  Scaffold Y Sequence
GAKKSKKRFSFKKSFKLSGFSFKKNKK

SEQ ID NO: 279 - protein, 26 aa, Artificial Sequence
  Scaffold Y Sequence
GAKKSKKRFSFKKSFKLSGFSFKKNK

SEQ ID NO: 280 - protein, 25 aa, Artificial Sequence
  Scaffold Y Sequence
GAKKSKKRFSFKKSFKLSGFSFKKN

SEQ ID NO: 281 - protein, 24 aa, Artificial Sequence
  Scaffold Y Sequence
GAKKSKKRFSFKKSFKLSGFSFKK

SEQ ID NO: 282 - protein, 23 aa, Artificial Sequence
  Scaffold Y Sequence
GAKKSKKRFSFKKSFKLSGFSFK

SEQ ID NO: 283 - protein, 22 aa, Artificial Sequence
  Scaffold Y Sequence
GAKKSKKRFSFKKSFKLSGFSF

SEQ ID NO: 284 - protein, 21 aa, Artificial Sequence
  Scaffold Y Sequence
GAKKSKKRFSFKKSFKLSGFS

SEQ ID NO: 285 - protein, 20 aa, Artificial Sequence
  Scaffold Y Sequence
GAKKSKKRFSFKKSFKLSGF

SEQ ID NO: 286 - protein, 19 aa, Artificial Sequence
  Scaffold Y Sequence
GAKKSKKRFSFKKSFKLSG

SEQ ID NO: 287 - protein, 18 aa, Artificial Sequence
  Scaffold Y Sequence
GAKKSKKRFSFKKSFKLS

SEQ ID NO: 288 - protein, 17 aa, Artificial Sequence
  Scaffold Y Sequence
GAKKSKKRFSFKKSFKL

SEQ ID NO: 289 - protein, 16 aa, Artificial Sequence
  Scaffold Y Sequence
GAKKSKKRFSFKKSFK

SEQ ID NO: 290 - protein, 15 aa, Artificial Sequence
  Scaffold Y Sequence
GAKKSKKRFSFKKSF

SEQ ID NO: 291 - skipped

SEQ ID NO: 292 - protein, 13 aa, Artificial Sequence
  Scaffold Y Sequence
GAKKSKKRFSFKK

SEQ ID NO: 293 - protein, 12 aa, Artificial Sequence
  Scaffold Y Sequence
GAKKSKKRFSFK

SEQ ID NO: 294 - protein, 11 aa, Artificial Sequence
  Scaffold Y Sequence
GAKKSKKRFSF

SEQ ID NO: 295 - protein, 10 aa, Artificial Sequence
  Scaffold Y Sequence
GAKKSKKRFS

SEQ ID NO: 296 - protein, 9 aa, Artificial Sequence
  Scaffold Y Sequence
GAKKSKKRF

SEQ ID NO: 297 - protein, 8 aa, Artificial Sequence
  Scaffold Y Sequence
GAKKSKKR

SEQ ID NO: 298 - protein, 7 aa, Artificial Sequence
  Scaffold Y Sequence
GAKKSKK

SEQ ID NO: 299 - protein, 29 aa, Artificial Sequence
  Scaffold Y Sequence
GAKKSKKRFSFKKSFKLSGFSFKKNKKEA

SEQ ID NO: 300 - protein, 29 aa, Artificial Sequence
  Scaffold Y Sequence
GAKKAKKRFSFKKSFKLSGFSFKKNKKEA

SEQ ID NO: 301 - protein, 28 aa, Artificial Sequence
  Scaffold Y Sequence
GAQESKKKKKKRFSFKKSFKLSGFSFKK

SEQ ID NO: 302 - protein, 27 aa, Artificial Sequence
  Scaffold Y Sequence
GAQESKKKKKKRFSFKKSFKLSGFSFK

SEQ ID NO: 303 - protein, 26 aa, Artificial Sequence
  Scaffold Y Sequence
GAQESKKKKKKRFSFKKSFKLSGFSF

SEQ ID NO: 304 - protein, 25 aa, Artificial Sequence
  Scaffold Y Sequence
GAQESKKKKKKRFSFKKSFKLSGFS

SEQ ID NO: 305 - protein, 24 aa, Artificial Sequence
  Scaffold Y Sequence
GAQESKKKKKKRFSFKKSFKLSGF

SEQ ID NO: 306 - protein, 23 aa, Artificial Sequence
  Scaffold Y Sequence
GAQESKKKKKKRFSFKKSFKLSG

SEQ ID NO: 307 - protein, 22 aa, Artificial Sequence
  Scaffold Y Sequence
GAQESKKKKKKRFSFKKSFKLS

SEQ ID NO: 308 - protein, 21 aa, Artificial Sequence
  Scaffold Y Sequence
GAQESKKKKKKRFSFKKSFKL

SEQ ID NO: 309 - protein, 20 aa, Artificial Sequence
  Scaffold Y Sequence
GAQESKKKKKKRFSFKKSFK

SEQ ID NO: 310 - protein, 19 aa, Artificial Sequence
  Scaffold Y Sequence
GAQESKKKKKKRFSFKKSF

SEQ ID NO: 311 - protein, 18 aa, Artificial Sequence
  Scaffold Y Sequence
GAQESKKKKKKRFSFKKS

SEQ ID NO: 312 - protein, 17 aa, Artificial Sequence
  Scaffold Y Sequence
GAQESKKKKKKRFSFKK

SEQ ID NO: 313 - protein, 16 aa, Artificial Sequence
  Scaffold Y Sequence
GAQESKKKKKKRFSFK

SEQ ID NO: 314 - protein, 15 aa, Artificial Sequence
  Scaffold Y Sequence
GAQESKKKKKKRFSF

SEQ ID NO: 315 - protein, 14 aa, Artificial Sequence
  Scaffold Y Sequence
GAQESKKKKKKRFS

SEQ ID NO: 316 - protein, 13 aa, Artificial Sequence
  Scaffold Y Sequence
GAQESKKKKKKRF

SEQ ID NO: 317 - protein, 12 aa, Artificial Sequence
  Scaffold Y Sequence
GAQESKKKKKKR

SEQ ID NO: 318 - protein, 11 aa, Artificial Sequence
  Scaffold Y Sequence
GAQESKKKKKK

SEQ ID NO: 319 - protein, 10 aa, Artificial Sequence
  Scaffold Y Sequence
GAQESKKKKK

SEQ ID NO: 320 - protein, 9 aa, Artificial Sequence
  Scaffold Y Sequence
GAQESKKKK

SEQ ID NO: 321 - protein, 8 aa, Artificial Sequence
  Scaffold Y Sequence
GAQESKKK

SEQ ID NO: 322 - protein, 7 aa, Artificial Sequence
  Scaffold Y Sequence
GAQESKK

SEQ ID NO: 323 - protein, 30 aa, Artificial Sequence
  Scaffold Y Sequence
GSQSSKKKKKKFSFKKPFKLSGLSFKRNRK

SEQ ID NO: 324 - protein, 29 aa, Artificial Sequence
  Scaffold Y Sequence
GSQSSKKKKKKFSFKKPFKLSGLSFKRNR

SEQ ID NO: 325 - protein, 28 aa, Artificial Sequence
  Scaffold Y Sequence
GSQSSKKKKKKFSFKKPFKLSGLSFKRN

SEQ ID NO: 326 - protein, 27 aa, Artificial Sequence
  Scaffold Y Sequence
GSQSSKKKKKKFSFKKPFKLSGLSFKR

SEQ ID NO: 327 - protein, 26 aa, Artificial Sequence
  Scaffold Y Sequence
GSQSSKKKKKKFSFKKPFKLSGLSFK

SEQ ID NO: 328 - protein, 25 aa, Artificial Sequence
  Scaffold Y Sequence
GSQSSKKKKKKFSFKKPFKLSGLSF

SEQ ID NO: 329 - protein, 24 aa, Artificial Sequence
  Scaffold Y Sequence
GSQSSKKKKKKFSFKKPFKLSGLS

SEQ ID NO: 330 - protein, 23 aa, Artificial Sequence
  Scaffold Y Sequence
GSQSSKKKKKKFSFKKPFKLSGL

SEQ ID NO: 331 - protein, 22 aa, Artificial Sequence
  Scaffold Y Sequence
GSQSSKKKKKKFSFKKPFKLSG

SEQ ID NO: 332 - protein, 21 aa, Artificial Sequence
  Scaffold Y Sequence
GSQSSKKKKKKFSFKKPFKLS

SEQ ID NO: 333 - protein, 20 aa, Artificial Sequence
  Scaffold Y Sequence
GSQSSKKKKKKFSFKKPFKL

SEQ ID NO: 334 - protein, 19 aa, Artificial Sequence
  Scaffold Y Sequence
GSQSSKKKKKKFSFKKPFK

SEQ ID NO: 335 - protein, 18 aa, Artificial Sequence
  Scaffold Y Sequence
GSQSSKKKKKKFSFKKPF

SEQ ID NO: 336 - protein, 17 aa, Artificial Sequence
  Scaffold Y Sequence
GSQSSKKKKKKFSFKKP

SEQ ID NO: 337 - protein, 16 aa, Artificial Sequence
  Scaffold Y Sequence
GSQSSKKKKKKFSFKK

SEQ ID NO: 338 - protein, 15 aa, Artificial Sequence
  Scaffold Y Sequence
GSQSSKKKKKKFSFK

SEQ ID NO: 339 - protein, 14 aa, Artificial Sequence
  Scaffold Y Sequence
GSQSSKKKKKKFSF

SEQ ID NO: 340 - protein, 13 aa, Artificial Sequence
  Scaffold Y Sequence
GSQSSKKKKKKFS

SEQ ID NO: 341 - protein, 12 aa, Artificial Sequence
  Scaffold Y Sequence
GSQSSKKKKKKF

SEQ ID NO: 342 - protein, 11 aa, Artificial Sequence
  Scaffold Y Sequence
GSQSSKKKKKK

SEQ ID NO: 343 - protein, 10 aa, Artificial Sequence
  Scaffold Y Sequence
GSQSSKKKKK

SEQ ID NO: 344 - protein, 9 aa, Artificial Sequence
  Scaffold Y Sequence
GSQSSKKKK

SEQ ID NO: 345 - protein, 8 aa, Artificial Sequence
  Scaffold Y Sequence
GSQSSKKK

SEQ ID NO: 346 - protein, 7 aa, Artificial Sequence
  Scaffold Y Sequence
GSQSSKK

SEQ ID NO: 347 - protein, 28 aa, Artificial Sequence
  Scaffold Y Sequence
GAKKAKKRFSFKKSFKLSGFSFKKNKKE

SEQ ID NO: 348 - protein, 27 aa, Artificial Sequence
  Scaffold Y Sequence
GAKKAKKRFSFKKSFKLSGFSFKKNKK

SEQ ID NO: 349 - protein, 26 aa, Artificial Sequence
  Scaffold Y Sequence
GAKKAKKRFSFKKSFKLSGFSFKKNK

SEQ ID NO: 350 - protein, 25 aa, Artificial Sequence
  Scaffold Y Sequence
GAKKAKKRFSFKKSFKLSGFSFKKN

SEQ ID NO: 351 - protein, 24 aa, Artificial Sequence
  Scaffold Y Sequence
GAKKAKKRFSFKKSFKLSGFSFKK

SEQ ID NO: 352 - protein, 23 aa, Artificial Sequence
  Scaffold Y Sequence
GAKKAKKRFSFKKSFKLSGFSFK

SEQ ID NO: 353 - protein, 22 aa, Artificial Sequence
  Scaffold Y Sequence
GAKKAKKRFSFKKSFKLSGFSF

SEQ ID NO: 354 - protein, 21 aa, Artificial Sequence
  Scaffold Y Sequence
GAKKAKKRFSFKKSFKLSGFS

SEQ ID NO: 355 - protein, 20 aa, Artificial Sequence
  Scaffold Y Sequence
GAKKAKKRFSFKKSFKLSGF

SEQ ID NO: 356 - protein, 19 aa, Artificial Sequence
  Scaffold Y Sequence
GAKKAKKRFSFKKSFKLSG

SEQ ID NO: 357 - protein, 18 aa, Artificial Sequence
  Scaffold Y Sequence
GAKKAKKRFSFKKSFKLS

SEQ ID NO: 358 - protein, 17 aa, Artificial Sequence
  Scaffold Y Sequence
GAKKAKKRFSFKKSFKL

SEQ ID NO: 359 - protein, 16 aa, Artificial Sequence
  Scaffold Y Sequence
GAKKAKKRFSFKKSFK

SEQ ID NO: 360 - protein, 15 aa, Artificial Sequence
  Scaffold Y Sequence
GAKKAKKRFSFKKSF

SEQ ID NO: 361 - protein, 14 aa, Artificial Sequence
  Scaffold Y Sequence
GAKKAKKRFSFKKS

SEQ ID NO: 362 - protein, 13 aa, Artificial Sequence
  Scaffold Y Sequence
GAKKAKKRFSFKK

SEQ ID NO: 363 - protein, 12 aa, Artificial Sequence
  Scaffold Y Sequence
GAKKAKKRFSFK

SEQ ID NO: 364 - protein, 11 aa, Artificial Sequence
  Scaffold Y Sequence
GAKKAKKRFSF

SEQ ID NO: 365 - protein, 10 aa, Artificial Sequence
  Scaffold Y Sequence
GAKKAKKRFS

SEQ ID NO: 366 - protein, 9 aa, Artificial Sequence
  Scaffold Y Sequence
GAKKAKKRF

SEQ ID NO: 367 - protein, 8 aa, Artificial Sequence
  Scaffold Y Sequence
GAKKAKKR

SEQ ID NO: 368 - protein, 7 aa, Artificial Sequence
  Scaffold Y Sequence
GAKKAKK

SEQ ID NO: 369 - protein, 94 aa, Artificial Sequence
  ESAT6 Protein Sequence
MTEQQWNFAGIEAAASAIQGNVTSIHSLDEGKQSLTKLAAAWGGSGSEAYQGVQQKWDATATELNNALQNLARTISEAGQAMASTEGNVTGMFA

SEQ ID NO: 370 - protein, 96 aa, Artificial Sequence
  TB10.4 Protein Sequence
MSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 371 - protein, 8 aa, Artificial Sequence
  Scaffold Y Sequence
  misc_feature (2)..(2): Xaa can be any naturally occurring amino acid
GXKLSKKK

SEQ ID NO: 372 - skipped

SEQ ID NO: 373 - protein, 5 aa, Artificial Sequence
  Linker
  misc_feature (5)..(5): wherein Xaa is any integer between 1-100
GGGGX

SEQ ID NO: 374 - protein, 4 aa, Artificial Sequence
  Linker
  misc_Feature (2)..(2): Wherein Xaa is any integer from 1 to 100
  misc_Feature (4)..(4): Wherein Xaa is any integer from 1 to 100
GXSX

SEQ ID NO: 375 - protein, 5 aa, Artificial Sequence
  Linker
  misc_feature (2)..(2): Wherein Xaa is any integer from 1 to 4
  misc_feature (4)..(4): Wherein Xaa  is 0 or 1
  misc_feature (5)..(5): Wherein Xaa is any integer from 1 to 50
GXSXX

SEQ ID NO: 376 - protein, 2 aa, Artificial Sequence
  Linker
  misc_feature (2)..(2): wherein Xaa can be any integer between 1-100
GN

SEQ ID NO: 377 - protein, 3 aa, Artificial Sequence
  wherein Xaa is an integer between 1 and 100
  misc_feature (3)..(3): Xaa can be any naturally occurring amino acid
GAX

SEQ ID NO: 378 - protein, 4 aa, Artificial Sequence
  Linker
  misc_feature (4)..(4): Wherein Xaa is an integer between 1 and 100
GGSX

SEQ ID NO: 379 - protein, 42 aa, Artificial Sequence [sequence omitted]

SEQ ID NO: 380 - protein, 6 aa, Artificial Sequence
  human amyloid-B protein fragment
AEFRHD

SEQ ID NO: 381 - protein, 2 aa, Artificial Sequence
  PolyGA
  misc_feature (1)..(2): Wherein the sequence is repeated between integers 10 to 20 times
GA

SEQ ID NO: 382 - protein, 13 aa, Artificial Sequence
  PADRE peptide
AKFVAAWTLKAAA

SEQ ID NO: 383 - protein, 14 aa, Artificial Sequence
  Tetanus fragment
QYIKANSKFIGITE

SEQ ID NO: 384 - protein, 15 aa, Artificial Sequence
  Diphtheria toxin fragment
QSIALSSLMVAQAIP

SEQ ID NO: 385 - protein, 119 aa, artificial sequence
  brain derived neurotrophic factor protein
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 386 - protein, 120 aa, artificial sequence
  nerve growth factor protein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 387 - protein, 119 aa, artificial sequence [sequence omitted]

SEQ ID NO: 388 - protein, 130 aa, artificial sequence
  neurotrophin-4 protein
GVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 389 - protein, 29 aa, artificial sequence
  RVG peptide
YTIWMPENPRPGTPCDIFTNSRGKRASNG

SEQ ID NO: 390 - protein, 14 aa, artificial sequence
  cyclized TAxI peptide
SACQSQSQMRCGGG

SEQ ID NO: 391 - protein, 7 aa, artificial sequence
  TAxI transport peptide #1
QSQSQMR

SEQ ID NO: 392 - protein, 7 aa, artificial sequence
  TAxI transport peptide #2
ASGAQAR

SEQ ID NO: 393 - protein, 14 aa, artificial sequence
  TAxI transport peptide #3
TSTAPHLRLRLTSR

SEQ ID NO: 394 - protein, 7 aa, artificial sequence
  nuclear localizing signal #1
PPKKRKV

SEQ ID NO: 395 - protein, 6 aa, artificial sequence
  nuclear localizing signal #2
PKKRKV

SEQ ID NO: 396 - protein, 19 aa, artificial sequence
  Angiopep-2
TFFYGGSRGKRNNFKTEEY

SEQ ID NO: 397 - protein, 39 aa, artificial sequence
  ApoB (3371 3409)
SSVIDALQYKLEGTTRLTRKRGLKLATALSLSNKFVEGS

SEQ ID NO: 398 - protein, 18 aa, artificial sequence
  ApoE (159 167)2
LRKLRKRLLLRKLRKRLL

SEQ ID NO: 399 - protein, 8 aa, artificial sequence
  Peptide-22
CMPRLRGC

SEQ ID NO: 400 - protein, 12 aa, artificial sequence
  THR
THRPPMWSPVWP

SEQ ID NO: 401 - protein, 12 aa, artificial sequence
  THR retro-enantio
PWVPSWMPPRHT

SEQ ID NO: 402 - protein, 9 aa, artificial sequence
  CRT
CRTIGPSVC

SEQ ID NO: 403 - protein, 30 aa, artificial sequence
  Leptin30
YQQILTSMPSRNVIQISNDLENLRDLLHVL

SEQ ID NO: 404 - protein, 29 aa, artificial sequence
  RVG29
YTIWMPENPRPGTPCDIFTNSRGKRASNG

SEQ ID NO: 405 - protein, 16 aa, artificial sequence
  CDX
GREIRTGRAERWSEKF

SEQ ID NO: 406 - protein, 18 aa, artificial sequence
  Apamin
CNCKAPETALCARRCQQH

SEQ ID NO: 407 - protein, 8 aa, artificial sequence
  MiniAp-4
KAPETALD

SEQ ID NO: 408 - protein, 2 aa, artificial sequence
  GSH
CG

SEQ ID NO: 409 - protein, 13 aa, artificial sequence
  G23
HLNILSTLWKYRC

SEQ ID NO: 410 - protein, 6 aa, artificial sequence
  g7
GFGFLS

SEQ ID NO: 411 - protein, 12 aa, artificial sequence
  TGN
TGNYKALHPHNG

SEQ ID NO: 412 - protein, 11 aa, artificial sequence
  TAT (47 57)
YGRKKRRQRRR

SEQ ID NO: 413 - protein, 18 aa, artificial sequence
  SynB1
RGGRLSYSRRRFSTSTGR

SEQ ID NO: 414 - protein, 21 aa, artificial sequence
  Diketopiperazines
GNYTCEVTELTREGETIIELK

SEQ ID NO: 415 - protein, 731 aa, artificial sequence; identical to SEQ ID NO: 2
  PhPro

SEQ ID NO: 416 - protein, 29 aa, artificial sequence
  Linker
GGSSGSGSGSGGGGSGGGGTGTSSSGTGT